(19) United States
(12) Patent Application Publication
(10) Pub. No.: US 2023/0123654 A1
(43) Pub. Date: Apr. 20, 2023

(54) COMPOSITIONS AND THERAPEUTIC USES OF CANNABIDIOL

(71) Applicants: Shreema MERCHANT, Surrey (CA); Manit PATEL, Surrey (CA)

(72) Inventors: Manit PATEL, Surrey (CA); Peter Charles RUBEN, Anmore (CA); Mohamed Amin FOUDA, Vancouver (CA); Mohammad-Reza GHOVANLOO, West Vancouver (CA); Vishal Anant JADHAV, Mumbai (IN); Dana A PAGE, Burnaby (CA); Koushik CHOUDHURY, Stockholm (SE); Rusinova RADDA, New York, NY (US); Tejas PHATERPEKAR, Burnaby (CA)

(73) Assignees: Shreema MERCHANT, Surrey, BC (CA); Manit PATEL, Surrey, BC (CA)

(21) Appl. No.: 17/800,563

(22) PCT Filed: Feb. 19, 2021
(86) PCT No.: PCT/IN2021/050159

§ 371 Date: Aug. 18, 2022

(30) Foreign Application Priority Data
Feb. 19, 2020 (IN) 202021007184
Mar. 29, 2020 (IN) 202021013770

(51) Int. Cl. A61K 31/05; A61K 31/7052; A61K 45/06

(52) U.S. Cl. CPC A61K 31/05; A61K 31/7052; A61K 45/06

(57) ABSTRACT

The invention provides various pharmaceutical composition comprising the new therapeutic agent cannabidiol that rescues the adversely affected sodium channels Nav1.5 and thus serves as a potential therapeutic agent for treating several cardiac disorders. The invention also provides various pharmaceutical composition employing the new therapeutic agent cannabidiol for abolishing or minimizing side effects of other therapeutic agents/drugs which induce, or which are likely to induce Long QT. The invention further provides pharmaceutical composition of cannabidiol for treating or avoiding inflammation induced by any other therapeutic agent or inflammation induced in any diseases or ailment such as Covid-19 and also inflammation induced by any vaccine such as Covid-19 vaccine.

### BACKGROUND OF THE ART

Cardiovascular complications are the main cause of mortality and morbidity in diabetic populations. High glucose levels (Hyperglycemia) is considered to be the cornerstone in the development of diabetes-evoked cardiovascular complications. The main mechanisms underlying these deleterious effects include oxidative stress, activation of pro-inflammatory, and inactivation of pro-survival pathways such as Akt, which eventually culminates in cell death.

Cardiovascular anomalies are strongly correlated with diabetes-induced morbidity and mortality (Matheus, Tannus, Cobas, Palma, Negrato & Gomes, 2013). These deleterious cardiovascular complications are mainly attributed to hyperglycemia/high glucose (Pistrosch, Natali & Hanefeld, 2011).

Table 1 provided by Grisanti summarizes the clinical studies examining the correlation between diabetes and arrhythmias and the study provides that there is a clear link between diabetes and cardiac arrhythmias. Grisanti has mentioned a few previous studies that have provided that Type 1 and type 2 diabetic patients have been identified as having slowed conduction velocity and an increased prevalence of prolonged QT interval. Napolitano et al have provided that Long QT syndrome (LQT) is a cardiac arrhythmogenic disorder, identified by a prolongation of the QT interval.

Voltage-gated sodium (Na+) channels have three main conformational states: closed, open and inactivated. Before an action potential occurs, the membrane is at its normal resting potential, and Na+ channels are in their deactivated state. In response to an increase of the membrane potential to about −55 mV, the activation gates open, allowing positively charged Na+ ions to flow into the cell through the channels, and causing the voltage across the cell membrane to increase (depolarization) to +30 mV.

At the peak of the action potential, when enough Na+ has entered the cell and the membrane potential has become high enough, the Na+ channels inactivate themselves by closing their inactivation gates. Closure of the inactivation gate prevents further ingress of the Na+ flow through the channel which in turn causes the membrane potential to stop rising. With its inactivation gate closed, the channel is said to be inactivated and the potential decreases back to its resting potential as the neuron repolarizes and subsequently hyperpolarizes itself. This decrease in voltage constitutes the falling phase of the action potential.

When the membrane voltage becomes low enough, the inactivation gate reopens, and the activation gate closes in a process called deactivation.

With the activation gate closed and the inactivation gate open, the Na+ channel is once again available and ready to contribute to another action potential.

Previous publications (Ghovanloo et al (2016), Estacion et al (2010), Cannon et al (2006)) have reported that Nav are hetero-multimeric proteins composed of a large ion conducting and voltage-sensing α-subunit and smaller β-subunits.

As reported by Ghovanloo (2016), the α-subunit is made up of a single transcript that includes four 6-transmembrane segment domains. Each structural domain can be divided into two functional sub-domains: the voltage-sensing domain (VSD) and the pore-domain (PD).

The Nav pore is the site of interaction for many pharmacological blockers (Lee (2012) and Gamal (2018)). The pore is surrounded by four intralipid fenestrations whose functional roles remain speculative (Pan (2018)).

Alterations in the biophysical properties of Nav1.5 play an important role in cardiac arrhythmogenesis (Ruan, Liu & Priori, 2009). However, the diabetes/high glucose/hyperglycaemia-induced changes in the biophysical properties of Nav1.5 are not well understood.

Thus, gating of Nav 1.5 is a complex phenomenon and it is adversely affected in hyperglycaemia and diabetes. Modulation of the same is not an easy task.

Yu et al and others have not provided, tested, treated or even suggested treatment of arrhythmia particularly in diabetic condition.

Christopher Ahern in his commentary What activates inactivation? mentions that inherited or acquired defects in sodium channel conductance are associated with a spectrum of electrical signalling disorders including cardiac arrhythmias (Wang et al., 1995; Valdivia et al., 2005), epilepsy, primary erythromelalgia (a peripheral pain disorder) (Yang et al., 2004), paroxysmal extreme pain disorder (Fertleman et al., 2006), hypokalemic periodic paralysis (Ptácek et al., 1991; Rojas et al., 1991), paramyotonia congenita (McClatchey et al., 1992), in addition to unexpected roles in migraine (Kahlig et al., 2008), autism (Weiss et al., 2003; Han et al., 2012a), sleep (Han et al., 2012b), and multiple sclerosis (Craner et al., 2004).

Although much is known about the pathogenesis of these diseases, few treatment options are available and much work is required to alleviate the conditions associated with voltage-gated sodium channels, particularly in Nav1.5 where the consequences of dysfunction are potentially fatal.

Shimizu et al have provided that LQT3 is caused by a gain-of-function in cardiac sodium channels that increases the depolarizing current throughout the action potential plateau. Yu et al have shown that cardiac sodium channels are associated with the pathogenesis of LQT in diabetic rats. Further they have shown that changes in Nav1.5 (sodium channel in cardiac muscle) function is correlated with LQT arrhythmia in diabetic rats.

From the studies of Yu et al, it is seen that Nav1.5 gating defects contribute to the development of arrhythmia in diabetic rats. Yu et al have selected Streptozotocin or streptozocin (INN, USP) (STZ) which is a naturally occurring alkylating antineoplastic agent and used in medical research to produce an animal model for hyperglycemia and Alzheimer's in a large dose, as well as type 2 diabetes or type 1 diabetes with multiple low doses.

Oxidative stress and activation of pro-inflammatory pathways are among the main pathways involved in diabetes/high glucose evoked cardiovascular abnormalities (Rajesh et al., 2010). Cardiac inflammation has a key role in the development of cardiovascular anomalies (Adamo, Rocha-Resende, Prabhu & Mann, 2020). Inhibition of inflammatory signalling pathways ameliorate cardiac consequences (Adamo, Rocha-Resende, Prabhu & Mann, 2020). Importantly, ion channels are crucial players in inflammation-induced cardiac abnormalities (Eisenhut & Wallace, 2011). Voltage-gated sodium channels (Nav) underlie phase 0 of the cardiac action potential (Balser, 1999; Ruan, Liu & Priori, 2009). Changes in the biophysical properties of the primary cardiac sodium channel, Nav1.5, are linked to diabetes induced cardiovascular abnormalities (Fouda, Ghovanloo & Ruben, 2020; Yu et al., 2018). However, the mechanisms underlying hyperglycemia-induced inflammation, and how inflammation provokes cardiac dysfunction, are not well understood.

Previous research has indicated the following:
1. Diabetes-induced QT prolongation predisposes to malignant ventricular arrhythmias (Ukpabi & Onwubere, 2017).
2. Moreover, LQT in diabetic patients make them three times more vulnerable to the risk of cardiac arrest (Whitsel et al., 2005).
3. Nav1.5 gain-of-function plays a crucial role in the development of LQT (Shimizu & Antzelevitch, 1999).
4. Diabetes-induced QT prolongation predisposes to malignant ventricular arrhythmias (Ukpabi & Onwubere, 2017).
5. LQT in diabetic patients make them three times more vulnerable to the risk of cardiac arrest (Whitsel et al., 2005).
6. Nav1.5 gain-of-function plays a crucial role in the development of LQT (Shimizu & Antzelevitch, 1999).
7. Hyperglycemia/high glucose is proinflammatory and that inflammation is a crucial player in the pathogenesis of cardiovascular anamolies (Fouda, Leffler & Abdel-Rahman, 2020; Tsalamandris et al., 2019).
8. Inflammation is a potential cause for developing LQT through direct effects on myocardial electric properties, including its effect on Nav, and indirect autonomic cardiac regulations (Lazzerini, Capecchi & Laghi-Pasini, 2015).
9. Inflammation alters the electrophysiological properties of cardiomyocytes Nav with an increase in INap leading to prolongation of APD (Shryock, Song, Rajamani, Antzelevitch & Belardinelli, 2013; Ward, Bazzazi, Clark, Nygren & Giles, 2006).
10. The activation of PK-A and PK-C and subsequent protein phosphorylation is among the key signalling pathways associated with inflammation (Karin, 2005) and hyperglycemia, resulting in many devastating diabetes-induced cardiac complications (Bockus & Humphries, 2015; Koya & King, 1998)
11. PK-A phosphorlylates S525 and S528, while PK-C phosphorylates S1503 in human Nav1.5 (Iqbal & Lemmens-Gruber, 2019).
12. There are conflicting reports regarding the effects of PK-A and PK-C activation on the voltage-dependence and kinetics of Nav1.5 gating. These differences could be attributed to different voltage protocols, different holding potentials, different concentrations or type of PK-activators, or different cell lines used in the various studies (Aromolaran, Chahine & Boutjdir, 2018; Iqbal & Lemmens-Gruber, 2019).
13. Both PK-A or PK-C destabilize Nav fast inactivation and hence increase INap, which is strongly correlated to prolonged APD (Astman, Gutnick & Fleidervish, 1998; Franceschetti, Taverna, Sancini, Panzica, Lombardi & Avanzini, 2000; Tateyama, Rivolta, Clancy & Kass, 2003).
14. CANNABIDIOL exhibits anti-inflammatory, anti-oxidant, and anti-tumor effects via inhibition of PK-A and PK-C signalling (Seltzer, Watters & MacKenzie, 2020).
15. Estradiol (E₂) directly affects Nav and exerts anti-inflammatory effects (lorga, Cunningham, Moazeni, Ruffenach, Umar & Eghbali, 2017; Wang, Garro & Kuehl-Kovarik, 2010).
16. Cardioprotective effects of Estradiol (E₂) by increasing angiogenesis, vasodilation, and decreasing oxidative stress and fibrosis (lorga, Cunningham, Moazeni, Ruffenach, Umar & Eghbali, 2017).
17. Many studies support the anti-arrythmic effects of Estradiol (E₂) because of its effects on the expression and function of cardiac ion channels (lorga, Cunningham, Moazeni, Ruffenach, Umar & Eghbali, 2017; Odening & Koren, 2014).
18. Estradiol (E₂) stabilizes Nav fast inactivation and reduces INap, similar to CANNABIDIOL (Wang, Garro & Kuehl-Kovarik, 2010).
19. Estradiol (E₂) reduces the oxidative stress and the inflammatory responses by inhibiting PK-A and PK-C-mediated signalling pathways (Mize, Shapiro & Dorsa, 2003; Viviani, Corsini, Binaglia, Lucchi, Galli & Marinovich, 2002).
20. LQT3 arrhythmia is a clinical complication of diabetes (Grisanti, 2018).

CANNABIDIOL is the main cannabinoid constituent of Cannabis sativa plant. It binds very weakly to CB1 and CB2 receptors. CANNABIDIOL does not induce psychoactive or cognitive effects and is well tolerated without side effects in humans, thus making it a putative therapeutic target. In the United States, the CANNABIDIOL drug Epidiolex was approved by the Food and Drug Administration in 2018 for the treatment of two epilepsy disorders: Dravet Syndrome and Lennox/Gasteaut Syndrome.

CANNABIDIOL is designated chemically as 2-[(1R,6R)-3-Methyl-6-(1-methylethenyl)-2-cyclohexen-1-yl]-5-pentyl-1,3-benzenediol. The chemical structure is as follows.

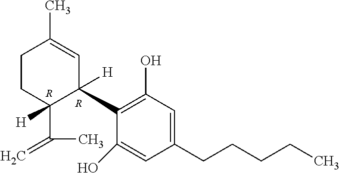

The U.S. Pat. No. 6,410,588 discloses the use of CANNABIDIOL to treat inflammatory diseases.

The PCT publication no. WO2001095899A2 relates to CANNABIDIOL derivatives and to pharmaceutical compositions comprising CANNABIDIOL derivatives being anti-inflammatory agents having analgesic, antianxiety, anticonvulsive, neuroprotective, antipsychotic and anticancer activity.

CANNABIDIOL is approved as an anti-seizure drug (Barnes, 2006; Devinsky et al., 2017). CANNABIDIOL lacks adverse cardiac toxicity and ameliorates diabetes/high glucose induced deleterious cardiomyopathy (Cunha et al., 1980; Izzo, Borrelli, Capasso, Di Marzo & Mechoulam, 2009; Rajesh et al., 2010). Rajesh et al is silent on the effects of CANNABIDIOL if any in arrhythmias and does not suggest the effect of CANNABIDIOL on inherited or acquired Long QT intervals.

In addition, CANNABIDIOL inhibits the production of pro-inflammatory cytokines in vitro and in vivo (Nichols & Kaplan, 2020).

### SUMMARY OF THE INVENTION

In the first aspect, the invention provides various pharmaceutical composition comprising the new therapeutic agent cannabidiol that rescues the adversely affected sodium channels Nav1.5 and thus serves as a potential therapeutic agent for treating several cardiac disorders. The invention further provides uses of these pharmaceutical compositions for treating various cardiac disorders. The invention also includes treating patients suffering from various cardiac disorders by administering suitable pharmaceutical compositions comprising cannabidiol.

In the first aspect, invention provides a pharmaceutical composition comprising therapeutically effective amount of cannabidiol for use in treatment of a cardiac disorder arising from gating defects in sodium channel Nav1.5.

The various cardiac disorders arising from gating defects in sodium channel Nav1.5 wherein the gating defects includes at least one from i) less likely to activate; ii) inability to fast inactivate; iii) unstable fast inactivation; iv) late or persistent sodium current and v) prolongation of action potential.

In the second aspect, the invention provides various pharmaceutical compositions employing the new therapeutic agent cannabidiol for treating various cardiac disorders induced by hyperglycemia or diabetic conditions. The invention also includes treating patients suffering from various cardiac disorders induced by hyperglycemia or diabetic conditions by administering suitable pharmaceutical compositions employing cannabidiol.

In the third aspect, the invention provides various pharmaceutical compositions employing the new therapeutic agent cannabidiol for avoiding or minimizing occurrence of cardiac disorders in a hyperglycaemic or diabetic population more prone to such disorders. The invention further provides uses of these pharmaceutical compositions for avoiding or minimizing cardiac disorders in a hypoglycemic or diabetic population and treating by administering pharmaceutical compositions employing new therapeutic agent cannabidiol to achieve the same.

The pharmaceutical composition of cannabidiol of the present invention are used in treatment of a cardiac disorder selected from one or more of long QT syndrome, long QTc syndrome, long QRS syndrome, cardiomyopathy, heart failure, arrhythmia, ischemia, Hypertrophic cardiomyopathy and Hypoxia myocardial ischemia, myocardial infarction (MI), arrhythmias of ischemic and non-ischemic origin, inflammation, vascular dysfunction, cardiomyopathy, cardiac remodeling, maladaptation, anginas of different types, drug induced heart failure, iatrogenic heart and vascular diseases.

In the fourth aspect, the invention provides various pharmaceutical compositions employing the new therapeutic agent cannabidiol for abolishing or minimizing side effects of other therapeutic agents/drugs which induce, or which are likely to induce Long QT. In this aspect, cannabidiol pharmaceutical composition enhances the safety profile of other therapeutic agents as well as enhance their application which were limited due to their side effects mainly Long QT interval.

The other therapeutic agent drugs which induce, or which are likely to induce Long QT are selected from opioid, azithromycin, chloroquine, hydroxychloroquine and antiviral. The antiviral is selected from oseltamivir phosphate, atazanavir sulphate and ribavirin.

In the fourth aspect, the pharmaceutical composition of new therapeutic agent cannabidiol are administered along with Covid-19 vaccine or any vaccine which is likely to induce LQT arrythmias. Accordingly, in this aspect invention provides a pharmaceutical composition comprising therapeutically effective amount of cannabidiol for use in avoiding or minimizing occurrence of a cardiac disorder arising from gating defects in sodium channel Nav1.5 wherein the gating defect includes at least one from i) less likely to activate; ii) inability to fast inactivate; iii) unstable fast inactivation; iv) late or persistent sodium current and v) prolongation of action potential; and wherein the gating defect is likely to be induced by administration of i) at least one other therapeutic agent or ii) Covid-19 vaccine.

In fifth aspect, the invention provides cannabidiol pharmaceutical compositions for Covid-19 treatment in two circumstances below:

1. where Covid-19 has
induced Long QT in patient or where Covid-19 is likely to induce Long QT in patients suffering from other comorbidities; and
2. where Covid-19
treatment uses any therapeutic agent or likely to use any therapeutic agent where such agent has induced or is likely to induce Long QT in patients.

The invention further provides pharmaceutical compositions of cannabidiol for uses in Covid-19 treatment where Long QT has been induced or is likely to be induced either due to Covid-19 or due to treatment of Covid 19 with any therapeutic agent likely to cause LQT and treating Covid-19 patients by administering pharmaceutical compositions employing new therapeutic agent cannabidiol alone or along with such other therapeutic agent likely to cause or has caused Long QT. Without limitations, these other therapeutic agents include antivirals, chloroquine, hydroxychloroquine and even nutraceuticals such as vitamins. These other therapeutic agents may also encompass but are not restricted to natural—organic or in-organic, ayurvedic, homeopathic, siddha and unani medicines.

In the sixth aspect, pharmaceutical compositions of cannabidiol are administered even to healthy population as a prophylactic therapeutic agent to avoid occurrence of any cardiac disorder where sodium channel gating properties are affected. Such administration to a healthy population is also done when there is likelihood of Covid-19 such as during epidemic or pandemic of Covid-19.

Additionally, under this aspect, cannabidiol pharmaceutical compositions are administered even to healthy population when there is likelihood of any epidemic or pandemic which is likely to induce Long QT.

Accordingly, in this aspect invention provides a pharmaceutical composition comprising therapeutically effective amount of cannabidiol for use in prophylaxis or prophylactic treatment for avoiding or minimizing occurrence of a cardiac disorder arising from gating defects in sodium channel Nav1.5.

In the seventh aspect, the invention provides various pharmaceutical compositions employing the new therapeutic agent cannabidiol to rescues the adversely affected sodium channels Nav1.5 from the effects of formation of reactive oxygen species and conditions produced further from these effects. Reactive oxygen species formation causes oxidative damage and leads to cytotoxicity. As a result, cell viability is reduced.

The invention further provides uses of these pharmaceutical compositions i) for reducing ROS formation and ii) for treating conditions produced due to formation of reactive oxygen species. The invention also includes treating patients suffering from i) effects ROS formation on Sodium channels Nav 1.5 and ii) conditions produced further from these effects by administering suitable pharmaceutical compositions employing cannabidiol.

Further under the eighth aspect, the invention provides pharmaceutical compositions of cannabidiol for treating or avoiding inflammation induced by any other therapeutic agent or inflammation induced in any diseases or ailment such as Covid-19 and also inflammation induced by any vaccine such as Covid-19 vaccine.

In the ninth aspect, the invention provides various pharmaceutical compositions employing the new therapeutic agent cannabidiol to rescues the adversely affected sodium channels Nav1.4 from the contractility dysfunction and conditions produced further from these effects such as muscle stiffness, pain, myotonia, gating-pore current in the VSD leading to periodic paralyses etc.

In the tenth aspect of the invention provides pharmaceutical compositions of the cannabidiol which is the new therapeutic agent for restoring electrophysiology of sodium channels thus avoiding, abolishing or minimizing happening of cardiac disorders which mainly happen due to late or persistent sodium channels, prolongation of action potential, Long QT arrhythmias etc.

### BRIEF DESCRIPTION OF THE DRAWINGS

FIG. 1A provides effect of gradual increasing of glucose concentration (10, 25, 50, 100, 150 mM) on the cell viability of untransfected or Nav1.5 transfected cells.

FIG. 1B provides effect of co-incubation of CANNABIDIOL (5 μM), lidocaine (1 mM) or Tempol (1 mM) or their vehicle on the cell viability of Nav1.5 transfected cells incubated in control (10 mM) or high glucose concentrations (50 or 100 mM).

FIG. 1C provides an effect of gradual increasing of glucose concentration (10, 25, 50, 100, 150 mM) or mannitol (100 mM) on the cell viability of mock transfected or Nav1.5 stable transfected cells.

FIG. 1D provides effect of co-incubation of CANNABIDIOL (1 or 5 μM), lidocaine (100 μM or 1 mM) or Tempol (100 μM or 1 mM) or their vehicle on the cell viability of Nav1.5 transfected cells incubated in high glucose concentrations (100 mM).

FIG. 1E provides the effect of co-incubation of CANNABIDIOL (5 μM) or its vehicle on cell viability of untransfected cells incubated in normal (10 mM) or high glucose concentrations (100 mM).

FIG. 2A provides the effect of gradual increasing of glucose concentration (10, 25, 50, 100, 150 mM) on ROS production of untransfected or Nav1.5 transfected cells.

FIG. 2B provides effect of co-incubation of CANNABIDIOL (5 μM), lidocaine (1 mM) or Tempol (1 mM) or their vehicle on ROS production of Nav1.5 transfected cells incubated in normal (10 mM) or high glucose concentrations (50 or 100 mM).

FIG. 2C provides an effect of gradual increasing of glucose concentration (10, 25, 50, 100, 150 mM) or mannitol (100 mM) on ROS production of mock transfected or Nav1.5 stable transfected cells.

FIG. 2D provides effect of co-incubation of CANNABIDIOL (1 or 5 μM), lidocaine (100 μM or 1 mM) or Tempol (100 μM or 1 mM) or their vehicle on ROS production of Nav1.5 transfected cells incubated in high glucose concentration (100 mM).

FIG. 2E provides the effect of co-incubation of CANNABIDIOL (5 μM) or its vehicle on ROS production of untransfected cells incubated in normal (10 mM) or high glucose concentrations (100 mM).

FIG. 3A provides the effect of high glucose (50 or 100 mM) on conductance curve of Nav1.5 transfected cells.

FIG. 3B provides effect of CANNABIDIOL (5 μM), lidocaine (1 mM) or Tempol (1 mM, perfusion or incubation) or their vehicle on the conductance curve of Nav1.5 transfected cells incubated in control (10 mM) glucose concentration.

FIG. 3C provides effect of CANNABIDIOL (5 μM), lidocaine (1 mM) or Tempol (1 mM, perfusion or incubation) or their vehicle on the conductance curve of Nav1.5 transfected cells incubated in 50 mM glucose for 24 hours.

FIG. 3D provides effect of CANNABIDIOL (5 μM), lidocaine (1 mM) or Tempol (1 mM, perfusion or incubation) or their vehicle on the conductance curve of Nav1.5 transfected cells incubated in 100 mM glucose for 24 hours.

FIG. 3E provides the effect of high glucose (25, 50 or 100 mM) or mannitol (100 mM) on the conductance curve of Nav1.5 transfected cells.

FIG. 3F provides effect of CANNABIDIOL (1 or 5 μM), lidocaine (100 μM or 1 mM) or Tempol (1 mM, perfusion or 100 μM or 1 mM incubation) or their vehicle on the conductance curve of Nav1.5 transfected cells incubated in high (100 mM) glucose concentration for 24 hours.

FIG. 3G provides representative families of macroscopic currents across conditions.

FIG. 4A provides an effect of high glucose (50 or 100 mM) on steady-state fast inactivation.

FIG. 4B provides effect of CANNABIDIOL (5 μM), lidocaine (1 mM) or Tempol (1 mM, perfusion or incubation) or their vehicle on SSFI of Nav1.5 transfected cells incubated in control (10 mM) glucose concentration.

FIG. 4C provides effect of CANNABIDIOL (5 μM), lidocaine (1 mM) or Tempol (1 mM, perfusion or incubation) or their vehicle on the steady-state fast inactivation of Nav1.5 transfected cells incubated in 50 mM glucose for 24 hours.

FIG. 4D provides effect of CANNABIDIOL (5 μM), lidocaine (1 mM) or Tempol (1 mM, perfusion or incubation) or their vehicle on the steady-state fast inactivation of Nav1.5 transfected cells incubated in 100 mM glucose for 24 hours.

FIG. 4E provides the effect of high glucose (25, 50 or 100 mM) or mannitol (100 mM) on the steady-state fast inactivation of Nav1.5 transfected cells.

FIG. 4F provides effect of CANNABIDIOL (1 or 5 μM), lidocaine (100 μM or 1 mM) or Tempol (1 mM, perfusion or 100 μM or 1 mM incubation) or their vehicle on the steady-state fast inactivation of Nav1.5 transfected cells incubated in high (100 mM) glucose concentration for 24 hours.

FIG. 5A provides an effect of high glucose (50 or 100 mM) on recovery from fast inactivation of Nav1.5 transfected cells.

FIG. 5B provides effect of CANNABIDIOL (5 μM), lidocaine (1 mM) or Tempol (1 mM, perfusion or incubation) or their vehicle on the recovery from fast inactivation of Nav1.5 transfected cells incubated in control (10 mM) glucose concentration.

FIG. 5C provides effect of CANNABIDIOL (5 μM), lidocaine (1 mM) or Tempol (1 mM, perfusion or incubation) or their vehicle on the recovery from fast inactivation of Nav1.5 transfected cells incubated in 50 mM glucose for 24 hours.

FIG. 5D provides effect of CANNABIDIOL (5 μM), lidocaine (1 mM) or Tempol (1 mM, perfusion or incubation) or their vehicle on the recovery from fast inactivation of Nav1.5 transfected cells incubated in 100 mM glucose for 24 hours.

FIG. 5E provides an effect of high glucose (25, 50 or 100 mM) or mannitol (100 mM) on recovery from fast inactivation of Nav1.5 transfected cells.

FIG. 5F provides effect of CANNABIDIOL (1 or 5 μM), lidocaine (100 μM or 1 mM) or Tempol (1 mM, perfusion or 100 μM or 1 mM incubation) or their vehicle on the recovery from fast inactivation of Nav1.5 transfected cells incubated in 100 mM glucose for 24 hours.

FIGS. 6A and 6B provide effect of CANNABIDIOL (5 μM), lidocaine (1 mM) or Tempol (1 mM, perfusion or incubation) or their vehicle on the percentage of persistent sodium currents of Nav1.5 transfected cells incubated in control, 50- or 100-mM glucose for 24 hours. *P<0.05 versus corresponding “Control” values.

FIGS. 6C and 6D provide effect of CANNABIDIOL (1 or 5 μM), lidocaine (100 or 1 mM) or Tempol (1 mM, perfusion or 100 μM or 1 mM incubation) or their vehicle on the percentage of persistent sodium currents of Nav1.5 transfected cells incubated in 100 mM glucose for 24 hours. *P<0.05 versus corresponding “Control” values. #P<0.05 versus corresponding “glucose 100 mM counterparts”.

FIG. 7A provides action potential duration of Nav1.5 transfected cells incubated in control, 50 or 100 mM glucose for 24 hours.

FIG. 7B provides effect of CANNABIDIOL (5 μM), lidocaine (1 mM) or Tempol (1 mM, perfusion or incubation) or their vehicle on the action potential duration of Nav1.5 transfected cells incubated in 100 mM glucose for 24 hours.

FIG. 8 provides a schematic of possible cellular events involved in the protective effect of CANNABIDIOL, lidocaine or Tempol against high glucose induced oxidative effects and cytotoxicity via affecting cardiac voltage-gated sodium channels (Nav1.5).

FIG. 9A AND 9B provide images of the rat diaphragm, cut into a hemi-diaphragm.

FIG. 9C provides reduction in contraction amplitude to ˜60% of control by CANNABIDIOL (100 μM) and to ˜20% of control by TTX (300 nM) FIGS. 9D, 9E and 9F provide representative traces of muscle contraction in control, CANNABIDIOL, and TTX respectively.

FIGS. 10A and 10B provide the effects of Cannabidiol on POPC membrane area per lipid and lipid diffusion in the molecular dynamics (MD) simulations of Cannabidiol on 1-palmitoyl-2-oleoyl-sn-glycero-3-phosphocholine (POPC).

FIG. 10C provides Cannabidiol density estimates as a function of membrane leaflet coordinate, where the lipid bilayer is centered at 0. It provides distribution of Cannabidiol into the membrane across a range of conditions. The distribution of phosphate groups is shown as solid lines, the distribution of CANNABIDIOL dotted lines.

FIG. 10D provides order parameter of lipid acyl chains estimated from the MD simulations. It is provided that CANNABIDIOL causes a slight ordering of the membrane methylenes in the plateau region of the palmitoyl chain (C3-C8).

FIGS. 10E, 10F, 10G and 18A, 18B and 18C provide results of molecular dynamics suggesting that CANNABIDIOL tends to localize preferentially between the phosphate head and the bottom end of the fatty chain, close to carbons 3-7 of the aliphatic chains of the POPC molecules

FIGS. 10F, 10G and 10H along with FIGS. 18A, 18B and 18C provide NMR data with POPC-d31 and POPC-d31/CANNABIDIOL in a 4:1 ratio in deuterium depleted water at three different temperatures (20, 30, and 40° C.).

FIGS. 11A, 11B, 11C and 11D provide average gramicidin current density from the ratio of current amplitude to the cell membrane capacitance (pA/pF) at −120, −80, 0, and +50 mV.

FIGS. 11A, 11B and 11C provide effects of 1 μM (inactivated Nav IC505) and 10 μM (˜resting Nav IC505) CANNABIDIOL, and 10 μM Triton X100 (TX100) as positive control on gramicidin-HEK cells.

FIG. 11C provides that TX100 altered the cationic gramicidin currents across all potentials (p<0.05). 11C also provides that CANNABIDIOL had the opposite effect to TX100, and slightly altered gramicidin currents at both 1 μM (p<0.05) and 10 μM (p>0.05).

FIGS. 11E, 11F, 11G and 11H provide results of gramicidin (gA)-based assay performed in lowered extracellular sodium [Na+=1 mM]. This experiment resulted in the same overall trends of altered gramicidin currents densities as the high [Na+] experiment, for both CANNABIDIOL and TX100.

FIGS. 12A and 12B provide molecular docking studies using the human Nav1.4 cryo-EM structure. The figures show CANNABIDIOL docked onto the Nav1.4 pore, supporting a possible interaction at the LA site.

FIG. 12A is a side-view of CANNABIDIOL docked into the pore of the human Nav1.4 structure. The structure is coloured by domain. DIV is coloured in deep blue.

FIG. 12B is a Zoomed-in side-view, F1586 is coloured in yellow.

FIGS. 12C, 12D, 12E and 12F provide biophysical characterization of F1586A compared with WT-Nav1.4.

FIGS. 12C and 12D provide representative families of macroscopic current traces from WT-Nav1.4 and F1586A.

FIG. 12E provides voltage-dependence of activation as normalized conductance plotted against membrane potential (Nav1.4: V1/2=−19.9±2.7 mV, z=2.8±0.3; F1586A: V1/2=−22.4±2.2 mV, z=3.0±0.3; n=5-7).

FIG. 12F provides that when biophysical characterization of F1586A is compared with WT-Nav1.4, the inactivation voltage-dependencies were almost identical (p>0.05). It provides Voltage-dependence of SSFI as normalized current plotted against membrane potential (Nav1.4: V1/2=−66.9±2.8 mV, z=−2.6±0.3; F1586A: V1/2=−63.3±3.0 mV, z=−3.5±0.3; n=8-9).

FIGS. 12G and 12H provide Lidocaine/CANNABIDIOL inhibition of Nav1.4 and F1586A from −110 mV (rest) at 1 Hz (Lidocaine-Nav1.4: Mean block=60.6±2.3%; Lidocaine-F1586A: Mean block=24.6±9.3%; CANNABIDIOL Nav1.4: Mean block=42.4±6.4%; CANNABIDIOL-F1586A: Mean block=25.3±4.8%; n=3-5).

FIG. 13A-13G provide CANNABIDIOL interactions with and through Nav fenestrations.

FIG. 13A provides side-view of CANNABIDIOL docked into the human Nav1.4 structure. The structure is coloured by domain, CANNABIDIOL is represented in purple.

FIG. 13B provides side-view of all four sides of human Nav1.4 (coloured by domain). Nav1.4 fenestrations are highlighted in red, along with the position of respective residues that were mutated into tryptophans (W).

FIG. 13C provides Computational mutagenesis of fenestrations results 2 full and 2 partial occlusions (paralleled domains).

FIG. 13D provides Lidocaine (1.1 mM) inhibition of Nav1.4 and WWWW from −110 mV (rest) at 1 Hz (Nav1.4: Mean block=60.6±2.3%; WWWW: Mean block=53.6±11.7%), flecainide (350 μM) inhibition (Nav1.4: Mean block=64.6±6.0%; WWWW: Mean block=76.4±11.3%), and CANNABIDIOL (10 μM) inhibition (Nav1.4: Mean block=42.4±6.4%; WWWW: Mean block=6.4±1.3%; n=3-5 panel-wide).

FIG. 13E provides CANNABIDIOL pathway through the Nav1.5 fenestration from side view, as predicted by MD simulations, red and blue correlate to CANNABIDIOL being inside and outside the fenestration, respectively.

FIG. 13F provides CANNABIDIOL pathway from top view of the channel.

FIG. 13G provides progressive snapshots of the movement of CANNABIDIOL over time from inside to outside the channel.

FIG. 14A-14D provide effects of CANNABIDIOL (1 μM) on Nav1.4 gating.

FIGS. 14A and 14B provide voltage-dependence of activation as normalized conductance plotted against membrane potential (Control: V1/2=−19.9±4.2 mV, z=2.8±0.3; CANNABIDIOL (CANNABIDIOL): V1/2=−14.3±4.2 mV, z=2.8±0.3; n=5) and normalized activating currents as a function of potential.

FIG. 14C provides voltage-dependence of SSFI plotted against membrane potential (Control: V1/2=−64.1±2.4 mV, z=−2.7±0.3; CANNABIDIOL (CANNABIDIOL): V1/2=−72.7±3.0 mV, z=−2.8±0.4; n=5-8).

FIG. 14D provides recovery from fast inactivation at: 500 ms (Control: τFast=0.0025±0.00069 s, τSlow=0.224±0.046 s; CANNABIDIOL (CANNABIDIOL): τFast=0.0048±0.00081 s; τSlow=0.677±0.054 s; n=5-7).

FIGS. 15A-15H provide effects of CANNABIDIOL (1 μM) on gating of a myotonia/hypoPP variant, P1158S.

FIGS. 15A and 15B provide voltage-dependence of activation as normalized conductance plotted against membrane potential, at pH7.4 (Control: V1/2=−30.0±3.3 mV, z=3.1±0.2; CANNABIDIOL: V1/2=−32.7±3.6 mV, z=2.9±0.2; n=7-8) and pH6.4 (Control: V1/2=−23.0±3.3 mV, z=2.9±0.2; CANNABIDIOL: V1/2=−21.1±3.3 mV, z=2.5±0.2; n=8).

FIGS. 15C and 15D provide Voltage-dependence of SSFI plotted against membrane potential at pH7.4 (Control: V1/2=−73.2±2.6 mV, z=2.9±0.2; CANNABIDIOL: V1/2=−83.0±2.6 mV, z=3.0±0.3; n=7) and pH6.4 (Control: V1/2=−68.4±3.0 mV, z=2.7±0.4; CANNABIDIOL: V1/2=−81.7±2.3 mV, z=2.7±0.3; n=5-9).

FIGS. 15E and 15F provide recovery from fast inactivation at 500 ms at pH7.3 (Control: τFast=0.0018±0.006 s, τSlow=0.15±0.6 s; CANNABIDIOL: τFast=0.24±0.07 s; τSlow=2.5±0.6 s; n=6-7) and pH6.4 (Control: τFast=0.065±0.04 s, τSlow=0.75±0.4 s; CANNABIDIOL: τFast=0.13±0.07 s; τSlow=0.62±0.1 s; n=4-7).

FIGS. 15G and 15H provide persistent currents measured from a 200 ms depolarizing pulse to 0 mV from a holding potential of −130 mV at pH7.4 (Control: Percentage=4.4±1.2%; CANNABIDIOL: Percentage=1.0±0.2%; n=4) and pH6.4 (Control: Percentage=4.4±2.1%; CANNABIDIOL: Percentage=5.4±1.2%; n=5-6).

FIGS. 16A-16F provide AP simulations of skeletal muscle action potentials in presence and absence of CANNABIDIOL, based on voltage-clamp data. Top of the figure show pulse protocol used for simulations, and a cartoon representation of P1158S-pH in-vitro/in-silico assay, where pH can be used to control the P1158S phenotype.

FIGS. 16A and 16B provide simulations in WT-Nav1.4 in presence and absence of CANNABIDIOL.

FIGS. 16C and 16D provide simulations of P1158S at pH6.4.

FIGS. 16E and 16F provide results from pH7.4.

FIG. 17 provides a cartoon representation of the mechanism and pathway through which CANNABIDIOL inhibits Nav1.4. Once CANNABIDIOL is exposed to the skeletal muscle, given its high lipophilicity, the majority of it gets inside the sarcolemma. Upon entering the sarcolemma, it localizes in the middle regions of the leaflet, and travels through the Nav1.4 fenestrations into the pore. Inside the pore mutation of the LA F1586A reduces CANNABIDIOL inhibition. CANNABIDIOL also alters the membrane rigidity, which promotes the inactivated state of the Nav channel, which adds to the overall CANNABIDIOL inhibitory effects. The net result is a reduced electrical excitability of the skeletal muscle, which—at least in part—contributes to a reduction in muscle contraction.

FIGS. 18A, 18B and 18C provide ²H NMR at different temperatures.

FIGS. 18A, 18B and 18C provide order parameters associated with POPC membranes at 20, 30, and 40° C.

FIGS. 19A-19C provide that CANNABIDIOL alters lipid bilayer properties in gramicidin-based fluorescence assay (GFA).

FIG. 19A provides fluorescence quench traces showing Tl+ quench of ANTS fluorescence in gramicidin containing DC22:1PC LUVs with no drug (control, black) and incubated with CANNABIDIOL for 10 min at the noted concentrations. The results for each drug represent 5 to 8 repeats (dots) and their averages (solid white lines).

FIG. 19B provides single repeats (dots) with stretched exponential fits (red solid lines).

FIG. 19C provides Florescence quench rates determined from the stretched exponential fits at varying concentrations of CANNABIDIOL (red) and TX100 (purple, from 43) normalized to quench rates in the absence of drug. Mean±SD, n=2 (for CANNABIDIOL (CANNABIDIOL)).

FIGS. 20A, 20B and 20C provide CANNABIDIOL interactions with DIV-S6, using isothermal titration calorimetry (ITC).

FIG. 20A provides representative ITC traces shown for titration of 100 mM lidocaine into 1 mM peptide or blank buffer.

FIG. 20B provides Representative ITC traces shown for titration of 40 mM CANNABIDIOL into 1 mM peptide or blank buffer.

FIGS. 20C and 20D provide (C) the blank condition subtracted heat of titration in protein condition is shown for lidocaine, and (D) CANNABIDIOL. A peak heat of 968.0±23.4 kcal*mol-1 was seen for lidocaine titration and a peak heat of 1022.2±160.6 kcal*mol-1 was seen for the CANNABIDIOL titration (n=3-4).

FIGS. 21A-21E provide Nav1.4 fenestration interactions with CANNABIDIOL.

FIGS. 21A to 21D provides that CANNABIDIOL posed in the human Nav1.4 structure using molecular docking.

FIG. 21E provides RMSD of the fenestration residues as a function of time in the absence (black) and the presence of CANNABIDIOL passing through the fenestration (red and green, two different simulation parameter sets). The similar RMSD profiles show that CANNABIDIOL's passage does not distort the structural integrity of the fenestration.

FIGS. 22A-22E provide that CANNABIDIOL stabilizes inactivation in the fenestration-occluded construct.

FIGS. 22A and 22B provide voltage-dependence of SSFI before and after control (extracellular (ECS) solution) (22A) and CANNABIDIOL (22B) in WWWW construct. The ECS experiment was performed to ensure that hyperpolarization shifts in the CANNABIDIOL condition are not due to possible confounding effects associated with fluoride in the internal (CsF) solutions.

FIGS. 22C and 22D provide representative families of inactivating currents before and after perfusion. CANNABIDIOL does not block peak currents but shifts the SSFI curve to the left.

FIG. 22E provides averaged hyperpolarization shift in the midpoint of SSFI before and after perfusion (Control=2.7±1.6 mV; CANNABIDIOL=24.0±6.6 mV, n=3-8).

FIG. 23A provides the effect of a cocktail of inflammatory mediators or 100 mM glucose or their vehicle (for 24 hours) on the conductance curve of Nav1.5 transfected cells with the insert showing the protocol (n=5, each).

FIG. 23B provides the effect of a cocktail of inflammatory mediators or 100 mM glucose or their vehicle (for 24 hours) on SSFI of Nav1.5 transfected cells with the insert showing the protocol (n=5, each).

FIG. 23C provides the effect of a cocktail of inflammatory mediators or 100 mM glucose or their vehicle (for 24 hours) on recovery from fast inactivation of Nav1.5 transfected cells with the insert showing the protocol (n=5, each).

FIG. 23D provides the effect of a cocktail of inflammatory mediators or 100 mM glucose or their vehicle (for 24 hours) on the percentage of persistent sodium currents of Nav1.5 transfected cells with the insert showing the protocol (n=5, each).

FIG. 23E provides representative families of macroscopic currents.

FIG. 23F provides representative persistent currents across conditions. Currents were normalized to peak current amplitude. Inset shows non-normalized currents.

FIG. 23G provides In silico action potential duration of Nav1.5 transfected cells incubated in inflammatory mediators or 100 mM glucose or the vehicle for 24 hours. *P<0.05 versus corresponding “Control” values.

FIG. 24A provides effect of inflammatory mediators (for 24 hours) or PK-A activator (CPT-cAMP; 1 for 20 minutes) or PK-C activator (PMA; 10 nM, for 20 minutes) on conductance curve Nav1.5 transfected cells with the insert showing the protocol (n=5, each).

FIG. 24B provides effect of inflammatory mediators (for 24 hours) or PK-A activator (CPT-cAMP; 1 for 20 minutes) or PK-C activator (PMA; 10 nM, for 20 minutes) on SSFI of Nav1.5 transfected cells with the insert showing the protocol (n=5, each).

FIG. 24C provides Effect of inflammatory mediators (for 24 hours) or PK-A activator (CPT-cAMP; 1 for 20 minutes) or PK-C activator (PMA; 10 nM, for 20 minutes) on recovery from fast inactivation of Nav1.5 transfected cells with the insert showing the protocol (n=5, each).

FIG. 24D provides Effect of inflammatory mediators (for 24 hours) or PK-A activator (CPT-cAMP; 1 for 20 minutes) or PK-C activator (PMA; 10 nM, for 20 minutes) on the percentage of persistent sodium currents of Nav1.5 transfected cells with the insert showing the protocol (n=5, each).

FIG. 24E provides representative families of macroscopic currents.

FIG. 24F provides representative persistent currents across conditions. Currents were normalized to peak current amplitude. Inset shows non-normalized currents. Representative persistent currents across conditions.

FIG. 24G provides effect of PK-A activator (CPT-cAMP; 1 μM for 20 minutes), PK-C activator (PMA; 10 nM, for 20 minutes) or inflammatory mediators (for 24 hours) on the In silico action potential duration of Nav1.5 transfected cells. *P<0.05 versus corresponding “Control” values.

FIG. 25A provides effect of PK-A inhibitor (H-89, 2 μM for 20 minutes) or PK-C inhibitor (Go 6983, 1 μM for 20 minutes) or their vehicle on the conductance curve Nav1.5 transfected cells incubated in the inflammatory mediators for 24 hours with the insert showing the protocol (n=5, each).

FIG. 25B provides effect of PK-A inhibitor (H-89, 2 μM for 20 minutes) or PK-C inhibitor (GO 6983, 1 μM for 20 minutes) or their vehicle on SSFI of Nav1.5 transfected cells incubated in the inflammatory mediators for 24 hours with the insert showing the protocol (n=5, each).

FIG. 25C provides Effect of PK-A inhibitor (H-89, 2 μM for 20 minutes) or PK-C inhibitor (GO 6983, 1 μM for 20 minutes) or their vehicle on recovery from fast inactivation of Nav1.5 transfected cells incubated in the inflammatory mediators for 24 hours with the insert showing the protocol (n=5, each).

FIG. 25D provides effect of PK-A inhibitor (H-89, 2 μM for 20 minutes) or PK-C inhibitor (Go 6983, 1 μM for 20 minutes) or their vehicle on the percentage of persistent sodium currents of Nav1.5 transfected cells incubated in the inflammatory mediators for 24 hours with the insert showing the protocol (n=5, each).

FIG. 25E provides representative families of macroscopic currents.

FIG. 25F provides representative persistent currents across conditions. Currents were normalized to peak current amplitude. Inset shows non-normalized currents. Representative persistent currents across conditions.

FIG. 25G provides effect of PK-A inhibitor (H-89, 2 μM for 20 minutes) or PK-C inhibitor (GO 6983, 1 μM for 20 minutes) on the In silico action potential duration of Nav1.5 transfected cells incubated in inflammatory mediators for 24 hours. *P<0.05 versus corresponding “Control/Veh” values. #P<0.05 versus corresponding “inflammatory mediators/Veh” values.

FIG. 26A provides effect of CANNABIDIOL (5 μM, perfusion) on the conductance curve of Nav1.5 transfected cells incubated with inflammatory mediators (24 hours) or PK-A activator (CPT-cAMP; 1 μM, for 20 minutes) or PK-C activator (PMA; 10 nM, for 20 minutes) with the insert showing the protocol (n=5, each).

FIG. 26B provides effect of CANNABIDIOL (5 μM, perfusion) on SSFI of Nav1.5 transfected cells incubated with inflammatory mediators (24 hours) or PK-A activator (CPT-cAMP; 1 μM, for 20 minutes) or PK-C activator (PMA; 10 nM, for 20 minutes) with the insert showing the protocol (n=5, each).

FIG. 26C provides effect of CANNABIDIOL (5 μM, perfusion) on recovery from fast inactivation of Nav1.5 transfected cells incubated with inflammatory mediators (24 hours) or PK-A activator (CPT-cAMP; 1 μM, for 20 minutes) or PK-C activator (PMA; 10 nM, for 20 minutes) with the insert showing the protocol (n=5, each).

FIG. 26D provides effect of CANNABIDIOL (5 μM, perfusion) on the percentage of persistent sodium currents of Nav1.5 transfected cells incubated with inflammatory mediators (24 hours) or PK-A activator (CPT-cAMP; 1 μM, for 20 minutes) or PK-C activator (PMA; 10 nM, for 20 minutes) with the insert showing the protocol (n=5, each).

FIG. 26E provides representative families of macroscopic currents.

FIG. 26F provides Representative persistent currents across conditions. Currents were normalized to peak current amplitude. Inset shows non-normalized currents. Representative persistent currents across conditions.

FIG. 26G provides effect of CANNABIDIOL (5 perfusion) on the In silico action potential duration of Nav1.5 transfected cells incubated in inflammatory mediators (24 hours) or PK-A activator (CPT-cAMP; 1 for 20 minutes) or PK-C activator (PMA; 10 nM, for 20 minutes). *P<0.05 versus corresponding “Control/Veh” values.

FIG. 27A provides the effect of Estradiol (E₂) (5 or 10 μM) on the conductance curve of Nav1.5 transfected cells incubated in 100 mM glucose (for 24 hours) with the insert showing the protocol (n=5, each).

FIG. 27B provides the effect of Estradiol (E₂) (5 or 10 μM) on SSFI of Nav1.5 transfected cells in 100 mM glucose (for 24 hours) with the insert showing the protocol (n=5, each).

FIG. 27C provides the effect of Estradiol (E₂) (5 or 10 μM) on recovery from fast inactivation of Nav1.5 transfected cells in 100 mM glucose (for 24 hours) with the insert showing the protocol (n=5, each).

FIG. 27D provides the effect of Estradiol (E₂) (5 or 10 μM) on the percentage of persistent sodium currents of Nav1.5 transfected cells in 100 mM glucose (for 24 hours) with the insert showing the protocol (n=5, each).

FIG. 27E provides representative families of macroscopic currents.

FIG. 27F provides representative persistent currents across conditions. Currents were normalized to peak current amplitude. Inset shows non-normalized currents. Representative persistent currents across conditions.

FIG. 27G provides the effect of Estradiol (E₂) (5 or 10 μM) on the In silico action potential duration of Nav1.5 transfected cells incubated in 100 mM glucose (for 24 hours). *P<0.05 versus corresponding “Control/Veh” values. #P<0.05 versus corresponding “100 mM glucose/Veh” values.

FIG. 28A provides effect of Estradiol (E₂) (5 or 10 μM) on conductance curve of Nav1.5 transfected cells incubated in inflammatory mediators (for 24 hours), PK-A activator (CPT-cAMP; 1 μM for 20 minutes) or PK-C activator (PMA; 10 nM, for 20 minutes) with the insert showing the protocol (n=5, each).

FIG. 28B provides effect of Estradiol (E₂) (5 or 10 μM) on SSFI of Nav1.5 transfected cells incubated in inflammatory mediators (for 24 hours), PK-A activator (CPT-cAMP; 1 μM for 20 minutes) or PK-C activator (PMA; 10 nM, for 20 minutes) with the insert showing the protocol (n=5, each).

FIG. 28C provides effect of Estradiol (E₂) (5 or 10 μM) on recovery from fast inactivation of Nav1.5 transfected cells incubated in inflammatory mediators (for 24 hours), PK-A activator (CPT-cAMP; 1 μM for 20 minutes) or PK-C activator (PMA; 10 nM, for 20 minutes) with the insert showing the protocol (n=5, each).

FIG. 28D provides effect of Estradiol (E₂) (5 or 10 μM) on the percentage of persistent sodium currents of Nav1.5 transfected cells incubated in inflammatory mediators (for 24 hours), PK-A activator (CPT-cAMP; 1 μM for 20 minutes) or PK-C activator (PMA; 10 nM, for 20 minutes) with the insert showing the protocol (n=5, each). FIG. 6E provides representative families of macroscopic currents.

FIG. 28F provides representative persistent currents across conditions. Currents were normalized to peak current amplitude. Inset shows non-normalized currents. Representative persistent currents across conditions.

FIG. 28G provides effect of Estradiol (E₂) (5 or 10 μM) on the In silico action potential duration of Nav1.5 transfected cells incubated in inflammatory mediators (for 24 hours), PK-A activator (CPT-cAMP; 1 μM for 20 minutes) or PK-C activator (PMA; 10 nM, for 20 minutes).

*P<0.05 versus corresponding “Control/Veh” values. #P<0.05 versus corresponding “inflammatory mediators/Veh” values.

FIG. 29—A schematic of possible cellular pathway involved in the protective effect of CANNABIDIOL. Estradiol (E₂) against high glucose induced inflammation and activation of PK-A and PK-C via affecting cardiac voltage-gated sodium channels (Nav1.5).

FIG. 30A—It shows conductance plotted as a function of membrane potential. It is found that incubation in inflammatory mediators for 24 hours significantly right-shifted V1/2 of activation (P=0.0015) (from −37.3±1.2 mV to −22.3±2.4 mV, n=4, each) and decreased z of activation curve (P=0.0034) (from 3.8±0.16 mV to 2.7±0.17 mV, n=4, each).

FIG. 30B—It shows normalized current amplitudes plotted as a function of pre-pulse potential Inflammatory mediators caused significant shifts in the positive direction in the V1/2 obtained from Boltzmann fits (P=0.0084) (from −92.3±3.4 mV to −77.1±1.7 mV, n=4, each).

FIG. 30C— It shows that incubation in inflammatory mediators significantly increased INap compared to control (inflammatory mediators: P<0.0001) (from 0.80±0.05 to 5.44±0.11).

FIGS. 30D and 30E—Representative families of macroscopic and persistent currents across conditions are provided.

FIG. 31A provides a plot of current amplitude plotted against time in milliseconds and the plot provides late sodium current when cells are incubated with azithromycin. Further perfusion of the cells with 5 μM CANNABIDIOL reduced the late current.

FIG. 31B provides a plot of current amplitude plotted against time in milliseconds and the plot provides late sodium current when cells are incubated with azithromycin. Further perfusion of the cells with 5 μM CANNABIDIOL reduced the late current. The cells employed are different.

FIG. 32A provides an effect on the conductance curve of Nav1.5 transfected cells incubated in control (10 mM glucose), high (100 mM) glucose, or high glucose and 5 uM CBD. High glucose incubation shifts the activation curve to the right. Co-incubation of CBD with high glucose rescues the activation.

FIG. 32B provides an effect on the normalized current curve of Nav1.5 transfected cells incubated in control (10 mM glucose), high (100 mM) glucose, or high glucose and 5 uM CBD. High glucose incubation shifts the steady-state inactivation to the right. Co-incubation of CBD with high glucose rescues the steady-state inactivation.

FIG. 32C provides an effect on the percentage of persistent sodium current of Nav1.5 transfected cells incubated in control (10 mM glucose), high (100 mM) glucose, or high glucose and 5 uM CBD. High glucose incubation enhanced persistent sodium current. Co-incubation with cannabidiol reduces late sodium current.

FIG. 32D provides an effect on action potential of Nav1.5 transfected cells incubated in control (10 mM glucose), high (100 mM) glucose, or high glucose and 5 uM CBD. High glucose incubation causes prolongation of action potential. Co-incubation with cannabidiol reduces action potential which is indistinguishable from control.

FIGS. 32E and 32F respectively provide current traces recorded during the activation protocol (32E) and the persistent current protocol (32F).

### DETAILED DESCRIPTION OF THE INVENTION

Sodium current passing through Nav (Sodium channel) initiates action potentials (AP) in neurons, myocardium, and skeletal muscles. Nav subtype predominantly expressed in skeletal muscles is Nav1.4 whereas Nav subtype predominantly expressed in cardiac muscles is Nav1.5.

The present invention provides compositions of a new therapeutic agent Cannabidiol which acts through its effects on sodium channels Nav1.5 and 1.4 to treat various cardiac disorders, inflammation and skeletal muscle disorders.

### Sodium Channel Nav1.5—a Molecular Target for Treating Cardiac Disorders.

In cardiac muscle, sodium currents contribute to the ventricular action potential. Normally, sodium channels activate and then rapidly inactivate and the remainder of the action potential is controlled by calcium and potassium channels.

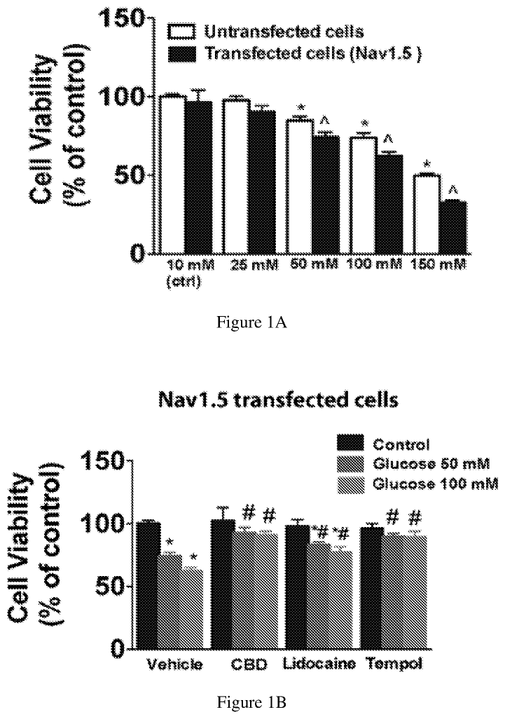

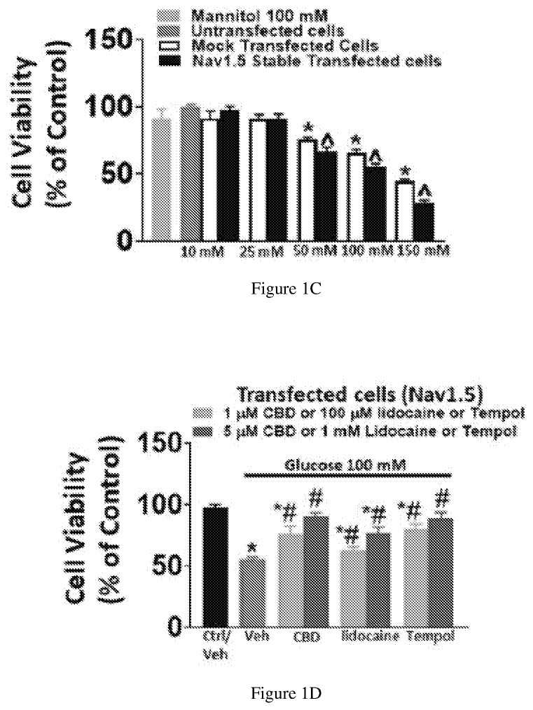

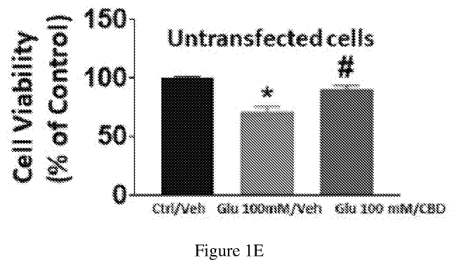

Nachimuthu et al under FIG. 1 provides five phases of cardiac depolarization and repolarization wherein the first two phases (phase 0 and phase 1) are respectively characterized by large inward currents of sodium ions (phase 0) and inactivation of depolarizing sodium current (phase 1).

Activation of sodium channels, steady state fast inactivation, stabilization of inactivation and recovery from inactivation are very essential for normal functioning of sodium channels. Unless Sodium channels recover from fast inactivation, they cannot get deactivated and unless they are deactivated, they are not ready to take part in further action potential.

Certain physiological conditions and certain induced conditions may affect normal functioning of sodium channels. These conditions affect the gating properties of sodium channels. Such channels whose gating properties are affected are also termed here as adversely affected sodium channels. Such sodium channels are less likely to activate at any given membrane potential. Additionally, if sodium channels do not inactivate properly, this causes a late sodium current or persistent sodium current that prolongs the action potential duration and delays repolarization. Delayed repolarization causes Long QT, which is an increase in time between the QRS complex and the T wave in the ECG.

The present invention provides pharmaceutical compositions of a new therapeutic agent which acts on the sodium channels and corrects defects in the gating properties of sodium channels. This is termed as rescue of adversely affected sodium channels to restore normal electrophysiology of these channels. The new therapeutic agent acts to abolish the late/persistent sodium current thereby preventing i) prolongation of action potential and ii) delayed repolarization. The new therapeutic agent thus provides a breakthrough therapy in Long QT arrhythmias and several cardiac dysfunctions which are caused due to a) defects in gating properties or b) hyperexcitability or c) arrhythmias or c) ailments which lead to arrhythmias. The new therapeutic agent is CANNABIDIOL.

These pharmaceutical compositions further include one or more pharmaceutical carriers appropriate for administration to an individual in need thereof. The pharmaceutical compositions are suitable for acting on at least one molecular target which is Nav1.5. These pharmaceutical compositions produce beneficial effects in one or more of the following pathogenesis of various cardiovascular disorders including, but not limited to, long QT syndrome, long QTc syndrome, long QRS syndrome, cardiomyopathy, heart failure, arrhythmia, ischemia, Heart Failure, Hypertrophic cardiomyopathy, Hypoxia myocardial ischemia, myocardial infarction (MI), arrhythmias of ischemic and non-ischemic origin, inflammation, vascular dysfunction, cardiomyopathy, cardiac remodelling, maladaptation, anginas of different types, drug induced heart failure, iatrogenic heart and vascular diseases, or any combination thereof. Ischemia. These pharmaceutical compositions produce beneficial effects in one or more of the following pathogenesis of various cardiovascular disorders including particularly, long QT syndrome, long QTc syndrome, long QRS syndrome, arrhythmia, ischemia, Heart Failure, Hypertrophic cardiomyopathy and Hypoxia.

As mentioned in a paper by Nachimuthu et al, more than 10 different types of congenital LQTS have been recognized [Hedley et al. 2009; Modell and Lehmann, 2006; Roden, 2008]. LQT1, LQT2, and LQT3 account for the majority of the cases of congenital LQTS. Further, Nachimuthu et al mentions that LQT3 accounts for 8-10% of cases [Schwartz et al. 2001; Splawski et al. 2000]. It is caused by mutations in the sodium channel gene (SCN5A) located on chromosome 3 at location 21-24. It is characterized by events occurring at rest or during sleep.

Sodium channel mutations are inherited, and therefore are present from birth leading to inherited Long QT3. Apart from inherited Long QT3, acquired Long QT may arise due to various conditions and agents. Nachimuthu et al provides a list of drugs leading to acquired Long QT in his article titled, “Drug-induced QT interval prolongation: mechanisms and clinical management”.

Besides several drugs, certain ailments/conditions also cause Long QT such as diabetes, hyperglycaemia, ischemia etc. Recent outbreak of Covid-19 has been found to induce Long QT and several cardiac disorders in patients.

Some of the several conditions where normal functioning of the sodium channel is affected is diabetes or hyperglycemia. In diabetic and hypoglycemic patients, such acquired long QT syndrome appears leading to cardiac complications.

Thus, whether inherited or acquired due to drugs or diseases, LQT is associated with problems in proper functioning of sodium channels. In other words, the gating properties of sodium channels are affected to induce hyperexcitability.

Thus, the inventors have found that the Sodium channel Nav1.5 is a molecular therapeutic target for alleviating the deleterious consequences of several cardiac disorders including hyperexcitability or other problems of gating properties of sodium channels to treat Long QT and arrhythmias.

The inventors have conducted several experiments to study electrophysiological changes in sodium channels to arrive at the present invention. First various mediators are used to induce gating changes in the sodium channels and such adversely affected sodium channels are treated with the new therapeutic agent to check whether the channels can be rescued. Sodium channels are produced by using Chinese hamster ovary.

Encoding the Nav1.5 α-Subunit, the β1-Subunit, and eGFP.

Encoding the Nav1.5 is done as follows. Chinese hamster ovary (CHO) was grown at pH 7.4 in filtered sterile F12 (Ham) nutrient medium (Life Technologies, Thermo Fisher Scientific, Waltham, Mass., USA), supplemented with 5% FBS and maintained in a humidified environment at 37° C. with 5% CO2. Cells were transiently co-transfected with the human cDNA encoding the Nav1.5 α-subunit, the β1-subunit, and eGFP. Transfection was done according to the PolyFect (Qiagen, Germantown, Md., USA) transfection protocol. A minimum of 8-hour incubation was allowed after each set of transfections. The cells were subsequently dissociated with 0.25% trypsin-EDTA (Life Technologies, Thermo Fisher Scientific).

These cells are ready to be subjected to various mediators that are likely to induce changes in the gating properties. If changes in the gating properties of sodium channel Nav1.5 are observed, they are adversely affected channels which can be used in further studies.

The present invention focusses on a new therapeutic agent to ascertain its potential effect in various cardiac disorders. The present invention aims to investigate effects of the new therapeutic agents by studying its action on Sodium channel Nav1.5 which is the major cardiac sodium channel isoform of the heart. To investigate such effects, first adversely affected sodium channels are produced which are treated with the therapeutic agent to check whether such adverse effects can be rescued. Adversely affected sodium channels are those whose gating properties are affected so that they have at least one problem that either they do not activate, or they do not inactivate properly, or they do not recover from inactivation to take part in further action potential etc.

The present inventors have surprisingly found two types of mediators to create adversely affected sodium channels Nav1.5. These mediators are high glucose conditions and inflammation.

In the first part of the study, high glucose conditions are employed as mediators to cause defects in the gating properties whereas in the second part of the study, inflammatory mediators are employed to mimic effects of inflammation on the gating properties. Further, the inventors have surprisingly found that the adversely affected sodium channels are rescued by the new therapeutic agents employed in the study and therefore these new therapeutic agents provide breakthrough in the treatment of various cardiac disorders involving such adversely affected sodium channel.

Apart from the electrophysiological changes, the inventors have also studied effects of formation of reactive oxygen species on sodium channels by action of one or more mediators. High glucose conditions have enhanced formation of reactive oxygen species which is also supported by another experiment where cell viability is measured and found to be reduced. The reactive oxygen species generated as a result of action of mediator on sodium channel are reduced by the new therapeutic agent. This is supported by enhanced cell viability due to new therapeutic agent. This research indicates that if any mediator induces formation of reactive oxygen species, the new therapeutic agent is able to cause reduction in the formation of reactive oxygen species thereby preventing oxidative damage. If reactive oxygen species become uncontrolled, this may lead to hyperexcitability, cytotoxicity and prolongation of action potential leading to Long QT arrythmias and other cardiac disorders.

### Human Cardiomyocytes

Apart from the encoded sodium channels, human cardiomyocytes are employed to check whether one or more mediators cause electrophysiological changes in human cardiomyocytes and if changes are observed whether new therapeutic agent rescues the cardiomyocytes from such changes.

### Human Cardiomyocytes Preparation is as Follows:

A frozen cryovial containing ≥1×106 cardiomyocytes (Cellular Dynamics International, kit 01434, Madison, Wis., USA) were thawed by immersing the frozen cryovial in a 37° C. water bath, transferring thawed cardiomyocytes into a 50-ml tube, and diluting them with 10 ml of ice-cold plating medium (iCell Cardiomyocytes Plating Medium (iCPM); Cellular Dynamics International, Madison, Wis., USA) (Ma et al., 2011). For single cell patch-clamp recordings, glass coverslips were coated with 0.1% gelatin (Cellular Dynamics International, Madison, Wis., USA) and placed into each well of a 24-well plate for an hour. This was followed by adding 1 ml of iCPM containing 40,000-60,000 cardiomyocytes to each coverslip. Plated cardiomyocytes were at a low density to permit culture as single cells and were stored in an environmentally controlled incubator maintained at 37° C. and 7% CO2. After 48 h, iCPM was replaced with a cell culture medium (iCell Cardiomyocytes Maintenance Medium (iCMM); Cellular Dynamics International, Madison, Wis., USA), which was exchanged every other day with the cardiomyocytes maintained on cover slips for 4 to 21 days before use (Ma et al., 2011).

These Human cardiomyocytes are ready to be subjected to various mediators that are likely to induce changes in the gating properties which can be reflected from the electrophysiological changes in these cells. If changes in the gating properties of Human cardiomyocytes are observed, they can be used in further studies involving therapeutic agent to check whether the therapeutic agent can rescue these electrophysiological changes.

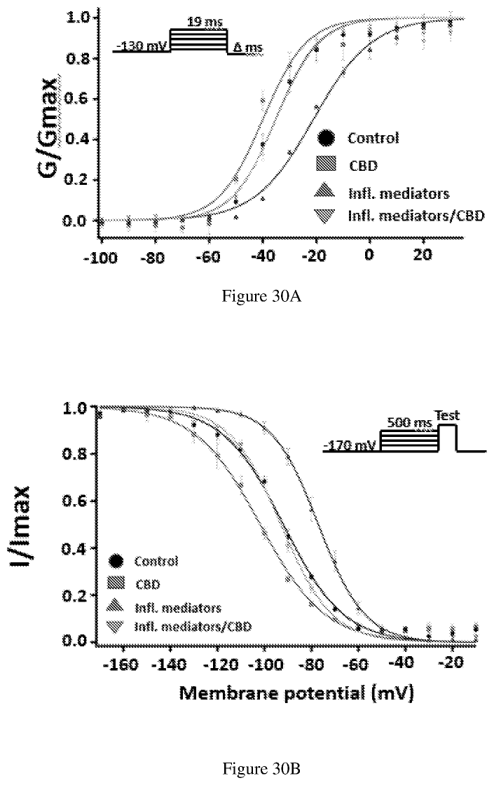

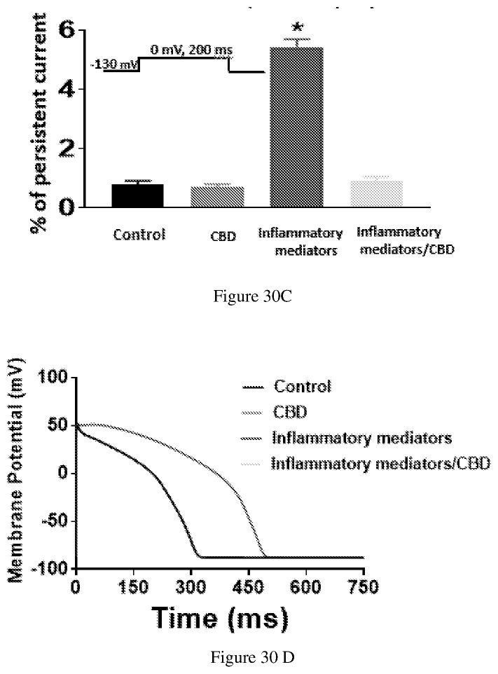

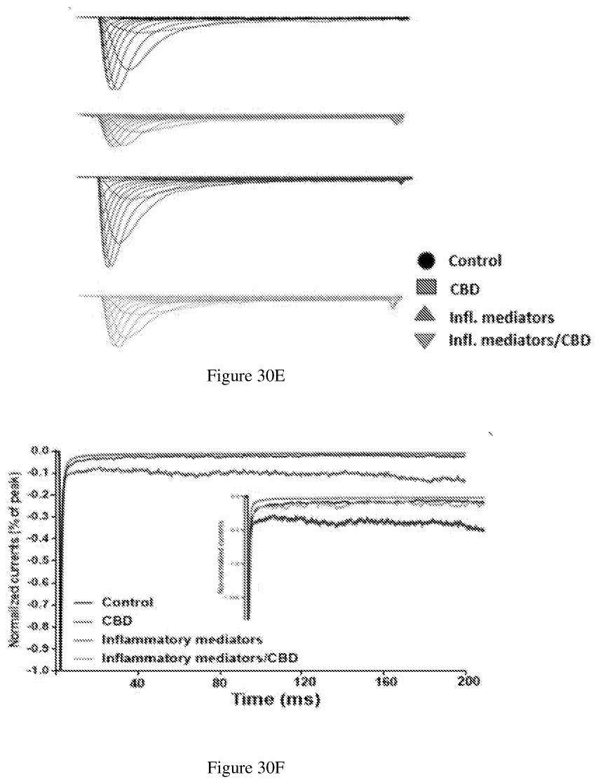

The inventors have found that human cardiomyocytes behaved exactly similar to encoded sodium channels. The mediators that affected the gating properties of the sodium channel Nav1.5 also affected in a similar manner electrophysiology of the cardiomyocytes (FIGS. 30A and 30D). The new therapeutic agent also rescued the electrophysiological changes in the human cardiomyocytes where such changes were induced by mediators.

### Selection of Mediators:

### First Part of the Study

One of the several conditions that affect gating properties of Nav (Sodium channels) is diabetes or hyperglycaemia.

As provided by Viskupicova et al (Viskupicova et al., 2015),

i) Hyperglycaemia is the most important factor in the onset and progress of diabetic complications; and
ii) High glucose concentrations are usually used as a model to mimic the in vivo situation of hyperglycaemia in diabetes and high glucose concentrations (up to 100 mM of D-glucose) have been previously used to mimic the human hyperglycaemia based on the used cell line.

Thus, high glucose seems to be one of the conditions to modulate gating properties of sodium channels. It has been found by the present inventors that high glucose adversely affects Nav1.5, the major cardiac sodium channel isoform of the heart, at least partially via oxidative stress. High glucose modulates the gating properties of the Nav1.5 to induce hyperexcitability. Thus, the inventors propose that the Nav1.5 could be a molecular therapeutic target for alleviating the deleterious consequences of diabetes/high glucose.

High glucose modulates the gating properties of the Nav1.5 to induce hyperexcitability. Hyperexcitability of sodium channels further lead to several cardiac ailments. Therefore, to mimic conditions of sodium channels in various cardiac ailments, use of high glucose concentrations (up to 100 mM of D-glucose) is considered.

The inventors have used high glucose concentrations (up to 100 mM of D-glucose) to mimic the human hyperglycaemic conditions/diabetic conditions.

Therefore, use of high glucose as a mediator serves two purposes as follows:

1. In hyperglycaemic or diabetic patients, sodium channels Nav1.5 are adversely affected. Since Nav1.5 is the major cardiac sodium channel isoform of the heart, such patients also have various cardiac ailments. Hence, high glucose concentrations (up to 100 mM of D-glucose) are used to mimic adversely affected sodium channels in various cardiac ailments.

2. High glucose concentrations (up to 100 mM of D-glucose) mimic the human hyperglycaemic conditions/diabetic conditions. Such patients are more prone to develop cardiac ailments. Hence, high glucose concentrations (up to 100 mM of D-glucose) are used to mimic adversely affected sodium channels in hyperglycaemic/diabetic patients which represent a population more prone to various ailments.

In the present invention, the inventors have surprisingly found that high glucose conditions affected all gating properties of sodium channels. Additionally, high glucose conditions have elicited oxidative stress and cytotoxicity. Formation of reactive oxygen species (ROS) manifest in number of ways leading to severe pathogenesis of Sodium channels Nav 1.5 resulting in cytotoxic effects and reducing cell viability, hyperexcitability and further into prolongation of action cardiac potential, LQTs and arrythmias. A build-up of reactive oxygen species in cells may cause damage to DNA, RNA, and proteins, and may cause cell death.

Further, inventors have tested several therapeutic agents including a new therapeutic agent on such adversely affected sodium channels to check whether these agents and particularly the new agent can rescue the channels from the effects of high glucose.

Apart from the new therapeutic agent CANNABIDIOL, standard therapeutic agents termed as reference compounds or reference which are employed for various cardiac ailments are also added in the study design where they act as a control. For example, lidocaine is employed in treatments of Ventricular Arrhythmias or Pulseless Ventricular Tachycardia (after defibrillation, attempts, CPR, and vasopressor administration). Another therapeutic agent/reference used is Tempol which is also used as a control. Tempol is an antioxidant and has been reported to reduce oxidative stress and to attenuate oxidative damage.

An anti-oxidant plays three major roles while reducing ROS and its effects.

(i) it directly scavenges ROS already formed; (ii) it inhibits further formation of ROS; and (iii) it removes or repairs the damage or modifications caused by ROS.

Use of tempol has been studied as a vasodilator in clinical trials. If these existing therapeutic agents show rescue of adversely affected sodium channels, any potential therapeutic agent must also show such rescue. The effects exhibited by the Control therapeutic agents also termed as reference compound or simply reference and the new therapeutic agent are compared to check performance of the new therapeutic agent. The new therapeutic agent CANNABIDIOL along with tempol are tested to check whether they can rescue high glucose induced cytotoxic effects and high glucose induced effects of ROS formation on Sodium channels Nav 1.5.

The inventors have surprisingly found that the new therapeutic agent CANNABIDIOL is at least as good as tempol in reducing effects and formation of reactive oxygen species thus minimizing, and completely abolishing the chances of hyperexcitability of these channels. This data is supported by cell viability data. Cell viability is greatly enhanced as a result of reduction in formation of reactive oxygen species. Since cytotoxic effects and reactive oxygen species are produced under variety of circumstances in the body, the new therapeutic agent CANNABIDIOL can be used to treat such conditions which if not controlled may lead to severe pathogenesis or even may lead to fatal conditions.

The inventors have surprisingly found that the new therapeutic agent CANNABIDIOL is able to rescue the adversely affected sodium channels that have shown electrophysiological changes such that they can activate, fast inactivate and recover from fast inactivation etc.

Additionally, the new therapeutic agent CANNABIDIOL is able to cause reduction in formation of reactive oxygen species thereby reduces oxidative stress and cytotoxicity and enhances cell viability.

The inventors provide various pharmaceutical compositions of the new therapeutic agent CANNABIDIOL which serve two functions:

1. They can rescue already affected gating property of sodium channels and hence crucial in treating various cardiac disorders and in hyperglycaemic or diabetic patients prone to cardiac disorders; and
2. They can prevent sodium channels from getting adversely affected by providing prophylactic effects thus i) avoiding or minimizing happening of cardiac disorders in a healthy population; and ii) avoiding or minimizing occurrence of cardiac disorders in diabetic or hyperglycaemic patients prone to such disorders.

Thus, the invention provides pharmaceutical compositions and therapeutic uses of the new therapeutic agent CANNABIDIOL.

1. to treat cardiac disorders or for prophylaxis of cardiac disorders i.e. to prevent happening of such cardiac disorders; and
2. to prophylactically treat diabetic or hyperglycaemic population more prone to cardiac disorders.

The several aspects of the invention are hereinbelow described.

Under the first aspect, the invention provides various pharmaceutical compositions employing the new therapeutic agent CANNABIDIOL. The inventors have surprisingly found that the new therapeutic agent CANNABIDIOL rescues the adversely affected sodium channels Nav1.5 and thus can serve as potential therapeutic agent for treating several cardiovascular disorders. The invention further provides uses of these pharmaceutical compositions for treating various cardiac disorders. The invention also includes treating patients suffering from various cardiac disorders by administering suitable pharmaceutical compositions employing CANNABIDIOL.

Accordingly, the invention provides a pharmaceutical composition comprising therapeutically effective amount of CANNABIDIOL for use in treatment of a cardiac disorder arising from gating defects in sodium channel Nav1.5.

The invention also provides a pharmaceutical composition comprising therapeutically effective amount of cannabidiol for use in treatment of a cardiac disorders arising from gating defects in sodium channel Nav1.5 wherein the gating defects includes at least one from i) less likely to activate; ii) inability to fast inactivate; iii) unstable fast inactivation; iv) late or persistent sodium current and v) prolongation of action potential.

In this aspect, invention further provides a pharmaceutical composition comprising therapeutically effective amount of cannabidiol for use in treatment of a cardiac disorders arising from gating defects in sodium channel Nav1.5 wherein the gating defect is selected from late or persistent sodium current and prolongation of action potential.

In this aspect, invention also provides, a method of treating cardiac disorder in a patient suffering from such disorder comprising administering a pharmaceutical composition comprising therapeutically effective amount of CANNABIDIOL wherein the cardiac disorder arises from gating defects in sodium channel Nav1.5.

The invention further provides a method of treating cardiac disorder in a patient suffering from such disorder comprising administering a pharmaceutical compositions comprising therapeutically effective amount of cannabidiol wherein the cardiac disorder arises from gating defects in sodium channel Nav1.5 wherein the gating defect includes at least one from i) less likely to activate; ii) inability to fast inactivate; iii) unstable fast inactivation; iv) late or persistent sodium current and v) prolongation of action potential.

Under the first aspect, invention also provides method of treating cardiac disorder in a patient suffering from such disorder comprising administering a pharmaceutical compositions comprising therapeutically effective amount of cannabidiol wherein the cardiac disorder arises from gating defect wherein the gating defect is selected from late or persistent sodium current and prolongation of action potential.

These pharmaceutical compositions further include one or more pharmaceutical carrier appropriate for administration to an individual in need thereof. The pharmaceutical compositions are suitable for acting on at least one molecular target which is Nav1.5. These pharmaceutical compositions produce beneficial effects in one or more of the following pathogenesis of various cardiovascular disorders including, but not limited to, long QT syndrome, long QTc syndrome, long QRS syndrome, cardiomyopathy, heart failure, arrhythmia, ischemia, Heart Failure, Hypertrophic cardiomyopathy and Hypoxia myocardial ischemia, myocardial infarction (MI), arrhythmias of ischemic and non-ischemic origin, inflammation, vascular dysfunction, cardiomyopathy, cardiac remodelling, maladaptation, anginas of different types, drug induced heart failure, iatrogenic heart and vascular diseases, or any combination thereof. These pharmaceutical compositions produce beneficial effects in one or more of the following pathogenesis of various cardiovascular disorders including particularly, long QT syndrome, long QTc syndrome, long QRS syndrome, arrhythmia, ischemia, Heart Failure, Hypertrophic cardiomyopathy and Hypoxia.

High glucose concentrations (up to 100 mM of D-glucose) are used to mimic adversely affected sodium channels in hyperglycaemic/diabetic patients which represent a population more prone to various cardiac ailments. Similar to a control therapeutic agent (reference compound or reference) such as lidocaine, if CANNABIDIOL rescues the adversely affected sodium channels Nav1.5, it is potential agent in preventing high glucose or diabetes induced cardiac disorders. The inventors have surprisingly found that the new therapeutic agent CANNABIDIOL rescues the adversely affected sodium channels Nav1.5 and thus can serve as potential therapeutic agent for treating several cardiovascular disorders that may be induced by hyperglycaemic or diabetic conditions.

Under the second aspect, the invention provides various pharmaceutical compositions employing the new therapeutic agent CANNABIDIOL for treating various cardiac disorders induced by hyperglycaemic or diabetic conditions. The invention also includes treating patients suffering from various cardiac disorders induced by hyperglycaemic or diabetic conditions by administering suitable pharmaceutical compositions employing CANNABIDIOL.

Accordingly, the invention provides a pharmaceutical composition comprising therapeutically effective amount of cannabidiol for use in treatment of a cardiac disorder arising from gating defects in sodium channel Nav1.5 wherein the gating defect is induced by hyperglycaemic or diabetic condition.

In this aspect, invention further provides a method of treating cardiac disorder in a patient suffering from such disorder comprising administering a pharmaceutical composition comprising therapeutically effective amount of cannabidiol wherein the cardiac disorder arises from gating defects in sodium channel Nav1.5 and wherein the gating defect is induced by hyperglycaemic or diabetic condition.

Under the third aspect, the invention provides various pharmaceutical compositions employing the new therapeutic agent CANNABIDIOL for avoiding or minimizing occurrence of cardiac disorders in a hyperglycaemic or diabetic population more prone to such disorders. The pharmaceutical compositions of the present invention are prophylactic in nature for hyperglycaemic or diabetic population prone to cardiac ailments and can be consumed by hyperglycaemic or diabetic population in their daily regime. The CANNABIDIOL levels in blood/plasma will help individual from getting affected by cardiac disorders or at least minimize such chances. The invention further provides uses of these pharmaceutical compositions for avoiding or minimizing cardiac disorders in a hypoglycemic or diabetic population and treating by administering pharmaceutical compositions employing new therapeutic agent CANNABIDIOL to achieve the same.

Accordingly, in this aspect invention provides a pharmaceutical composition comprising a therapeutically effective amount of cannabidiol for use in avoiding or minimizing occurrence of a cardiac disorder arising from gating defects in sodium channel Nav1.5 wherein the gating defect is prone to be induced by hyperglycaemic or diabetic condition.

Under the third aspect, invention further provides, a method of avoiding or minimizing occurrence of a cardiac disorder comprising administering a pharmaceutical composition comprising therapeutically effective amount of cannabidiol wherein the cardiac disorder arises from gating defects in sodium channel Nav1.5 wherein the gating defect is prone to be induced by hyperglycaemic or diabetic condition

Thus, the patients who may receive pharmaceutical compositions of the new therapeutic agent CANNABIDIOL under the first three aspects are summarized below:

TABLE 1
Patient population under first three aspects

| | |
|---|---|
| First | Patients having cardiac ailments due to defects in gating |
| aspect | properties of sodium channels. |
| Second | Patients having hyperglycaemia and/diabetes as well as |
| aspect | cardiac ailments due to defects in gating properties of |
| | sodium channels where such cardiac ailments are induced |
| | by hyperglycaemia and/diabetes. Patients having |
| | hyperglycaemia and/diabetes and cardiac ailments due to |
| | defects in gating properties of sodium channels where such |
| | cardiac ailments are induced by hyperglycaemia and/ |
| | diabetes. |
| Third | Patients having hyperglycaemia and/diabetes but not |
| aspect | cardiac ailments, but they are prone to developing cardiac |
| | ailments. |

Since the pharmaceutical compositions of the present invention employing new therapeutic agent CANNABIDIOL help in abolishing or minimizing hyperexcitability of sodium channels Nav 1.5 and thereby abolishing or minimizing prolongation of action potential and Long QT intervals, these pharmaceutical compositions are employed along with other drugs/medicines which induce Long QT intervals.

Under the fourth aspect, the invention provides various pharmaceutical compositions employing the new therapeutic agent CANNABIDIOL for abolishing or minimizing side effects of other therapeutic agents/drugs which induce, or which are likely to induce Long QT. In this aspect, CANNABIDIOL pharmaceutical compositions enhance safety profile of other therapeutic agents as well as enhance their application which were limited due to their side effects mainly Long QT interval.

The invention further provides uses of these pharmaceutical compositions for avoiding or minimizing side effects of other therapeutic agents/drugs which induce, or which are likely to induce Long QT and treating by administering pharmaceutical compositions employing new therapeutic agent CANNABIDIOL to achieve the same.

Accordingly, invention provides a pharmaceutical composition comprising therapeutically effective amount of cannabidiol for use in treatment of a cardiac disorder arising from gating defect in sodium channel Nav1.5 wherein the gating defect includes at least one from i) less likely to activate; ii) inability to fast inactivate; iii) unstable fast inactivation; iv) late or persistent sodium current and v) prolongation of action potential; and wherein the gating defect arises due to treatment with another therapeutic agent.

In this aspect, invention further provides a method of treating cardiac disorder in a patient suffering from such disorder comprising administering a pharmaceutical composition comprising therapeutically effective amount of cannabidiol wherein the cardiac disorder arises from gating defects in sodium channel Nav1.5 and wherein the gating defect arises in such patient due to treatment with another therapeutic agent.

Covid-19 vaccines are reported to induce severe side effects in some individuals leading to Long QT arrythmias.

Under the fourth aspect, the pharmaceutical compositions of new therapeutic agent CANNABIDIOL are administered along with Covid-19 vaccine or any vaccine which is likely to induce LQT arrythmias.

Accordingly, invention provides a pharmaceutical composition comprising therapeutically effective amount of cannabidiol for use in avoiding or minimizing occurrence of a cardiac disorder arising from gating defects in sodium channel Nav1.5 wherein the gating defect includes at least one from i) less likely to activate; ii) inability to fast inactivate; iii) unstable fast inactivation; iv) late or persistent sodium current and v) prolongation of action potential; and wherein the gating defect is likely to be induced by administration of i) at least one other therapeutic agent or ii) Covid-19 vaccine.

In this aspect, invention also provides a method of avoiding or minimizing occurrence of a cardiac disorder comprising administering a pharmaceutical composition comprising therapeutically effective amount of cannabidiol wherein the cardiac disorder arises from gating defects in sodium channel Nav1.5 and wherein the gating defect is likely to be induced by administration of i) at least one other therapeutic agent or ii) Covid-19 vaccine.

The human cardiac sodium channel (hNav1.5, encoded by the SCN5A gene) is critical for action potential generation and propagation in the heart. Drug-induced sodium channel inhibition decreases the rate of cardiomyocyte depolarization and consequently conduction velocity.

Warner B. and Hoffmann P (Warner B. and Hoffmann P; 2002) in their article titled “Investigation of the potential of clozapine to cause torsade de pointes” mentions that inhibition of cardiac sodium channels can mitigate hERG channel blocking effects of drugs as shown for the antipsychotic compound clozapine.

Zequn Z et al provides examples of the drugs that may induce long QT and/or cardiotoxicity possibly due to hERG mitigation and include chloroquine, hydroxychloroquine, azithromycin, and lopinavir/ritonavir,

Examples of agents prolonging QT include agents but are not limited to drugs such as Azithromycin, Baloxavir, Lopinavir and Ritonavir; Neuraminidase inhibitors (eg. Oseltamivir), Remdesivir; anti-malarials such as Chloroquine phosphate, hydroxychloroquine; supporting agents such as Sarilumab, Sirolimus, Tocilizumab and other agents such as ACE Inhibitors, Angiotensin II Receptor Blockers (ARBs), Ibuprofen, Indomethacin and Niclosamide.

Opioids also induce long QT. Kuryshev et al (Kuryshev et al 2010) mentions that Methadone, a synthetic opioid for treatment of chronic pain and withdrawal from opioid dependence, has been linked to QT prolongation, potentially fatal torsades de pointes, and sudden cardiac death.

Thus, treatment with opioids and particularly Methadone can include combining their compositions with pharmaceutical composition of the present invention.

The present inventors have demonstrated effects of Azithromycin on Sodium channel Nav1.5 wherein they incubated cells heterologously expressing Nav1.5 in 10 μM Azithromycin and observed an increase in late sodium current compared to control (no Azithromycin incubation) cells. Further the inventors perfused the cells showing Azithromycin-induced late sodium current with 5 μM CANNABIDIOL and observed that the late current was reduced. Thus, CANNABIDIOL rescues the proarrhythmic effects of Azithromycin and, thus, can be a useful adjuvant therapy in conditions that call for treatment with macrolide antibiotics, possibly including COVID-19.

Under a fifth aspect, the invention provides CANNABIDIOL pharmaceutical compositions for Covid-19 treatment in two circumstances below:

1. where Covid-19 has induced Long QT in patient or where Covid-19 is likely to induce Long QT in patients suffering from other comorbidities; and
2. where Covid-19 treatment uses any therapeutic agent or likely to use any therapeutic agent where such agent has induced or is likely to induce Long QT in patients.

The invention further provides pharmaceutical compositions of CANNABIDIOL for uses in Covid-19 treatment where Long QT has been induced or is likely to be induced either due to Covid-19 or due to treatment of Covid 19 with any therapeutic agent likely to cause LQT and treating Covid-19 patients by administering pharmaceutical compositions employing new therapeutic agent CANNABIDIOL alone or along with such other therapeutic agent likely to cause or has caused Long QT. Thus, the fifth aspect covers pharmaceutical compositions of CANNABIDIOL which can be administered in Covid-19 treatment. Such pharmaceutical compositions may have CANNABIDIOL alone or CANNABIDIOL and the therapeutic agent useful in Covid-19 treatment. Without limitations, these other therapeutic agents include antivirals, chloroquine, hydroxychloroquine and even neutraceuticals such as vitamins. These other therapeutic agents may also encompass but are not restricted to natural—organic or in-organic, ayurvedic, homeopathic, siddha and unani medicines.

Accordingly, invention provides a pharmaceutical composition comprising therapeutically effective amount of cannabidiol for use in avoiding or minimizing occurrence of a cardiac disorder arising from gating defects in sodium channel Nav1.5 wherein the gating defect is likely to be induced in Covid-19 epidemic or pandemic.

In this aspect, invention also provides a method of avoiding or minimizing occurrence of a cardiac disorder comprising administering a pharmaceutical composition comprising therapeutically effective amount of cannabidiol wherein the cardiac disorder arises from gating defects in sodium channel Nav1.5 wherein the gating defect is likely to be induced in Covid-19 epidemic or pandemic.

In treatment of Covid-19, CANNABIDIOL can be simultaneously or sequentially administered with one or more other therapeutic agent such as an antiviral drug. Particularly, CANNABIDIOL is administered simultaneously or sequentially with chloroquine/hydroxychloroquine and optionally azithromycin.

The inventors have found that the role of CANNABIDIOL in treating Covid-19 would be multi-fold. CANNABIDIOL is considered safe for chronic use and is cardioprotective in nature. It can reduce cytokines and acts against inflammation. Most importantly, it reduces late sodium current, prolongation of action potential and LQT and can prevent/rescue hyperexcitability of cardiac ion channels. CANNABIDIOL can rescue LQT induced in patients suffering from Covid-19. Further, it can enhance safety profile of the treatment which recommends administration of therapeutic agents for treating Covid-19 although such therapeutic agents are capable of causing LQT which will enable masses to receive Covid treatment in best possible manner.

Additionally, some of the Covid-19 vaccines are also reported to induce LQTs. Thus, CANNABIDIOL can be administered along with vaccines which will enable masses to receive Covid vaccines in best possible manner.

In the sixth aspect, pharmaceutical compositions of CANNABIDIOL are administered even to healthy population as a prophylactic therapeutic agent to avoid occurrence of any cardiac disorder where sodium channel gating properties are affected.

Such administration to a healthy population is also done when there is likelihood of Covid-19 such as during epidemic or pandemic of Covid-19.

Additionally, under this aspect, CANNABIDIOL pharmaceutical compositions are administered even to healthy population when there is likelihood of any epidemic or pandemic which is likely to induce Long QT.

Accordingly, invention provides a pharmaceutical composition comprising therapeutically effective amount of cannabidiol for use in prophylaxis or prophylactic treatment for avoiding or minimizing occurrence of a cardiac disorder arising from gating defects in sodium channel Nav1.5.

In this aspect, invention also provides a method of prophylaxis or prophylactic treatment for avoiding or minimizing occurrence of a cardiac disorder comprising administering a pharmaceutical composition comprising therapeutically effective amount of cannabidiol wherein the cardiac disorder arises from gating defects in sodium channel Nav1.5.

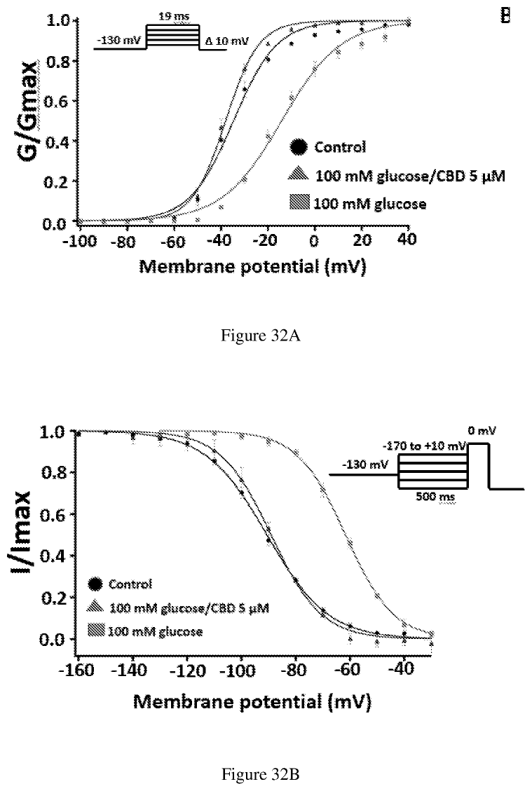

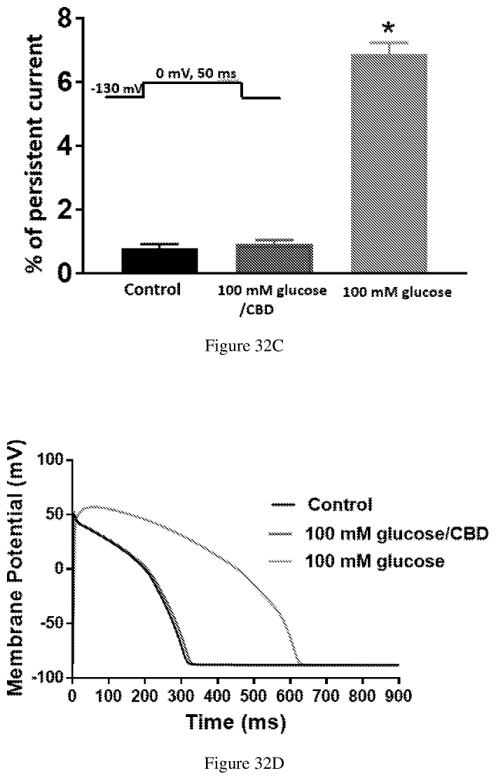

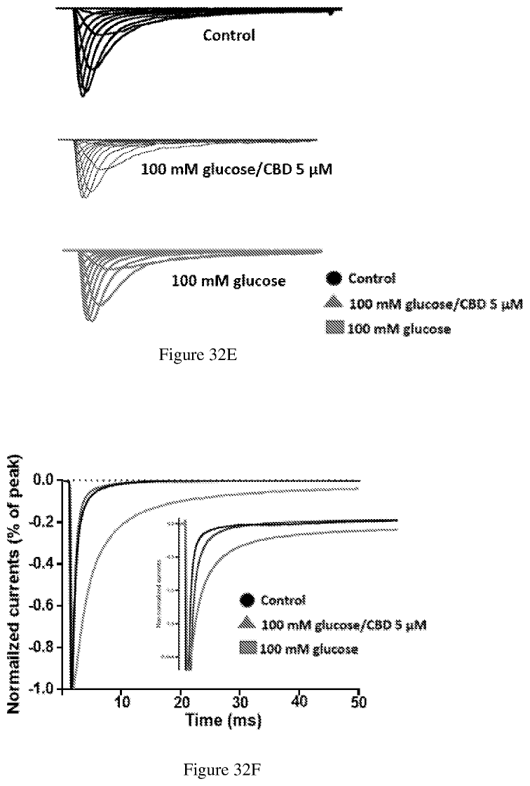

The inventors of the present invention studied various electrophysiological changes in sodium channels Nav1.5 under a condition to mimic another condition where cannabidiol compositions are used for prophylaxis or prophylactic treatment. The process is described under example 41 and data is provided under FIGS. 32A-32E. High glucose incubation shifts the activation and steady-state inactivation curves to the right (FIGS. 32A and 32B, red data) and increases late sodium current (FIG. 32C, red data). These changes predict prolongation of the ventricular action potential (FIG. 32D). Co-incubation of Cannabidiol (CBD) with high glucose rescues the activation and steady-state inactivation curves (FIGS. 32A and 32B, blue data) and reduces late sodium current (FIG. 32C) to control values (data shown in black). Co-incubation with CBD is predicted to rescue action potential prolongation (FIG. 32D). FIGS. 32E and 32F show current traces recorded during the activation protocol (32E) and the persistent current protocol (32F). It is surprisingly found that Co-incubation with Cannabidiol rescues the late sodium current to amplitudes indistinguishable from control.

Under the seventh aspect, the invention provides various pharmaceutical compositions employing the new therapeutic agent CANNABIDIOL to rescue the adversely affected sodium channels Nav1.5 from the effects of formation of reactive oxygen species and conditions produced further from these effects. Reactive oxygen species formation causes oxidative stress and/or damage and leads to cytotoxicity. As a result, cell viability is reduced.

The invention further provides uses of these pharmaceutical compositions i) for reducing ROS formation and ii) for treating conditions produced due to formation of reactive oxygen species. The invention also includes treating patients suffering from i) effects ROS formation on Sodium channels Nav 1.5 and ii) conditions produced further from these effects by administering suitable pharmaceutical compositions employing CANNABIDIOL.

First part of this study helps the present inventors to arrive at the various aspects of the invention described above.

Second part of the study focuses on the electrophysiological changes in the gating properties of the sodium channel induced by another mediator, inflammation. While second part of the study focusses on the effects of inflammation on gating properties of the sodium channel nav1.5 and rescuing of the channels by the new therapeutic agent, it is understood that even high glucose conditions can and do cause inflammation.

The first and second parts of the study are not mutually exclusive. High glucose induces inflammation. Inflammation induced by any means such as whether disease or therapeutic agent or vaccine or any other factor, produce gating defects in sodium channels similar to those by high glucose.

Both the first and second parts of the study do not restrict in anyway the use of any mediators, but they merely indicate happening of two different preconditions leading to adversely affected sodium channels which are rescued by the new therapeutic agent CANNABIDIOL. Many other preconditions may also lead to gating defects in sodium channel Nav1.5 causing late or persistent sodium current, prolongation of action potential and LQTs and arrythmias. The pharmaceutical compositions of the new therapeutic agent aim to rescue such changes and aims to restore normal electrophysiology thus abolishing or minimizing happening of cardiac disorders.

In the second part of the study under the eighth aspect, the invention provides various pharmaceutical compositions employing the new therapeutic agent CANNABIDIOL for

1. abolishing or minimizing inflammation induced alteration in the gating properties of Nav1.5; and
2. rescuing sodium channels Nav1.5 from inflammation induced alteration in the gating properties.
3. The invention further provides uses of these pharmaceutical compositions of CANNABIDIOL for avoiding, abolishing or minimizing inflammation induced defects in the gating properties of Nav1.5 (alteration in the gating properties of Nav1.5) and treating to rescue channels or restore electrophysiology by administering pharmaceutical compositions employing new therapeutic agent CANNABIDIOL.

Further under the eighth aspect, the invention provides pharmaceutical compositions of CANNABIDIOL for treating or avoiding inflammation induced by any other therapeutic agent or inflammation induced in any diseases or ailment such as Covid-19 and inflammation induced by any vaccine such as Covid-19 vaccine.

Accordingly, invention provides a pharmaceutical composition comprising therapeutically effective amount of cannabidiol for use in treatment of a cardiac disorder arising from gating defect in sodium channel Nav1.5 induced or likely to be induced by inflammation.

In this aspect, invention further provides a method of treating cardiac disorder in a patient suffering from such disorder comprising administering a pharmaceutical composition comprising therapeutically effective amount of cannabidiol wherein the cardiac disorder arises from gating defects in sodium channel Nav1.5 induced or likely to be induced by inflammation.

Variants of the Nav subtype predominantly expressed in skeletal muscles is Nav1.4. Pathogenic conditions of Sodium channels Nav1.4 lead to contractility dysfunction. Although this condition is not considered lethal, it can be life-limiting due to the multitude of contractility problems it can cause, including stiffness and pain.

Under the ninth aspect, the invention provides various pharmaceutical compositions employing the new therapeutic agent CANNABIDIOL to rescues the adversely affected sodium channels Nav1.4 from the contractility dysfunction and conditions produced further from these effects such as muscle stiffness, pain, myotonia, gating-pore current in the VSD leading to periodic paralyses etc.

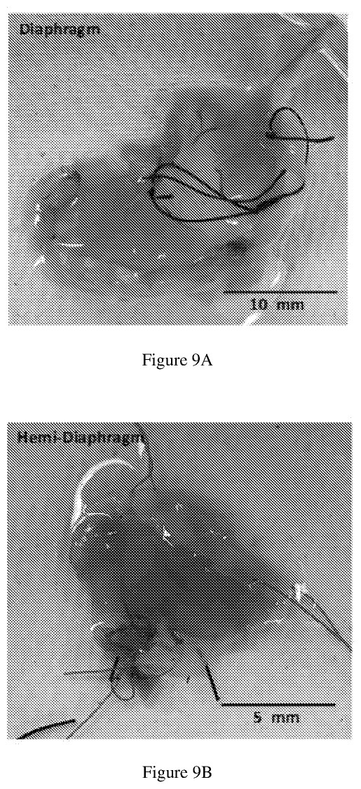

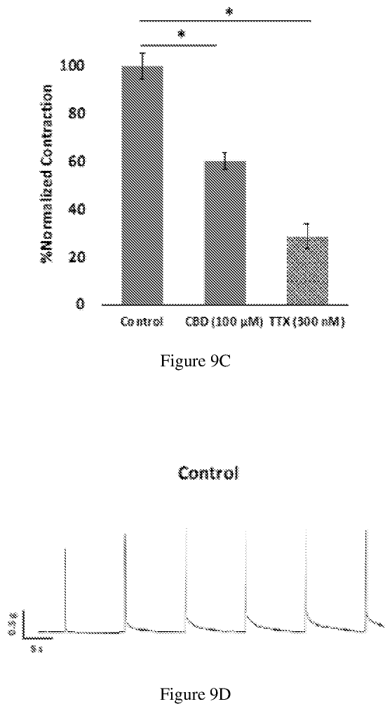

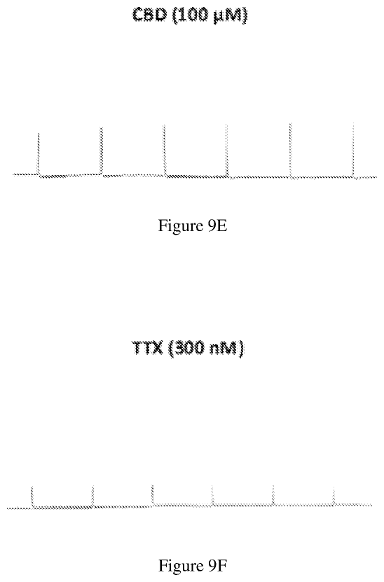

A rat diaphragm muscle is surgically removed, and muscle contractions evoked by phrenic nerve stimulation with electrodes are measured. Further, at a saturating concentration of 100 μM of CANNABIDIOL, muscle contractions evoked by phrenic nerve stimulation with electrodes are measured. The inventors have surprisingly found that CANNABIDIOL reduced contraction amplitude to ˜60% of control (p<0.05) (FIG. 9C). To confirm this, a known blocker is tested for a similar action as a control therapeutic agent or reference compound. A 300 nM saturating concentration of tetrodotoxin (TTX), a potent blocker of selected Nav channels (IC50˜10-30 nM on TTX-sensitive channels 39) is used. It is found that TTX reduced contraction to ˜20% of control (p<0.05) (FIG. 1C) confirming that CANNABIDIOL's contraction reduction is in part due to activity at Nav1.4.

Yet another aspect, a tenth aspect of the invention provides pharmaceutical compositions of the CANNABIDIOL which is the new therapeutic agent for restoring electrophysiology of sodium channels thus avoiding, abolishing or minimizing happening of cardiac disorders which mainly happen due to late or persistent sodium channels, prolongation of action potential, Long QT arrythmias etc.

The pharmaceutical compositions of CANNABIDIOL are provided as a solo pharmaceutical composition or in a combined form along with other therapeutic agents. In combined form, CANNABIDIOL can be provided in a separate pharmaceutical composition along with pharmaceutical composition of the other therapeutic agent or in the same pharmaceutical composition as that of the other therapeutic agent.

Pharmaceutical compositions of CANNABIDIOL comprise at least one pharmaceutically acceptable ingredient. CANNABIDIOL is reported to have extremely low solubility in water and it is photosensitive. On degradation it is likely to produce Tetrahydrocannabinol.

CANNABIDIOL pharmaceutical compositions according to the present invention preferably employ an agent capable of imparting solubility and/or stability. The pharmaceutical compositions may further employ an ingredient enhancing bioavailability of CANNABIDIOL.

Additionally, the process of preparing dosage forms of CANNABIDIOL should be carefully designed such that it should not cause degradation of CANNABIDIOL. The various pharmaceutical compositions and processes used to prepare those pharmaceutical compositions are provided under the tenth aspect.

All aspects are described hereinbelow in great details by various experimentation and results.

First part of the study employed high glucose conditions which adversely affected sodium channels Nav1.5. These channels are further subjected to following studies,

A. Electrophysiological experiments.
B. Cell viability studies;
C. Reactive Oxygen species measurement.

Further, therapeutic agents such as Lidocaine and Tempol and a new therapeutic agent CANNABIDIOL are employed in the experiments to check if these therapeutic agents can rescue the adversely affected sodium channels. Hence, the channels after treatment with therapeutic agents are again subjected to above mentioned studies/experiments and the difference between the two studies are recorded and presented under various figures provided.

Electrophysiological experiments further involved following:

a. Activation;
b. Steady state fast inactivation;
c. Recovery from fast inactivation;
d. Late or persistent sodium currents; and
e. Action potential modelling;

### Electrophysiology

Electrophysiology, an electrical recording technique that enables the measurement of the flow of ions (ion current) in biological tissues is employed. Whole cell patch clamp technique as described under example 3 is employed.

Using this technique, inventors examined the effects of glucose at four concentrations (10 (normal), 25 (high), 50 (higher), and 100 mM (higher)) on Nav1.5 activation by measuring peak channel conductance between −130 and +80 mV.

### Activation Protocol

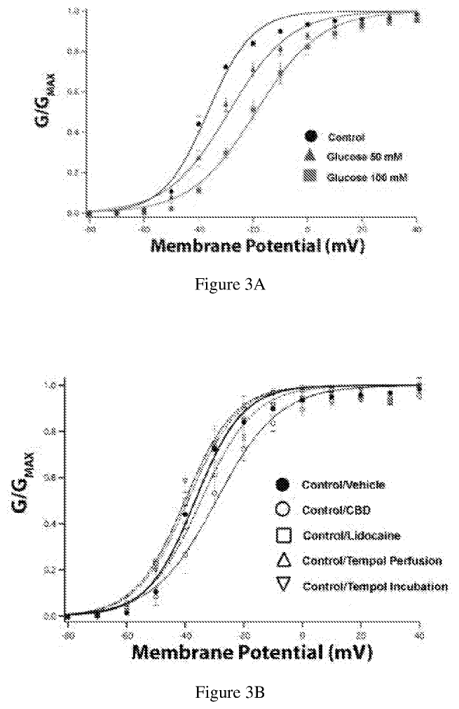

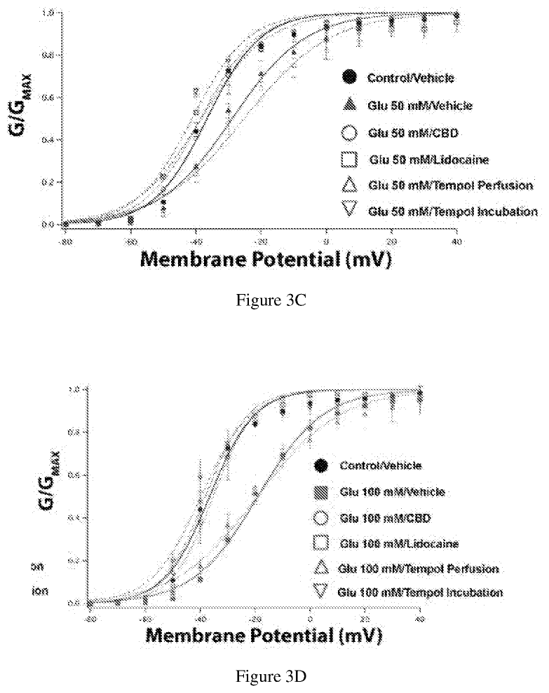

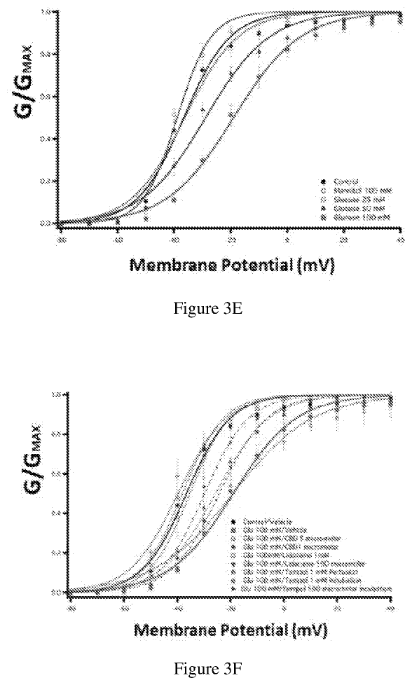

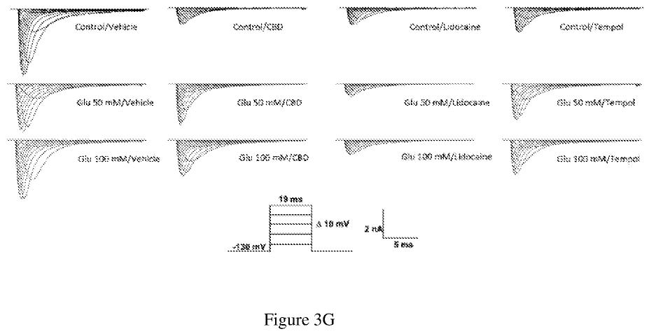

FIG. 3A provides the Nav1.5 conductance plotted as a function of membrane potential. High glucose (50 or 100 mM) significantly shifted the Nav1.5 midpoint (V) of activation in the positive direction in a concentration-dependent manner (50 mM: P=0.01; 100 mM: P=0.001) with no significant effect elicited by 25 mM glucose or mannitol (100 mM, osmotic control) (FIG. 3E and Table 1). This suggests that higher glucose concentrations make Nav1.5 less likely to activate at any given membrane potential.

To determine whether the change in Nav1.5 activation due to glucose incubation could be rescued, inventors measured channel conductance in the presence of CANNABIDIOL, lidocaine, or Tempol and found that none of CANNABIDIOL (perfusion), lidocaine (perfusion), or Tempol (perfusion or incubation) exerted any significant effect on voltage-dependence of activation of Nav1.5 under the control condition (10 mM glucose) (P>0.05) (FIG. 3B and Table 1).

Perfusion of CANNABIDIOL (1 or 5 μM), lidocaine (100 μM or 1 mM), or co-incubation of Tempol (100 μM or 1 mM) (for 24 hours) abolished the high glucose (50 or 100 mM)-elicited shifts of V and the apparent valence of activation in a concentration-dependent manner (FIG. 3C, 3D, 3F and Table 1). Tempol perfusion had no effects on high glucose-evoked alterations in Nav1.5 activation (FIGS. 3C and 3D). CANNABIDIOL and lidocaine may work on the level of Nav1.5 in the membrane, and hence, may not need the long exposures required by Tempol. Perfusion of CANNABIDIOL reduced the current density of Nav1.5 with no significant difference between the control condition (from −2.05±0.61 to −0.87±0.23 nA/pF) or the high glucose (50 or 100 mM) (from −2.40±0.85 to −1.19±0.46 nA/pF or from −2.86±0.76 to −0.95±0.29 nA/pF, respectively). This study suggests that CANNABIDIOL can rescue change in Nav1.5 activation due to glucose incubation.

### Steady-State Fast Inactivation (SSFI)

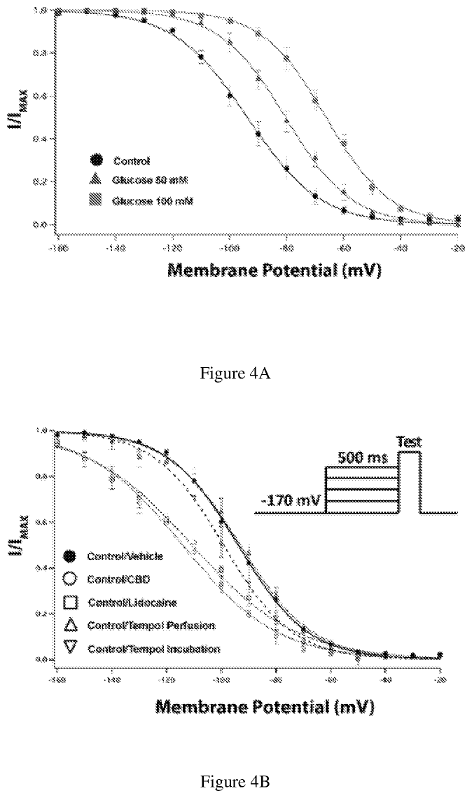

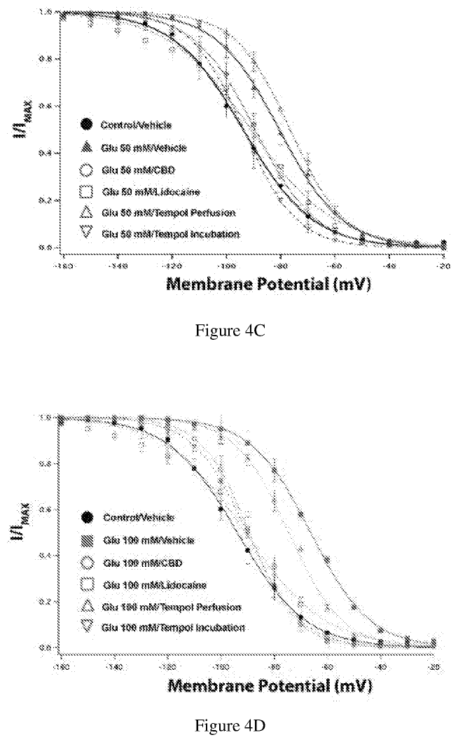

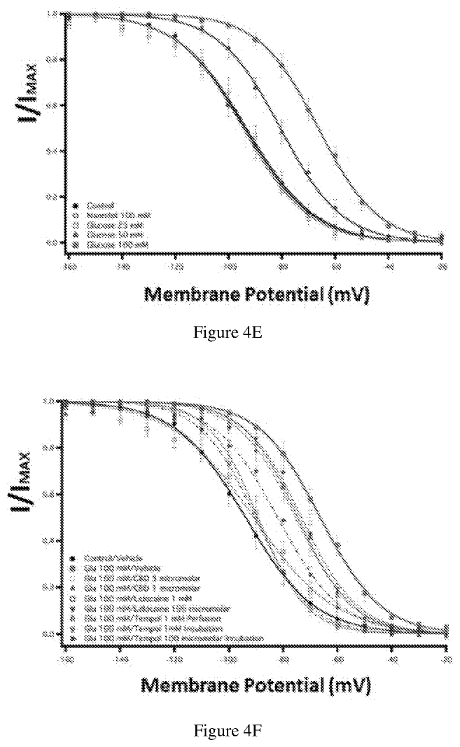

As provided in FIG. 4A, voltage-dependence of steady state fast inactivation (SSFI) as normalized current is plotted against membrane potential for control, mannitol and 3 glucose concentration to establish if higher glucose concentration adversely affects steady state fast inactivation and it has been surprisingly observed that higher glucose concentration right shifted the steady state fast inactivation.

Higher glucose (50 or 100 mM) caused significant positive shifts in the V obtained from Boltzmann function fits at high glucose (50 mM: P=0.019; 100 mM: P=0.001) (FIG. 4A and Table 2). These shifts suggest a gain-of-function in the voltage-dependence of Nav1.5 SSFI and suggest that, at any given membrane potential, Nav1.5 is less likely to inactivate at higher glucose concentrations. This may lead to prolongation of action potential (hyperexcitability) which could result in long QT3 arrhythmia. High glucose (25 mM) or the mannitol (100 mM, osmotic control for high glucose) had no effects on the voltage-dependence of Nav1.5 steady-state fast inactivation (FIG. 4E and table 2).

To determine whether the destabilized SSFI in Nav1.5 could be rescued, the inventors measured inactivation in the presence of CANNABIDIOL, lidocaine, or Tempol. It is found that both CANNABIDIOL and lidocaine shifted the inactivation curves to the left (FIG. 4B). However, neither perfusion nor incubation with Tempol caused a significant left shift of SSFI of Nav1.5 under the control condition (FIG. 4B).

Next, the inventors performed the same experiments after incubation in 50 or 100 mM glucose. Both CANNABIDIOL (1 or 5 μM) and lidocaine (100 μM or 1 mM) shifted the inactivation curves to the left in a concentration-dependent effect (FIG. 4D and Table 2).

Interestingly, although the Tempol perfusion did not change the high glucose-induced effects on SSFI, Tempol (100 μM or 1 mM) incubation concentration-dependently shifted the curve to the left (FIG. 4D and Table 2).

Recovery from Fast Inactivation

Unless Sodium channels recover from fast inactivation, they cannot get deactivated and unless they are deactivated, they are not ready to take part in further action potential. Thus, one of the key biophysical features of sodium channels is the kinetics at which they recover from inactivated states.

To measure fast inactivation recovery, inventors held channels at −130 mV to ensure channels were fully at rest, then pulsed the channels to 0 mV for 500 ms, and allowed different time intervals at −130 mV to measure recovery as a function of time.

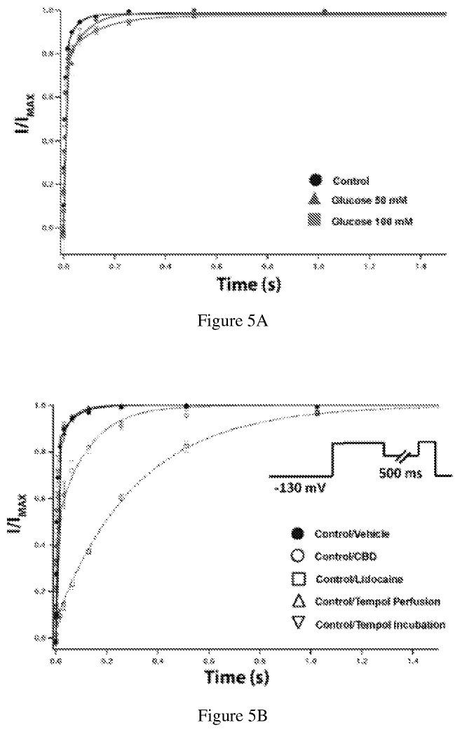

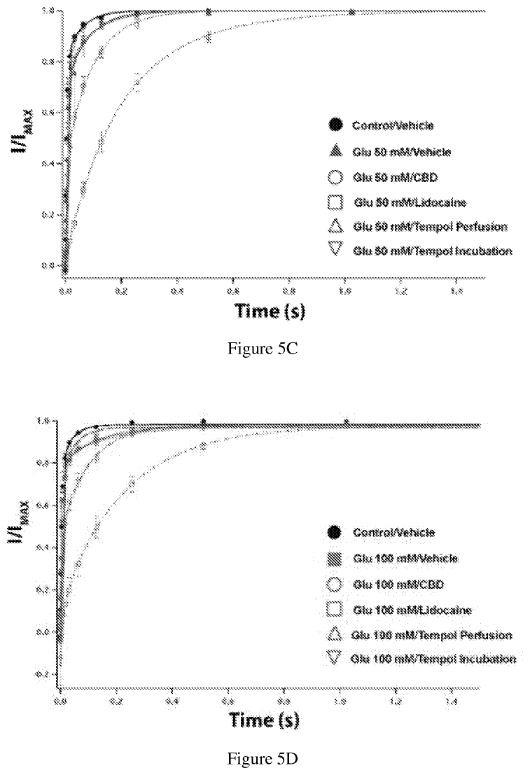

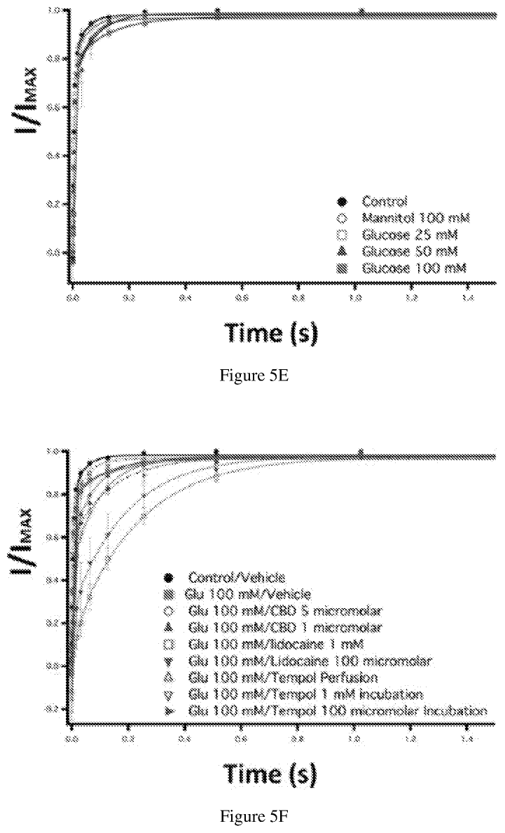

Recovery from fast inactivation is provided under FIG. 5A-5F where the normalized current is plotted against a range of recovery durations. As provided in FIG. 5A, effect of high glucose (25, 50 or 100 mM) or mannitol (100 mM) on recovery from fast inactivation of Nav1.5 transfected cells is seen. It is found that incubation in high glucose significantly (P<0.05, though with a relatively small magnitude of difference) increase the slow component of fast inactivation recovery when compared to control (FIG. 5A, FIG. 5E and table 3).

In addition, CANNABIDIOL, lidocaine, or co-incubation with Tempol significantly (P=0.0032, P<0.0001, or P=0.0013, respectively) increased, in a concentration-dependent effect, the time constant of the slow component of recovery from fast inactivation regardless of the glucose concentration (control or high concentration) (FIG. 5B, FIG. 5F and Table 3). However, only lidocaine, but not CANNABIDIOL or Tempol, increased the time constant of the fast component of recovery from fast inactivation regardless of the glucose concentration (FIG. 5B and Table 3). These findings suggest that glucose causes a slight loss-of-function to the fast inactivation recovery of Nav1.5, and the tested compounds further stabilized the inactivated state of the channel (Ghovanloo, Shuart, Mezeyova, Dean, Ruben & Goodchild, 2018; Nuss, Tomaselli & Marban, 1995; Wang, Mi, Lu, Lu & Wang, 2015).

### Sodium Persistent Currents

Stabilization of fast inactivation and recovery from fast inactivation is desired. An increased sodium persistent current is a manifestation of destabilized fast inactivation and hence undesired. Large persistent sodium currents are associated with a range of pathological conditions, including LQT3 (Ghovanloo, Abdelsayed & Ruben, 2016; Wang et al., 1995). To determine the effects of glucose on the stability of Nav1.5 inactivation, the channels were held at −130 mV, followed by a depolarizing pulse to 0 mV for 200 ms to elicit persistent currents.

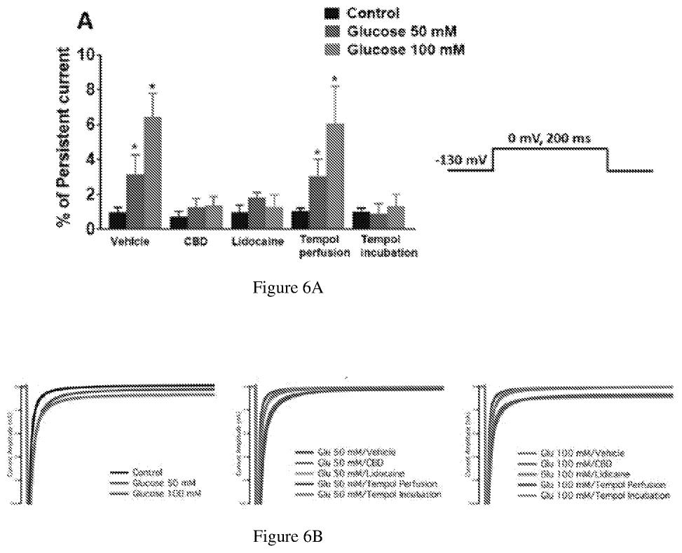

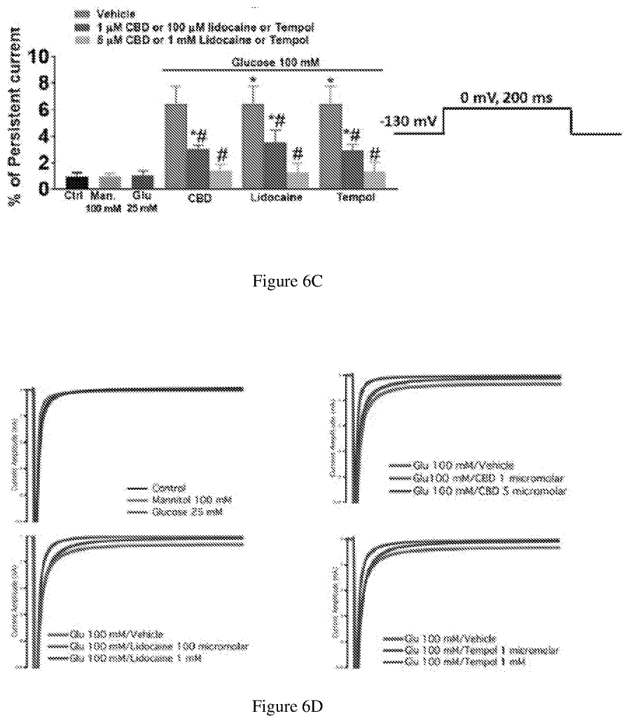

As seen from FIG. 6A, incubation in high glucose (50 or 100 mM) significantly (50 mM: P=0.003; 100 mM: P=0.001) increased persistent currents compared to control. On the other hand, neither glucose (25 mM) nor mannitol (100 mM) had any effects on persistent currents compared to control (FIG. 6C).

Although perfusion of CANNABIDIOL, lidocaine or Tempol (perfusion or incubation) had no effect on the small persistent currents in the control condition (FIG. 6A), each of the three compounds significantly concentration dependently reduced the high-glucose (50 or 100 mM)-induced increase in Persistent sodium currents (FIG. 6C and Table 4). In contrast, Tempol perfusion had no effect on high glucose (50 or 100 mM)-elicited increased persistent currents (FIG. 6A and Table 4). Reduction of the exaggerated persistent currents at high glucose by CANNABIDIOL is consistent with the previous reports in neuronal sodium channels (Ghovanloo, Shuart, Mezeyova, Dean, Ruben & Goodchild, 2018; Patel, Barbosa, Brustovetsky, Brustovetsky & Cummins, 2016).

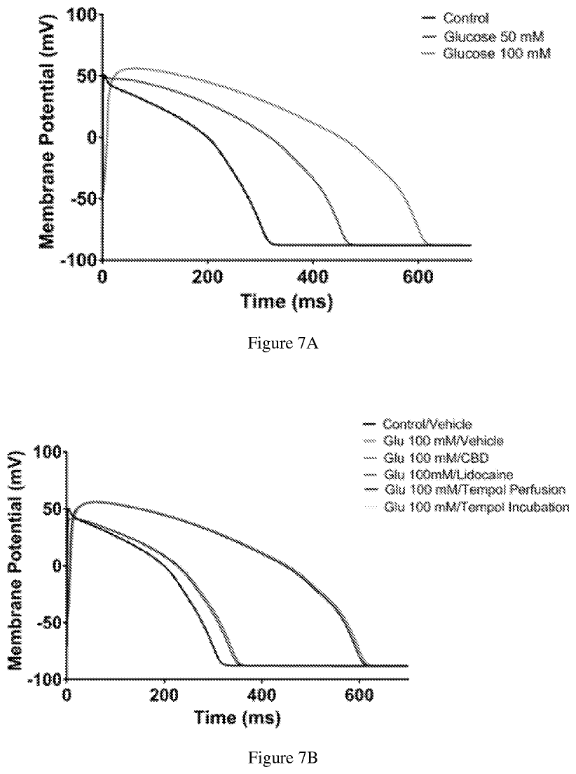

Further, FIG. 7A provides action potential model simulation. Incubation in high glucose caused a concentration dependent prolongation of the action potential duration from ˜300 ms to ˜450 ms in 50 mM glucose, and to >600 ms in 100 mM glucose (FIG. 7A). As reported by Nachimuthu et al. this increased action potential duration could potentially lead to the prolongation of the QT interval.

Further, to establish whether CANNABIDIOL and others rescue the high glucose-elicited prolongation of the action potential duration to nearly that of the control, the incubation in glucose is done in presence of CANNABIDIOL, lidocaine, or Tempol. The simulation results suggest that CANNABIDIOL, lidocaine, or incubation (but not perfusion) with Tempol rescues prolongation of action potential condition (FIG. 7B). This reduction in the predicted excitability is consistent with the anti-excitatory effects attributed to the compounds used, in particular CANNABIDIOL and lidocaine (Ghovanloo, Shuart, Mezeyova, Dean, Ruben & Goodchild, 2018; Nuss, Tomaselli & Marban, 1995).

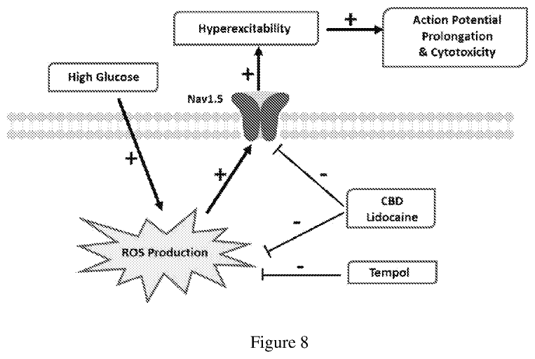

FIG. 8 provides a schematic of possible cellular events involved in the protective effect of CANNABIDIOL, lidocaine or Tempol against high glucose induced oxidative effects and cytotoxicity via affecting cardiac voltage-gated sodium channels (Nav1.5).

Thus, it has been observed in case of CANNABIDIOL that it has:

1. enhanced cell viability in higher glucose environment;
2. exerted ions complete reduction of ROS levels in higher concentration;
3. rescued the change in Nav1.5 activation due to glucose incubation wherein because of higher glucose concentrations Nav1.5 is less likely to activate at any given membrane potential;
4. rescued Nav1.5 which otherwise is less likely to inactivate at higher glucose concentrations which may lead to prolongation of action potential (hyperexcitability) which could result in long QT3 arrhythmia. The findings of the inventors implicate the role of Nav1.5 in high glucose induced hyperexcitability and cytotoxicity, via oxidative stress, which could lead to LQT3 arrythmia (FIG. 8);
5. significantly concentration dependently reduced the high-glucose (50 or 100 mM)-induced increase in persistent sodium currents. Persistent sodium currents is a manifestation of destabilized fast inactivation;
6. rescues Nav1.5 from prolongation of the action potential caused by high glucose.

The invention under the fourth aspect provides pharmaceutical compositions of CANNABIDIOL, a sodium channel modulator to reverse/prevent drug induced LQT thus enabling patients with LQT or patients susceptible to LQT to receive best possible treatment.

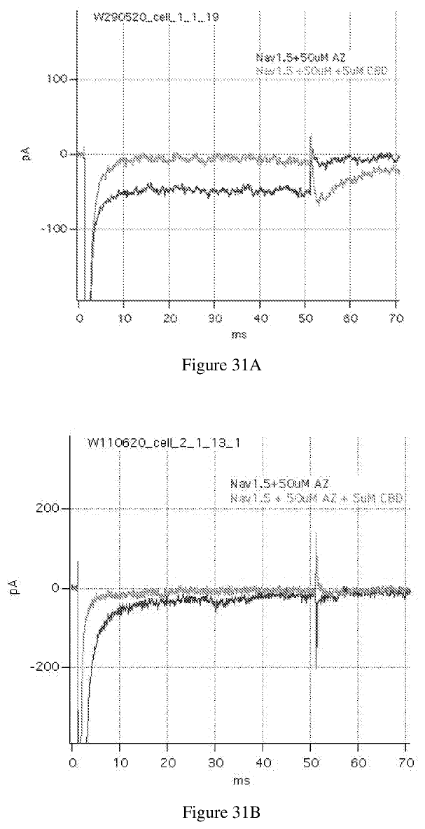

The present inventors have demonstrated effects of Azithromycin on Sodium channel Nav1.5 wherein they incubated cells heterologously expressing Nav1.5 in 10 μM Azithromycin and observed an increase in late sodium current compared to control (no Az incubation) cells. FIG. 31A provides a plot of current amplitude plotted against time in milliseconds and the plot provides late sodium current when cells are incubated with Azithromycin. Further the inventors perfused the cells showing Azithromycin-induced late sodium current with 5 μM CANNABIDIOL and observed that the late current was reduced. Thus, CANNABIDIOL rescues the proarrhythmic effects of Azithromycin and, thus, may be a useful as adjuvant therapy in conditions that call for treatment with macrolide antibiotics, possibly including COVID-19.

FIG. 31B provides the same experiment conducted on different cells.

The pharmaceutical compositions of CANNABIDIOL are proposed to be administered simultaneously or sequentially with the other drug or combination of drugs wherein at least one such drug is likely to produce drug induced LQT.

Simultaneous administration of CANNABIDIOL includes administering CANNABIDIOL along with at least one drug capable of inducing LQT. The CANNABIDIOL can be added in the same pharmaceutical composition as that of such other drug or CANNABIDIOL can be present in different dosage form but administered simultaneously or sequentially at the same time when the other drug is administered. Administering at the same time as the term appears here means that the CANNABIDIOL is physically administered when the other dug is physically administered and it also means that CANNABIDIOL is administered in presence of other drug in biological environment or the other drug is administered when CANNABIDIOL is in biological environment.

Sequential administration means that CANNABIDIOL and the other drug are not physically administered together but they are administered with some time gapin between. The sequential administration is preferred when CANNABIDIOL is likely to physically interact with the other drug. It is also preferred when the frequency of administration of CANNABIDIOL does not match with the frequency of administration of other such drug.

In simultaneous administration, whether CANNABIDIOL can be administered in same or different dosage form will depend on several factors such as various pharmacokinetic factors, compatibility of CANNABIDIOL with the other drug, compatibility of the other drug with a desired inactive ingredient of CANNABIDIOL, high doses of both CANNABIDIOL and the other drug not capable of combining them in one dosage form etc. The desired inactive ingredient present in CANNABIDIOL pharmaceutical composition usually is an agent which enhances solubility of CANNABIDIOL such as binder, surfactant, solubilizer, disintegrant, solvent etc.

This aspect provides pharmaceutical compositions of CANNABIDIOL and that other therapeutic agent in the same or different pharmaceutical composition to facilitate simultaneous and/or also sequential administration to suit different dosing frequency and/or route of administrations of the two drugs.

Nachimuthu et al provides a list of drugs leading to acquired Long QT in his article titled, “Drug-induced QT interval prolongation: mechanisms and clinical management”. The present invention enhances safety profile of all such drugs and any other drug not listed there but with a same or similar safety concern by co-administering pharmaceutical compositions of CANNABIDIOL.

Where co-administration involves simultaneous administration, the two pharmaceutical compositions viz. CANNABIDIOL pharmaceutical composition and pharmaceutical composition of other therapeutic agent causing long QT can be formulated in a single pharmaceutical composition or two separate pharmaceutical compositions administered together.

A single formulation without limitation can be for example a bi-layered or a tri-layered tablet, capsules having different mixtures where each mixture may have one active or capsule having two types of pellets/beads/granules/slugs etc. each having a different therapeutic agent or a liquid having two actives etc.

The single formulation can be for example a bi-layered or a tri-layered tablet having following combinations,

1. two therapeutic agents in a bi-layered tablet where each layer before compression has one therapeutic agent or one layer before compression is a layer having ingredients other than actives and both the therapeutic agents are in the other layer before compression;
2. two therapeutic agents in a tri-layered tablet where each layer has one therapeutic agent and a third layer contains non-actives wherein the third layer can be on side or in between the two layers having actives (active ingredients) or one or more layers before compression is a layer having ingredients other than actives.
3. three therapeutic agents in a tri-layered tablet where each layer has one therapeutic agent or one or more layers before compression is a layer having ingredients other than actives.

When it is not possible to provide two or more actives such as CANNABIDIOL and Long QT inducing therapeutic agent(s) in a single pharmaceutical composition due to any reasons such as without limitation different stability profile, different route of administration, large doses leading to bulkier formulation, or different dosing frequency etc., the CANNABIDIOL pharmaceutical compositions are provided with the pharmaceutical compositions of the other therapeutic agent in kit forms. Hence, under this aspect, the pharmaceutical compositions in kit forms are provided.

According to the fourth aspect of the invention, the reduction in QT makes CANNABIDIOL an ideal agent that can be used with all drugs capable of inducing LQT. This will enhance safety profile of any treatment, any drug or combination of drugs that would otherwise cause LQT.

Recently several studies are being conducted including in vitro studies and clinical trials involving at least one antimalarial such as chloroquine and hydroxy chloroquine to explore potential treatment of Covid-19 optionally with Azithromycin. Both chloroquine/hydroxy chloroquine and azithromycin are known to produce drug induced LQT. In Gaurett's study, some patients were specifically excluded from the study including those with QT prolongation.

CANNABIDIOL is a potential therapeutic agent to rescue/prevent drug induced LQT and enhance safety profile of any treatment causing such prolongation of QT interval. This action of CANNABIDIOL is proposed to be mediated through modulation of the gating properties of one or more cardiac ion channels including sodium channels Nav 1.5. Thus, CANNABIDIOL is proposed as a therapeutic in treatment of Covid-19. Recently the inventors have found that CANNABIDIOL reduces the pro-arrhythmic effects of Azithromycin. The persistent sodium currents/late current produced by azithromycin are reduced by action of CANNABIDIOL which is shown in FIGS. 31A and 31B. From these results, it is apparent that CANNABIDIOL rescues the proarrhythmic effects of Azithromycin and, thus, may be a useful adjuvant therapy in conditions that call for treatment with macrolide antibiotics, possibly including COVID-19.

The invention covers various pharmaceutical composition that can be used for various treatments including antiviral treatments and particularly covering treatments for recent outbreak of Covid-19.

CANNABIDIOL pharmaceutical compositions can be administered when cardio-protection is needed in any treatment or when there is a need to reduce inflammation or need to reduce cytokine storm and most importantly when there is a need to rescue/reverse or avoid LQT whether inherited or acquired due to certain health conditions and/or drug induced.

The present invention provides pharmaceutical compositions and methods to enhance safety profile of any treatment including an antiviral treatment particularly including treatment for Covid-19 wherein the treatment includes administration of one or more drugs that may cause drug induced LQTs.

In treatment of Covid-19, CANNABIDIOL can be simultaneously or sequentially administered with one or more antiviral drugs. Particularly, CANNABIDIOL is administered simultaneously or sequentially with chloroquine/hydroxychloroquine and optionally azithromycin.

Under the fifth aspect, the invention provides pharmaceutical compositions of CANNABIDIOL for treating Covid-19. The inventors propose that Role of CANNABIDIOL in treating Covid-19 is multi-fold. CANNABIDIOL is considered safe for chronic use and is cardioprotective in nature. It can reduce cytokines and act as anti-inflammatory agent. Most importantly, it reduces LQT and can prevent/rescue hyperexcitability of cardiac ion channels. In this way, it can enhance safety profile of the treatment which recommends administration of drugs for treating Covid-19 but capable of causing LQT and enables masses to receive best possible treatment.

Additionally, some of the Covid-19 vaccines are also reported to induce LQTs CBD has the potential to enhance safety and efficacy of COVID-19 vaccines Thus, CANNABIDIOL can be administered along with vaccines which will enables masses to receive Covid-19 vaccines in best possible manner.

In the sixth aspect, CANNABIDIOL pharmaceutical compositions are administered even to healthy population as a prophylactic therapeutic agent to avoid happening of any cardiac disorder where sodium channel gating properties are affected.

Such administration to healthy population is also done when there is likelihood of Covid-19 such as during epidemic or pandemic of Covid-19.

Additionally, under this aspect, CANNABIDIOL pharmaceutical compositions are administered even to healthy population when there is likelihood of any epidemic or pandemic which is likely to induce Long QT.

In this aspect, invention also provides a method of prophylaxis or prophylactic treatment for avoiding or minimizing occurrence of a cardiac disorder comprising administering a pharmaceutical composition comprising therapeutically effective amount of cannabidiol wherein the cardiac disorder arises from gating defects in sodium channel Nav1.5.

The inventors of the present invention studied various electrophysiological changes in sodium channels Nav1.5 under a condition to mimic another condition where cannabidiol compositions are used for prophylaxis or prophylactic treatment. To mimic such a condition, it is necessary that at least one condition should be chosen where sodium channels are not adversely affected at the time when cannabidiol is added. Thus, co-incubation of sodium channels in cannabidiol (5 μM) and high glucose is chosen as a condition against all previous conditions where sodium channels were incubated in high glucose conditions for 24 hrs to induce gating defect before action of cannabidiol.

The process is described under example 41 and data is provided under FIGS. 32A-32E. The Chinese hamster ovary (CHO) cells transiently transfected with SCN5A, were incubated in control (10 mM glucose), high (100 mM) glucose, or high glucose and 5 uM CBD. High glucose incubation shifts the activation and steady-state inactivation curves to the right (FIGS. 32A and 32B, red data) and increases late sodium current (FIG. 32C, red data). These changes predict prolongation of the ventricular action potential (FIG. 32D). Co-incubation of Cannabidiol (CBD) with high glucose rescues the activation and steady-state inactivation curves (FIGS. 32A and 32B, blue data) and reduces late sodium current (FIG. 32C) to control values (data shown in black). Co-incubation with CBD is predicted to rescue action potential prolongation (FIG. 32D). FIGS. 32E and 32F show current traces recorded during the activation protocol (32E) and the persistent current protocol (32F). It is surprisingly found that Co-incubation with Cannabidiol rescues the late sodium current to amplitudes indistinguishable from control.

Under the seventh aspect, the invention provides various pharmaceutical compositions employing the new therapeutic agent CANNABIDIOL to rescue the adversely affected sodium channels Nav1.5 from the effects of formation of reactive oxygen species and conditions produced further from these effects. Reactive oxygen species formation causes oxidative damage and leads to cytotoxicity. As a result, cell viability is reduced. The invention further provides uses of these pharmaceutical compositions i) for reducing ROS formation and ii) for treating conditions produced due to formation of reactive oxygen species. The invention also includes treating patients suffering from i) effects of cytotoxicity and effects of ROS formation on Sodium channels Nav 1.5 and ii) conditions produced further from these effects by administering suitable pharmaceutical compositions employing CANNABIDIOL.

The results of cell viability studies and ROS measurement correlate well. The new therapeutic agent rescues sodium channel Nav1.5 from effects of reactive oxygen species and enhances cell viability.

### 2. Cell Viability Studies

To establish cytotoxicity caused by glucose, the current Inventors used Chinese hamster ovary (CHO) cells transiently co-transfected with cDNA encoding Nav1.5 α-subunit under control and high glucose conditions (50 or 100 mM for 24 hours). The experiment as provided under example 1 was conducted to establish the concentration-dependent cytotoxicity caused by glucose in the selected model system. The CHO cells were seeded at 50,000 cells/ml in a 96-well plate for 24 hours then treatments were started in normal (10 mM) or elevated (25-150 mM) glucose concentrations for another 24 hours in presence and absence of different treatments (CANNABIDIOL (1 or 5 μM), lidocaine (100 μM or 1 mM), Tempol (100 μM or 1 mM) or their vehicle). At the end of the incubation period (24 hours), cell viability was measured by MTS cell proliferation assay kit with absorbance measured at 495 nm in accordance with manufacturer's instructions (Abcam, ab197010, Toronto, Canada).

As provided in FIG. 1A, exposures to higher-than-normal glucose (i.e. >10 mM) for 24 hours caused a concentration-dependent reduction in cell viability (FIG. 1A). Moreover, the cells transfected with Nav1.5 exhibited a greater reduction in viability compared to untransfected cells (P<0.05) at glucose concentrations of 50, 100, or 150 mM (FIG. 1A). These findings suggest that high glucose levels reduce cell viability, with this effect being more pronounced upon Nav1.5 transfection. FIG. 1B provides cell viability of transfected cells which are exposed to/treated with i) vehicle, ii) CANNABIDIOL, iii) Lidocaine and iv) Tempol. As provided in FIG. 1B, cell viability of transfected cells treated with CANNABIDIOL is found best amongst all at high glucose concentrations of 50 and 100 mM.

To ensure that the reduction in cell viability is indeed due to the presence of Nav1.5 and not a by-product of the stress induced on cells by the transient transfection process, the viability of cells stably transfected with Nav1.5 to blank cells that underwent the transient transfection procedure without adding the cDNA for Nav1.5 (mock transfected) were compared. It was observed that the stably-transfected Nav1.5 cells exhibited a greater reduction in cell viability compared to mock transfected cells (P<0.05) at glucose concentrations of 50, 100, or 150 mM (FIG. 1C).

To ensure that there are no confounding effects imposed by loss of osmolarity, experiments were performed in the presence of mannitol (100 mM for 24 hours) as osmotic control for high glucose. (FIG. 1C). Mannitol (100 mM) had no significant effect on the cell viability of the stably transfected Nav1.5 compared to the untransfected or the mock transfected cells (FIG. 1C).

Further, as provided in FIG. 1D, to determine the possibility to pharmacologically attenuate the reduction in cell viability at high glucose, inventors co-incubated cells at different glucose concentrations with CANNABIDIOL, lidocaine, or Tempol. It was observed that co-incubation with CANNABIDIOL (5 μM) for 24 hours provides results better than an antiarrhythmic drug Lidocaine. CANNABIDIOL (5 μM) attenuated the reduction in cell viability at high glucose conditions (50 or 100 mM); however, lidocaine (1 mM) only partially reduced the glucose-elicited cytotoxicity (FIG. 1D). Co-incubation with the antioxidant Tempol (1 mM) showed similar results to CANNABIDIOL (FIG. 1D). This effect of CANNABIDIOL is also seen even in case of untransfected cells (FIG. 1E).

Lidocaine as a reference has been in two concentrations viz. 100 μM and 1 mM in the present studies whereas CANNABIDIOL has been used in much lower concentrations viz. 1 μM and 5 μM. The above results are quite encouraging for considering future role of CANNABIDIOL for improving cardiac health by acting as Nav1.5 modulator. This action is even more pronounced than the current antiarrhythmic lidocaine.

One of the key manifestations of high glucose levels is reactive oxygen species (ROS) formation. To determine whether the cell viability data from the previous experiments correlate with increased ROS formation, the inventors measured ROS levels using DCF fluorescence after incubation for 24 hours in elevated glucose concentrations (25-150 mM).

### 3. ROS Measurement

High glucose evoked cell death associated with elevation in reactive oxygen species (ROS). High glucose-induced increase in ROS production is correlated to apoptosis and cell death (Fouda & Abdel-Rahman, 2017; Fouda, El-Sayed & Abdel-Rahman, 2018).

Previous studies have reported that oxidative stress affects the biophysical properties of Nav1.5 through lipoxidation of the cell membrane and/or the inhibition of Nav1.5 trafficking to the cell membrane (Liu et al., 2013; Nakajima et al., 2010). Moreover, Yu et al has correlated changes in Nav1.5 function with LQT arrythmia in diabetic rats (Yu et al., 2018). The inventors of the present invention through experiments and findings from the experiments propose that high glucose, at least partly through oxidative stress, alters Nav1.5 function and leads to cytotoxicity and arrythmia.

Measurement of oxidative stress was done using 2′,7′-dichlorofluorescein diacetate (DCFH-DA), a detector of ROS (Korystov et al., Free radical research 43: 149-155, 2009). Fluorescence intensity was measured 30 min after the reaction initiation using a microplate fluorescence reader set at excitation 485 nm/emission 530 nm according to the manufacturer (Abcam, ab113851, Toronto, Canada). The ROS level was determined as relative fluorescence units (RFU) of generated DCF using standard curve of DCF (Fouda et al., The Journal of Pharmacology and Experimental Therapeutics 361: 130-139, 2017, Fouda et al., The Journal of Pharmacology and Experimental Therapeutics 364: 170-178. 2018).

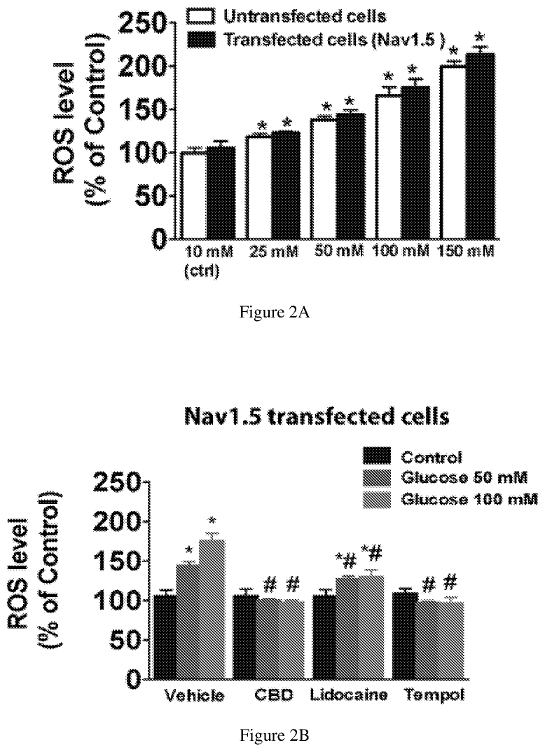

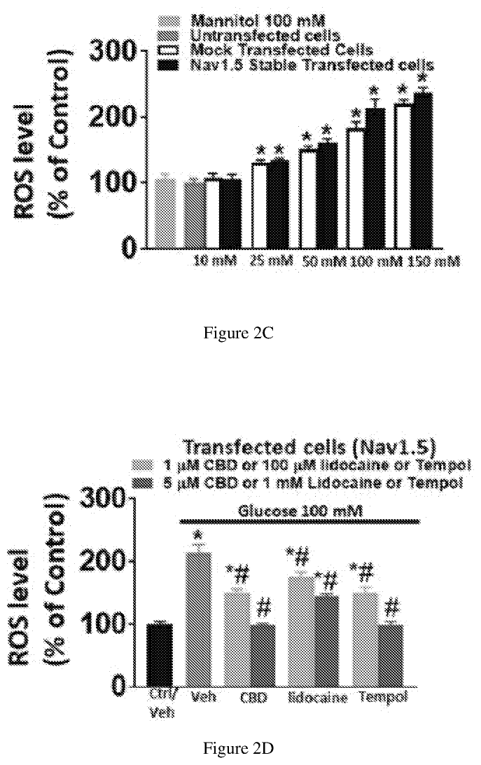

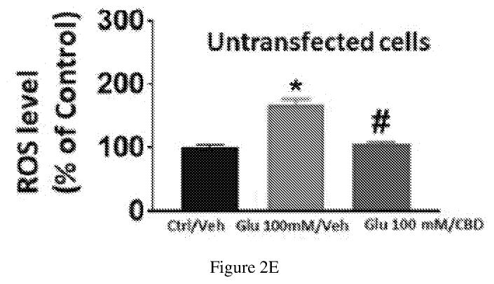

As provided in FIG. 2A, DCF fluorescence intensity showed a glucose concentration-dependent increase in the ROS level with no significant difference between the untransfected or the Nav1.5 transfected cells (FIG. 2A).

To determine the possibility to pharmacologically attenuate the reduction in ROS at high glucose, co-incubation of CANNABIDIOL (1 or 5 μM) or Tempol (100 μM or 1 mM) or lidocaine (100 μM or 1 mM) for 24 hours in transfected cells were carried out (FIG. 2B). It has been observed that higher concentrations of CANNABIDIOL or Tempol exert complete reduction of ROS levels (FIG. 2B and FIG. 2D), whereas lidocaine (100 μM or 1 mM) reduce ROS in a concentration-dependent manner and exerts only partial ROS reduction (FIG. 2B and FIG. 2D).

### Second Part of the Study

In addition to and apart from high glucose conditions wherein such high glucose conditions produced two following effects,

1. high glucose concentrations (up to 100 mM of D-glucose) mimicked adversely affected sodium channels in various cardiac ailments; and
2. high glucose concentrations (up to 100 mM of D-glucose) mimicked adversely affected sodium channels in hyperglycaemic/diabetic patients which represent a population more prone to various cardiac ailments,
other major condition which leads to cardiovascular disorders is inflammation.

Several researchers have reported role of inflammation in cardiac disorders as follows:

1. Cardiac inflammation has a key role in the development of cardiovascular anomalies (Adamo, Rocha-Resende, Prabhu & Mann, 2020);
2. Inhibition of inflammatory signalling pathways ameliorate cardiac consequences (Adamo, Rocha-Resende, Prabhu & Mann, 2020);
3. Importantly, ion channels are crucial players in inflammation-induced cardiac abnormalities (Eisenhut & Wallace, 2011).

However, the mechanisms underlying hyperglycemia-induced inflammation, and how inflammation provokes cardiac dysfunction, are not well understood.

R. G. Pertwee has also demonstrated that CANNABIDIOL is well tolerated without significant effects even when chronically administered in humans.

Since the new therapeutic agent CANNABIDIOL has rescued sodium channels Nav1.5 which is the major cardiac sodium channel isoform of the heart from the deleterious effects of high glucose, it is worth investigating further whether.

1. sodium channels Nav1.5 are affected by inflammation; and
2. CANNABIDIOL can rescue sodium channels Nav1.5 from the effects of inflammation.

Activation of sodium channels, steady state fast inactivation, stabilization of inactivation and recovery from inactivation are very essential for normal functioning of sodium channels. Unless Sodium channels recover from fast inactivation, they cannot get deactivated and unless they are deactivated, they are not ready to take part in further action potential.

The present inventors investigated effects of inflammation on sodium channels Nav1.5. They further investigated whether the new therapeutic agent CANNABIDIOL can rescue these channels from such effects.

To investigate effects of inflammation on sodium channels Nav1.5, high glucose condition and various inflammatory mediators are used.

For inflammation to affect sodium channels Nav1.5, at least one of the following effects should be observed due to action of these mediators;

1. sodium channels Nav1.5 are less likely to activate at any resting membrane potential;
2. sodium channels Nav1.5 do not inactivate i.e.; fast inactivation is affected;
3. recovery from fast inactivation is affected i.e., they are not ready to take part in further action potential.

In this study, apart from high glucose, a cocktail of inflammatory mediators provided by Akin et al (Akin et al., 2019) containing bradykinin (1 μM), PGE-2 (10 μM), histamine (10 μM), 5-HT (10 μM), and adenosine 5′-triphosphate (15 μM) is employed to induce inflammation and the effect of inflammation on the sodium channels Nav1.5 is investigated.

Further, as reported by Karin (Karin, 2005), one of the key signalling pathways involved in inflammation is the activation of protein kinase A (PK-A) or protein kinases C (PK-C) and subsequent protein phosphorylation. The activation of protein kinases (A and C) in a body in response to an inflammation can be mimicked by use of protein kinase activators.

Thus, effects of inflammatory mediators as well as activators of protein kinases on sodium channels Nav1.5 are investigated to mimic effects of inflammation and inflammatory pathways by using high glucose, cocktail of inflammatory mediators and protein kinase activators.

It is pertinent to test whether inflammatory mediators and activators of protein kinases indeed affect sodium channels Nav1.5 and if they do, whether these channels are rescued from their deleterious effects by anti-inflammatory compounds.

An anti-inflammatory agent is an agent which abolishes or minimizes/reduces inflammation. Just as Lidocaine and tempol are used as control in earlier experiments to compare performance of a new therapeutic agent CANNABIDIOL, protein kinase inhibitors are employed as control in the present study to compare anti-inflammatory effect of CANNABIDIOL on sodium channels Nav1.5. These inhibitors are capable of reversing/inhibiting inflammatory mediators.

Main purpose of the second part of the present study was to investigate effects of inflammation on Sodium channel Nav1.5 which is the major cardiac sodium channel isoform of the heart to provide therapeutic agents that can abolish or minimize or prophylactically prevent occurrence of cardiac dysfunction due to inflammation. Nevertheless, inflammation plays a bigger role in several other conditions.

Several researchers have shown following effects of hormones and particularly E2 on inflammation:

1. Gonadal hormones have crucial roles in the inflammatory responses (El-Lakany, Fouda, El-Gowelli, El-Gowilly & El-Mas, 2018; El-Lakany, Fouda, El-Gowelli & El-Mas, 2020);
2. Estrogen (E2), the main female sex hormone, acts via genomic and non-enomic mechanisms to inhibit inflammatory cascades (Murphy, Guyre & Pioli, 2010);
3. Clinically, postmenopausal females exhibited higher levels of TNF-α in reponse to endotoxemia compared with pre-menopausal women (Moxley, Stern, Carlson, Estrada, Han & Benson, 2004).
4. E2 stabilizes Nav fast inactivation and reduces the late sodium currents (Wang, Garro & Kuehl-Kovarik, 2010), similar to CANNABIDIOLeffects on Nav1.5 (Fouda, Ghovanloo & Ruben, 2020).

Thus, E2 seems to be one of the promising candidate to rescue sodium channels Nav1.5 from the deleterious effects of inflammation. Keeping this in mind, the present inventors designed a study protocol to cover following studies:

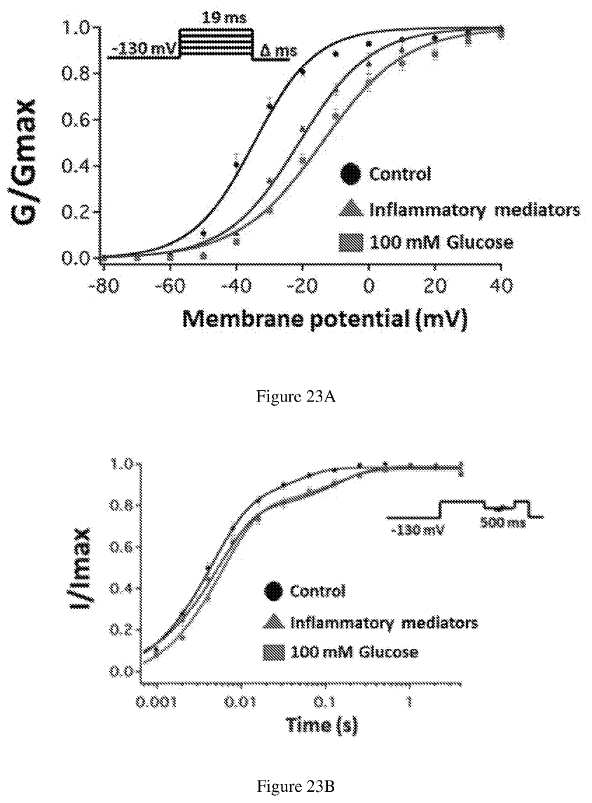

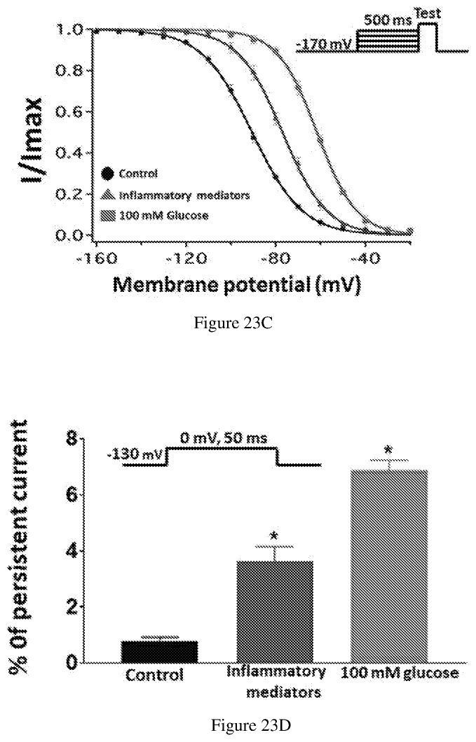

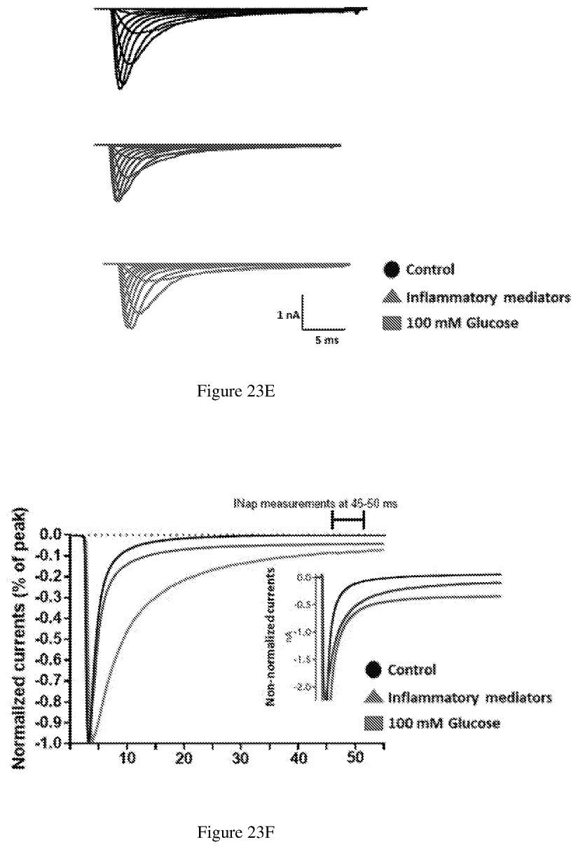

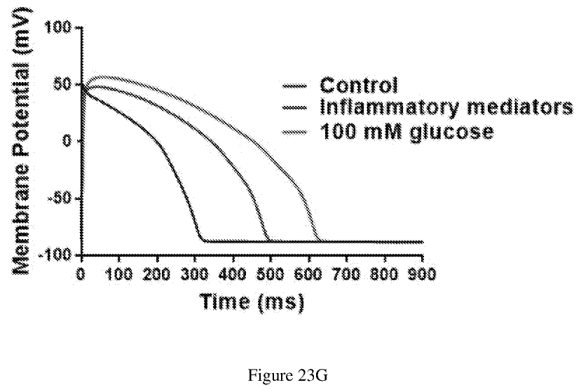

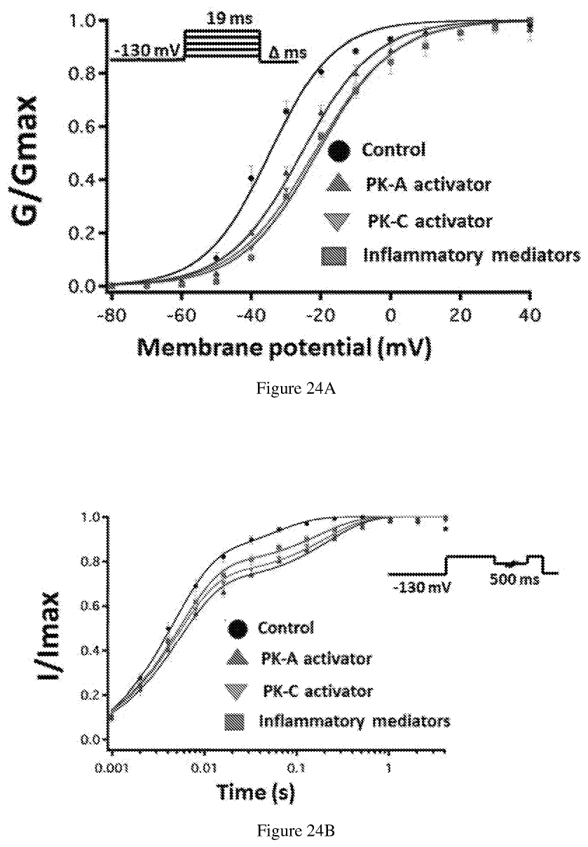

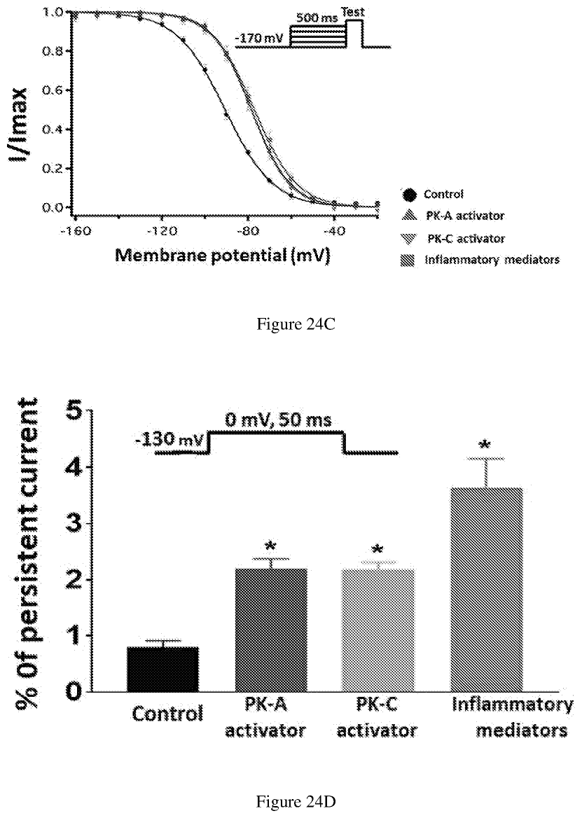

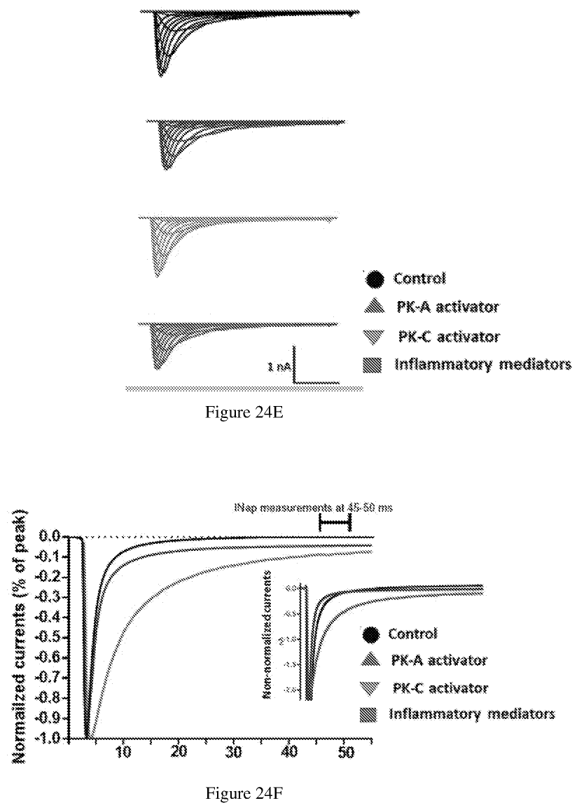

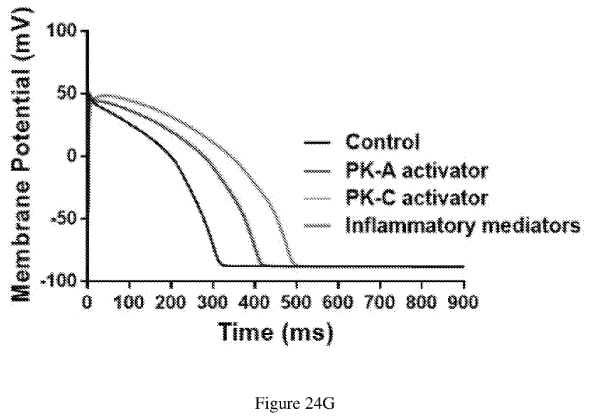

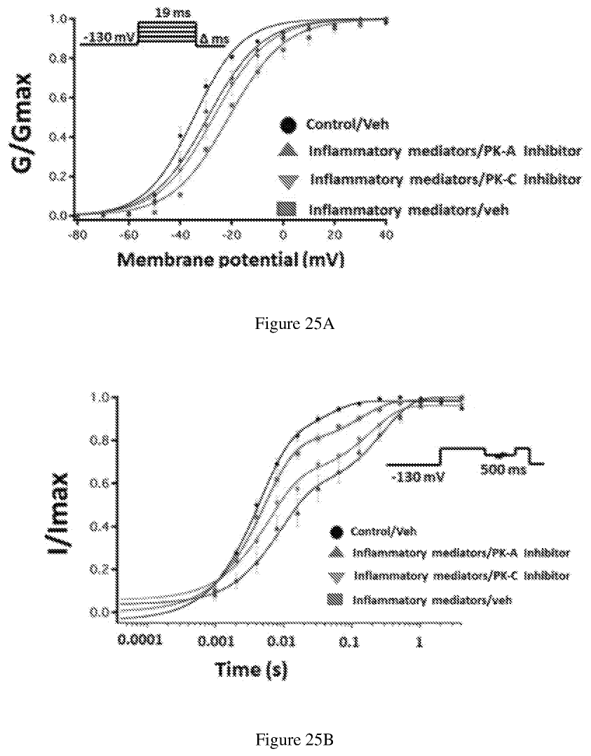

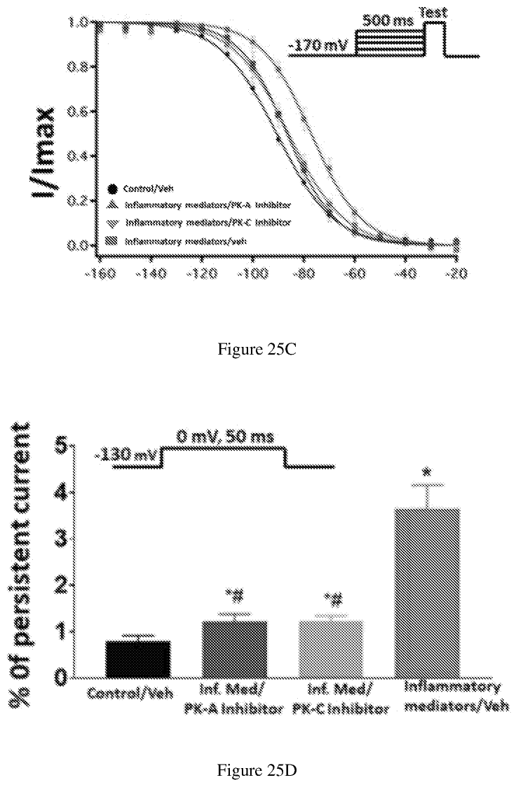

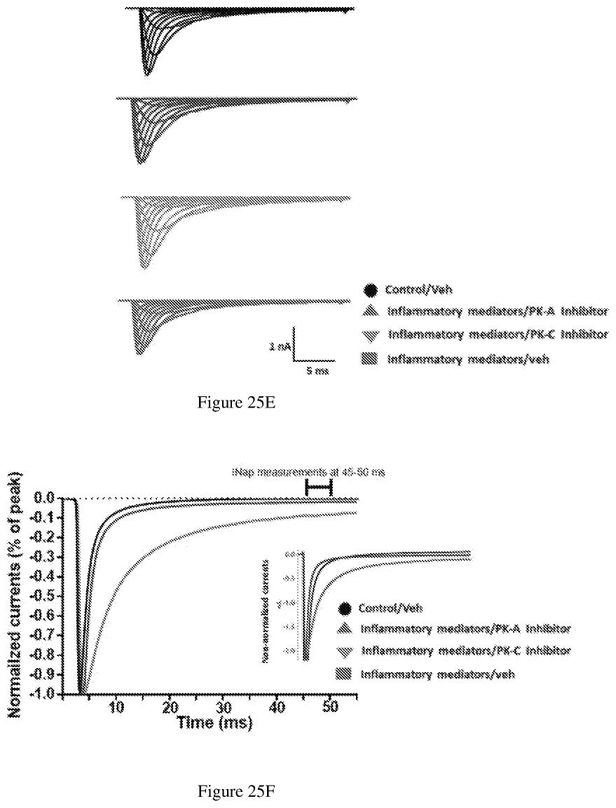

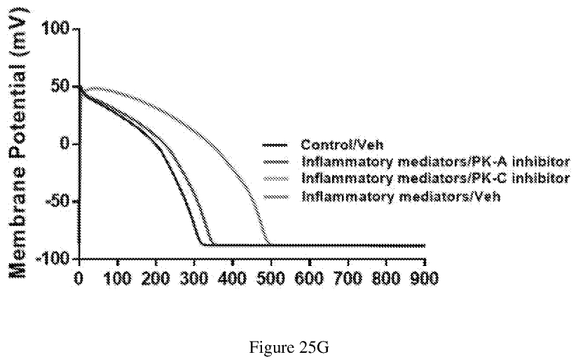

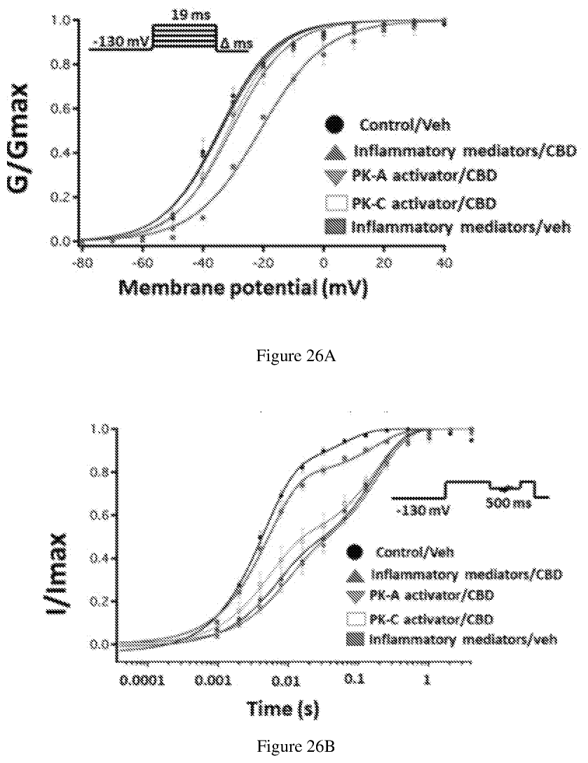

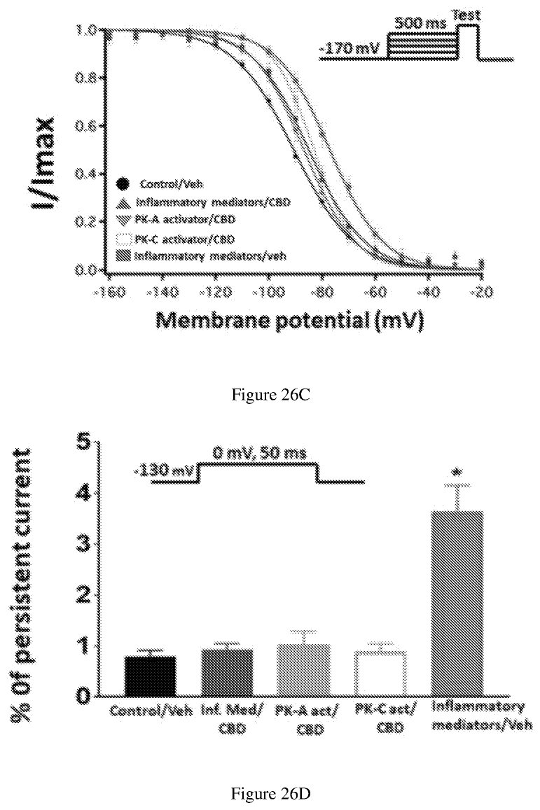

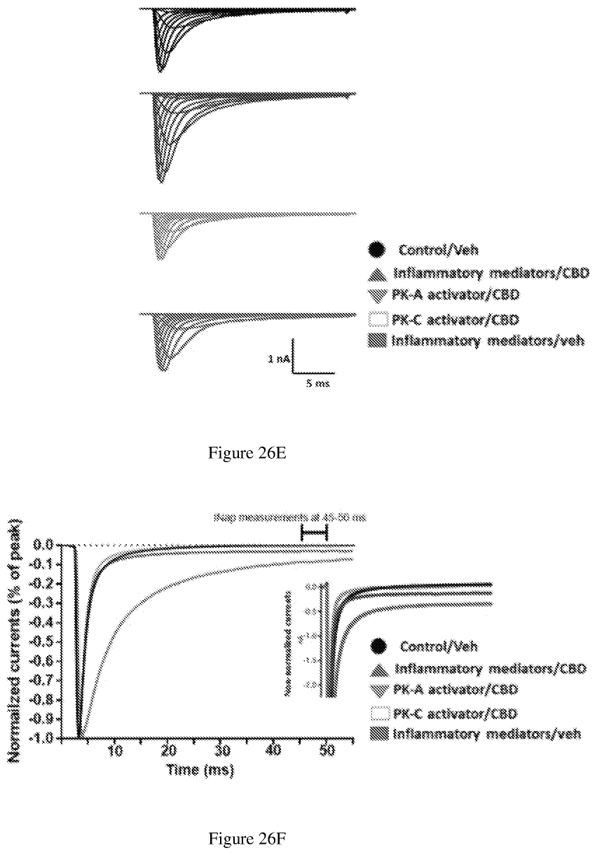

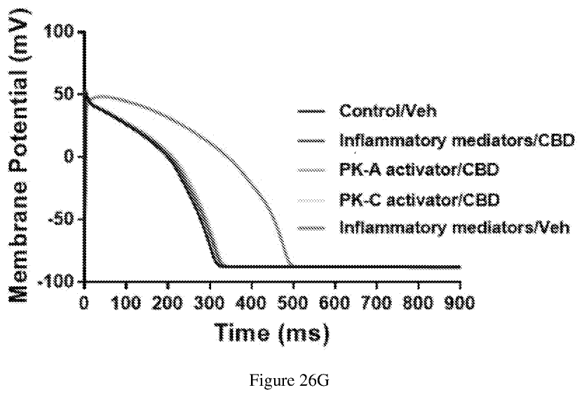

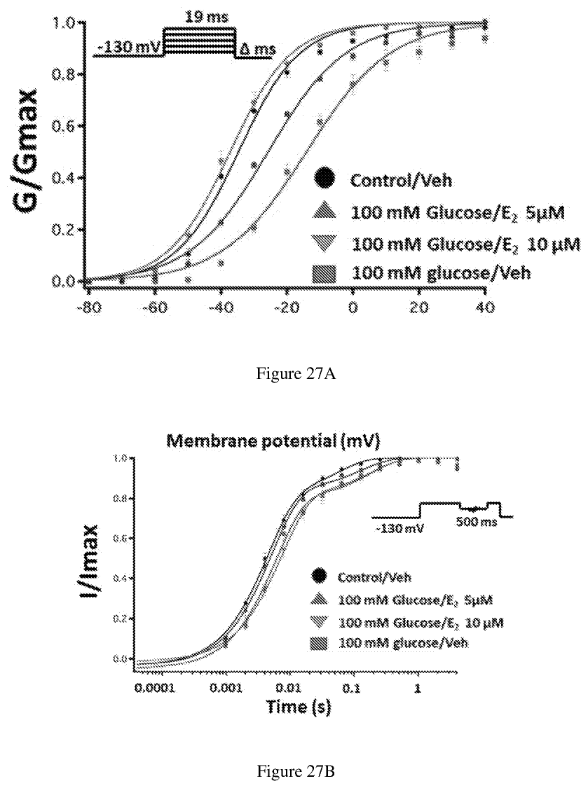

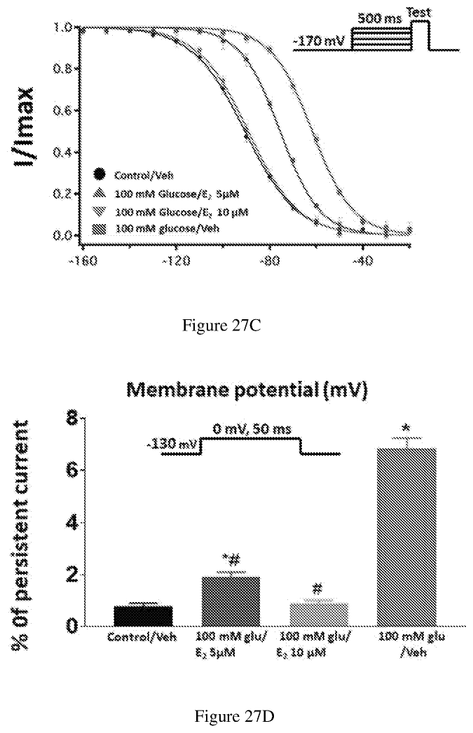

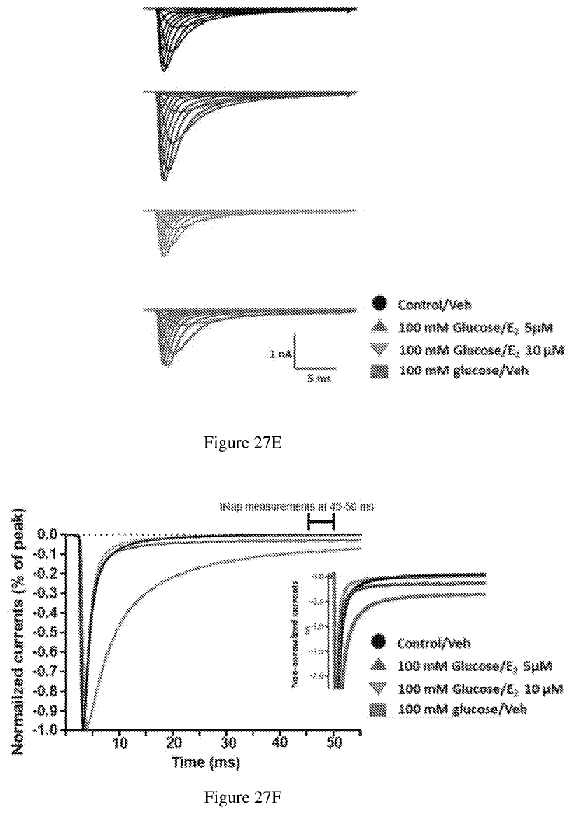

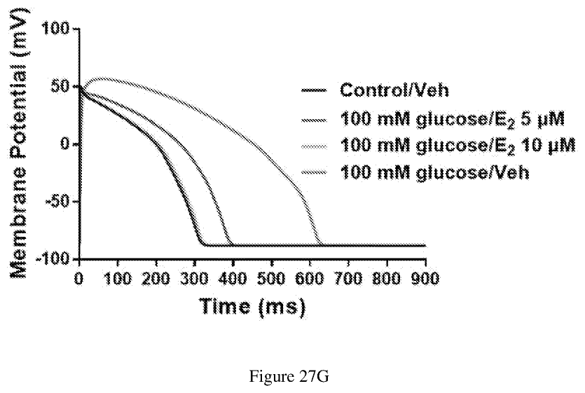

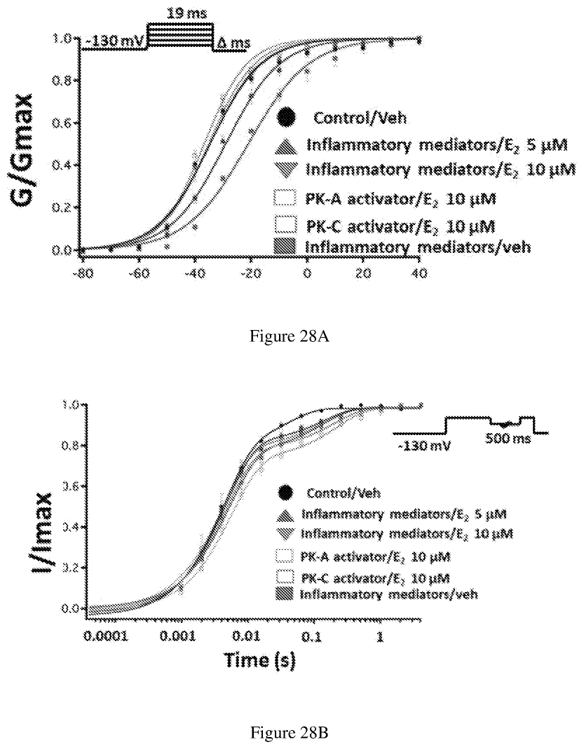

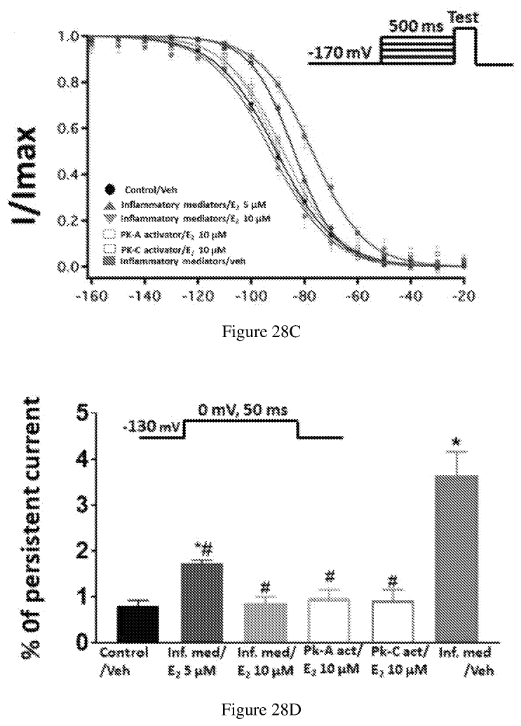

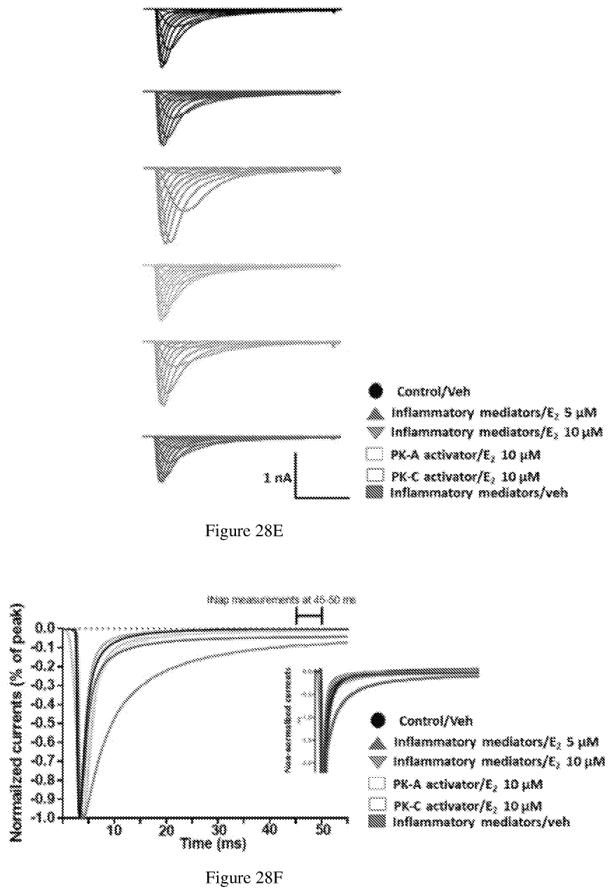

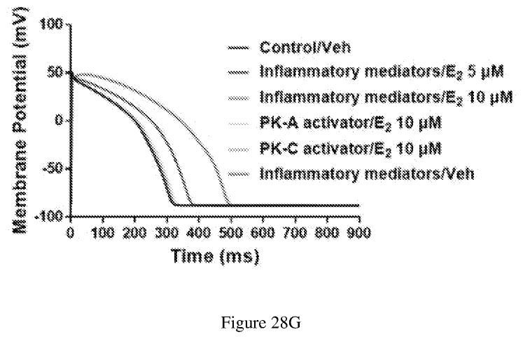

1. Comparison of study of electrophysiological changes induced by high glucose and inflammatory mediators on sodium channels Nav1.5 to check whether inflammatory mediators produce the same effects on the channels as that of high glucose (FIGS. 23A-23G);
2. Comparison of study of electrophysiological changes induced by inflammatory mediators and Protein kinase activators on sodium channels Nav1.5 to check whether Protein kinase activators produce the same changes on the channels as that of inflammatory mediators, (FIGS. 24A-24G);
3. Comparison of study of electrophysiological changes induced by the inflammatory mediators alone and inflammatory mediators along with protein kinase inhibitors on sodium channels Nav1.5 to check effects of “protein kinase inhibitors as control” on inflammation i.e whether protein kinase inhibitors can rescue the induced changes, (FIGS. 25A-25G);
4. Comparison of study of electrophysiological changes induced by the inflammatory mediators alone and inflammatory mediators along with CANNABIDIOL; and also, electrophysiological changes induced by the protein kinases activators along with CANNABIDIOL to check effects of CANNABIDIOL in rescuing deleterious effects produced by inflammatory mediators and activators of protein kinases, (FIGS. 26A-26G);
5. Comparison of study of electrophysiological changes induced by the high glucose alone and high glucose along with two concentrations of E2 to check effects of E2 in rescuing deleterious effects produced by high glucose (FIGS. 27A-27G);
6. Comparison of study of electrophysiological changes induced by the inflammatory mediators alone and inflammatory mediators along with two different concentrations of E2; and also, electrophysiological changes induced by the Protein kinases activators along with high concentration of E2 to check effects of E2 in rescuing deleterious effects produced by the inflammatory mediators and activators of protein kinases, (FIGS. 28A-28G);
7. Comparison of study of electrophysiological changes if any induced in human cardiomyocytes by CANNABIDIOL alone, inflammatory mediators alone and inflammatory mediators along with CANNABIDIOL; to check effects of CANNABIDIOL in rescuing deleterious effects produced by inflammatory mediators (FIGS. 30A-30G);
Hence, in addition to the inflammatory mediators (cocktail of inflammatory mediators), activator of protein kinase A (PK-A) and activator of protein kinases C (PK-C) are also employed in the study to check whether activator of protein kinase A (PK-A) or activator of protein kinases C (PK-C) affect the gating properties Nav1.5.

The new therapeutic agents CANNABIDIOL and Estradiol (E2) are also tested along with the protein kinase inhibitors which are employed as reference compound or a control.

In the present study, the invention provides various pharmaceutical compositions employing the new therapeutic agent CANNABIDIOL for

1. abolishing, or minimizing inflammation induced alteration in the gating properties of Nav1.5; and
2. rescuing sodium channels Nav1.5 from inflammation induced alteration in the gating properties.
3. The invention further provides uses of these pharmaceutical compositions of CANNABIDIOL for avoiding, abolishing or minimizing inflammation induced defects in the gating properties of Nav1.5 (alteration in the gating properties of Nav1.5) and treating to rescue channels or restore electrophysiology by administering pharmaceutical compositions employing new therapeutic agent CANNABIDIOL.

Further, the invention provides pharmaceutical compositions of CANNABIDIOL for treating or avoiding inflammation induced by any other therapeutic agent or inflammation induced in any diseases or ailment such as Covid-19 and also inflammation induced by any vaccine such as Covid-19 vaccine.

Accordingly, invention provides a pharmaceutical composition comprising therapeutically effective amount of cannabidiol for use in treatment of a cardiac disorder arising from gating defect in sodium channel Nav1.5 induced or likely to be induced by inflammation.

The invention further provides a method of treating cardiac disorder in a patient suffering from such disorder comprising administering a pharmaceutical composition comprising therapeutically effective amount of cannabidiol wherein the cardiac disorder arises from gating defects in sodium channel Nav1.5 induced or likely to be induced by inflammation.

### Results of Electrophysiological Experiments and Action Potential Modelling

The inventors have surprisingly found following,

1. Inflammatory mediators alter the gating properties of Nav1.5 similar to high glucose.
2. Activation of PK-A and PK-C mediates the inflammatory mediators induced alteration in the gating properties of Nav1.5.
3. CANNABIDIOL rescues the Nav1.5 gating changes of inflammatory mediators, activation of PK-A or PK-C.
4. E2 rescues the high glucose-induced alterations in Nav1.5 gating via PK-A and PK-C pathway.

This aspect is described hereinbelow in great details.

The present study aims to investigate following:

1. whether inflammation and subsequent activation of PK-A and PK-C mediate the high glucose-induced electrophysiological changes of Nav1.5 in a manner consistent with the gating defects that underlie long-QT arrhythmia; and
2. whether CANNABIDIOL and Estradiol rescue the high glucose induced Nav1.5 gating defects through, at least partly, this signalling pathway.

To study whether Inflammation and subsequent activation of PK-A and PK-C mediate the high glucose-induced electrophysiological changes of Nav1.5, the effect of perfusing PK-A inhibitor (H-89) or PK-C inhibitor (Gö 6983) are examined on Nav1.5 that had been incubated for 24 hours in either inflammatory mediators or vehicle.

CANNABIDIOL and Estradiol (E₂) are the main drugs of interest in the present study. The inventors hypothesized that inflammation could mediate the high glucose-induced biophyscial changes on Nav1.5 through protein phosphorylation by protein kinases A and C and that this signalling pathway is, at least partly, involved in the cardiprotective effects of CANNABIDIOL and E2.

To investigate the above hypothesis, the present study involves transiently co-transfecting Chinese hamster ovarian (CHO) cells with cDNA encoding human Nav1.5 α-subunit under control and adding to it, a cocktail of inflammatory mediators or 100 mM glucose conditions (for 24 hours) and further subjecting it to electrophysiological experiments and action potential modelling. The detailed procedure is provided under various examples. Electrophysiology is an electrical recording technique that enables the measurement of the flow of ions (ion current) in biological tissues. Whole cell patch clamp technique as described under example 2 is employed.

Activation Protocol: It comprises measuring peak current amplitude at test pulse voltages ranging from −130 to +80 mV in increments of 10 mV for 19 ms to determine the voltage-dependence of activation. The detailed process followed is provided under example 3.

Steady state fast inactivation protocols: It comprises measuring/determining voltage-dependence of fast-inactivation and involves preconditioning the channels to a hyperpolarizing potential of −130 mV and then eliciting pre-pulse potentials from −170 to +10 mV in increments of 10 mV for 500 ms, which is followed by a 10 ms test pulse during which the voltage is stepped to 0 mV. The detailed process followed is provided under example 4.

### Fast Inactivation Recovery

Unless Sodium channels recover from fast inactivation, they cannot get deactivated and unless they are deactivated, they are not ready to take part in further action potential. Thus, one of the key biophysical features of sodium channels is the kinetics at which they recover from inactivated states.

To measure fast inactivation recovery, first channels are fast inactivated during a 500 ms depolarizing step to 0 mV. Recovery is measured during a 19 ms test pulse to 0 mV following −130 mV recovery pulse for durations between 0 and 1.024 s. The detailed process followed is provided under example 5.

### Persistent Current Protocols

It comprises measuring late sodium current between 45 and 50 ms during a 50 ms depolarizing pulse to 0 mV from a holding potential of −130 mV. The detailed process followed is provided under example 6.

### Action Potential Modeling

The model selected shall be such that it shall account for activation voltage-dependence, steady-state fast inactivation voltage-dependence, persistent sodium currents, and peak sodium currents (compound conditions). A modified version of the O'Hara-Rudy model programmed in Matlab (O'Hara et al. 2011, PLoS Comput. Bio) is used to simulate action potential. The detailed process followed is provided under example 7.

Drug preparations involving preparations of CANNABIDIOL (CANNABIDIOL), Gö 6983, H-89, adenosine CPT-cAMP or PMA are described under example 8.

### Results of Electrophysiological Experiments and Action Potential Modelling

Following outcomes are obtained from the present study.

1. Inflammatory mediators alter the gating properties of Nav1.5 similar to high glucose indicating that inflammation brings the same effects in the gating properties of sodium channels Nav1.5.

2. Activation of PK-A and PK-C mediates the inflammatory mediators induced alteration in the gating properties of Nav1.5.

3. CANNABIDIOL rescues the Nav1.5 gating changes of inflammatory mediators, activation of PK-A or PK-C.

4. E2 rescues the high glucose-induced alterations in Nav1.5 gating via PK-A and PK-C pathway.

### 1. Inflammatory Mediators Alter the Gating Properties of Nav1.5 Similar to High Glucose

In electrophysiological experiments, whole-cell voltage-clamp method is used to measure gating in human Nav1.5, and to test the effects of incubating for 24 hours in either a cocktail of inflammatory mediators (as provided in Akin et al., 2019) or 100 mM glucose (as provided in Fouda, Ghovanloo & Ruben, 2020).

Peak channel conductance was measured between −130 and +80 mV in the presence of inflammatory mediators to determine whether the high glucose induced-changes in Nav1.5 activation (Fouda, Ghovanloo & Ruben, 2020) are, at least partly, mediated through inflammation.

FIG. 23A shows conductance plotted as a function of membrane potential. High glucose (100 mM) significantly shifted the Nav1.5 midpoint (V1/2) of activation in the positive direction (P=0.0002). Additionally, the slope (apparent valence, z) of the activation curves showed a significant decrease in 100 mM glucose (P=0.007) (FIG. 23A and Table 6). This decrease in slope suggests a reduction in activation charge sensitivity. It is found that incubation in inflammatory mediators for 24 hours, similar to 100 mM glucose, significantly right-shifted V1/2 of activation (P=0.001) and decreased z of activation curve (P=0.03) (FIG. 23A and Table 6). This suggests that both 100 mM glucose or inflammatory mediators decrease the probability of Nav1.5 activation.

As provided by West et al (West, Patton, Scheuer, Wang, Goldin & Catterall, 1992), DIII-IV linker mediates fast inactivation within a few milliseconds of Nav activation. When normalized current amplitudes are plotted as a function of pre-pulse potential as provided in FIG. 23B, 100 mM glucose or inflammatory mediators caused significant shifts in the positive direction in the V1/2 obtained from Boltzmann fits (100 mM glucose: P<0.0001; inflammatory mediators: P=0.001) (FIG. 23B and Table 7). These shifts suggest a loss-of-function in fast inactivation and suggest that high glucose or inflammatory mediators decrease the probability of steady-state fast inactivation in Nav1.5.

To study effects of 100 mM glucose or inflammatory mediators on recovery from fast inactivation, channels are held at −130 mV to ensure channels are fully at rest, then pulsed the channels to 0 mV for 500 ms, and allowed different time intervals at −130 mV to measure recovery as a function of time. It is found that incubation in 100 mM glucose or inflammatory mediators significantly (P<0.05) increase the slow component of fast inactivation recovery when compared to control, without affecting the fast component of recovery (FIG. 23C and Table 8).

Next, effects of 100 mM glucose or inflammatory mediators on the stability of Nav1.5 inactivation are studied. An increased persistent sodium current (INap) is a manifestation of destabilized fast inactivation (Goldin, 2003). Large INap is associated with a range of pathological conditions, including LQT3 (Ghovanloo, Abdelsayed & Ruben, 2016; Wang et al., 1995). To determine the effects of glucose or inflammatory mediators on the stability of Nav1.5 inactivation, the channels are held at −130 mV, followed by a depolarizing pulse to 0 mV for 50 ms to elicit persistent currents (Abdelsayed, Peters & Ruben, 2015; Abdelsayed, Ruprai & Ruben, 2018). FIG. 23D shows that incubation in 100 mM glucose or inflammatory mediators significantly increased INap compared to control (100 mM glucose: P<0.0001; inflammatory mediators: P<0.0001).

FIG. 23E provides representative families of macroscopic currents and FIG. 23F provides representative persistent currents across conditions.

Action potential modelling—O'Hara-Rudy model is used to simulate cardiac action potentials (AP) (O'Hara, Virag, Varro & Rudy, 2011). The model was modified using the results of present experiments and the effects of the tested compounds on the measured biophysical properties of activation (midpoint and apparent valence), steady-state fast inactivation (midpoint), recovery from fast inactivation, and persistent sodium current amplitude. The original model parameters were adjusted to correspond to the control results from the patch-clamp experiments, and the subsequent magnitude shifts in the simulations of other conditions were performed relative to the control parameters (Fouda, Ghovanloo & Ruben, 2020).

FIG. 23G shows that modifying the model with data obtained from incubation in 100 mM glucose or inflammatory mediators prolonged the simulated AP duration (APD) from ˜0.300 ms to ˜0.500 ms (inflammatory mediators) and to >600 ms (100 mM glucose).

This increased Action Potential Duration potentially leads to the prolongation of the QT interval (Nachimuthu, Assar & Schussler, 2012). Despite the similarity between 100 mM glucose and inflammatory mediators-induced changes on Nav1.5, their responses are not exactly the same (FIG. 23). This could be attributed to the concentration-dependent effects of high glucose on electrophysiological properties of Nav1.5 (Fouda, Ghovanloo & Ruben, 2020). The reason for selecting 100 mM glucose concentration is to ensure a sufficiently large window to detect readout signals throughout the study.

### 2. Activation of PK-A and PK-C Mediates the Inflammatory Mediators Induced Alteration in the Gating Properties of Nav1.5

One of the key signalling pathways involved in inflammation is the activation of protein kinase A (PK-A) or protein kinases C (PK-C) and subsequent protein phosphorylation (Karin, 2005).

To pharmocologically investigate the role of PK-A or PK-C signalling pathways in the inflammation-evoked gating changes of Nav1.5, Nav1.5 currents are recorded at room temperature in the absence or after a 20 minute perfusion of a PK-C activator (PMA; 10 nM (Hallaq, Wang, Kunic, George, Wells & Murray, 2012)) or PK-A activator (CPT-cAMP; 1 μM (Gu, Kwong & Lee, 2003; Ono, Fozzard & Hanck, 1993)).

Following observations are noted:

1. PMA or CPT-cAMP significantly shifted the Nav1.5 V_{1/2} of activation in the positive direction (PMA: P=0.0003; CPT-Camp: P=0.0007) (FIG. 24A and table 6).

2. PMA or CPT-Camp significantly reduced the effective valence (z) of the activation curves (PMA: P=0.002; CPT-Camp: P=0.007) (FIG. 24A and Table 6).

3. PMA or CPT-cAMP caused significant right-shifts in the V_{1/2} of SSFI (PMA: P=0.0008; CPT-Camp: P=0.0005) (FIG. 24B and Table 7).

4. PMA or CPT-cAMP significantly (P<0.05) increase the slow component of fast inactivation recovery when compared to control (FIG. 24C and Table 8).

5. PMA or CPT-cAMP significantly (PMA: P<0.0001; CPT-Camp: P<0.0001) increased INap compared to control.

All above effects are similar to those of glucose and inflammatory mediators (FIG. 23A, FIG. 24A, and Table 6).

Representative families of macroscopic and persistent currents across conditions are shown in FIGS. 24E and 24F.

6. Similar to 100 mM glucose and inflammatory mediators, the data from PK-A (CPT-cAMP) or PK-C (PMA) activator experiments shows that the in silico APD increased from ˜300 ms to ˜400 ms (FIG. 24G).

It was necessary to examine the effect of perfusing PK-A inhibitor or PK-C inhibitor to ensure that the effects of the inflammatory mediators on Nav1.5 are indeed mediated and accordingly, the effect of perfusing PK-A inhibitor (H-89, 2 μM for 20 minutes (Wang et al., 2013)) or PK-C inhibitor (Go 6983, 1 μM for 20 minutes (Wang et al., 2013)) are examined on Nav1.5 that had been incubated for 24 hours in either inflammatory mediators or vehicle.

Following observations are noted.

1. Although H-89 or Gö 6983 had no significant effects on Nav1.5 gating under control conditions (Table 6-9), H-89 or GO 6983 reduced the inflammatory mediator-induced shifts in V_{1/2} (H-89: P=0.0108; GO 6983: P=0.0203) (FIG. 25A and Table 6).

2. In addition, H-89 or Gö 6983 rescued the inflammatory mediator-induced shift in Nav1.5 SSFI (FIG. 25B and Table 7).

3. Moreover, H-89 or GO 6983 (P=0.0041, or P=0.0017, respectively) further increased the time constant of the slow component of recovery from fast inactivation when compared to inflammatory mediators (FIG. 25C and Table 8).

4. As shown in FIG. 25D, H-89 or GO 6983 (P<0.0001) incompletely reduced the inflammatory mediator-induced increase in the persistent currents (Table 9).

Representative families of macroscopic and persistent currents across conditions are shown (FIGS. 25E and 25F).

Representative families of macroscopic and persistent currents across conditions are shown (FIGS. 25E and 25F).

5. Importantly, in silico APD using the data from inhibitors of PK-A (H-89) or PK-C (Gö 6983) reduced the inflammatory mediators-induced simulated APD prolongation (FIG. 25G).

### 3. CANNABIDIOL Rescues the Nav1.5 Gating Changes of Inflammatory Mediators, Activation of PK-A or PK-C

CANNABIDIOL and Estradiol E₂ are the main drugs of interest in the present study. The inventors hypothesized that inflammation could mediate the high glucose-induced biophysical changes on Nav1.5 through protein phosphorylation by protein kinases A and C and that this signalling pathway is, at least partly, involved in the cardioprotective effects of CANNABIDIOL (CANNABIDIOL) and Estradiol (E2).

Previous experimentation by the inventors demonstrated that CANNABIDIOL rescues high glucose-induced dysfunction in Nav1.5 (Fouda et al., 2020).

As H-89 or GO 6983 exhibited remarkable counter actions on inflammatory mediator-induced effects, it encouraged inventors to test the effects of CANNABIDIOL on the biophysical properties of Nav1.5 in the presence of inflammatory mediators, PK-C activator (PMA), or PK-A activator (CPT-cAMP). To determine whether the observed changes to activation and SSFI imparted by inflammatory mediators, or if activation of PK-A or PK-C could be rescued by CANNABIDIOL, peak sodium currents are measured in the presence of CANNABIDIOL.

Following observations are noted:

1. CANNABIDIOL (5 μM) perfusion abolished the effects of inflammatory mediators, PMA, or CPT-cAMP, including shifts of V_{1/2} of activation, z of activation and the V_{1/2} of SSFI (FIGS. 26A and 26B and Table 6 and 7).

2. CANNABIDIOL significantly increased the time constant of the slow component of recovery from fast inactivation regardless of the concurrent treatment (inflammatory mediators, PMA or CPT-cAMP) (FIG. 26C and Table 8).

3. CANNABIDIOL reduced the increase in INap caused by inflammatory mediators, PMA or CPT-cAMP (FIG. 26D and Table 9)

Representative macroscopic and persistent currents are shown in FIGS. 26E and 26F.

4. The O'Hara-Rudy model results also suggest that CANNABIDIOL rescues the prolonged in silico APD caused by inflammatory mediators or activators of PK-A or PK-C-induced to nearly that of the control condition (FIG. 26G). The reduction in APD is consistent with the anti-excitatory effects of CANNABIDIOL (Ghovanloo, Shuart, Mezeyova, Dean, Ruben & Goodchild, 2018).

### 4. E₂ Rescues the High Glucose-Induced Alterations in Nav1.5 Gating Via PK-A and PK-C Pathway

It is investigated whether E₂ rescues the high-glucose induced changes in biophysical properties of Nav1.5 given that E₂ previously was shown to affect Nav in addition to its anti-inflammatory role (Iorga, Cunningham, Moazeni, Ruffenach, Umar & Eghbali, 2017; Wang, Garro & Kuehl-Kovarik, 2010).

Following observations are noted:

1. The inventors first tested the effects of E₂ (5 or 10 μM) under control conditions and found that E₂ exerted no significant effects on Nav1.5 gating (Tables 6-9). In contrast, FIG. 27 shows that perfusing E₂ (5 or 10 μM) for at least 10 minutes into the bath solution (Möller & Netzer, 2006; Wang et al., 2013) abolished the shifts elicited by high glucose (100 mM, for 24 hours, including V_{1/2}, z of activation and the V_{1/2} of SSFI in a concentration-dependent manner (FIG. 27A, 27B and Table 6 and 7).

2. On the other hand, it was found that E₂ (5 or 10 μM) had no significant effect on 100 mM glucose-induced slight increase in the slow component of fast inactivation recovery (FIG. 27C and Table 8).

3. However, E₂ significantly reduced the 100 mM glucose-induced increase in INap in a concentration-dependent manner (FIG. 27D and Table 9).

4. E₂ reduction of the glucose-exacerbated INap is consistent with previous reports of similar effects in neuronal sodium channels (Wang, Garro & Kuehl-Kovarik, 2010).

5. FIG. 27G shows that O'Hara-Rudy model results suggest that E₂, in a concentration-dependent manner, rescues the prolonged in silico APD caused by 100 mM glucose (FIG. 27G).

6. The inventors tested whether E₂ (5 or 10 μM) rescues the effects of inflammatory mediators, PK-C activator (PMA), or PK-A activator (CPT-cAMP) on the gating properties of Nav1.5. FIG. 28 shows that concurrent addition of E₂ abolished the effects of inflammatory mediators on activation and SSFI in a concentration-dependent manner (FIG. 28A, 28B and Table 6 and 7).

8. Similarly, E₂ concentration-dependently rescued PMA or CPT-cAMP-elicited effects on activation and SSFI (FIG. 28A, 28B and Table 6 and 7).

9. Although E₂ (5 or 10 μM) had no significant effect on the slight increase in the slow component of fast inactivation recovery caused by inflammatory mediators, PMA, or CPT-cAMP (FIG. 28C and Table 8),

10. E₂ significantly reduced the increase in INap in a concentration-dependent manner (FIG. 28D and Table 9;

Representative currents are shown in FIGS. 28E and 28F.

11. In addition, E₂ concentration-dependently rescues the prolonged in silico APD caused by inflammatory mediators or activators of PK-A or PK-C-induced to nearly that of the control condition (FIG. 28G).

### Discussion and Conclusion

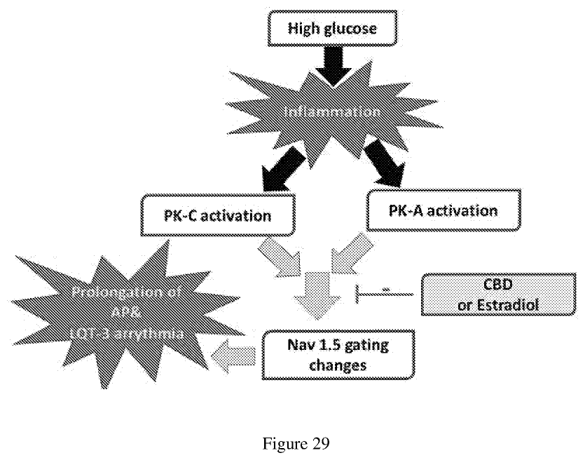

The experiments conducted in the present study address for the first time, the inflammation/PK-A and PK-C signalling pathway to mediate high glucose-induced cardiac anomalies (FIG. 29).

The results obtained in various electrophysiological and action potential modelling suggest and conclude that CANNABIDIOL and E₂ may exert their cardioprotective effects against high glucose, at least partly, through this signalling pathway.

This conclusion is based on the following findings:

(i) Similar to high glucose, inflammatory mediators elicited right shifts in the voltage-dependence of activation and inactivation and exacerbated persistent currents. Increased persistent currents prolong the simulated action potential duration;
(ii) Activators of PK-A and PK-C reproduced the high glucose- and inflammation-induced changes in Nav1.5 gating;
(iii) Inhibitors of PK-A and PK-C reduced, to a great extent, the high glucose- and inflammation-induced changes in Nav1.5 gating;
(iv) CANNABIDIOL or E₂ rescued the effects of high glucose, inflammatory mediators, or PK-A or PK-C activators.

The results suggest a role of Nav1.5 in high glucose induced hyperexcitability, via inflammation and subsequent activation of PK-A and PK-C, which could lead to LQT3-type arrhythmia (FIG. 29). In addition, these findings suggest possible therapeutic effects for CANNABIDIOL in high glucose-provoked cardiac dysfunction in diabetic patients, especially those post-menopauses.

Considering the coorelation between diabetes and LQT as provided by Ukpabi & Onwubere, 2017 and Whitsel et al., 2005; and also considering crucial role of Nav1.5 gain-of-function in the development of LQT as taught by Shimizu & Antzelevitch, 1999, the inventors found that inflammatory mediators replicated the high glucose-induced changes in Nav1.5 gating which is similar to those correlated with LQT3 in diabetic rats as taught in Yu et al (Yu et al., 2018). This finding is consistent with other reports showing that hyperglycemia/high glucose is proinflammatory and that inflammation is a crucial player in the pathogenesis of cardiovascular anamolies (Fouda, Leffler & Abdel-Rahman, 2020; Tsalamandris et al., 2019).

Inflammation alters the electrophysiological properties of cardiomyocytes Nav with an increase in INap leading to prolongation of APD which is similar to finding of the present inventors as provided in FIG. 23. The present study along with previous research support hypothesis of the present inventors that high glucose, at least partly through induction of inflammation, alters Nav1.5 gating and leads to LQT arrhythmia.

As provided in FIGS. 25 and 26, present research suggest that activation of PK-A or PK-C replicated high glucose- and inflammation-induced gating changes in Nav1.5 gating, whereas inhibition of PK-A or PK-C abolished those changes. This finding suggests that PK-A and PK-C may be downstream effectors of inflammation in high glucose-induced cardiac complications.

Although there are conflicting reports regarding the effects of PK-A and PK-C activation on the voltage-dependence and kinetics of Nav1.5 gating, these differences could be attributed to different voltage protocols, different holding potentials, different concentrations or type of PK-activators, or different cell lines used in the various studies (Aromolaran, Chahine & Boutjdir, 2018; Iqbal & Lemmens-Gruber, 2019). Despite this discrepancy, both PK-A or PK-C destabilize Nav fast inactivation and hence increase INap, which is strongly correlated to prolonged APD as shown in FIG. 29. (Astman, Gutnick & Fleidervish, 1998; Franceschetti, Taverna, Sancini, Panzica, Lombardi & Avanzini, 2000; Tateyama, Rivolta, Clancy & Kass, 2003).

The present study on PK-A and PK-C modulators prompted inventors to test whether CANNABIDIOL affects the biophysical properties of Nav1.5 through this pathway. The inventors thereafter investigated the possible protective effect of CANNABIDIOL against the deleterious effects of high glucose through this signalling pathway because as reported by Fouda et al CANNABIDIO protects against high glucose-induced gating changes in Nav1.5 (Fouda, Ghovanloo & Ruben, 2020). In addition, as reported by Rajesh et al, CANNABIDIOL attenuates the diabetes-induced inflammation and subsequent cardiac fibrosis through inhibition of phosphorylation enzymes (such as MAPKs) (Rajesh et al., 2010). The results obtained in the present study suggest that CANNABIDIOL alleviates the inflammation/activation of PK-A or PK-C induced biophysical changes as provided in Figure. 27. The findings of the present research are consistent with the anti-inflammatory, antioxidant, and anti-tumor effects of CANNABIDIOL via inhibition of PK-A and PK-C signalling (Seltzer, Watters & MacKenzie, 2020). The incomplete protective effects of PK-A and PK-C inhibitors compared to the CANNABIDIO effect against the inflammation-induced gating changes in Nav1.5 could be attributed to the combined CANNABIDIOL direct inhibitory effect on Nav1.5 and its indirect inhibitory actions on both PK-A and PK-C (FIGS. 26, 27 and 28).

Further, role of E₂ is investigated in the present study. E₂ directly affects Nav and exerts anti-inflammatory effects as reported by Iorga et al and Wang et al.

Other reports by various researchers show the cardioprotective effects of E₂ by increasing angiogenesis, vasodilation, and decreasing oxidative stress and fibrosis (lorga, Cunningham, Moazeni, Ruffenach, Umar & Eghbali, 2017). many studies support the anti-arrythmic effects of E₂ because of its effects on the expression and function of cardiac ion channels (lorga, Cunningham, Moazeni, Ruffenach, Umar & Eghbali, 2017; Odening & Koren, 2014).

Two key findings on E₂ are important:

1. E₂ stabilizes Nav fast inactivation and reduces INap, similar to CANNABIDIOL (Wang, Garro & Kuehl-Kovarik, 2010); and
2. E₂ reduces the oxidative stress and the inflammatory reponses by inhibiting PK-A and PK-C-mediated signalling pathways (Mize, Shapiro & Dorsa, 2003; Viviani, Corsini, Binaglia, Lucchi, Galli & Marinovich, 2002).

The inventors found that E₂, similar to CANNABIDIOL (CANNABIDIOL), rescues the effects of high-glucose, inflammation, and activation of PK-A or PK-C (FIGS. 27-29).

In conclusion, the present study provides that inflammation and the subsequent activation of PK-A and PK-C correlate with the high glucose-induced electrophysiological changes in Nav1.5 gating (FIG. 29). In silico, these gating changes result in prolongation of simulated action potentials leading to LQT3 arrhythmia, which is a clinical complication of diabetes (Grisanti, 2018). CANNABIDIOL and E₂, through inhibition of this signalling pathway, ameliorate the effects of high glucose and the resultant clinical condition.

Thus, in conclusion, the present study finds following:
Inflammation and subsequent activation of PK-A and PK-C mediate the high glucose-induced electrophysiological changes of Nav1.5 in a manner consistent with the gating defects that underlie long-QT arrhythmia.
CANNABIDIOL and Estradiol rescue the high glucose induced Nav1.5 gating defects through, at least partly, this signalling pathway.

The inventors have found that Inflammation/PK-A and PK-C signalling pathway is a potential therapeutic target to prevent arrhythmias associated with diabetes and further propose CANNABIDIOL as an alternate therapeutic approach to prevent cardiac complications in diabetic especially postmenopausal population due to the decreased levels of the cardioprotective estrogen, especially in diabetic postmenopausal populations.

The invention further provides uses of these pharmaceutical compositions of CANNABIDIOL for avoiding, abolishing or minimizing inflammation induced defects in the gating properties of Nav1.5 (alteration in the gating properties of Nav1.5) and treating to rescue channels or restore electrophysiology by administering pharmaceutical compositions employing new therapeutic agent CANNABIDIOL.

Further under this eighth aspect, the invention provides pharmaceutical compositions of CANNABIDIOL for treating or avoiding or minimizing inflammation induced by any other therapeutic agent or inflammation induced in any diseases or ailment such as Covid-19 and also inflammation induced by any vaccine such as Covid-19 vaccine.

Simultaneous administration of CANNABIDIOL includes administering CANNABIDIOL along with at least one therapeutic agent/drug capable of inducing inflammation. The CANNABIDIOL can be added in the same pharmaceutical composition as that of such other drug or CANNABIDIOL can be present in different dosage form but administered simultaneously or sequentially at the same time when the other drug is administered. Administering at the same time as the term appears here means that the CANNABIDIOL is physically administered when the other dug is physically administered, and it also means that CANNABIDIOL is administered in presence of other drug in biological environment or the other drug is administered when CANNABIDIOL is in biological environment.

CANNABIDIOL can be either administered alone or can be co-administered along with E₂ in suitable pharmaceutical formulations/pharmaceutical compositions discussed below.

### Role of CANNABIDIOL on Sodium Channels Nav1.4.

Under the ninth aspect, the invention provides various pharmaceutical compositions employing the new therapeutic agent CANNABIDIOL to rescues the adversely affected sodium channels Nav1.4 from the contractility dysfunction and conditions produced further from these effects such as muscle stiffness, pain, myotonia, gating-pore current in the VSD leading to periodic paralyses etc.

Accordingly, invention provides a pharmaceutical compositions comprising therapeutically effective amount of cannabidiol for use in treatment of a skeletal muscle disorder arising from adversely affected sodium channel Nav1.4.

In this aspect, invention further provides a method of treating skeletal muscle disorder in a patient suffering from such disorder comprising administering a pharmaceutical compositions comprising therapeutically effective amount of cannabidiol wherein the skeletal muscle disorder arises from adversely affected sodium channel Nav1.4.

### Sodium Channel Nav1.4 is a Molecular Target for CANNABIDIOL

Variants of the Nav subtype predominantly expressed in skeletal muscles, Nav1.4, lead to contractility dysfunction. Most Nav1.4 variants depolarize the sarcolemma; however, this depolarization can result in either hyper- or hypo-excitability in phenotype (Cannon et al (2006)). Nav-channelopathies which change membrane excitability underlie clinical syndromes. Hyperexcitable muscle channelopathies are classified as either non-dystrophic myotonias or periodic paralyses (Lehmann-Horn et al (2008)). Most of these channelopathies arise from sporadic de-novo or autosomal dominant mutations in SCN4A (Ghovanloo (2018)).

The majority of gain-of-function (GOF) Nav1.4 variants result in myotonic syndromes. Myotonia is defined by a delayed relaxation after muscle contraction (Lehmann-Horn (1995) and Tan (2011)). In myotonia, there is an increase in muscle membrane excitability in which even a brief voluntary contraction can lead to a series of APs that can persist for several seconds after motor neuron activity is terminated. This phenomenon is perceived as muscle stiffness (Tan (2019)). The global prevalence of non-dystrophic myotonias is ˜1/100,000 (Emery (1991)). Although this condition is not considered lethal, it can be life-limiting due to the multitude of contractility problems it can cause, including stiffness and pain (Vicart (2005).

A cationic leak (gating-pore current in the VSD) with characteristics similar to the ω-current in Shaker potassium channels has been shown to cause periodic paralyses (Jiang (2018)). This mechanism indicates that periodic paralyses can be caused by a severe form of GOF in Nav1.4 (Wu, F (2011)) and (Tombola (2005)).

Thus, there is a greater need to develop therapeutics to reduce i) skeletal muscle contractility, ii) myotonia, iii) gating-pore current in the VSD leading to periodic paralyses, iv) muscle stiffness and pain.

There are few therapeutics developed for skeletal muscle conditions and for treating myotonias and periodic paralyses and they mostly rely on drugs developed for other conditions, including local-anesthetics (LA). Myotonia treatment is focused on reducing the involuntary AP bursts (Vicart (2005) and Desaphy (2004)).

The inventors through various studies conducted propose that the Nav1.4 could be a molecular therapeutic target for reducing skeletal muscle contractility, myotonia, gating-pore current in the VSD leading to periodic paralyses, muscle stiffness and pain.

Further, through various studies, inventors of the present invention found that CANNABIDIOL reduces skeletal muscle contraction. Since skeletal muscle contraction is related to Nav1.4, the full modulatory mechanism and effects of CANNABIDIOL on Nav1.4 are investigated. It is found that CANNABIDIOL alters membrane rigidity and penetrates into the Nav pore through fenestrations. Finally, it is proposed that CANNABIDIOL may alleviate myotonia via its direct and indirect effects on Nav1.4.

### Effect of CANNABIDIOL on Rat Diaphragm Muscle Contraction

To determine whether CANNABIDIOL reduces skeletal muscle contractions, the inventors studied action of CANNABIDIOL on rat diaphragm muscle. The muscle is surgically removed. The muscle contractions evoked by phrenic nerve stimulation are measured. FIG. 9A-B provide images of the diaphragm, cut into a hemi-diaphragm. Electrodes are used to stimulate the phrenic nerve and the muscle contraction is measured using a force transducer, at a saturating concentration of 100 μM of CANNABIDIOL, reasoning that if CANNABIDIOL reduces muscle contraction, then a saturating concentration should provide a large enough window to detect any potential reduction in contraction. The results suggested that CANNABIDIOL reduces contraction amplitude to ˜60% of control (p<0.05) (FIG. 9C).

This action of CANNABIDIOL on the skeletal muscle may be due to its blocking action for sodium channel Nav1.4. To confirm this, a known blocker is tested for a similar action. A 300 nM saturating concentration of tetrodotoxin (TTX), a potent blocker of selected Nav channels (IC50˜10-30 nM on TTX-sensitive channels 39) is used. It is found that TTX reduced contraction to ˜20% of control (p<0.05) (FIG. 1C) suggesting that CANNABIDIOL's contraction reduction could be in part due to activity at Nay. Representative traces of muscle contraction in control, CANNABIDIOL, and TTX are shown in FIG. 9D-F. These results collectively support the idea that inhibition of Nav could reduce skeletal muscle contraction, and that CANNABIDIOL's reduction of muscular contraction is due, at least in part, to its effect on Nav as previously suggested in Ghovanloo (2018).

### Molecular Dynamic (MD) Simulation Study of CANNABIDIOL

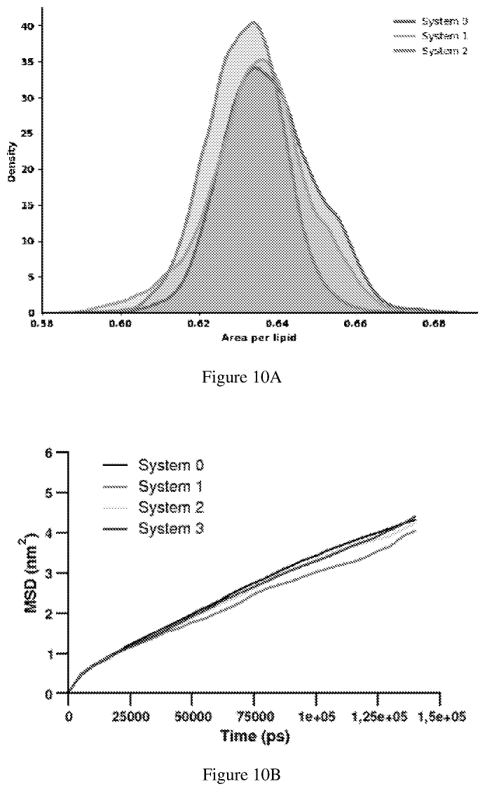

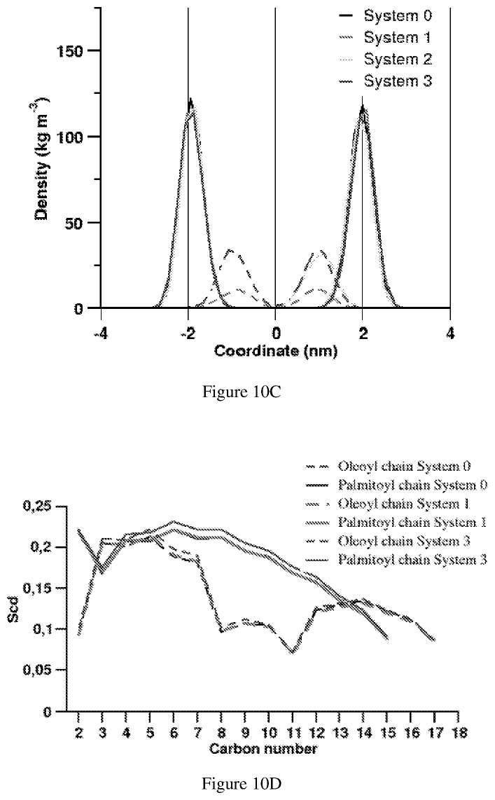

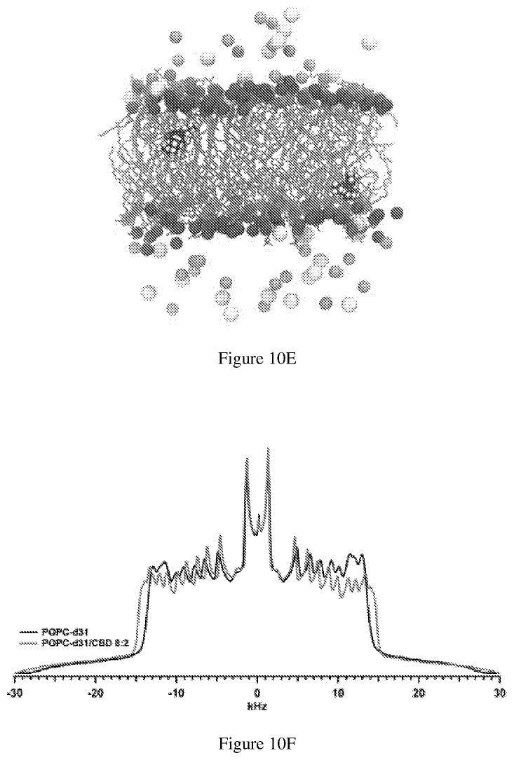

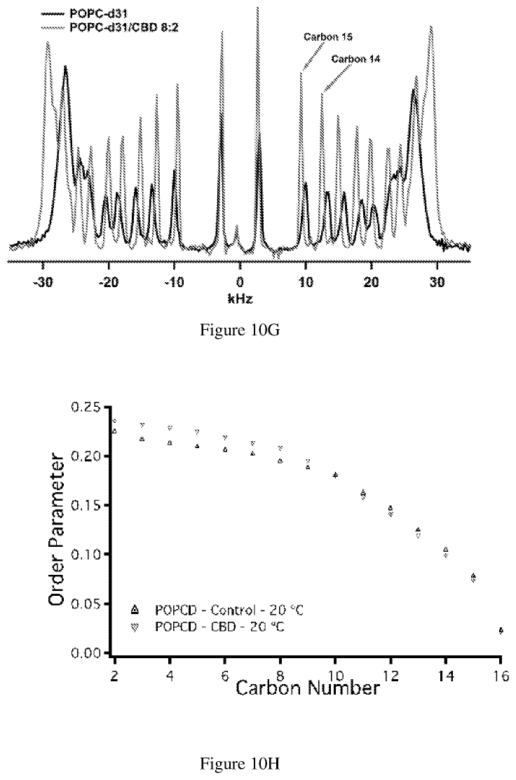

Further studies involve finding out the mechanism by which CANNABIDIOL acts as a Nav blocker. One study involved finding out whether CANNABIDIOL alters membrane rigidity and thereby indirectly inhibits Nav 1.4. In this, molecular dynamic (MD) simulation study of CANNABIDIOL (in mM concentration in membrane) is performed on 1-palmitoyl-2-oleoyl-sn-glycero-3-phosphocholine (POPC) lipid membranes in the hundred nanosecond range. MD results indicate that in both symmetrical (i.e. the same number of CANNABIDIOL molecules in both leaflets of the membrane) and asymmetrical (i.e. CANNABIDIOL in a single leaflet), there are no substantial changes in the area per lipid. The FIGS. 10A-E provide effects of CANNABIDIOL on POPC membrane via MD simulation. FIG. 10C particularly provides CANNABIDIOL density estimates as a function of membrane leaflet coordinate, where the lipid bilayer is centered at 0. It provides distribution of CANNABIDIOL into the membrane across a range of conditions. The dotted lines in FIG. 10C represents CANNABIDIOL distribution. These results show, in symmetrical conditions, that there are two density peaks in both negative and positive coordinate ranges with an almost perfect overlap, and that CANNABIDIOL localizes in the area just between the lipid headgroups and the membrane center, close to the lipid headgroup region.

In the asymmetrical condition with 3 CANNABIDIOL molecules initially placed in one of the two leaflets, however, there is only a single peak in the positive coordinate range. The MD results also show that CANNABIDIOL molecules tend to be able to come in contact and detach rapidly and, occasionally, move towards the water molecules outside the lipid. However, CANNABIDIOL then quickly moves back down into the lipid. This suggests, within the hundreds of nanoseconds timeframe of simulations, that CANNABIDIOL does not diffuse across the two leaflets but, instead, tends to localize mostly in the leaflet where it was initially placed. Overall, the MD results suggest that CANNABIDIOL tends to localize preferentially between the phosphate head and the bottom end of the fatty chain, close to carbons 3-7 of the aliphatic chains of the POPC molecules (FIG. 10E).

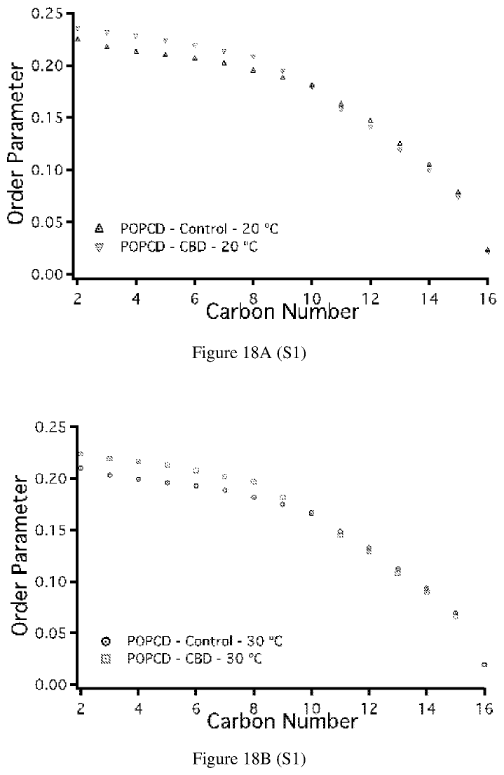

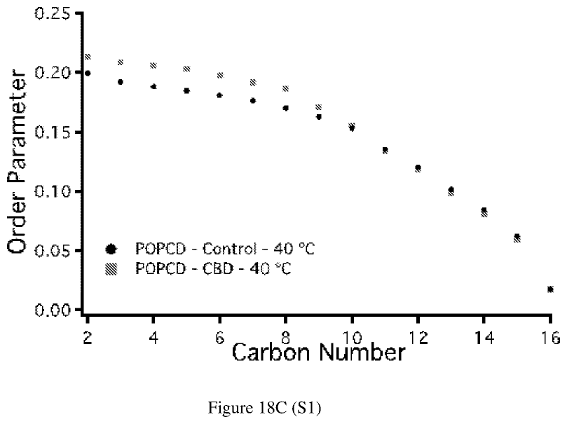

The MD predictions regarding CANNABIDIOL localization are further functionally tested by performing NMR studies. NMRs are performed as provided in Lafleur (1989) with POPC-d31 and POPC-d31/CANNABIDIOL in a 4:1 ratio in deuterium depleted water at three different temperatures (20, 30, and 40° C.) (FIG. 10F-H; FIG. 18A-C). The NMR results were in striking agreement with the MD predictions of acyl chain order parameters and suggested that CANNABIDIOL causes an ordering of the C2-C8 methylenes, and a slight disordering from C10-C15, in a temperature-dependent manner.

Effect of CANNABIDIOL on Membrane Rigidity in HEK Cells by Gramicidin (gA)-Based Assay

To find effect of CANNABIDIOL on membrane rigidity, a gramicidin (gA)-based assay is used. Gramicidin channels are made up of dimers, with each monomer residing in one of the membrane leaflets. These channels preferentially conduct cationic (Na+ and K+) currents when two monomers dimerize to form a continuous pore across the membrane. Thus, dimerization is directly related to membrane rigidity Lundbek (2004).

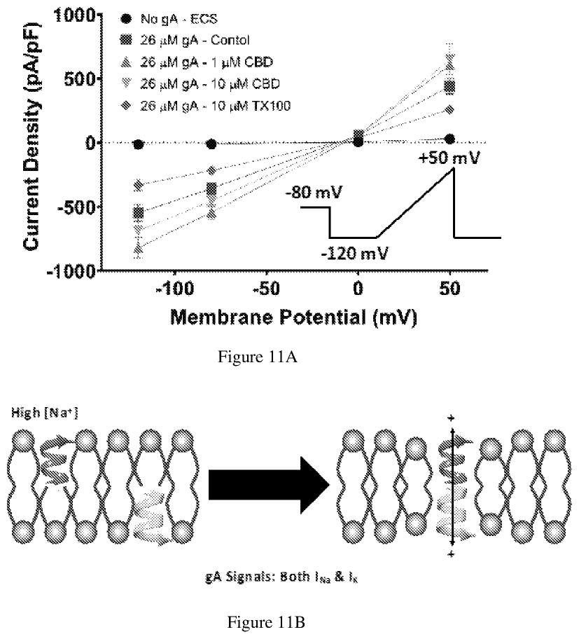

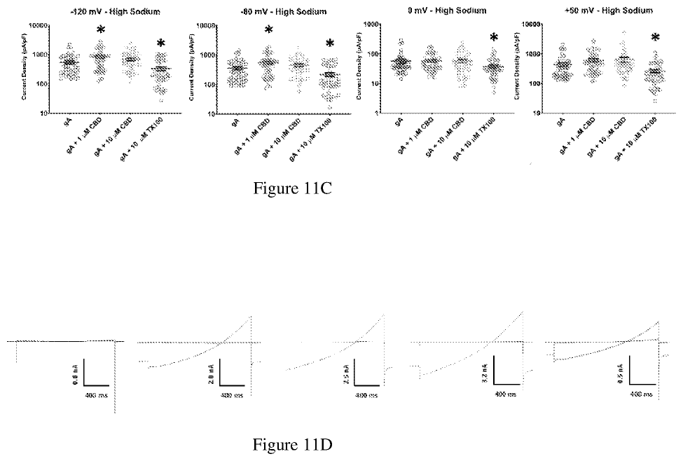

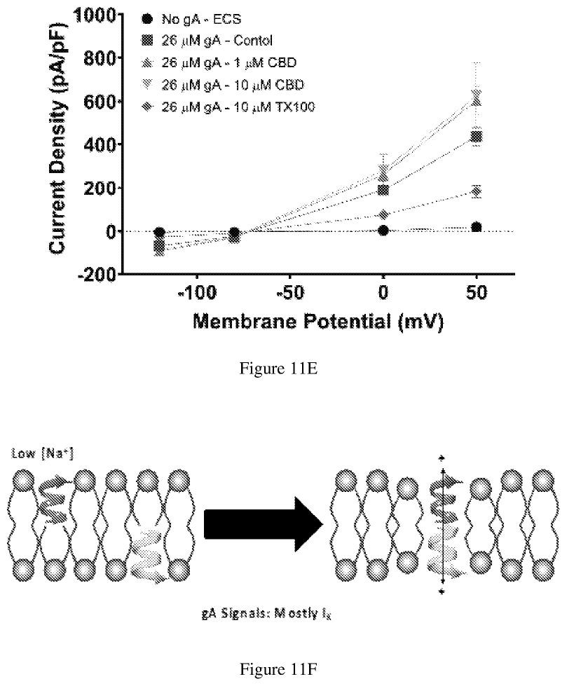

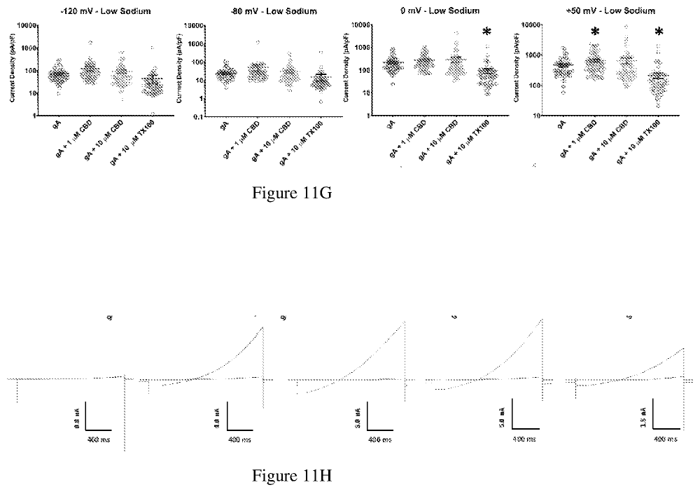

The whole-cell voltage-clamp of untransfected HEK cells in the absence and presence of 26 μM gramicidin are used. The gramicidin currents are measured in standard high sodium [Na+=140 mM] extracellular solution using a ramp protocol. The cells are clamped at −80 mV, close to the K+ equilibrium potential (EK+). Then, cells are hyperpolarized the to −120 mV and the clamp is ramped to +50 mV, which is close to ENa+. FIGS. 11A-D provide average gramicidin current density from the ratio of current amplitude to the cell membrane capacitance (pA/pF) at −120, −80, 0, and +50 mV. As shown in the figures, at negative potentials, gramicidin channels conduct inward currents and, as the membrane potential becomes more positive, the current becomes outward with the reversal potential (Erev) being close to 0 mV. The effects of 1 μM (˜inactivated Nav IC505) and 10 μM (˜resting Nav IC505) CANNABIDIOL, and 10 μM Triton X100 (TX100) as positive control are measured on gramicidin-HEK cells (FIG. 11A-C). TX100 is a detergent and has been shown to change membrane rigidity and hence to alter gramicidin current amplitude (Lundbek (2004)). The findings indicate that TX100 altered the cationic gramicidin currents across all potentials (p<0.05) (FIG. 11C). However, CANNABIDIOL had the opposite effect to TX100, and slightly altered gramicidin currents at both 1 μM (p<0.05) and 10 μM (p>0.05). Interestingly, although CANNABIDIOL's trend of altering gramicidin currents was the same at both concentrations, CANNABIDIOL's effects were more variable at 10 μM than 1 μM, and this variability resulted in lack of statistical significance at 10 μM (FIG. 11C). Speculatively, this may be due to gramicidin and high CANNABIDIOL causing a deterioration in HEK cell health over the timescales of voltage-clamp experiments. The dimerization of gramicidin channels indicates pore formation across the cell membrane. These pores are analogous to puncturing holes into the cell. To ensure that the currents are indeed gramicidin currents and there is no potential leak current component, the same experiment is conducted in lowered extracellular sodium [Na+=1 mM]. This experiment resulted in the same overall trends of altered gramicidin currents densities as the high [Na+] experiment, for both CANNABIDIOL and TX100 (FIG. 11E-H).

Overall, the gramicidin assay results of CANNABIDIOL altering membrane rigidity is consistent with and confirms the hypothesis regarding CANNABIDIOL potentially exerting some of its effects via the membrane.

### CANNABIDIOL Alters Rigidity in Gramicidin-Based Fluorescence Assay (GFA)

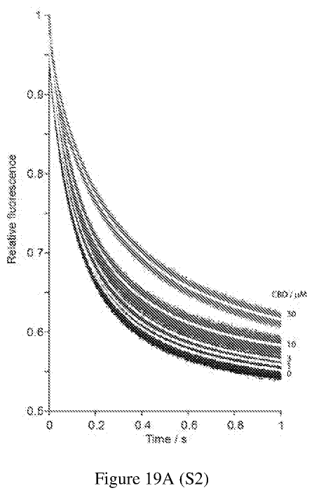

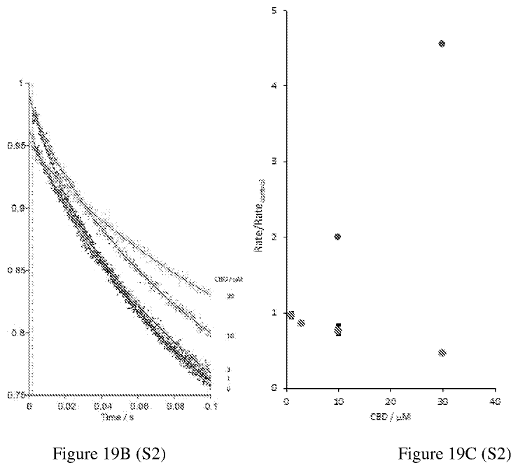

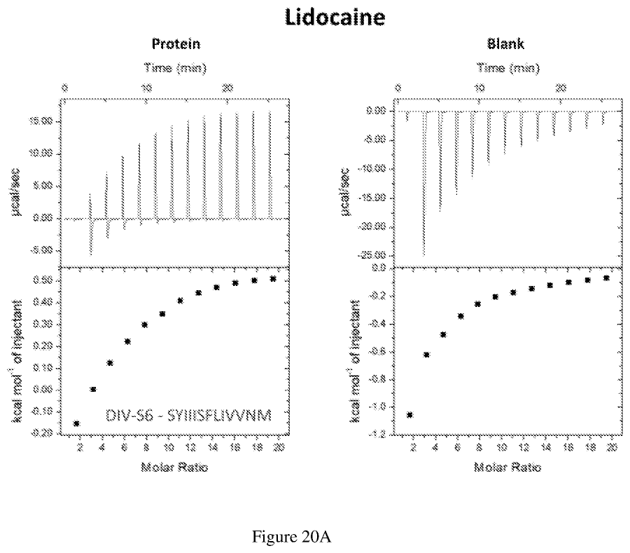

Ingólfsson et al provided (Ingólfsson 2010)) gramicidin-based fluorescence assay for determining small molecules potential for modifying lipid bilayer properties. To further explore CANNABIDIOL's possible effects on membrane rigidity, inventors tested its effects on lipid bilayer properties at concentrations where CANNABIDIOL has acute effects on Nav using a GFA. GFA takes advantage of the gramicidin channels' unique sensitivity to changes in bilayer properties. This assay as provided in FIG. 19 confirmed that CANNABIDIOL alters the gramicidin signal, and hence alters membrane rigidity.

CANNABIDIOL Interacts with the Nay Local-Anesthetic Site

It is previously reported that CANNABIDIOL displays an approximately 10-fold state-dependence in Nav inhibition (Ghovanloo (2018)), a property similar to classic pore-blockers (Kuo (1994) and Bean (1983)). Ghovanloo tested CANNABIDIOL inhibition from the inactivated-state in a Nav1.1 pore-mutant (F1763A-LA mutant) and the results suggested a relatively small ˜2.5-fold drop in potency.

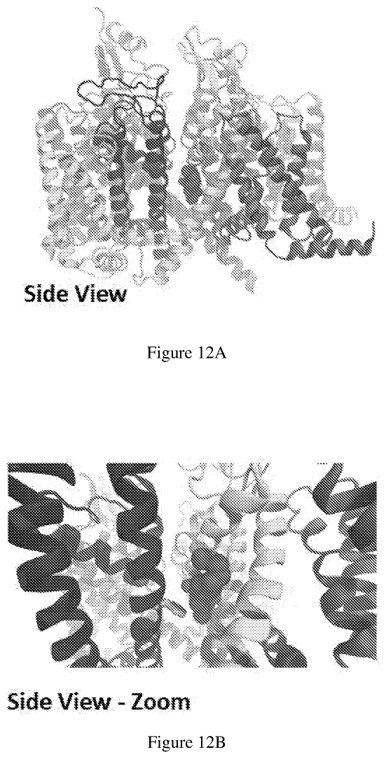

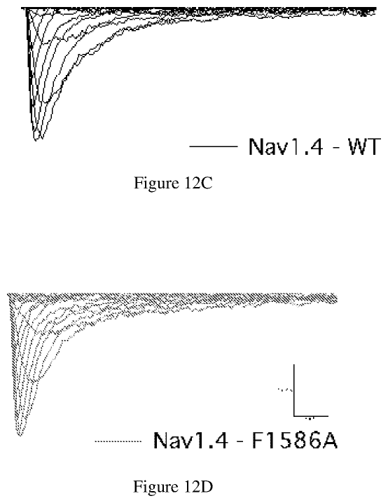

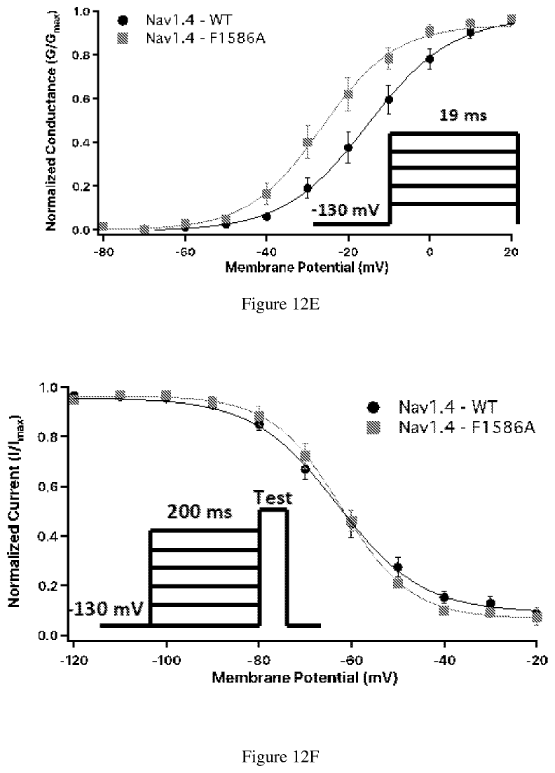

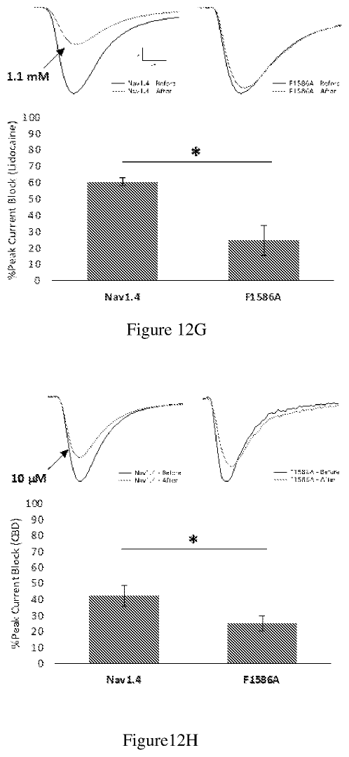

To further explore possible CANNABIDIOL interactions at the pore, molecular docking study is performed using Pan's (Pan (2018)) human Nav1.4 cryo-EM structure. FIGS. 12A-B provide CANNABIDIOL docked onto the Nav1.4 pore, supporting a possible interaction at the LA site. Further, to functionally test the docking result, inventors mutated the LA Nav1.4 F1586 into alanine and performed voltage-clamp. FIG. 12C-F provide biophysical characterization of F1586A compared with WT-Nav1.4. It is found that both channels have similar biophysical properties and, most importantly, the inactivation voltage-dependences were almost identical (p>0.05) (FIG. 12F), suggesting that at any given potential both F1586A and Nav1.4 would have the same availability; therefore, pharmacological experiments could be performed using the same voltage-protocols on both channels.

In contrast to neuronal Nays that have inactivation midpoints (V1/2) of ˜−65 mV in neurons with resting membrane potentials (RMP) that are also ˜−65 mV, Nav1.4 has a V1/2 of ˜−67 mV skeletal muscle fibers with RMP of ˜−90 mV. This indicates that, whereas neuronal Nays are almost always half-inactivated at RMP, Nav1.4 is almost always fully available at RMP.

Therefore, to get closer to physiological conditions (FIG. 12G-H), inhibition of Nav1.4 from rest (−110 mV holding-potential to 0 mV test-pulse at 1 Hz) by lidocaine as positive control and CANNABIDIOL are measured. The results suggest that 1.1 mM (resting IC50 on Nav1.446) lidocaine blocks ˜60% of INa in WT, and ˜20% in F1586A (p=0.020) whereas 10 μM CANNABIDIOL blocks ˜45% INa in WT and ˜25% in F1586A (p=0.037). Hence, there is a 3-fold difference between lidocaine's inhibition of WT vs. F1586A, and a smaller 1.5-fold difference for CANNABIDIOL inhibition. This suggests that while CANNABIDIOL may interact with the Nav pore similar to lidocaine, CANNABIDIOL's interaction at the pore is likely not as critical a determinant of its INa inhibition compared with lidocaine.

CANNABIDIOL Interacts with DIV-S6

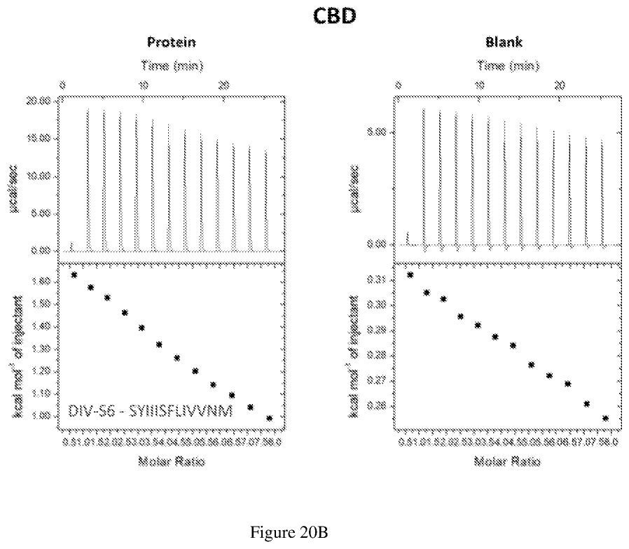

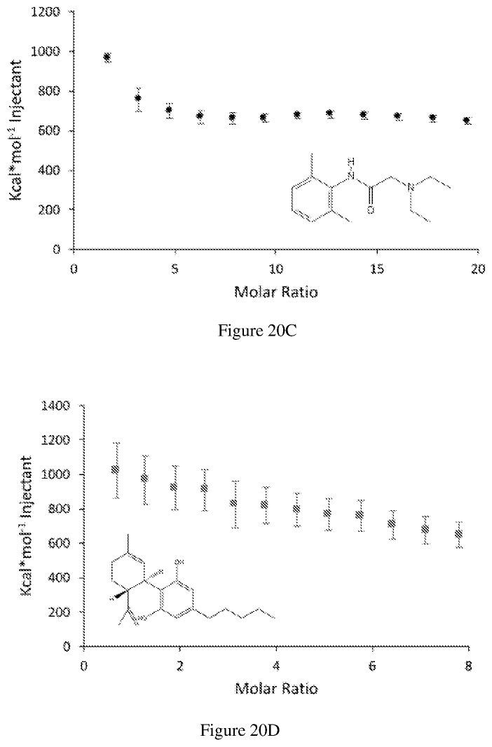

Since CANNABIDIOL's INa inhibition was less dependent on interactions in the local-anesthetic site than a well-established pore-blocker like lidocaine, it is further investigated using isothermal titration calorimetry. whether CANNABIDIOL interacts with the DIV-S6 (which includes F1586) or if it is inert, CANNABIDIOL interactions are compared to lidocaine. It is found that both lidocaine and CANNABIDIOL interact with the protein segment; however, the nature of this interaction is different between the two compounds, possibly due to a variation in physicochemical properties (FIG. 20).

CANNABIDIOL Penetrates into the Pore Through Fenestrations

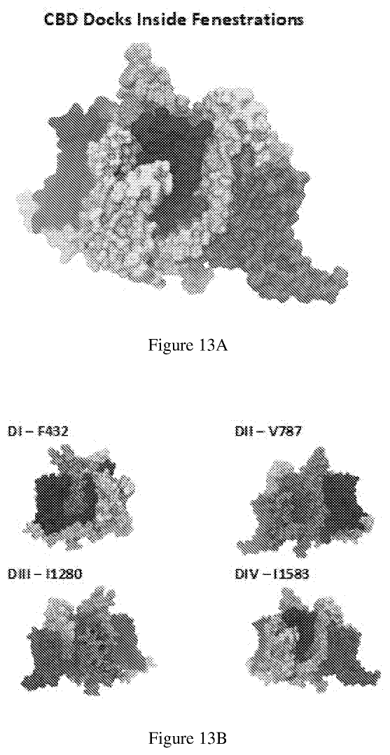

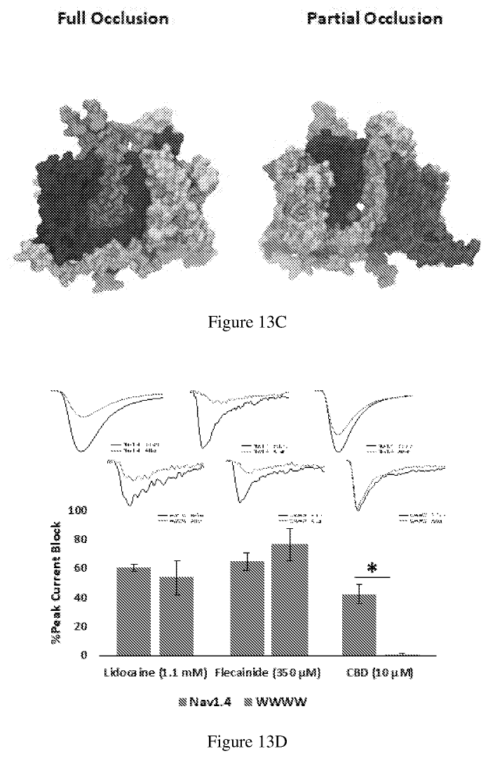

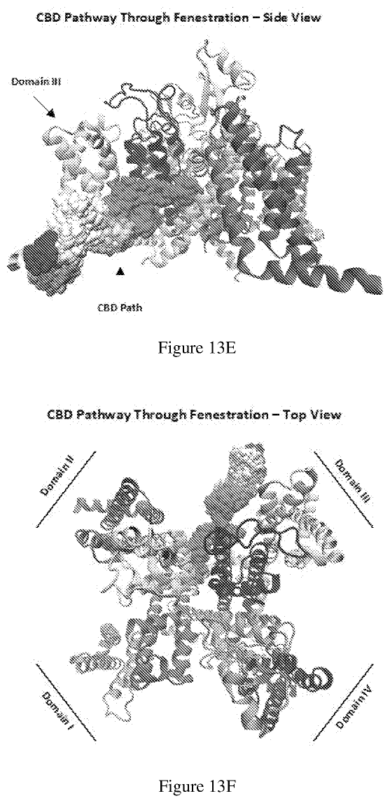

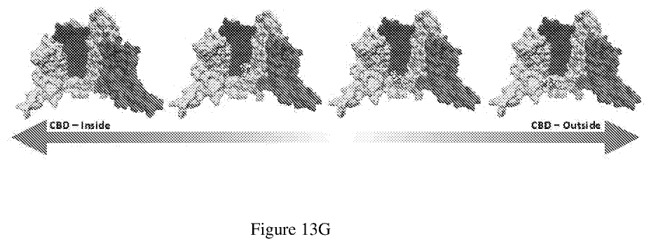

As reported by Gamal El-Din (2018), LAs block bacterial Nays in their resting-state by entering the pore through fenestrations in a size-dependent manner (i.e. smaller LAs get through more readily). Here, it is sought to determine whether it is possible to block CANNABIDIOL's access to the human Nav1.4 pore from the lipid phase of the membrane by occluding fenestrations. Since it is previously found that (Ghovanloo (2018)) CANNABIDIOL is highly lipid-bound (99.6%), and since MD results provide that it preferentially localized in the hydrophobic part of the membrane, just below the lipid headgroups, therefore, it is reasoned that once CANNABIDIOL partitions into the membrane, a pathway to the Nav pore is available through the intramembrane fenestrations. To test this idea, inventors scrutinized the docking pose of CANNABIDIOL in the human Nav1.4 and observed its localization close to the fenestrations (FIG. 13A; FIGS. 20A-D).

Next, 4 residues (DI-F432, DII-V787, DIII-I1280, and DIV-I1583) are identified that partially or fully occluded the fenestrations when mutated to W, as predicted by computational mutagenesis and structural minimization (partial versus full occlusion is due to structural asymmetry of mammalian Nays) (FIG. 13B-C).

Inventors measured resting-state block of 1.1 mM lidocaine, 350 μM flecainide, and 10 μM CANNABIDIOL from −110 mV on the WWWW construct. The results suggest that lidocaine (p>0.05) and flecainide (p>0.05), but not CANNABIDIOL (p<0.05) blocked the WWWW mutant the same as WT (FIG. 13D). This is an interesting result considering CANNABIDIOL is larger than lidocaine, but slightly smaller than flecainide. CANNABIDIOL's abolished block of WWWW relative to WT-Nav1.4 may be due to the vast difference in its lipophilicity compared to lidocaine (Log D˜1) and flecainide (Log D˜1.7). Overall, these results are consistent with the hypothesis regarding CANNABIDIOL's pathway from the membrane, through the fenestrations, and into the pore.

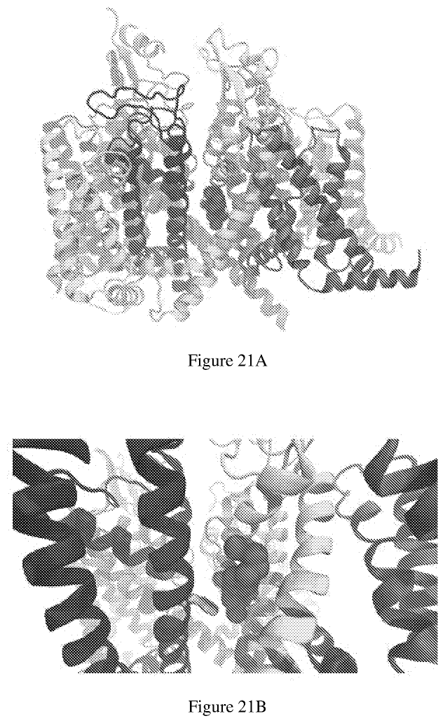

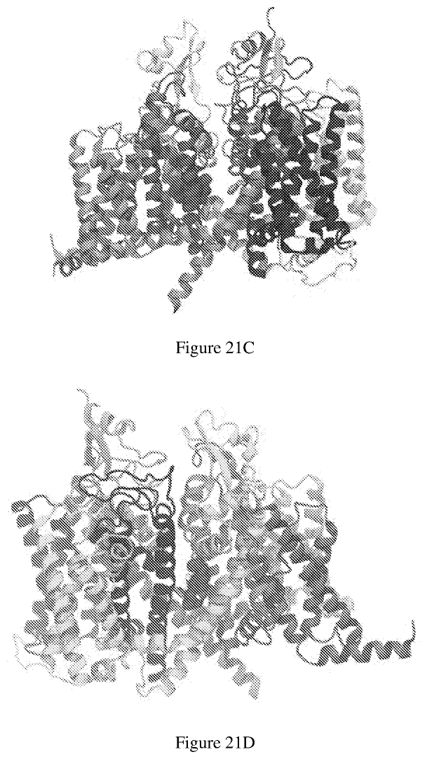

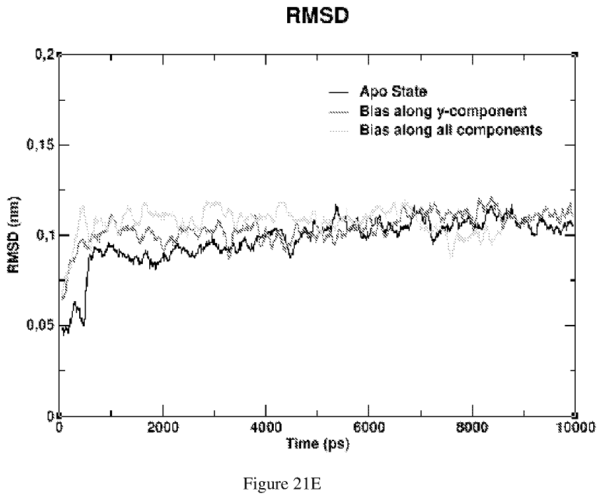

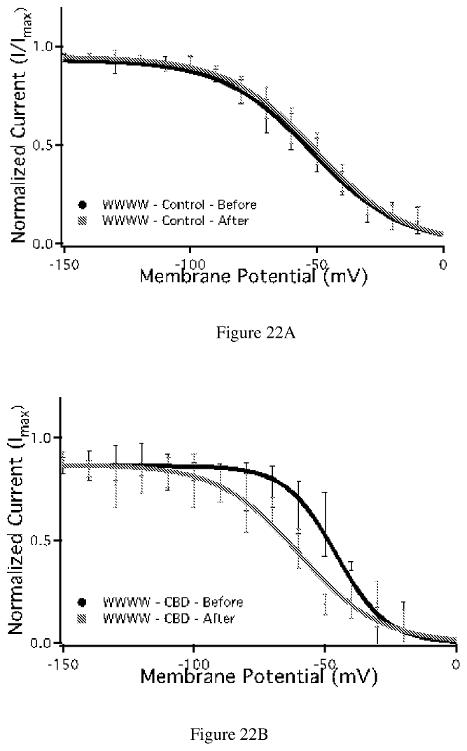

To visualize the possible pathway CANNABIDIOL follows through Nav1.4 fenestrations and into the pore at an atomistic resolution, MD simulations are performed in which inventors encouraged CANNABIDIOL to detach from its binding-site (FIG. 13E-G; FIG. 21E). These results demonstrate that CANNABIDIOL can enter its binding-site in the pore through the fenestration without major reorganization of the channel structure.

CANNABIDIOL does not Affect Nav1.4 Activation but Stabilizes the Inactivated-State

Ghovanloo (2018) characterized the effects of CANNABIDIOL on Nav1.1 gating. It is reported by Ghovanloo that ˜IC50 levels of CANNABIDIOL reduced channel conductance, did not change the voltage-dependence of activation, hyperpolarized steady-state fast-inactivation (SSFI), and slowed recovery from fast (300 ms) and slow (10 s) inactivation. De Petrocellis (2011) reports CANNABIDIOL's inhibition of resurgent sodium currents. These results suggested that CANNABIDIOL prevents the opening of a majority of Nays. However, those channels that still open, activate with unchanged voltage-dependence and are more likely to inactivate the overall effect is a reduction in excitability (Ghovanloo (2018)).

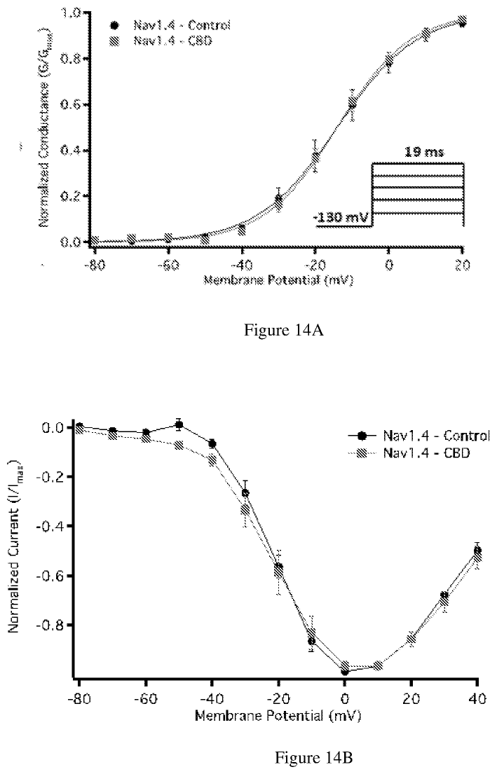

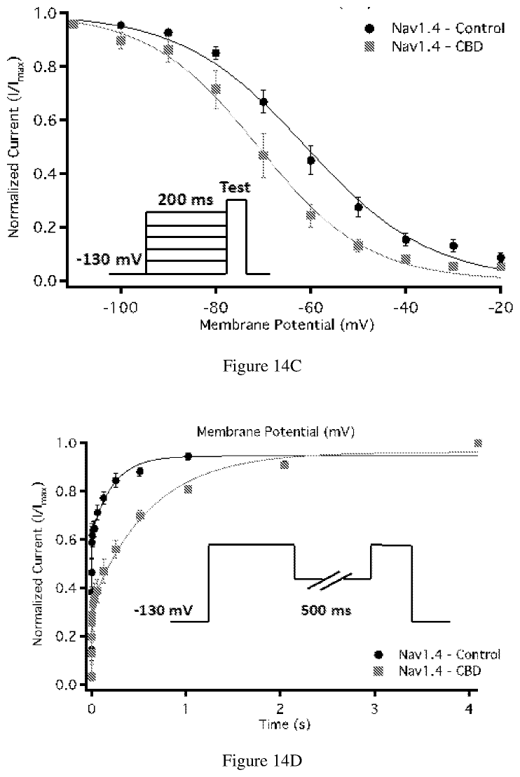

During this research, it is hypothesized that CANNABIDIOL's non-selectivity in INa inhibition suggests non-selectivity in modulating Nav gating. To test this idea, Nav1.4 activation in presence and absence of 1 μM CANNABIDIOL is assessed by measuring peak channel conductance at membrane potentials between −100 and +80 mV (FIG. 14A). CANNABIDIOL did not significantly affect V1/2 or apparent valence (z) of activation (p>0.05). Normalized Nav1.4 currents as a function of membrane potential are shown in FIG. 14B. These results indicate that, as with Nav1.1, CANNABIDIOL does not alter Nav1.4 activation.

Further, the voltage-dependence of SSFI is examined using a standard 200 ms pre-pulse voltage protocol. Normalized current amplitudes were plotted as a function of pre-pulse voltage (FIG. 14C). These results mimicked previous observations of Ghovanloo (2018) in Nav1.1, in that CANNABIDIOL left-shifted the Nav1.4 inactivation curve (p<0.05).

To measure recovery from inactivation, Nav1.4 is held at −130 mV to ensure channels are fully available, then the channels are pulsed to 0 mV for 500 ms and then different time intervals are allowed at −130 mV to measure recovery as a function of time. As previously observed in Nav1.1, CANNABIDIOL slowed the Nav1.4 recovery from inactivation (p<0.05), suggesting that it takes longer for CANNABIDIOL to come off the channels than the time taken by the channels to recover from inactivation (FIGS. 14E and F). Collectively, these results support the hypothesis that CANNABIDIOL non-selectively modulates Nav gating, and further suggests that CANNABIDIOL reduces Nav1.4 excitability.

### CANNABIDIOL Hyperpolarizes SSFI in Nav1.4-WWWW

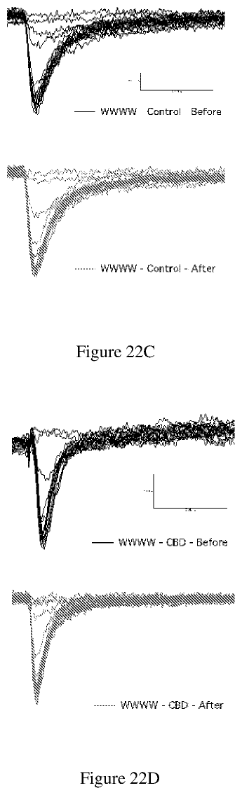

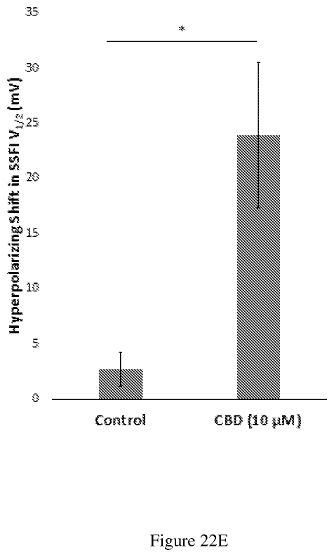

To determine a possible association between membrane rigidity and stabilized inactivation, the effects of CANNABIDIOL are measured before and after compound perfusion in the WWWW mutant. It is found that although CANNABIDIOL does not inhibit peak INa, it hyperpolarizes the SSFI curve, suggesting CANNABIDIOL's modulation of membrane rigidity is at least in part responsible for stabilizing Nav inactivation (FIG. 22).

CANNABIDIOL Effects on a pH-Sensitive Mixed Myotonia/hypoPP Nav1.4-Mutant, P1158S (DIII-S4-S5)

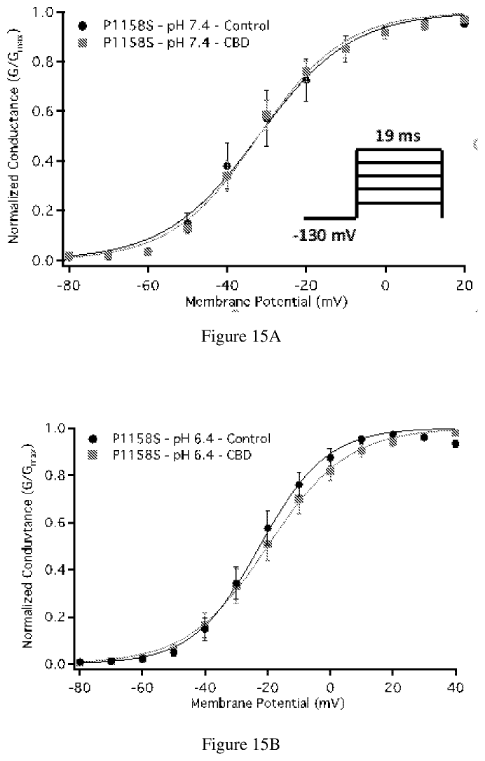

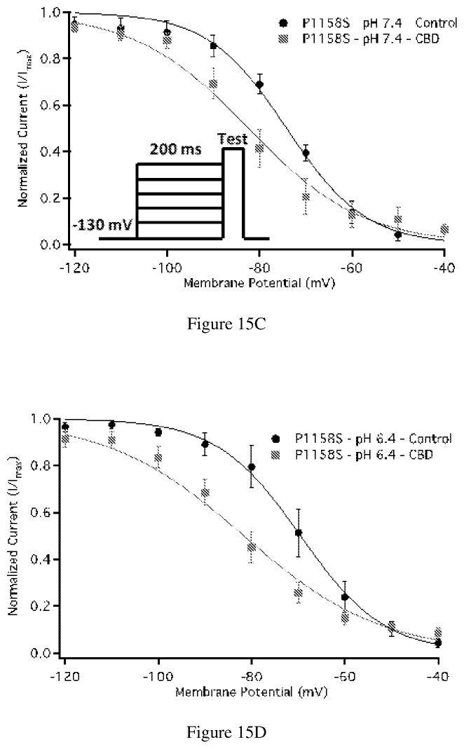

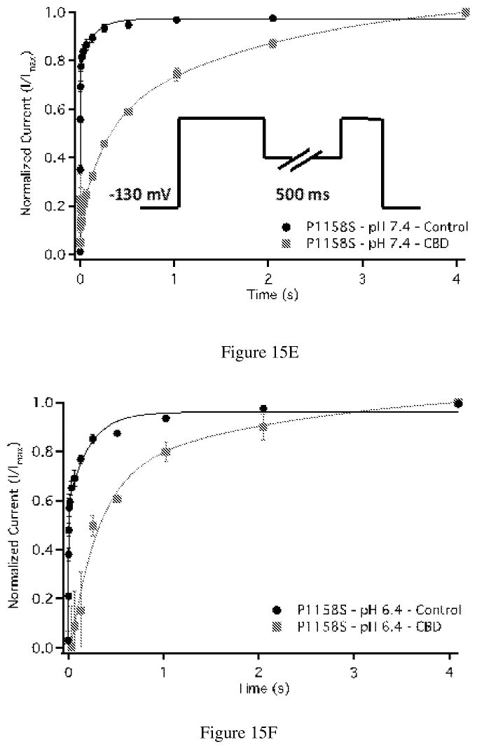

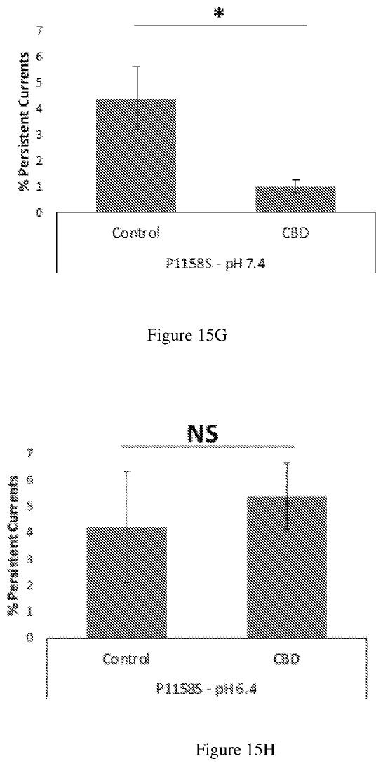

CANNABIDIOL's role to ameliorate a skeletal muscle GOF condition is investigated. It is recently discovered that the P1158S mutation in Nav1.4 increases the channel's pH-sensitivity (Ghovanloo and Abdelsayed (2018)). Interestingly, the P1158S gating displays pH-dependent shifts that, using AP modeling, are predicted to correlate with the phenotypes associated with this variant. Therefore, the relationship between pH and P1158S could be used as an in-vitro/in-silico assay of Nav1.4 hyperexcitability (to model moderate to severe GOF). This assay is used as a model to investigate CANNABIDIOL effects on skeletal muscle hyperexcitability. Effects of 1 μM CANNABIDIOL (pKa=9.64) on P1158S at pH6.4 (myotonia-triggering) and pH7.4 (hypoPP-triggering) are tested. FIG. 15 provides CANNABIDIOL effects on P1158S at low and high pH. Interestingly, the lack of selectivity in CANNABIDIOL gating modulation by CANNABIDIOL also exists in P1158S at both pHs. CANNABIDIOL did not change activation (p>0.05), but hyperpolarized inactivation (p<0.05) and slowed recovery from inactivation (p<0.05) (FIG. 15A-F). Consistent with previous results (Ghovanloo (2018) and Ghovanloo (2016)) where CANNABIDIOL inhibited persistent INa, CANNABIDIOL also reduced the exacerbated persistent INa associated with P1158S at pH7.4 (p<0.05) (FIG. 15G). Persistent INa reduction could not be detected at pH6.4 (p>0.05) (FIG. 15H) because both low pH (Ghovanloo, and Abdelsayed; and Ghovanloo, and Peters) and CANNABIDIOL reduce current amplitude to levels such that differences in small current amplitudes could not be resolved above background noise.

AP Model Predicts CANNABIDIOL Reduces Myotonia, but not hypoPP in the P1158S-pH Assay

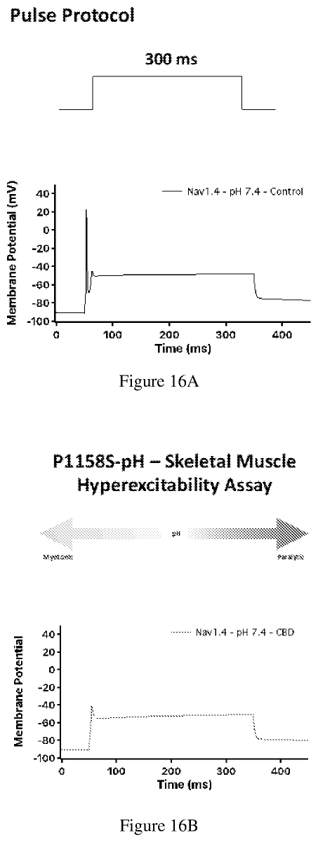

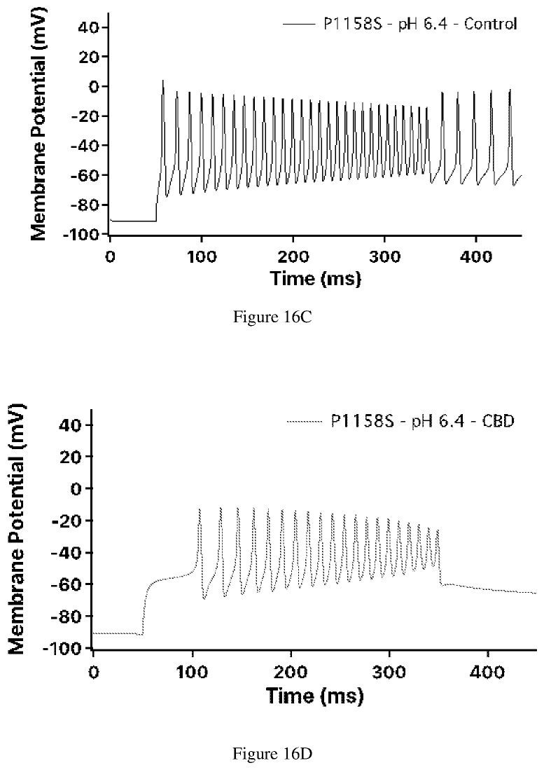

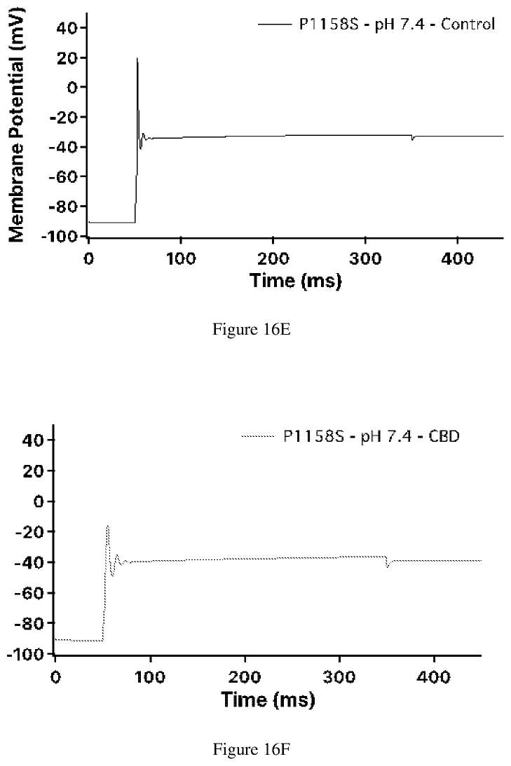

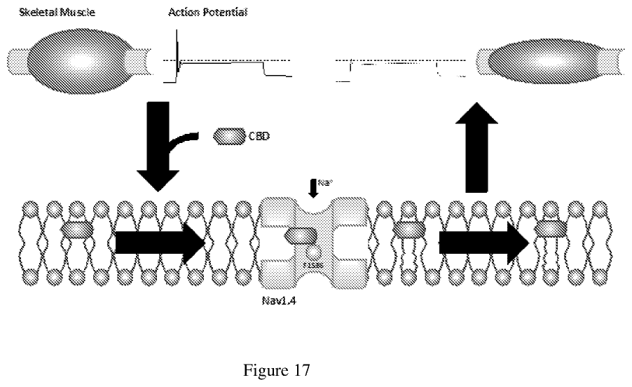

In this study, inventors used the gating changes from the patch-clamp experiments with WT and P1158S (both control and CANNABIDIOL (1 μM)) to model the skeletal muscle AP49. The simulations were run using a 50 μA/cm2 stimulus. The simulation pulse started at 50 ms and stopped at 350 ms (FIG. 16A). During this pulse, the WT channels activated at 50 ms and fired a single AP. The channels remained inactivated until the stimulus was removed at 350 ms and then the membrane potential recovered back to its resting value (FIG. 16A). CANNABIDIOL reduced the AP amplitude (FIG. 16B), consistent with CANNABIDIOL effects observed in different neuron types as provided in Ghovanloo (2018) and Khan (2018). As provided in FIG. 16C, at pH 6.4, P1158S displayed a continuous train of APs for the entire stimulation period. After the stimulus was removed, P1158S showed a progressive series of after-depolarizations of the membrane potential. Such series of after-depolarizations is a characteristic of a myotonic burst (Cannon (2015)). Interestingly, the CANNABIDIOL-mediated shifts at pH6.4 in P1158S reduced the simulated AP amplitudes for the entirety of the pulse duration, delayed onset of first AP, consistent with CANNABIDIOL preventing Nav opening, and abolished the post-pulse myotonic after-depolarizations (FIG. 16D). At pH7.4, P1158S fired a single AP, followed by a period where membrane potential remained depolarized around −35 mV, even post-stimulus termination (FIG. 16E). This inability to repolarize holds the Nays in an inactivated state and is consistent with the periodic paralysis phenotype. In contrast to the myotonic phenotype, CANNABIDIOL did not alleviate the hypoPP phenotype in P1158S-pH in-vitro/in-silico assay (FIG. 16F), consistent with its slowing of recovery from fast inactivation.

The skeletal muscle contractility complications arise due to pathogenic variants of the skeletal muscle sodium channel, Nav1.4. From all above studies, CANNABIDIOL has emerged as a therapeutic agent to alleviate skeletal muscle contractility complications, and as an agent which alters membrane rigidity and penetrates into the Nav pore through fenestrations. The studies performed using various ex-vivo, in-vitro, and in-silico techniques suggested that CANNABIDIOL alleviates myotonia via its direct and indirect effects on Nav1.4. CANNABIDIOL's inhibition of Nav currents (and possibly other ionic currents) is, at least in part, mediated through changing lipid bilayer rigidity.

Thus, CANNABIDIOL in suitable pharmaceutical compositions can be administered to alleviate

i) Skeletal muscle contractility;
ii) myotonia via its direct and indirect effects on Nav1.4
iii) muscle stiffness.

### Possible Clinical Applications for CANNABIDIOL in Skeletal Muscle Disorders

CANNABIDIOL has produced significantly positive effects on molecular target Nav 1.5 and is set to rescue the target from the deleterious effects of higher glucose. Higher glucose concentrations are usually used as a model to mimic the in vivo situation of hyperglycaemia in diabetes and therefore, CANNABIDIOL is set to rescue the molecular target Nav1.5 from various effects exerted on the said sodium channel in situation of hyperglycaemia in diabetes.

Hyperglycaemia and Diabetes affect gating properties of sodium channel Nav1.5 in one or more ways. Either the sodium channel remains hyperexcited and is unable to get inactivated in desired time or it is not recovered from inactivation to be available for further action potential. Sometimes it fails to activate at any given membrane potential. Hyperglycaemia and Diabetes also cause cytotoxicity and affect cell viability of sodium channel. These conditions also enhance ROS levels and thus cause cytotoxicity.

Thus, CANNABIDIOL through its favourable antioxidant and sodium channel inhibitory effects, protects against high-glucose induced arrhythmia and cytotoxicity.

Skeletal muscle hyperexcitability disorders have historically received less attention than disorders in other tissues, including the brain. Drugs most commonly used for myotonia include compounds developed for other conditions, such as anti-convulsants and anti-arrhythmics (Alfonsi (2007) and Trip (2008)), which may cause unwanted, off-target side-effects. Hence, another therapeutic approach has been lifestyle modifications. For instance, myotonic patients may modify their lifestyles to avoid triggers like potassium ingestion or cold temperatures. Treatment of hypoPP is usually achieved using oral potassium ingestion and by avoiding dietary carbohydrates and sodium.

Cannabinoids have long been used to alleviate muscular problems. A study is performed to find out whether CANNABIDIOL reduces skeletal contraction in rat diaphragm muscle. As CANNABIDIOL is a poly-pharmacology compound, one may not conclude with certainty that the observed contraction reduction is due to INa inhibition alone, but the similarity to TTX results suggest that INa block is sufficient to reduce contraction, and therefore CANNABIDIOL's activity at Nav1.4 could be a part of the mechanism in this reduction.

To explore a possible use for CANNABIDIOL in myotonia and hypoPP, it is tested in an in-vitro/in-silico assay. The results suggest that CANNABIDIOL may alleviate the myotonic but not the hypoPP phenotype.

Jurkat-Rott reports that in the 1990s, the term ion channelopathies was coined and defined for disorders that are caused by malfunction or altered regulation of ion channel proteins. Therefore, they may be either hereditary (for example by mutations in ion channel genes) or acquired (for example by autoantibodies). Since then, over 50 channelopathies in human beings have been described, 12 of which affect skeletal muscle. Of these, five are caused by mutations in its voltage-gated sodium channel, NaV1.4: potassium-aggravated myotonia (PAM), paramyotonia congenita (PMC), hyperkalemic periodic paralysis (HyperPP), hypokalemic periodic paralysis (HypoPP), and a form of congenital myasthenic syndrome (CMS). The inventors further propose that even if Nav1.4 is not mutated but has any acquired condition affecting its function, CANNABIDIOL pharmaceutical compositions will provide promising treatment. Such conditions may include impact due to therapies.

In conclusion, results of various experiments suggest that CANNABIDIOL inhibition of Nav1.4 has at least two components: altered membrane rigidity and pore block. Nav1.4 inhibition could contribute to CANNABIDIOL reducing skeletal muscle contractions and may have potential therapeutic value against myotonia.

Veterinary applications: Suitable CANNABIDIOL Pharmaceutical compositions can be prepared for Veterinary applications for mammals particularly pets such as goat, dogs, cats etc. Myotonic or Fainting goat where the condition is characterised by myotonia congenita, a hereditary condition which may cause it to stiffen or fall over when startled can be treated with pharmaceutical compositions of CANNABIDIOL.

From a broader perspective, the proposed mechanism may hold true for other compounds that are similar to CANNABIDIOL in modulating Nays or other channels with similar structures.

A suitable dose of one or more cannabinoids is from 0.00001 mg/kg of body weight to 4000 mg/kg of body weight for each cannabinoid. The suitable dose can also be 0.00001 to 1000 mg/kg of body weight or 0.01 to 500 mg/kg of body weight. The preferred dose can be 0.01 to 100 mg/kg of body weight or from 0.01 to 10 mg/kg of body weight.

The dose will depend on the nature and status of human or animal patient health. It will also depend on age and comorbidities if any. Further, dose will depend on type of pharmaceutical composition for example, whether oral or parenteral or topical.

Following pharmaceutical formulations/pharmaceutical compositions are described for better understanding of the invention and they do not limit scope of the invention in any way.

Under the tenth aspect, the invention provides various pharmaceutical compositions of CANNABIDIOL employed in several aspects from 1-9.

The dosage form can be preferably oral but also one or more or all drugs can be administered parenterally when an urgent treatment is expected or when the patient is not capable of receiving an oral treatment. Formulations can be administered via any suitable administration route. For example, the formulations (and/or pharmaceutical compositions) can be administered to the subject in need thereof orally, intravenously, intramuscularly, intravaginally, intraperitoneally, rectally, parenterally, intraocularly, topically, intranasally, subcutaneously or by otic route. Suitable oral dosage forms include tablets—sublingual, buccal, effervescent, chewable; troches, lozenges, dispersible powders or granules and dragees; capsules, solutions, suspensions, syrups, lozenges, medicated gums, buccal gels or patches. Tablets can be made using compression or molding techniques well known in the art. The other dosage forms can also be prepared by 3Dimensional (3D) or 4D printing and also by Carbon graphene loaded nano-particles and micro-particles. Gelatin or non-gelatin capsules can be formulated as hard or soft capsule shells, which can encapsulate liquid, solid, and semi-solid fill materials, using techniques well known in the art.

When patient can receive oral treatment, the treatment may involve giving an oral tablet or capsule of CANNABIDIOL along with pharmaceutical composition of any drug with which administration of CANNABIDIOL is desirable due to multiple roles of CANNABIDIOL. Any drug which can induce LQT, any drug which can trigger cytokines or inflammation or any drug which may have any adverse effect on heart, are likely candidates. Antibiotics such as macrolide antibiotics are one such preferred candidates. Drugs like chloroquine and hydroxy chloroquine which also induce LQT and which have been recently discovered for their potential to act against Covid-19 are also likely candidates. Any such therapeutic agent can be administered before, with or after CANNABIDIOL. Sometimes it is possible to combine the other therapeutic agent in the same pharmaceutical composition as that of CANNABIDIOL. In an embodiment, a tri-layered tablet containing CANNABIDIOL, chloroquine/hydroxychloroquine and azithromycin can be prepared as provided in the examples. When the therapy is given to children or elderly, instead of solid oral dosage forms, liquid orals can be preferred.

CANNABIDIOL can be administered with another Therapeutic agent's suspension or solution. When cannabidiol is administered with the other therapeutic agent, such administration can be simultaneous/concomitant or sequential and to serve some purpose.

Cannabidiol can be administered in the form of suitable composition with other therapeutic agents which induce long QT or inflammation to avoid inducing gating defects in sodium channel by such other therapeutic agent.

Cannabidiol and the other therapeutic agent can be combined in the same composition or can be provided in different compositions. The factors which determine whether they should or should not be combined in a single composition are vast but without limitation include doses, solubility, stability, compatibility, bioavailability, route of administration, dosing frequency, half life etc.

When combining in a single composition is not possible, the cannabidiol compositions can be provided in a kit form with the compositions of other therapeutic agents.

Owing to reduction in reactive oxygen species, Cannabidiol compositions reduce oxidative stress/damage and can be employed with any agent or condition which induces oxidative stress/damage and inflammation.

Cannabidiol compositions enhance the safety profile of another therapeutic agent or therapy and they can be administered in any existing therapy for example, along with vaccines particularly Covid-19 vaccines which are known to induce several side effects including cardiac side effects and inflammation.

As an alternative to oral therapy or to avoid oral route when needed, nasal therapy such as nasal spray can be prepared as provided in the examples. The said formulation can be administered via the nasal route as nasal drops or as nasal spray using appropriate medical device. The said formulation can be administered via inhalation with or without the aid of a medical device, metered or unmetered, and/or via nebulization.

As another alternative, buccal or sublingual sprays can also be made.

For patients for whom injectables are essential, CANNABIDIOL and other therapeutic agents can be administered as injectables.

Whenever it is possible to combine CANNABIDIOL with the existing therapy of other therapeutic agents (such as already marketed chloroquin/hydroxychloroquine pharmaceutical compositions, only CANNABIDIOL pharmaceutical compositions should be prepared as provided under examples but sometimes when such treatment is not available or when there is a need to combine CANNABIDIOL in certain form with chloroquine/hydroxychloroquine, even corresponding antiviral pharmaceutical compositions are provided under examples. Examples of some therapeutic agents which can be administered with CANNABIDIOL include oseltamivir phosphate, atazanavir sulphate and ribavirin.

Pharmaceutical compositions of CANNABIDIOL containing pharmacologically effective concentration of CANNABIDIOL are provided which can alleviate several effects of pathogenic sodium channel Nav1,4 such as skeletal muscle contractility complications, myotonia, muscle stiffness and pain and inherited as well as acquired LOTs.

Pharmaceutical compositions of CANNABIDIOL containing pharmacologically effective concentration of CANNABIDIOL are provided which rescue sodium channels from most of the adverse effects of high glucose observed in hyperglycaemia and diabetes.

These pharmaceutical compositions further include one or more pharmaceutical carrier appropriate for administration to an individual in need thereof. The pharmaceutical compositions are suitable for acting on at least one molecular target which is Nav1.5. These pharmaceutical compositions would produce beneficial effects in one or more of the following pathogenesis of various cardiovascular disorders including, but not limited to, long QT syndrome, long QTc syndrome, long QRS syndrome, cardiomyopathy, heart failure, arrhythmia, myocardial ischemia, myocardial infarction (MI), arrhythmias of ischemic and non-ischemic origin, inflammation, vascular dysfunction, cardiomyopathy, cardiac remodelling, maladaptation, anginas of different types, drug induced heart failure, iatrogenic heart and vascular diseases, or any combination thereof.

The individual in need thereof can have or can be suspected of having elongated QT interval, a symptom thereof, and/or a related complication thereof including but not limited to high glucose elicited oxidative stress and cytotoxicity.

Pharmaceutical compositions can be administered via any suitable administration route. For example, they can be administered to the subject in need thereof orally, sublingually, buccally, intravenously, intramuscularly, intravaginally, intraperitoneally, rectally, parenterally, intraocularly, topically, transdermally, intranasally, or subcutaneously. Other suitable routes are described herein.

Various pharmaceutical compositions are hereinbelow described.

### Oral Pharmaceutical Compositions

The pharmaceutical compositions are designed to modify, alter and particularly improve solubility of CANNABIDIOL. CANNABIDIOL has good lipid solubility but its aqueous solubility is poor. Thus, pharmaceutical compositions of CANNABIDIOL may contain soluble or disintegrating excipients or binders and particularly those excipients which enhance solubility of CANNABIDIOL in water or in a solvent used in case of liquid preparations. The pharmaceutical compositions may additionally contain stabilizer, anti-oxidant, sweetener, flavours and colourants.

Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful.

Binders can impart cohesive qualities to a solid dosage formulation, and thus can ensure that a tablet or bead or granule remains intact after the formation of the dosage forms.

Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and magnesium aluminum silicate (Veegum®), and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders.

Lubricants can be included to facilitate tablet manufacture. Suitable lubricants include, but are not limited to, magnesium stearate, calcium stearate, stearic acid, glycerol behenate, polyethylene glycol, talc, and mineral oil. A lubricant can be included in a tablet formulation to prevent the tablet and punches from sticking in the die. The lubricant can be chosen from such slippery solids as talc, magnesium and calcium stearate, stearic acid and hydrogenated vegetable oils.

Disintegrants can be used to facilitate dosage form disintegration or “breakup” after administration, and generally include, but are not limited to, starch, sodium starch glycolate, sodium carboxymethyl starch, sodium carboxymethylcellulose, hydroxypropyl cellulose pregelatinized starch, clays, cellulose, alginine, gums or cross-linked polymers, such as cross linked PVP (Polyplasdone® XL from GAF Chemical Corp).

Stabilizers can be used to inhibit or retard drug-pharmaceutical composition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

Solubilizers may contain surfactants. Suitable surfactants can be anionic, cationic, amphoteric or non-ionic surface-active agents. Suitable anionic surfactants include, but are not limited to, those containing carboxylate, sulfonate and sulfate ions.

### Delayed Release/Sustained Release/Extended Release Pharmaceutical Compositions

Delayed release dosage pharmaceutical compositions containing the Nav 1.5 channel modulator as described herein can be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

Alternative to a delayed release delivery, the pharmaceutical compositions can also be prepared in a sustained release, or an extended release, or a combined sustained release and extended release fraction dosage form, or in an immediate release dosage form, or a combined sustained release fraction and immediate release fraction dosage form, or a combination thereof.

The pharmaceutical compositions where release is modified can be formulated as matrix preparations, coated preparation, multilayer or tablet in tablet preparations, osmotic preparations etc. The pharmaceutical compositions containing the Nav 1.5 channel modulator as described herein can be coated with a suitable coating material, for example, to delay release once the particles have passed through the acidic environment of the stomach. Suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings can be formed with a different ratio of water-soluble polymer, water insoluble polymers and/or pH dependent polymers, with or without water insoluble/water soluble non polymeric excipient, to produce the desired release profile. The coating can be performed on a dosage form (matrix or simple) which includes, but is not limited to, tablets (compressed with or without coated beads), capsules (with or without coated beads), beads, particle pharmaceutical compositions, “ingredient as is” formulated as, but not limited to, suspension form or as a sprinkle dosage form.

Additionally, the coating material can contain conventional carriers such as plasticizers, pigments, colorants, glidants, stabilization agents, pore formers and surfactants. Optional pharmaceutically acceptable excipients include, but are not limited to, diluents, binders, lubricants, disintegrants, colorants, stabilizers, and surfactants.

Diluents, also referred to as “fillers,” can be used to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful.

Binders can impart cohesive qualities to a solid dosage formulation, and thus can ensure that a tablet or bead or granule remains intact after the formation of the dosage forms.

Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and magnesium aluminum silicate (Veegum®), and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders.

Lubricants can be included to facilitate tablet manufacture. Suitable lubricants include, but are not limited to, magnesium stearate, calcium stearate, stearic acid, glycerol behenate, polyethylene glycol, talc, and mineral oil. A lubricant can be included in a tablet formulation to prevent the tablet and punches from sticking in the die. The lubricant can be chosen from such slippery solids as talc, magnesium and calcium stearate, stearic acid and hydrogenated vegetable oils.

Disintegrants can be used to facilitate dosage form disintegration or “breakup” after administration, and generally include, but are not limited to, starch, sodium starch glycolate, sodium carboxymethyl starch, sodium carboxymethylcellulose, hydroxypropyl cellulose pregelatinized starch, clays, cellulose, alginine, gums or cross-linked polymers, such as cross linked PVP (Polyplasdone® XL from GAF Chemical Corp). Stabilizers can be used to inhibit or retard drug-pharmaceutical composition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

### Parenteral Pharmaceutical Compositions

The Nav 1.5 channel modulator can be formulated for parenteral delivery, such as injection or infusion, in the form of a solution or suspension. The formulation can be administered via any route, such as, the blood stream or directly to the organ or tissue to be treated.

Parenteral formulations can be prepared as aqueous pharmaceutical compositions using techniques known in the art. Typically, such pharmaceutical compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes such as Self Micro-emulsifying Drug Delivery Systems (SMEDDS) and or Micellar.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the Nav 1.5 channel modulator as described herein can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants can be anionic, cationic, amphoteric or non-ionic surface-active agents. Suitable anionic surfactants include, but are not limited to, those containing carboxylate, sulfonate and sulfate ions. Suitable anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Suitable cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Suitable nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-p-alanine, sodium N lauryl-p-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation can also contain an antioxidant to prevent degradation of Nav 1.5 channel modulator. The formulation can be buffered to a pH of 3-8 for parenteral administration upon reconstitution. Suitable buffers include, but are not limited to, phosphate buffers, acetate buffers, and citrate buffers.

Water-soluble polymers can be used in the pharmaceutical compositions for parenteral administration. Suitable water-soluble polymers include, but are not limited to, polyvinylpyrrolidone, dextran, carboxymethylcellulose, and polyethylene glycol. Sterile injectable solutions can be prepared by incorporating the Nav 1.5 channel modulator in the required amount in the appropriate solvent or dispersion medium with one or more of the excipients listed above, as required, followed by filtered sterilization. Dispersions can be prepared by incorporating the various sterilized Nav 1.5 channel modulator into a sterile vehicle which contains the basic dispersion medium and the required other ingredients from those listed above. Sterile powders for the preparation of sterile injectable solutions can be prepared by vacuum-drying and freeze-drying techniques, which yields a powder of the Nav 1.5 channel modulator plus any additional desired ingredient from a previously sterile-filtered solution thereof. The powders can be prepared in such a manner that the particles are porous in nature, which can increase dissolution of the particles. Methods for making porous particles are well known in the art.

Pharmaceutical formulations for parenteral administration can be in the form of a sterile aqueous solution or suspension of particles formed from one or more Nav 1.5 channel modulator. Acceptable solvents include, for example, water, Ringer's solution, phosphate buffered saline (PBS), and isotonic sodium chloride solution. The formulation can also be a sterile solution, suspension, or emulsion in a nontoxic, parenterally acceptable diluent or solvent such as 1,3-butanediol.

In some instances, the formulation can be distributed or packaged in a liquid form. In other embodiments, formulations for parenteral administration can be packed as a solid, obtained, for example by lyophilization of a suitable liquid formulation. The solid can be reconstituted with an appropriate carrier or diluent prior to administration.

Solutions, suspensions, or emulsions for parenteral administration can be buffered with an effective amount of buffer necessary to maintain a pH suitable for ocular administration. Suitable buffers include, but are not limited to, acetate, borate, carbonate, citrate, and phosphate buffers.

Solutions, suspensions, or emulsions for parenteral administration can also contain one or more tonicity agents to adjust the isotonic range of the formulation. Suitable tonicity agents include, but are not limited to, glycerin, mannitol, sorbitol, sodium chloride, and other electrolytes. Solutions, suspensions, or emulsions for parenteral administration can also contain one or more preservatives to prevent bacterial contamination of the ophthalmic preparations.

Suitable preservatives include, but are not limited to, polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions, use of nanotechnology including nano-formulations for parenteral administration can also contain one or more excipients, such as dispersing agents, wetting agents, and suspending agents.

### Topical and Transdermal Pharmaceutical Compositions

The Nav 1.5 channel modulator as described herein can be formulated for topical administration. Nav 1.5 channel modulator can have a formula according to the ones mentioned herein. Suitable dosage forms for topical administration include creams, ointments, salves, sprays, gels, lotions, emulsions, liquids, and transdermal patches. The formulation can be formulated for transmucosal, transepithelial, transendothelial, or transdermal administration. The topical formulations can contain one or more chemical penetration enhancers, membrane permeability agents, membrane transport agents, emollients, surfactants, stabilizers, and combination thereof.

In some embodiments, the Nav 1.5 channel modulator can be administered as a liquid formulation, such as a solution or suspension, a semi-solid formulation, such as a lotion or ointment, or a solid formulation. In some embodiments, the Nav 1.5 channel modulator can be formulated as liquids, including solutions and suspensions, such as eye drops or as a semi-solid formulation, such as ointment or lotion for topical application to the skin, to the mucosa, such as the eye, to the vagina, or to the rectum.

The formulation can contain one or more excipients, such as emollients, surfactants, emulsifiers, penetration enhancers, and the like.

Suitable emollients include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In some embodiments, the emollients can be ethylhexylstearate and ethylhexyl palmitate.

Suitable surfactants include, but are not limited to, emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In some embodiments, the surfactant can be stearyl alcohol.

Suitable emulsifiers include, but are not limited to, acacia, metallic soaps, certain animal and vegetable oils, and various polar compounds, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In some embodiments, the emulsifier can be glycerol stearate.

Suitable classes of penetration enhancers include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols).

Suitable emulsions include, but are not limited to, oil-in-water, water-in-oil emulsions or multiple emulsions. Either or both phases of the emulsions can include a surfactant, an emulsifying agent, and/or a liquid non-volatile non-aqueous material. In some embodiments, the surfactant can be a non-ionic surfactant. In other embodiments, the emulsifying agent is an emulsifying wax. In further embodiments, the liquid non-volatile non-aqueous material is a glycol. In some embodiments, the glycol is propylene glycol. The oil phase can contain other suitable oily pharmaceutically acceptable excipients. Suitable oily pharmaceutically acceptable excipients include, but are not limited to, hydroxylated castor oil or sesame oil can be used in the oil phase as surfactants or emulsifiers.

Lotions containing the Nav 1.5 channel modulator as described herein are also provided. In some embodiments, the lotion can be in the form of an emulsion having a viscosity of between 100 and 1000 centistokes. The fluidity of lotions can permit rapid and uniform application over a wide surface area. Lotions can be formulated to dry on the skin leaving a thin coat of their medicinal components on the skin's surface.

Creams containing the Nav 1.5 channel modulator as described herein are also provided. The cream can contain emulsifying agents and/or other stabilizing agents. In some embodiments, the cream is in the form of a cream having a viscosity of greater than 1000 centistokes, typically in the range of 20,000-50,000 centistokes. Creams, as compared to ointments, can be easier to spread and easier to remove.

One difference between a cream and a lotion is the viscosity, which is dependent on the amount/use of various oils and the percentage of water used to prepare the formulations. Creams can be thicker than lotions, can have various uses, and can have more varied oils/butters, depending upon the desired effect upon the skin. In some embodiments of a cream formulation, the water-base percentage can be about 60% to about 75% and the oil base can be about 20% to about 30% of the total, with the other percentages being the emulsifier agent, preservatives and additives for a total of 100%.

Ointments containing the Nav 1.5 channel modulator as described herein and a suitable ointment base are also provided. Suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments).

Pastes typically differ from ointments in that they contain a larger percentage of solids. Pastes are typically more absorptive and less greasy that ointments prepared with the same components.

Also described herein are gels containing the Nav 1.5 channel modulator as described herein, a gelling agent, and a liquid vehicle. Suitable gelling agents include, but are not limited to, modified celluloses, such as hydroxypropyl cellulose and hydroxyethyl cellulose; Carbopol® homopolymers and copolymers; thermo-reversible gels and combinations thereof.

Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents can be selected for their ability to dissolve the drug. Other additives, which can improve the skin feel and/or emolliency of the formulation, can also be incorporated. Such additives include, but are not limited, isopropyl myristate, ethyl acetate, C12- C15 alkyl benzoates, mineral oil, squalane, cyclomethicone, capric/caprylic triglycerides, and combinations thereof.

Also described herein are foams that can include the Nav 1.5 channel modulator as described herein. Foams can be an emulsion in combination with a gaseous propellant. The gaseous propellant can include hydrofluoroalkanes (HFAs). Suitable propellants include HFAs such as 1,1,1,2-tetrafluoroethane (HFA 134a) and 1,1,1,2,3,3,3 heptafluoropropane (HFA 227), but mixtures and admixtures of these and other HFAs that are currently approved or can become approved for medical use are suitable. The propellants can be devoid of hydrocarbon propellant gases, which can produce flammable or explosive vapors during spraying. Furthermore, the foams can contain no volatile alcohols, which can produce flammable or explosive vapors during use. Buffers can be used to control pH of a pharmaceutical composition. The buffers can buffer the pharmaceutical composition from a pH of about 4 to a pH of about 7.5, from a pH of about 4 to a pH of about 7, or from a pH of about 5 to a pH of about 7. In some embodiments, the buffer can be triethanolamine.

Preservatives can be included to prevent the growth of fungi and microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

In certain embodiments, the formulations can be provided via continuous delivery of one or more formulations to a patient in need thereof. For topical applications, repeated application can be done or a patch can be used to provide continuous administration of the Nav 1.5 channel modulator over an extended period of time.

### Enteral Formulations

The Nav 1.5 channel modulator as described herein can be prepared in enteral formulations, such as for oral administration. The Nav 1.5 channel modulator can be a compound according to the ones mentioned herein or pharmaceutical salt thereof. Suitable oral dosage forms include tablets—sublingual, buccal, effervescent, chewable; troches, lozenges, dispersible powders or granules and dragees; capsules, solutions, suspensions, syrups, lozenges, medicated gums, buccal gels or patches. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations containing the Nav 1.5 channel modulator as described herein can be prepared using pharmaceutically acceptable carriers. As generally used herein “carrier” includes, but is not limited to, diluents, preservatives, binders, lubricants, disintegrators, swelling agents, fillers, stabilizers, and combinations thereof. Polymers used in the dosage form include, but are not limited to, suitable hydrophobic or hydrophilic polymers and suitable pH dependent or independent polymers. Suitable hydrophobic and hydrophilic polymers include, but are not limited to, hydroxypropyl methylcellulose, hydroxypropyl cellulose, hydroxyethyl cellulose, carboxy methylcellulose, polyethylene glycol, ethyl-cellulose, microcrystalline cellulose, polyvinyl pyrrolidone, polyvinyl alcohol, polyvinyl acetate, and ion exchange resins. “Carrier” also includes all components of the coating pharmaceutical composition which can include plasticizers, pigments, colorants, stabilizing agents, and glidants.

Formulations containing the Nav 1.5 channel modulator as described herein can be prepared using one or more pharmaceutically acceptable excipients, including diluents, preservatives, binders, lubricants, disintegrators, swelling agents, fillers, stabilizers, and combinations thereof.

### Additional Active Agents

In some embodiments, an amount of one or more additional active agents are included in the pharmaceutical compositions containing the Nav 1.5 channel modulator or pharmaceutical salt thereof. Suitable additional active agents include, but are not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytic s, antipsychotics, analgesics, antispasmodics, anti-inflammatories, anti-histamines, anti-infectives, and chemotherapeutics.

Other suitable additional active agents include, but are not limited to, statins, cholesterol lowering drugs, glucose lowering drugs. The Nav 1.5 channel modulator can be used as a monotherapy or in combination with other active agents for treatment of metabolic disorder (diabetes, high cholesterol, hyperlipidemia, high-triglycerides).

Other suitable therapeutic agents also include those which induce gating defects in sodium channel Nav1.5 and Nav1.4. These therapeutic agents are those which induce or which are likely to induce long QT or inflammation. Such agents are previously described but they also cover all such therapeutic agents whose side effects are or can be corrected by Cannabidiol.

### Dose of CANNABIDIOL

Dose of CANNABIDIOL preparations is crucial since it is observed that it may have concentration dependent effects of Nav 1.5 and it is desired that the pharmaceutical compositions are produced in multiple strengths.

A suitable dose of 0.1 mg/kg of body weight to 4000 mg/kg of body weight. The suitable dose can also be 0.1 to 1000 mg/kg of body weight or 0.1 to 500 mg/kg of body weight. The preferred dose can be 0.1 to 100 mg/kg of body weight or from 0.1 to 20 mg/kg of body weight.

The dose will depend on the nature and status of cardiac health. It will also depend on age. Further, dose will depend on type of pharmaceutical composition for example, whether oral or parenteral or topical.

Tables 2-9: Following tables provide the actual readings recorded in experiments involving steady state activation, steady state fast inactivation, recovery from fast inactivation and persistent currents in both first and second parts of the study where first part employed high glucose conditions whereas second part employed inflammation as mediator for inducing gating defects in sodium channels Nav1.5.

### Tables

TABLE 2
Steady State Activation

| | GV − V_{1/2} | GV − z | |
|---|---|---|---|
| | (mV) | (slope) | n |
| Control | | | |
| Control/Vehicle | −38.2 ± 0.9 | 3.7 ± 0.1 | 6 |
| Control/CBD [symbol] | −31.1 ± 3.1 | 3.1 ± 0.2 | 6 |
| Control/Lidocaine [symbol] | −39.9 ± 5. [symbol] | 3.6 ± 0.2 | 5 |
| Control/Tempol [symbol] Perfusion | −36.1 ± 2.2 | 3.9 ± 0.6 | 5 |
| Control/Tempol [symbol] Incubation | −41.9 ± 3.2 | 4.4 ± 0.5 | 7 |
| Mannitol ( [symbol] ) | −39.2 ± 1.6 | 5.1 ± 0.8 | 7 |
| Glucose 25 mM | −37.4 ± 1.2 | 3.1 ± 0.2 | 5 |
| Glucose 50 mM | | | |
| Glucose 50 mM/Vehicle | −30.7 ± 2.2 | 2.9 ± 0.3 | 6 |
| Glucose 50 mM/CBD [symbol] | −39.1 ± 2.8 | 4.7 ± 0.9 | 6 |
| Glucose 50 mM/Lidocaine [symbol] | −39.4 ± 4.0 | 5.5 ± 0.9 | 8 |
| Glucose 50 mM/Tempol [symbol] Perfusion | −27.6 ± 3.9 | 2.6 ± 0.2 | 6 |
| Glucose 50 mM/Tempol [symbol] Incubation | −43.1 ± 0.5 | 4.0 ± 0.6 | 5 |
| Glucose 100 mM | | | |
| Glucose 100 mM/Vehicle | −18.9 ± 2.9 | 2.8 ± 0.2 | 9 |
| Glucose 100 mM/CBD [symbol] | −39.9 ± 1.0 | 3.7 ± 0.6 | 5 |
| Glucose 100 mM/CBD [symbol] | −26.9 ± 1.0 | 2.8 ± 0.2 | 5 |
| Glucose 100 mM/Lidocaine [symbol] | −36.3 ± 3.6 | 4.2 ± 0.8 | 5 |
| Glucose 100 mM/Lidocaine [symbol] | −24.7 ± 1.7 | 3.1 ± 0.1 | 5 |
| Glucose 100 mM/Tempol [symbol] Perfusion | −23.1 ± 2.2 | 2.2 ± 0.1 | 5 |
| Glucose 100 mM/Tempol [symbol] Incubation | −41.1 ± 2.4 | 4.3 ± 1.0 | 5 |
| Glucose 100 mM/Tempol [symbol] Incubation | −30.2 ± 1.4 | 3.4 ± 0.3 | 5 |

[symbol]

indicates data missing or illegible when filed

TABLE 3
Steady State Fast Inactivation

| | SSF [symbol] − V_{1/2} | SSF [symbol] − z | |
|---|---|---|---|
| | (mV) | (slope) | [symbol] |
| Control | | | |
| Control/Vehicle | −94.3 ± 2.5 | −2.2 ± 0.2 | [symbol] |
| Control/CBD [symbol] | −113.3 ± 6.2 | −1.7 ± 0.1 | [symbol] |
| Control/Lidocaine [symbol] | −111.7 ± [symbol] | −1.5 ± 0.1 | [symbol] |
| Control/Tempol [symbol] Perfusion | −94.1 ± 3.0 | −2.2 ± 0.2 | [symbol] |
| Control/Tempol [symbol] Incubation | −98.6 ± 2.8 | −2.2 ± 0.1 | [symbol] |
| Mannitol ( [symbol] ) | −93.6 ± 2.3 | −2.1 ± 0.3 | [symbol] |
| Glucose [symbol] | −94.8 ± 3.5 | −2.2 ± 0.2 | [symbol] |
| Glucose 50 mM | | | |
| Glucose 50 mM/Vehicle | −80.7 ± 3.9 | −2.6 ± 0.2 | [symbol] |
| Glucose 50 mM/CBD [symbol] | −89.6 ± 2.5 | −2.5 ± 0.3 | [symbol] |
| Glucose 50 mM/Lidocaine [symbol] | −96.7 ± 5.4 | −1.5 ± 0.5 | [symbol] |
| Glucose 50 mM/Tempol [symbol] Perfusion | −76.7 ± 2.4 | −2.7 ± 0.1 | [symbol] |
| Glucose 50 mM/Tempol [symbol] Incubation | −94.3 ± 2.7 | −2.9 ± 0.2 | [symbol] |
| Glucose 100 mM | | | |
| Glucose 100 mM/Vehicle | −66.8 ± 2.6 | −2.7 ± 0.2 | [symbol] |
| Glucose 100 mM/CBD [symbol] | −89.5 ± 2.7 | −3.1 ± 0.3 | [symbol] |
| Glucose 100 mM/CBD [symbol] | −75.0 ± 2.4 | −2.4 ± 0.1 | [symbol] |
| Glucose 100 mM/Lidocaine [symbol] | −91.7 ± 2.4 | −1.6 ± 0.1 | [symbol] |
| Glucose 100 mM/Lidocaine [symbol] | −73.0 ± 2.0 | −2.7 ± 0.3 | [symbol] |
| Glucose 100 mM/Tempol [symbol] Perfusion | −71.8 ± 3.8 | −3.0 ± 0.2 | [symbol] |
| Glucose 100 mM/Tempol [symbol] Incubation | −90.5 ± 2.8 | −2.8 ± 0.2 | [symbol] |
| Glucose 100 mM/Tempol [symbol] Incubation | −79.9 ± 2.1 | −2.3 ± 0.2 | [symbol] |

[symbol]

indicates data missing or illegible when filed

TABLE 4
Time constants for the recovery from fast inactivation

| | τ_{fast} | τ_{slow} | n |
|---|---|---|---|
| Control | | | |
| Control/Vehicle | 0.007 ± 0.001 | 0.007 ± 0.001 | 6 |
| Control/CBD [symbol] | 0.007 ± 0.002 | 0.104 ± 0.025 | 6 |
| Control/Lidocaine [symbol] | 0.276 ± 0.030 | 0.307 ± 0.022 | 4 |
| Control/Tempol [symbol] Perfusion | 0.006 ± 0.001 | 0.027 ± 0.005 | 6 |
| Control/Tempol [symbol] Incubation | 0.004 ± 0.001 | 0.054 ± 0.012 | 4 |
| Mannitol ( [symbol] ) | 0.020 ± 0.015 | 0.058 ± 0.016 | 5 |
| Glucose [symbol] | 0.006 ± 0.001 | 0.091 ± 0.019 | 6 |
| Glucose 50 mM | | | |
| Glucose 50 mM/Vehicle | 0.015 ± 0.011 | 0.070 ± 0.006 | 7 |
| Glucose 50 mM/CBD [symbol] | 0.006 ± 0.001 | 0.081 ± 0.009 | 6 |
| Glucose 50 mM/Lidocaine [symbol] | 0.160 ± 0.031 | 0.232 ± 0.029 | 5 |
| Glucose 50 mM/Tempol [symbol] Perfusion | 0.008 ± 0.001 | 0.104 ± 0.021 | 6 |
| Glucose 50 mM/Tempol [symbol] Incubation | 0.004 ± 0.001 | 0.055 ± 0.009 | 5 |
| Glucose 100 mM | | | |
| Glucose 100 mM/Vehicle | 0.007 ± 0.001 | 0.082 ± 0.026 | 9 |
| Glucose 100 mM/CBD [symbol] | 0.005 ± 0.001 | 0.101 ± 0.006 | 5 |
| Glucose 100 mM/CBD [symbol] | 0.020 ± 0.013 | 0.104 ± 0.019 | 6 |
| Glucose 100 mM/Lidocaine [symbol] | 0.178 ± 0.020 | 0.336 ± 0.042 | 8 |
| Glucose 100 mM/Lidocaine [symbol] | 0.032 ± 0.025 | 0.212 ± 0.013 | 5 |
| Glucose 100 mM/Tempol [symbol] Perfusion | 0.005 ± 0.001 | 0.065 ± 0.032 | 5 |
| Glucose 100 mM/Tempol [symbol] Incubation | 0.005 ± 0.001 | 0.065 ± 0.020 | 5 |
| Glucose 100 mM/Tempol [symbol] Incubation | 0.009 ± 0.003 | 0.118 ± 0.011 | 5 |

[symbol]

indicates data missing or illegible when filed

TABLE 5
Persistent Current

| | Persistent I [symbol] | n |
|---|---|---|
| Control | | |
| Control/Vehicle | 0.97 ± 0.11 | 7 |
| Control/CBD 5 μM | 0.72 ± 0.13 | 6 |
| Control/Lidocaine 1 mM | 0.97 ± 0.17 | 5 |
| Control/Tempol 1 mM Perfusion | 1.06 ± 0.07 | 5 |
| Control/Tempol 1 mM Incubation | 1.03 ± 0.08 | 5 |
| Mannitol (100 mM) | 0.97 ± 0.10 | 7 |
| Glucose 25 mM | 1.06 ± 0.12 | 5 |
| Glucose 50 mM | | |
| Glucose 50 mM/Vehicle | 3.16 ± 0.42 | 7 |
| Glucose 50 mM/CBD 5 μM | 1.26 ± 0.20 | 5 |
| Glucose 50 mM/Lidocaine 1 mM | 1.82 ± 0.11 | 5 |
| Glucose mM/Tempol 1 mM Perfusion | 3.04 ± 0.35 | 8 |
| Glucose 50 mM/Tempol 1 mM Incubation | 0.90 ± 0.22 | 5 |
| Glucose 100 mM | | |
| Glucose 100 mM/Vehicle | 6.41 ± 0.61 | 9 |
| Glucose 100 mM/CBD 5 μM | 1.38 ± 0.20 | 7 |
| Glucose 100 mM/CBD 1 μM | 3.03 ± 0.12 | 6 |
| Glucose 100 mM/Lidocaine | 1.31 ± 0.25 | 7 |
| Glucose 100 mM/Lidocaine 100 μM | 3.50 ± 0.36 | 5 |
| Glucose 100 mM/Tempol 1 mM Perfusion | 6.07 ± 0.82 | 7 |
| Glucose 100 mM/Tempol 1 mM Incubation | 1.33 ± 0.26 | 5 |
| Glucose 100 mM/Tempol 100 μM Incubation | 2.93 ± 0.18 | 5 |

[symbol]

indicates data missing or illegible when filed

TABLE 6
Steady state activation

| | GV − V_{1/2} | GV − z | |
|---|---|---|---|
| | (mV) | (slope) | n |
| Control | | | |
| Control/Vehicle | −36.2 ± 1.6 | 3.3 ± 0.2 | 5 |
| Vehicle/H-89 | −39.6 ± 1.4 | 3.2 ± 0.1 | 5 |
| Vehicle/Gö 6983 | −37.7 ± 0.7 | 3.1 ± 0.1 | 5 |
| Glucose (100 mM) | | | |
| 100 mM glucose/Vehicle | −16.6 ± 2.8 | 2.5 ± 0.1 | 5 |
| Inflammatory mediators (IM) | | | |
| IM/Vehicle | −22.3 ± 2.4 | 2.7 ± 0.2 | 5 |
| IM/H-89 | −32.7 ± 1.4 | 2.8 ± 0.2 | 5 |
| IM/Gö 6983 | −31.7 ± 1.6 | 2.7 ± 0.1 | 5 |
| CPT- cAMP | −25.2 ± 0.5 | 2.5 ± 0.1 | 5 |
| PMA | −22.6 ± 1.6 | 2.5 ± 0.1 | 5 |
| CANNABIDIOL(5 μM) | | | |
| IM/CANNABIDIOL(CANNABIDIOL) | −39.1 ± 2.8 | 3.6 ± 0.1 | 5 |
| CPT- | −33.1 ± 0.6 | 3.4 ± 0.1 | 5 |
| cAMP/ | | | |
| CANNABIDIOL(CANNABIDIOL) | | | |
| PMA/CANNABIDIOL(CANNABIDIOL) | −35.3 ± 0.9 | 3.3 ± 0.1 | 5 |
| E₂ | | | |
| E₂ 5 μM/vehicle | −34.8 ± 1.5 | 3.1 ± 0.1 | 5 |
| E₂ 10 μM/vehicle | −34.3 ± 0.9 | 3.0 ± 0.1 | 5 |
| E₂ 5 μM/glucose 100 mM | −27.3 ± 0.7 | 2.4 ± 0.1 | 5 |
| E₂ 10 μM/glucose 100 mM | −37.9 ± 1.4 | 3.5 ± 0.1 | 5 |
| E₂ 5 μM/IM | −29.8 ± 1.3 | 2.8 ± 0.1 | 5 |
| E₂ 10 μM/IM | −35.7 ± 2.0 | 3.5 ± 0.1 | 5 |
| E₂ 10 μM/CPT-cAMP | −37.7 ± 0.6 | 3.6 ± 0.1 | 5 |
| E₂ 10 μM/PMA | −35.9 ± 1.5 | 3.4 ± 0.2 | 5 |

TABLE 7
Steady state fast inactivation

| | SSFI | SSFI − z | |
|---|---|---|---|
| | V_{1/2} (mV) | (slope) | n |
| Control | | | |
| Control/Vehicle | −90.9 ± 1.8 | −2.6 ± 0.1 | 5 |
| Vehicle/H-89 | −89.3 ± 1.9 | −2.7 ± 0.1 | 5 |
| Vehicle/Gö 6983 | −88.6 ± 2.1 | −3.0 ± 0.1 | 5 |
| Glucose (100 mM) | | | |
| 100 mM glucose/Vehicle | −61.7 ± 2.6 | −2.9 ± 0.1 | 5 |
| Inflammatory mediators (IM) | | | |
| IM/Vehicle | −77.1 ± 1.7 | −2.6 ± 0.1 | 5 |
| IM/H-89 | −86.4 ± 2.8 | −2.9 ± 0.2 | 5 |
| IM/Gö 6983 | −87.1 ± 2.0 | −2.4 ± 0.2 | 5 |
| CPT- cAMP | −79.4 ± 1.1 | −3.0 ± 0.1 | 5 |
| PMA | −76.4 ± 1.7 | −2.9 ± 0.2 | 5 |
| CANNABIDIOL(5 μM) | | | |
| IM/CANNABIDIOL(CANNABIDIOL) | −85.9 ± 1.5 | −2.6 ± 0.3 | 5 |
| CPT- | −86.8 ± 2.3 | −2.9 ± 0.2 | 5 |
| cAMP/ | | | |
| CANNABIDIOL(CANNABIDIOL) | | | |
| PMA/CANNABIDIOL(CANNABIDIOL) | −85.7 ± 1.2 | −2.9 ± 0.1 | 5 |
| E₂ | | | |
| E₂ 5 μM/vehicle | −87.4 ± 2.1 | −2.8 ± 0.1 | 5 |
| E₂ 10 μM/vehicle | −87.6 ± 2.1 | −3.0 ± 0.2 | 5 |
| E₂ 5 μM/glucose 100 mM | −75.5 ± 1.9 | −2.8 ± 0.2 | 5 |
| E₂ 10 μM/glucose 100 mM | −91.1 ± 3.6 | −2.8 ± 0.1 | 5 |
| E₂ 5 μM/IM | −81.1 ± 2.1 | −2.8 ± 0.1 | 5 |
| E₂ 10 μM/IM | −92.6 ± 0.8 | −2.6 ± 0.1 | 5 |
| E₂ 10 μM/CPT-cAMP | −89.3 ± 1.9 | −2.6 ± 0.1 | 5 |
| E₂ 10 μM/PMA | −88.7 ± 0.6 | −2.3 ± 0.2 | 5 |

TABLE 8
Time constants for the recovery from fast inactivation

| | τ_{fast} (s) | τ_{slow} (s) | n |
|---|---|---|---|
| Control | | | |
| Control/Vehicle | 0.006 ± 0.001 | 0.006 ± 0.001 | 5 |
| Vehicle/H-89 | 0.007 ± 0.001 | 0.006 ± 0.001 | 5 |
| Vehicle/Gö 6983 | 0.006 ± 0.001 | 0.010 ± 0.002 | |
| Glucose (100 mM) | | | |
| 100 mM glucose/Vehicle | 0.008 ± 0.002 | 0.111 ± 0.03 | 5 |
| Inflammatory mediators (IM) | | | |
| IM/Vehicle | 0.005 ± 0.001 | 0.123 ± 0.002 | 5 |
| IM/H-89 | 0.010 ± 0.002 | 0.303 ± 0.036 | 5 |
| IM/Gö 6983 | 0.008 ± 0.002 | 0.304 ± 0.031 | 5 |
| CPT- cAMP | 0.006 ± 0.001 | 0.168 ± 0.009 | 5 |
| PMA | 0.005 ± 0.001 | 0.175 ± 0.005 | 5 |
| CANNABIDIOL(5 μM) | | | |
| IM/CANNABIDIOL(CANNABIDIOL) | 0.008 ± 0.001 | 0.209 ± 0.020 | 5 |
| CPT- | 0.009 ± 0.001 | 0.207 ± 0.004 | 5 |
| cAMP/CANNABIDIOL(CANNABIDIOL) | | | |
| PMA/CANNABIDIOL(CANNABIDIOL) | 0.006 ± 0.001 | 0.218 ± 0.014 | 5 |
| E₂ | | | |
| E₂ 5 μM/vehicle | 0.006 ± 0.001 | 0.011 ± 0.002 | 5 |
| E₂ 10 μM/vehicle | 0.006 ± 0.001 | 0.010 ± 0.002 | 5 |
| E₂ 5 μM/glucose 100 mM | 0.005 ± 0.001 | 0.148 ± 0.009 | 5 |
| E₂ 10 μM/glucose 100 mM | 0.008 ± 0.002 | 0.228 ± 0.015 | 5 |
| E₂ 5 μM/IM | 0.005 ± 0.001 | 0.182 ± 0.015 | 5 |
| E₂ 10 μM/IM | 0.005 ± 0.001 | 0.262 ± 0.015 | 5 |
| E₂ 10 μM/CPT-cAMP | 0.007 ± 0.001 | 0.222 ± 0.008 | 5 |
| E₂ 10 μM/PMA | 0.007 ± 0.001 | 0.233 ± 0.006 | 5 |

TABLE 9
Persistent current

| | Percentage of | |
|---|---|---|
| | persistent I_{Na} | n |
| Control | | |
| Control/Vehicle | 0.80 ± 0.05 | 5 |
| Vehicle/H-89 | 0.82 ± 0.07 | 5 |
| Vehicle/Gö 6983 | 0.84 ± 0.08 | 5 |
| Glucose (100 mM) | | |
| 100 mM glucose/Vehicle | 6.86 ± 0.17 | 5 |
| Inflammatory mediators (IM) | | |
| IM/Vehicle | 3.64 ± 0.23 | 5 |
| IM/H-89 | 1.21 ± 0.07 | 5 |
| IM/Gö 6983 | 1.22 ± 0.06 | 5 |
| CPT- cAMP | 2.20 ± 0.08 | 5 |
| PMA | 2.18 ± 0.06 | 5 |
| CANNABIDIOL(5 μM) | | |
| IM/CANNABIDIOL(CANNABIDIOL) | 0.93 ± 0.05 | 5 |
| CPT- | 1.04 ± 0.11 | 5 |
| cAMP/CANNABIDIOL(CANNABIDIOL) | | |
| PMA/CANNABIDIOL(CANNABIDIOL) | 0.88 ± 0.07 | 5 |
| E₂ | | |
| E₂ 5 μM/vehicle | 0.85 ± 0.06 | 5 |
| E₂ 10 μM/vehicle | 0.91 ± 0.06 | 5 |
| E₂ 5 μM/glucose 100 mM | 1.92 ± 0.09 | 5 |
| E₂ 10 μM/glucose 100 mM | 0.89 ± 0.06 | 5 |
| E₂ 5 μM/IM | 1.73 ± 0.03 | 5 |
| E₂ 10 μM/IM | 0.85 ± 0.06 | 5 |
| E₂ 10 μM/CPT-cAMP | 0.95 ± 0.09 | 5 |
| E₂ 10 μM/PMA | 0.90 ± 0.11 | 5 |

Following examples do not limit in any way scope of the invention.

### EXAMPLES

### First Part of the Study

### Example 1—Cell Viability Studies

Cell Culture: Chinese hamster ovary (CHO) were grown at pH 7.4 in filtered sterile F12 (Ham) nutrient medium (Life Technologies, Thermo Fisher Scientific, Waltham, Mass., USA), supplemented with 5% FBS and maintained in a humidified environment at 37° C. with 5% CO2. Cells were transiently co-transfected with the human cDNA encoding the Nav1.5 α-subunit, the β1-subunit, and eGFP. Transfection was done according to the PolyFect (Qiagen, Germantown, Md., USA) transfection protocol. A minimum of 8-hour incubation was allowed after each set of transfections. Then, the cells were dissociated with 0.25% trypsin-EDTA (Life Technologies, Thermo Fisher Scientific) and plated on sterile coverslips under normal (10 mM) or elevated glucose concentrations (25-150 mM) for 24 hours prior to electrophysiological or biochemical experiments. To optimize the glucose concentration that would mimic the diabetic/hyperglycemia conditions in CHO cells, the MTS cell viability assay was used to check the viability of CHO cells at different glucose concentrations.

To ensure that there are no confounding effects imposed by loss of osmolarity, experiments were also performed in the presence of mannitol (100 mM for 24 hours) as osmotic control for high glucose, in accordance with reported studies (El-Remessy et al., Investigative ophthalmology & visual science 44: 3135-3143, 2003; Fouda et al., The Journal of pharmacology and experimental therapeutics 361: 130-139, 2017; Sharifi et al., Neuroscience letters 459: 47-51, 2009).

Determination of cell viability—To establish the concentration-dependent cytotoxicity caused by glucose, CHO cells were seeded at 50,000 cells/ml in a 96-well plate for 24 hours (90% confluence), then treatments were started in normal (10 mM) or elevated (25-150 mM) glucose concentrations for another 24 hours in presence and absence of different treatments [CANNABIDIOL (1 or 5 μM), lidocaine (100 μM or 1 mM), Tempol (100 μM or 1 mM) or their vehicle]. At the end of the incubation period (24 hours), cell viability was measured by MTS cell proliferation assay kit with absorbance measured at 495 nm in accordance with manufacturer's instructions (Abeam, ab 197010, Toronto, Canada).

The results of cell viability studies are provided in FIGS. 1A-1E and discussed in the specification.

### Example 2—ROS Measurement

Oxidative stress level was measured using 2′,7′-dichlorofluorescein diacetate (DCFH-DA), a detector of ROS (Korystov et al., Free radical research 43: 149-155, 2009). Fluorescence intensity was measured 30 min after the reaction initiation using a microplate fluorescence reader set at excitation 485 nm/emission 530 nm according to the manufacturer (Abcam, ab113851, Toronto, Canada). The ROS level was determined as relative fluorescence units (RFU) of generated DCF using standard curve of DCF (Fouda et al., The Journal of pharmacology and experimental therapeutics 361: 130-139, 2017, Fouda et al., The Journal of pharmacology and experimental therapeutics 364: 170-178. 2018).

The results of ROS studies are provided in FIGS. 2A-2E and discussed in the specification.

### Example 3—Electrophysiology

Whole-cell patch clamp recordings were implemented using extracellular solution composed of NaCl (140 mM), KCl (4 mM), CaCl2 (2 mM), MgCl2 (1 mM), HEPES (10 mM). Extracellular solution was titrated to pH 7.4 with CsOH. Pipettes were fabricated with a P-1000 puller using borosilicate glass (Sutter Instruments, CA, USA), dipped in dental wax to reduce capacitance, then thermally polished to a resistance of 1.0-1.5 MΩ. Pipettes were filled with intracellular solution, containing: CsF (120 mM), CsCl (20 mM), NaCl (10 mM), HEPES (10 mM) titrated to pH 7.4. All recordings were made using an EPC-9 patch-clamp amplifier (HEKA Elektronik, Lambrecht, Germany) digitized at 20 kHz via an ITC-16 interface (Instrutech, Great Neck, N.Y., USA). Voltage clamping and data acquisition were controlled using PatchMaster/FitMaster software (HEKA Elektronik, Lambrecht, Germany) running on an Apple iMac. Current was low-pass-filtered at 5 kHz. Leak subtraction was automatically done using a P/4 procedure following the test pulse. Gigaohm seals were allowed to stabilize in the on-cell configuration for 1 min prior to establishing the whole cell configuration. Series resistance was less than 5 MΩ for all recordings. Series resistance compensation up to 80% was used when necessary. All data were acquired at least 5 min after attaining the whole-cell configuration, and cells were allowed to incubate 5 min after drug application prior to data collection. Before each protocol, the membrane potential was hyperpolarized to −130 mV to insure complete removal of both fast-inactivation and slow-inactivation. Leakage and capacitive currents were subtracted with a P/4 protocol. All experiments were conducted at 22° C.

### Example 4: Activation Protocols

To determine the voltage-dependence of activation, inventors measured the peak current amplitude at test pulse voltages ranging from −130 to +80 mV in increments of 10 mV for 19 ms. Channel conductance (G) was calculated from peak INa:

where GNa is conductance, INa is peak sodium current in response to the command potential V, and ENa is the Nernst equilibrium potential. The midpoint and apparent valence of activation were derived by plotting normalized conductance as a function of test potential. Data were then fitted with a Boltzmann function:
where G/Gmax is normalized conductance amplitude, Vm is the command potential, z is the apparent valence, e0 is the elementary charge, V1/2 is the midpoint voltage, k is the Boltzmann constant, and T is temperature in K.

The results of steady state activation are provided in FIGS. 3A-3G and table 2 and discussed in the specification.

### Example 5: Steady State Fast Inactivation Protocols

The voltage-dependence of fast-inactivation was measured by preconditioning the channels to a hyperpolarizing potential of −130 mV and then eliciting pre-pulse potentials that ranged from −170 to +10 mV in increments of 10 mV for 500 ms, followed by a 10 ms test pulse during which the voltage was stepped to 0 mV. Normalized current amplitude as a function of voltage was fit using the Boltzmann function:

where Imax is the maximum test pulse current amplitude. z is apparent valency, e0 is the elementary charge, Vm is the prepulse potential, V1/2 is the midpoint voltage of SSFI, k is the Boltzmann constant, and T is temperature in K.

The results of Steady state fast inactivation studies are provided in FIGS. 4A-4F and table 3 and discussed in the specification.

### Example 6: Fast Inactivation Recovery

Channels were fast inactivated during a 500 ms depolarizing 1 step to 0 mV. Recovery was measured during a 19 ms test pulse to 0 mV following −130 mV recovery pulse for durations between 0 and 1.024 s. Time constants of fast inactivation were derived using a double exponential equation:

where I is current amplitude, Iss is the plateau amplitude, α1 and α2 are the amplitudes at time 0 for time constants τ1 and τ2, and t is time.

The results of fast inactivation recovery are provided in FIGS. 5A-5F and table 4 and discussed in the specification.

### Example 7: Persistent Current Protocols

Late sodium current was measured between 145 and 150 ms during a 200 ms depolarizing pulse to 0 mV from a holding potential of −130 mV. Fifty pulses were averaged to increase signal to noise ratio.

The results of persistent current studies are provided in FIGS. 6A-6D and table 5 and discussed in the specification.

### Example 8: Action Potential Modeling

Action potentials were simulated using a modified version of the Action potential modeling programmed in Matlab (O'Hara et al., PLoS computational biology 7: e1002061, 2011). The modified gating INa parameters were in accordance with the biophysical data obtained from whole-cell patch-clamp experiments in this study for various conditions. The model accounted for activation voltage-dependence, steady-state fast inactivation voltage-dependence, persistent sodium currents, and peak sodium currents (compound conditions).

The results of studies on prolongation of action potential are provided in FIGS. 7A-7B and discussed in the specification.

### Example 9: Drug Preparations

CANNABIDIOL was purchased from Toronto Research Chemicals in powder form. Other compounds (e.g. lidocaine, Tempol, D-glucose or mannitol) were purchased from Sigma-Aldrich (ON, Canada). Powdered CANNABIDIOL, lidocaine or Tempol were dissolved in 100% DMSO to create stock. The stock was used to prepare drug solutions in extracellular solutions at various concentrations with no more than 0.5% total DMSO content.

As an alternative to synthesized CANNABIDIOL, biosynthtically prepared CANNABIDIOL can be used.

### Example 10: Data Analysis and Statistics

The data and statistical analysis comply with the British Journal of Pharmacology recommendations on experimental design and analysis in pharmacology (Curtis et al., British journal of pharmacology 175: 987-993 2018). Studies were designed to generate groups of equal size, using randomisation and blinded analysis. Normalization was performed in order to control the variations in sodium channel expression and inward current amplitude and in order to be able to fit the recorded data with a Boltzmann function (for voltage-dependences) or an exponential function (for time courses of inactivation). Fitting and graphing were done using FitMaster software (HEKA Elektronik, Lambrecht, Germany) and Igor Pro (Wavemetrics, Lake Oswego, Oreg., USA). Statistical analysis consisted of one-way ANOVA (endpoint data) along with post hoc testing of significant findings along with Student's t-test and Tukey's test using Prism 7 software (Graphpad Software Inc., San Diego, Calif.). Values are presented as mean±SEM with probability levels less than 0.05 considered significant.

### Example 11: Rat Diaphragm Preparation

Four 4-week old male Sprague Dawley rats (Charles River, Raleigh site) were euthanized. The rat phrenic hemi-diaphragm preparation was isolated according to the method described by Bulbring (1946). A fan-shaped muscle with an intact phrenic nerve was isolated from the left side and transferred to a container with Krebs' solution (NaCl 95.5, KCl 4.69, CaCl2 2.6, MgSO4.7H2O 1.18, KH2PO4 2.2, NaHCO3 24.9, and glucose 10.6 mM) and aerated with carbogen (95% oxygen and 5% carbon dioxide). All experimental protocols were approved by the Animal Care and Use Committees. Contraction experiment was performed using a Radnoti Myograph system.

The data and results of studies are provided in FIGS. 9A-9F in the specification.

### Example 12: Molecular Docking

Docking of CANNABIDIOL into the cryo-EM structure of hNav1.4 (PDB ID: 6AGF was carried out using Autodock Vina. CANNABIDIOL was downloaded in PDB format from Drugbank. To dock CANNABIDIOL into Nav1.4 a large search volume of 32 Å×44 Å×26 Å was considered, that enclosed nearly the whole of the pore domain and parts of VSD. This yielded the top 9 best binding poses of CANNABIDIOL ranked by mean energy score.

### Example 13: MD Simulation Systems Preparation

A homogenous lipid bilayer consisting of 188 POPC was prepared using the CHARMM-GUI Membrane builder. Three different systems were created: one with two CANNABIDIOL molecules, each one placed in each leaflet of the bilayer, one with three CANNABIDIOLs, all of them placed in the upper leaflet and the third with six CANNABIDIOLs, of which three placed in the upper leaflet and three placed in the lower leaflet. CANNABIDIOL was placed manually into the bilayer, with the polar headgroup of CANNABIDIOL facing the lipid headgroups. Lipid molecules with at least one atom within 2 Å of a CANNABIDIOL were manually deleted. A control simulation without any CANNABIDIOL was also prepared. The system was hydrated by adding two ˜25 Å layers of water to both sides of the membrane. Lastly, the system was inonized with 150 mM NaCl.

The best docked position obtained from Autodock Vina was used as the starting structure. The starting system was embedded into POPC lipid bilayer. The system was hydrated by adding two ˜25 Å layers of water to both sides of the membrane. Lastly, the system was ionized with 150 mM NaCl. This system is defined as the Nav1.4-CANNABIDIOL-lipid system.

The data and results of studies are provided in FIGS. 10A-10H in the specification.

### Example 14: MD Simulations

Adiabatic biased molecular dynamics (ABMD) simulations were performed using GROMACS 201863 patched with Plumed-2.1.5 to study the interaction of CANNABIDIOL with Nav1.4. ABMD is a simulation method in which a time dependent biasing harmonic potential is applied to drive the system towards a target system. along a predefined collective variable. Whenever the system moves closer towards the target system along the collective variable, the harmonic potential is moved to this new position, resulting in pushing the system towards the final state. The bias potential was applied along the distance between the center of masses of CANNABIDIOL and F1586. The biasing potential was applied in two ways. One along the y-component of distance and other along all components of distance. MD simulations for the lipid-CANNABIDIOL system were performed using GROMACS version 2018.4. The CHARMM36 forcefield was used to describe the protein, lipid bilayer, and the ions. CANNABIDIOL was parameterised using the SWISS-PARAM software. The TIP3P water model was used to describe the water molecules. The systems were minimised for 5000 steps using steepest descent and equilibrated with constant number of particles, pressure and temperature (NPT) for at least 450 ps for the lipid-CANNABIDIOL system and 36 ns for the Nav1.4-CANNABIDIOL-lipid system, during which the position restraints were gradually released according to the default CHARMM-GUI protocol. During equilibration, a time step of 2 fs was used, pressure was maintained at 1 bar through Berendsen pressure coupling, temperature was maintained at 300 K through Berendsen temperature coupling with the protein, membrane and solvent coupled and LINCs algorithm was used to constrain the bonds containing hydrogen. For long range interactions, periodic boundary conditions and particle mesh Ewald (PME) were used For short range interactions, a cut-off of 12 Å was used. Finally, unrestrained production simulations were run for 150 ns for each of the lipid-CANNABIDIOL system and 10 ns for the Nav1.4-CANNABIDIOL-lipid system, using Parinello-Rahaman pressure coupling and Nose-Hoover temperature coupling.

The data and results of studies are provided in FIGS. 10A-10H in the specification.

### Example 15: 2H NMR Lipid Analysis

1-palmitoyl-2-oleoyl-sn-glycero-3-phosphocholine (POPC-d31, sn-1 chain perdeuterated) was obtained from Avanti Polar Lipids (Alabaster, Ala.). The POPC-d31: CANNABIDIOL sample was prepared with ˜50 mg lipid and 3.4 mg of CANNABIDIOL for a ratio of POPC/CANNABIDIOL 8:2. The two samples, pure POPC-d31 and POPC-d31:CANNABIDIOL (8:2), were dissolved in Bz/MeOH 4:1 (v/v) and freeze-dried. After hydration with excess amounts of deuterium-depleted water (ddw), five freeze-thaw-vortex cycles were done between liquid nitrogen (−196° C.) and 60° C. to create multilamellar dispersions (MLDs).

Deuterium 2H NMR experiments were performed on a TacMag Scout spectrometer at 46.8 MHz using the quadrupolar echo technique1. The spectra were produced from −20,000 two-pulse sequences. 90° pulse lengths were set to 3.1 μs, inter-pulse spacing was 50 μs, dwell time was 2 μs, and acquisition delays were 300 ms. Data were collected using quadrature with Cyclops eight-cycle phase cycling. The spectra were dePaked to extract the smoothed order parameter profiles of the POPC sn-1 chain in the presence or absence of CANNABIDIOL. Samples were run at 20, 30, and 40° C., left to equilibrate at each temperature for 20 mins before measurements were taken. The data and results of studies are provided in FIGS. 10F-10H in the specification.

### Example 16: Cell Culture

Chinese Hamster Ovary (CHOK1) cells were transiently co-transfected with cDNA encoding eGFP and the β1-subunit and either WT-Nav1.4 (GenBank accession number: NM_000334) or any of the mutant α-subunits. Transfection was done according to the PolyFect transfection protocol. After each set of transfections, a minimum of 8-hour incubation was allowed before plating on sterile coverslips. Human Embryonic Kidney (HEK293) cells were used for gramicidin membrane rigidity assay.

### Example 17 Automated Patch-Clamp-Gramicidin Membrane Rigidity Assay

Automated patch-clamp recording was performed on untransfected HEK cells. Currents were measured in the whole-cell configuration using a Qube-384 (Sophion A/S, Copenhagen, Denmark) automated voltage-clamp system. Intracellular solution contained (in mM): 120 CsF, 10 NaCl, 2 MgCl2, 10 HEPES, adjusted to pH7.2 with CsOH. The extracellular recording solution for the high sodium experiment contained (in mM): 140 NaCl, 3 KCl, 1 MgCl2, 1.5 CaCl2, 10 HEPES, adjusted to pH7.4 with NaOH. For the low sodium experiment the external solution sodium concentration was lowered to 1 mM with NMDG as NaCl replacement. Liquid junction potentials calculated to be ˜7 mV were not adjusted for. Currents were low-pass-filtered at 5 kHz and recorded at 25 kHz sampling frequency. Series resistance compensation was applied at 100%. The measurements were obtained at room temperature which corresponds to 27±2° C. at the recording chamber. Appropriate filters for cell membrane resistance (typically >500 MΩ) and series resistance (<10 MΩ) were used. Gramicidin was dissolved in 100% DMSO, and the final concentration of 26 μM.

The data and results of studies are provided in FIGS. 11A-11H in the specification.

### Example 18: Gramicidin-Fluorescence Membrane Rigidity Assay

1,2-dierucoyl-sn-glycero-3-phosphocholine (DC22:1PC) were from Avanti Polar Lipids (Alabaster, Ala.). CANNABIDIOL was from Sigma-Aldrich (St. Louis, Mo.). 8-Aminonaphthalene-1,3,6-trisulfonate (ANTS) was from Invitrogen Life Technologies (Grand Island, N.Y.). Gramicidin D was from (Sigma Aldrich).

GFA: Large unilamellar vesicles (LUVs) were made from DC22:1PC as described previously 71. Briefly, phospholipids in chloroform and gA in methanol (1000:1 lipid:gA weight ratio) were mixed. Quench rates were obtained by fitting the quench time course from each mixing reaction with a stretched exponential 43:
(Eq. 1)
   and evaluating the quench rate at 2 ms (the instrumental dead time is ˜1.5 ms):
(Eq. 2)
   To test drug effects on the lipid bilayer CANNABIDIOL was equilibrated with the LUVs for 10 min at 25° C. before acquiring quench time courses. Each measurement consisted of (4-8) individual mixing reactions, and the rates for each mixing reaction were averaged and normalized to the control rate in the absence of drug.

The data and results of studies are provided in FIGS. 19A-19C in the specification.

### Example 19: Manual Patch-Clamp

Whole-cell patch-clamp recordings were performed in an extracellular solution containing (in mM): 140 NaCl, 4 KCl, 2 CaCl2, 1 MgCl2, 10 HEPES or MES (pH6.4). Solutions were adjusted to pH6.4 and 7.4 with CsOH. Pipettes were filled with intracellular solution, containing (in mM): 120 CsF, 20 CsCl, 10 NaCl, 10 HEPES. In some experiments lower sodium concentration of 1 mM (intracellular) was used to boost driving force, and hence current size. All recordings were made using an EPC-9 patch-clamp amplifier (HEKA Elektronik, Lambrecht, Germany) digitized at 20 kHz via an ITC-16 interface (Instrutech, Great Neck, N.Y., USA). Voltage-clamping and data acquisition were controlled using PatchMaster/FitMaster software (HEKA Elektronik, Lambrecht, Germany) running on an Apple iMac. Current was low-pass-filtered at 10 kHz. Leak subtraction was performed automatically by software using a P/4 procedure following the test pulse. Giga-ohm seals were allowed to stabilize in the on-cell configuration for 1 min prior to establishing the whole-cell configuration. Series resistance was less than 5 MΩ for all recordings. Series resistance compensation up to 80% was used when necessary. All data were acquired at least 1 min after attaining the whole-cell configuration. Before each protocol, the membrane potential was hyperpolarized to −130 mV to ensure complete removal of both fast-inactivation and slow-inactivation. All experiments were conducted at 22±2° C. Analysis and graphing were done using FitMaster software (HEKA Elektronik) and Igor Pro (Wavemetrics, Lake Oswego, Oreg., USA). All data acquisition and analysis programs were run on an Apple iMac (Apple Computer).

Some cDNA constructs produced small ionic currents. To ensure, the recorded currents were indeed construct-produced currents and not endogenous background currents, untransfected cells were patched and compared to transfected cells. The untransfected CHOK1 cells, which were exclusively used for cDNA expression, produced no endogenous sodium currents.

### Example 20: Activation Protocol

To determine the voltage-dependence of activation, the peak current amplitude is measured at test pulse potentials ranging from −100 mV to +80 mV in increments of +10 mV for 20 ms. Channel conductance (G) was calculated from peak INa:

GNa=INa/V−ENa (Eq. 3)

where GNa is conductance, INa is peak sodium current in response to the command potential V, and ENa is the Nernst equilibrium potential. Calculated values for conductance were fit with the Boltzmann equation:

G/Gmax=1/(1+exp[−ze0[Vm−V1/2]/kT]) (Eq. 4)

where G/Gmax is normalized conductance amplitude, Vm is the command potential, z is the apparent valence, e0 is the elementary charge, V1/2 is the midpoint voltage, k is the Boltzmann constant, and T is temperature in K.

### Example 21: Steady-State Fast-Inactivation Protocol

The voltage-dependence of fast-inactivation was measured by preconditioning the channels to a hyperpolarizing potential of −130 mV and then eliciting pre-pulse potentials that ranged from −170 to +10 mV in increments of 10 mV for 500 ms, followed by a 10 ms test pulse during which the voltage was stepped to 0 mV. Normalized current amplitudes from the test pulse were fit as a function of voltage using the Boltzmann equation:

I/Imax=1/(1+exp(−ze0(VM−V1/2)/kT) (Eq. 5)

where Imax is the maximum test pulse current amplitude.

### Example 22: Persistent Current Protocol

Persistent current was measured between 145 and 150 ms during a 200 ms depolarizing pulse to 0 mV from a holding potential of −130 mV. Pulses were averaged to increase signal-to-noise ratio.

### Example 23: Recovery from Fast-Inactivation Protocol

Channels were fast-inactivated during a 500 ms depolarizing step to 0 mV, and recovery was measured during a 19 ms test pulse to 0 mV following a −130 mV recovery pulse for durations between 0 and 4 s. Time constants of fast-inactivation recovery showed two components and were fit using a double exponential equation:

I=Iss+α1exp(−t/τ1)+α2exp(−t/τ2) (Eq. 6)

where I is current amplitude, Iss is the plateau amplitude, α1 and α2 are the amplitudes at time 0 for time constants τ1 and τ2, and t is time.

### Example 24: Isothermal Titration Calorimetry

The peptide with the following sequence: SYIIISFLIVVNM (from Nav1.4 DIV-S6) was synthesized by GenScript. It was solubilized in DMSO and diluted to a final concentration of 1 mM with the final buffer containing by percentage each of the following components: 10% DMSO, 60% acetonitrile, 30% ITC buffer. Acetonitrile was required to solubilize the peptide. The ITC buffer contained 50 mM HEPES pH7.2 and 150 mM KCl. Each of CANNABIDIOL and Lidocaine were solubilized in DMSO and diluted to a final concentration of 40 mM and 100 mM, respectively in the same final buffer as the peptide.

Each titrant was injected into the peptide containing sample cell 13 times each with a volume of 3 μM with the exception of the first injection which was 0.4 μM. Stirring speed was set at 750 rpm.

### Example 25: Action Potential Modeling

Skeletal AP modeling was based on a model developed by Cannon et al., (1993). All APs were programmed and run using Python. The modified parameters were based on electrophysiological results obtained from whole-cell patch-clamp experiments. The model accounted for activation voltage-dependence, SSFI voltage-dependence, and persistent INa. The WT pH7.4 model uses the original parameters from the model. P1158S models were programmed by shifting parameters from the original Cannon model by the difference between the values in P1158S experiments at a given pH/CANNABIDIOL.

### Example 26: Statistics

A one-factor analysis of variance (ANOVA) was used to compare the mean responses. Post-hoc tests using the Tukey Kramer adjustment compared the mean responses between channel variants across conditions. A level of significance α=0.05 was used in all overall post-hoc tests, and effects with p-values less than 0.05 were considered to be statistically significant. All values are reported as means±standard error of means (SEM) for n samples. Power analysis with α=0.05 was performed and yielded Analysis was performed in JMP version 14.

### Example 27: Preparation of Cell Culture of Nav1.5 and Action of Inflammatory Mediators

Chinese hamster ovary cells (CHO) (RRID: CVCL_0214) were grown at pH 7.4 in filtered sterile F12 (Ham's) nutrient medium (Life Technologies, Thermo Fisher Scientific, Waltham, Mass., USA), supplemented with 5% FBS and maintained in a humidified environment at 37° C. with 5% CO2. Cells were transiently co-transfected with the human cDNA encoding the Nav1.5 α-subunit, the β1-subunit, and eGFP. Transfection was done according to the PolyFect (Qiagen, Germantown, Md., USA) transfection protocol. A minimum of 8-hour incubation was allowed after each set of transfections. The cells were subsequently dissociated with 0.25% trypsin-EDTA (Life Technologies, Thermo Fisher Scientific) and plated on sterile coverslips under normal (10 mM) or elevated glucose concentrations (100 mM) (Fouda, Ghovanloo & Ruben, 2020) or a cocktail of inflammatory mediators (Akin et al., 2019) containing bradykinin (1 PGE-2 (10 μM), histamine (10 μM), 5-HT (10 μM), and adenosine 5′-triphosphate (15 μM) for 24 hours prior to electrophysiological experiments.

### Example 28: Electrophysiology

Whole-cell patch clamp recordings were made using an extracellular solution composed of NaCl (140 mM), KCl (4 mM), CaCl2 (2 mM), MgCl2 (1 mM), HEPES (10 mM). The extracellular solution was titrated to pH 7.4 with CsOH. Pipettes were fabricated with a P-1000 puller using borosilicate glass (Sutter Instruments, CA, USA), dipped in dental wax to reduce capacitance, then thermally polished to a resistance of 1.0-1.5 MΩ. Pipettes were filled with intracellular solution, containing: CsF (120 mM), CsCl (20 mM), NaCl (10 mM), HEPES (10 mM) titrated to pH 7.4. All recordings were made using an EPC-9 patch-clamp amplifier (HEKA Elektronik, Lambrecht, Germany) digitized at 20 kHz via an ITC-16 interface (Instrutech, Great Neck, N.Y., USA). Voltage clamping and data acquisition were controlled using PatchMaster/FitMaster software (HEKA Elektronik, Lambrecht, Germany) running on an Apple iMac (Cupertino, Calif.). Current was low-pass-filtered at 5 kHz. Leak subtraction was automatically done using a P/4 procedure following the test pulse. Gigaohm seals were allowed to stabilize in the on-cell configuration for 1 min prior to establishing the whole-cell configuration. Series resistance was less than 5 MΩ for all recordings. Series resistance compensation up to 80% was used when necessary. All data were acquired at least 5 min after attaining the whole-cell configuration, and cells were allowed to incubate 5 min after drug application prior to data collection. Before each protocol, the membrane potential was hyperpolarized to −130 mV to insure complete removal of both fast-inactivation and slow-inactivation. Leakage and capacitive currents were subtracted with a P/4 protocol. All experiments were conducted at 22° C.

### Example 29: Activation Protocols

To determine the voltage-dependence of activation, the peak current amplitude is measured at test pulse voltages ranging from −130 to +80 mV in increments of 10 mV for 19 ms. Channel conductance (G) was calculated from peak INa:

GNa=INa/(V−ENa) (Eq. 1)

where GNa is conductance, INa is peak sodium current in response to the command potential V, and ENa is the Nernst equilibrium potential. The midpoint and apparent valence of activation were derived by plotting normalized conductance as a function of test potential. Data were then fitted with a Boltzmann function:

G/Gmax=1/(1+exp(−ze0(Vm−V1/2)/kT) (Eq. 2)

where G/Gmax is normalized conductance amplitude, Vm is the command potential, z is the apparent valence, e0 is the elementary charge, V1/2 is the midpoint voltage, k is the Boltzmann constant, and T is temperature in K.

### Example 30: Steady State Fast Inactivation Protocols

The voltage-dependence of fast-inactivation was measured by preconditioning the channels to a hyperpolarizing potential of −130 mV and then eliciting pre-pulse potentials that ranged from −170 to +10 mV in increments of 10 mV for 500 ms, followed by a 10 ms test pulse during which the voltage was stepped to 0 mV. Normalized current amplitude as a function of voltage was fit using the Boltzmann function:

I/Imax=1/(1+exp(−ze0(VM−V1/2)/kT) (Eq. 3)

where Imax is the maximum test pulse current amplitude. z is apparent valency, e0 is the elementary charge, Vm is the prepulse potential, V1/2 is the midpoint voltage of SSFI, k is the Boltzmann constant, and T is temperature in K.

### Example 31: Fast Inactivation Recovery

Channels were fast inactivated during a 500 ms depolarizing step to 0 mV. Recovery was measured during a 19 ms test pulse to 0 mV following −130 mV recovery pulse for durations between 0 and 1.024 s. Time constants of fast inactivation were derived using a double exponential equation:

I=Iss+α1exp(−t/τ1)+α2exp(−t/τ2) (Eq. 4)

where I is current amplitude, Iss is the plateau amplitude, α1 and α2 are the amplitudes at time 0 for time constants τ1 and τ2, and t is time.

### Example 32: Persistent Current Protocols

Late sodium current was measured between 45 and 50 ms during a 50 ms depolarizing pulse to 0 mV from a holding potential of −130 mV. Fifty pulses were averaged to increase signal to noise ratio (Abdelsayed, Peters & Ruben, 2015; Abdelsayed, Ruprai & Ruben, 2018).

### Example 33: Action Potential Modeling

Action potentials were simulated using a modified version of the O'Hara-Rudy model programmed in Matlab (O'Hara et al. 2011, PLoS Comput. Bio). The code that was used to produce model is available online from the Rudy Lab website (http://rudylab.wustl.edu/research/cell/code/Allcodes.html). The modified gating INa parameters were in accordance with the biophysical data obtained from whole-cell patch-clamp experiments in this study for various conditions. The model accounted for activation voltage-dependence, steady-state fast inactivation voltage-dependence, persistent sodium currents, and peak sodium currents (compound conditions).

### Example 34: Drug Preparations

CANNABIDIOL was purchased from Toronto Research Chemicals (Toronto, Ontario) in powder form. Other compounds (e.g. 17β-Estradiol (E2), bradykinin, PGE-2, histamine, 5-HT, adenosine 5′-triphosphate, D-glucose, Gö 6983 (PKC inhibitor), H-89 (PKA inhibitor), 8-(4-chlorophenylthio) adenosine-3′,5′-cyclic monophosphate (CPT-cAMP; PKA activator) or PMA (PKC activator)) were purchased from Sigma-Aldrich (ON, Canada). Powdered CANNABIDIOL (CANNABIDIOL), Gö 6983, H-89, adenosine CPT-cAMP or PMA were dissolved in 100% DMSO to create stock. The stock was used to prepare drug solutions in extracellular solutions at various concentrations with no more than 0.5% total DMSO content.

### Work on Human Cardiomyocytes (Examples 35-40)

### Example 35: Preparation of Cell Culture of Human Cardiomyocytes and Action of Mediators

Single vials containing ≥1×106 cardiomyocytes (Cellular Dynamics International, kit 01434, Madison, Wis., USA) were thawed by immersing the frozen cryovial in a 37° C. water bath, transferring thawed cardiomyocytes into a 50-ml tube, and diluting them with 10 ml of ice-cold plating medium (iCell Cardiomyocytes Plating Medium (iCPM); Cellular Dynamics International, Madison, Wis., USA) (Ma et al., 2011). For single cell patch-clamp recordings, glass coverslips were coated with 0.1% gelatin (Cellular Dynamics International, Madison, Wis., USA) and placed into each well of a 24-well plate for an hour. This was followed by adding 1 ml of iCPM containing 40,000-60,000 cardiomyocytes to each coverslip. Plated cardiomyocytes were at a low density to permit culture as single cells and were stored in an environmentally controlled incubator maintained at 37° C. and 7% CO2. After 48 h, iCPM was replaced with a cell culture medium (iCell Cardiomyocytes Maintenance Medium (iCMM); Cellular Dynamics International, Madison, Wis., USA), which was exchanged every other day with the cardiomyocytes maintained on cover slips for 4 to 21 days before use (Ma et al., 2011). Cardiomyocytes were incubated in a cocktail of inflammatory mediators (Akin et al., 2019) containing bradykinin (1 μM), PGE-2 (10 μM), histamine (10 μM), 5-HT (10 μM), and adenosine 5′-triphosphate (15 μM) or the vehicle for 24 hours prior to electrophysiological experiments.

### Example 36: Electrophysiology

Whole-cell patch clamp recordings were made using an extracellular solution composed of NaCl (50 mM), CaCl2 (1.8 mM), MgCl2 (1 mM), CsCl2 (110 mM), glucose (10 mM), HEPES (10 mM) and nifedipine (0.001 mM) (Ma et al., 2011). The extracellular solution was titrated to pH 7.4 with CsOH. Pipettes were fabricated with a P-1000 puller using borosilicate glass (Sutter Instruments, CA, USA), dipped in dental wax to reduce capacitance, then thermally polished to a resistance of 2.0-3.5 MΩ. Pipettes were filled with intracellular solution, containing: CsCl2 (135 mM), NaCl (10 mM), CaCl2 (2 mM), EGTA (5 mM), HEPES (10 mM) and Mg-ATP (5 mM) titrated to pH 7.2 with CsOH (Ma et al., 2011). All recordings were made using an EPC-9 patch-clamp amplifier (HEKA Elektronik, Lambrecht, Germany) digitized at 20 kHz via an ITC-16 interface (Instrutech, Great Neck, N.Y., USA). Voltage clamping and data acquisition were controlled using PatchMaster/FitMaster software (HEKA Elektronik, Lambrecht, Germany) running on an Apple iMac (Cupertino, Calif.). Current was low-pass-filtered at 5 kHz. Gigaohm seals were allowed to stabilize in the on-cell configuration for 1 min prior to establishing the whole-cell configuration. Series resistance was less than 5 MΩ for all recordings. All experiments were conducted at 22° C.

### Example 37: Activation Protocols

To determine the voltage-dependence of activation, the peak current amplitude at test pulse voltages ranging from −130 to +80 mV in increments of 10 mV for 19 ms were measured. Channel conductance (G) was calculated from peak INa:

GNa=INa/(V−ENa) (Eq. 1)

where GNa is conductance, INa is peak sodium current in response to the command potential V, and ENa is the Nernst equilibrium potential. The midpoint and apparent valence of activation were derived by plotting normalized conductance as a function of test potential. Data were then fitted with a Boltzmann function:

G/Gmax=1/(1+exp(−ze0(Vm−V1/2)/kT) (Eq. 2)

where G/Gmax is normalized conductance amplitude, Vm is the command potential, z is the apparent valence, e0 is the elementary charge, V1/2 is the midpoint voltage, k is the Boltzmann constant, and T is temperature in K.

### Example 38: Steady State Fast Inactivation Protocols

The voltage-dependence of fast-inactivation was measured by preconditioning the channels to a hyperpolarizing potential of −130 mV and then eliciting pre-pulse potentials that ranged from −170 to +10 mV in increments of 10 mV for 500 ms, followed by a 10 ms test pulse during which the voltage was stepped to 0 mV. Normalized current amplitude as a function of voltage was fit using the Boltzmann function:

I/Imax=1/(1+exp(−ze₀(V_{M}−V_{1/2})/kT) (Eq. 3)

where Iₘₐₓ is the maximum test pulse current amplitude. z is apparent valency, e₀ is the elementary charge, Vm is the prepulse potential, V_{1/2} is the midpoint voltage of SSFI, k is the Boltzmann constant, and T is temperature in K.

### Example 39: Persistent Current Protocols

Late sodium current was measured between 145 and 150 ms during a 200 ms depolarizing pulse to 0 mV from a holding potential of −130 mV

### Example 40: Action Potential Modeling

Action potentials were simulated using a modified version of the O'Hara-Rudy model programmed in Matlab (O'Hara et al. 2011, PLoS Comput. Bio). The code that was used to produce model is available online from the Rudy Lab website (http://rudylab.wustl.edu/research/cell/code/Allcodes.html). The modified gating INa parameters were in accordance with the biophysical data obtained from whole-cell patch-clamp experiments in this study for various conditions. The model accounted for activation voltage-dependence, steady-state fast inactivation voltage-dependence, persistent sodium currents, and peak sodium currents (compound conditions).

Tables 2-5 provide actual readings taken in above experiments.

### Example 41: CANNABIDIOL Reduces the Pro-Arrhythmic Effects of Azithromycin

Cell Culture: Chinese hamster ovary (CHO) were grown at pH 7.4 in filtered sterile F12 (Ham) nutrient medium (Life Technologies, Thermo Fisher Scientific, Waltham, Mass., USA), supplemented with 5% FBS and maintained in a humidified environment at 37° C. with 5% CO2. Cells were transiently co-transfected with the human cDNA encoding the Nav1.5 α-subunit, the β1-subunit, and eGFP. Transfection was done according to the PolyFect (Qiagen, Germantown, Md., USA) transfection protocol. A minimum of 8-hour incubation was allowed after each set of transfections. Then, the cells were dissociated with 0.25% trypsin-EDTA (Life Technologies, Thermo Fisher Scientific) and the cells were incubated heterologously expressing Nav1.5 in 10 μM Az and observed an increase in late sodium current compared to control (no Az incubation) cells. The cells showing AZ-induced late sodium current are further perfused cells with 5 μM CANNABIDIOL and it was observed that the late current was reduced. From these preliminary results, we conclude that CANNABIDIOL rescues the proarrhythmic effects of AZ and, thus, may be a useful adjuvant therapy in conditions that call for treatment with macrolide antibiotics, possibly including COVID-19.

Following examples illustrate various pharmaceutical compositions of the present invention without restricting scope of the invention.

Example 42 - Formulation No. 1: AZITHROMYCIN FILM COATED TABLETS

| A | TABLET CORE | |
|---|---|---|
| 1 | Azithromycin dihydrate, USP equivalent to | 50 mg to 400 mg |
| | azithromycin | |
| 2 | Croscarmellose sodium | 5% of the total core |
| | | weight |
| 3 | Dibasic calcium phosphate anhydrous | 40% of the total core |
| | | weight |
| 4 | Pregelatinized starch | 10% of the total core |
| | | weight |
| 5 | Magnesium stearate | 0.5% of the total core |
| | | weight |
| B | FILM- COATING | |
| | Consisting of Hypromellose, polyethylene | 2.0-2.5% of the total |
| | glycol, and titanium dioxide reconstituted to | core weight |
| | 10% w/w dispersion in water-Iso-propyl | |
| | alcohol blend *or Iso-propyl alcohol* | |
| | * = Evaporates during tablet coating and is not | |
| | present substantially in the final product - The | |
| | film coated tablet. | |

| C | PROCESS: |
|---|---|
| | Co-sift Azithromycin dihydrate, croscarmellose sodium, dibasic calcium |
| | phosphate anhydrous and pre-gelatinised starch through ASTM # 40 |
| | mesh twice. Label it as Mix A. |
| | Sift and collect separately the magnesium stearate through ASTM # 40 |
| | in a polybag. |
| | Transfer the Mix A to a V-blender of appropriate size allowing 60% of |
| | its occupancy. Blend at 15 RPM for 10 minutes. Label it as Mix B. |
| | Add the pre-sifted magnesium stearate to the Mix B in the blender and |
| | continue to blend at 10 RPM for 2 minutes. |
| | Unload the final blend into a double LDPE polybag lined with a black |
| | polybag on the outermost side. Displace the air inside each bag and tie |
| | each one with a nylon tag. Keep 5 dessicant pillow pouches (each with |
| | 100 gm capacity) in the second outer bag before tying it up with a nylon |
| | tie. Finally, put the double polybag with the Mix into a black polybag |
| | and secure with a nylon tie. Label the final bag as “Lubricated Blend |
| | ready for compression”. Use appropriate compression tooling to |
| | compress the Lubricated Blend into biconvex tablets of appropriate |
| | hardness so that the percent friability is less than 0.5% w/w and the |
| | disintegration time (DT) is not more than 15 minutes. |
| | Note: All activity from dispensing of the Ingredients to the tablet |
| | compression and storage has to be carried out under strict control of |
| | ambient temperature and humidity conditions viz. Temperature of Not |
| | More Than (NMT) 18 degree centigrade and % Relative Humidity |
| | (% RH) of NMT 40%. |
| | Further coat the tablets in an appropriate tablet coater/coating machine |
| | with the hydro-alcoholic or alcoholic tablet coating dispersion of |
| | appropriate sprayable consistency to achieve a weight gain of 2.0-2.5% |
| | w/w on the tablet core. |
| | Fill the film-coated tablets into appropriate well-filled, opaque white/ |
| | coloured containers (of appropriate material) so that there is minimum |
| | head space along with appropriate protectants against moisture and |
| | oxygen. |

Example 42A Formulation No. 1A:
AZITHROMYCIN FILM COATED TABLETS

| A | TABLET CORE | |
|---|---|---|
| 1 | Azithromycin dihydrate, USP equivalent to | 400 mg to 800 mg |
| | azithromycin | |
| 2 | Croscarmellose sodium | 5% of the total core |
| | | weight |
| 3 | Dibasic calcium phosphate anhydrous | 40% of the total core |
| | | weight |
| 4 | Pregelatinized starch | 10% of the total core |
| | | weight |
| 5 | Magnesium stearate | 0.5% of the total |
| | | core weight |
| B | FILM- COATING | |
| | Consisting of Hypromellose, polyethylene | 2.0-2.5% of the |
| | glycol, and titanium dioxide reconstituted to 10% | total core weight |
| | w/w dispersion in water-Iso-propyl alcohol blend | |
| | *or Iso-propyl alcohol* | |
| | * = Evaporates during tablet coating and is not | |
| | present substantially in the final product - The | |

| | film coated tablet. |
|---|---|
| C | PROCESS: |
| | Co-sift Azithromycin dihydrate, croscarmellose sodium, dibasic calcium |
| | phosphate anhydrous and pre-gelatinised starch through ASTM # 40 |
| | mesh twice. Label it as Mix A. |
| | Sift and collect separately the magnesium stearate through ASTM # 40 |
| | in a polybag. |
| | Transfer the Mix A to a V-blender of appropriate size allowing 60% of |
| | its occupancy. Blend at 15 RPM for 10 minutes. Label it as Mix B. |
| | Add the pre-sifted magnesium stearate to the Mix B in the blender and |
| | continue to blend at 10 RPM for 2 minutes. |
| | Unload the final blend into a double LDPE polybag lined with a black |
| | polybag on the outermost side. Displace the air inside each bag and tie |
| | each one with a nylon tag. Keep 5 dessicant pillow pouches (each with |
| | 100 gm capacity) in the second outer bag before tying it up with a nylon |
| | tie. Finally, put the double polybag with the Mix into a black polybag |
| | and secure with a nylon tie. Label the final bag as “Lubricated Blend |
| | ready for compression”. Use appropriate compression tooling to |
| | compress the Lubricated Blend into biconvex tablets of appropriate |
| | hardness so that the percent friability is less than 0.5% w/w and the |
| | disintegration time (DT) is not more than 15 minutes. |
| | Note: All activity from dispensing of the Ingredients to the tablet |
| | compression and storage has to be carried out under strict control of |
| | ambient temperature and humidity conditions viz. Temperature of Not |
| | More Than (NMT) 18 degree centigrade and % Relative Humidity |
| | (% RH) of NMT 40%. |
| | Further coat the tablets in an appropriate tablet coater/coating machine |
| | with the hydro-alcoholic or alcoholic tablet coating dispersion of |
| | appropriate sprayable consistency to achieve a weight gain of 2.0-2.5% |
| | w/w on the tablet core. |
| | Fill the film-coated tablets into appropriate well-filled, opaque white/ |
| | coloured containers (of appropriate material) so that there is minimum |
| | head space along with appropriate protectants against moisture and |
| | oxygen. |

Example 43 - Formulation No. 2: CANNABIDIOL FILM COATED TABLETS

| A | TABLET CORE | |
|---|---|---|
| 1 | CANNABIDIOL | 0.1 mg to 100 mg or |
| | | 100 mg to 200 mg |
| 2 | Microcrystalline cellulose (MCC PH 105) | 40% of the total core |
| | | weight |
| 3 | CELLULOSE | 2% of the total core |
| | METHYLHYDROXYPROPYL 5CPS | weight |
| 4 | COLLOIDAL SILICON DIOXIDE | 2% of the total core |
| | | weight |
| 5 | POLYVINYL PYROLLIDONE (PVP | 2% of the total core |
| | K29/32) | weight |
| 6 | MAGNESIUM STEARATE | 0.5% of the total core |
| | | weight |
| B | FILM- COATING | |
| | Consists of Polyvinyl alcohol, polyethylene | 2.0-2.5% of the total |
| | glycol, Talc, Opacifier, lecithin reconstituted | core weight |
| | to 10% w/w dispersion in water-Iso-propyl | |
| | alcohol blend *or Iso-propyl alcohol* | |
| | * = Evaporates during tablet coating and is not | |
| | present substantially in the final product - The | |
| | film coated tablet. | |
| C | PROCESS: | |

Co-sift CANNABIDIOL and MCC PH 105, Cellulose methyl
hydroxypropyl and polyvinyl - pyrollidone through ASTM # 40 mesh
twice. Label it as Mix A.
Sift individually the colloidal silicon dioxide and the magnesium stearate
through ASTM # 40 and collect in separate polybags.
Transfer the Mix A to a V-blender of appropriate size allowing 60% of
its occupancy. Blend at 15 RPM for 10 minutes. Label it as Mix B.
Add the pre-sifted colloidal silicon dioxide the Mix B in the blender and
continue to blend at 10 RPM for 5 minutes. Label it as Mix C.
Add the pre-sifted magnesium stearate to the Mix C in the blender and
continue to blend at 10 RPM for 2 minutes.
Unload the final blend into a double LDPE polybag lined with a black
polybag on the outermost side. Displace the air inside each bag and tie
each one with a nylon tag. Keep 5 dessicant pillow pouches (each with
100 gm capacity) in the second outer bag before tying it up with a nylon
tie. Finally, put the double polybag with the Mix into a black polybag
and secure with a nylon tie. Label the final bag as “Lubricated Blend
ready for compression”. Use appropriate compression tooling to
compress the Lubricated Blend into biconvex tablets of appropriate
hardness so that the percent friability is less than 0.5% w/w and the
disintegration time (DT) is not more than 15 minutes.
Further coat the tablets in an appropriate tablet coater/coating machine
with the hydro-alcoholic or alcoholic tablet coating dispersion of
appropriate sprayable consistency to achieve a weight gain of 2.0-2.5%
w/w on the tablet core.
Fill the film-coated tablets into appropriate well-filled, opaque white/
coloured containers (of appropriate material) so that there is minimum
head space along with appropriate protectants against moisture and
oxygen.

Example 44 - Formulation No. 3: CANNABIDIOL CAPSULES

| A | CORE INGREDIENTS | |
|---|---|---|
| 1 | CANNABIDIOL | 0.1 mg to 100 mg or 100 mg to |
| | | 200 mg |
| 2 | Microcrystalline cellulose (MCC | 40% of the total capsule core |
| | PH 105) | weight |
| 3 | CELLULOSE | 2% of the total capsule core |
| | METHYLHYDROXYPROPYL 5CPS | weight |
| 4 | COLLOIDAL SILICON DIOXIDE | 2% of the total capsule core |
| | | weight |
| 5 | POLYVINYL PYROLLIDONE (PVP | 2% of the total capsule core |
| | K29/32) | weight |
| 6 | MAGNESIUM STEARATE | 0.5% of the total capsule core |
| | | weight |
| B | ENCAPSULATION | |
| | Consisting of opaque, coloured, | |
| | Hydroxy-propyl methyl cellulose | |
| | (HPMC) of appropriate size viz. 00el to | |
| | 5 to encompass or encapsulate the | |
| | ingredients. | |
| C | PROCESS: | |

Co-sift CANNABIDIOL and MCC PH 105, cellulose methyl
hydroxypropyl and polyvinyl - pyrollidone through ASTM # 40 mesh
twice. Label it as Mix A.
Sift individually the colloidal silicon dioxide and the magnesium
stearate through ASTM # 40 and collect in separate polybags.
Transfer the Mix A to a V-blender of appropriate size allowing 60% of
its occupancy. Blend at 15 RPM for 10 minutes. Label it as Mix B.
Add the pre-sifted colloidal silicon dioxide the Mix B in the blender and
continue to blend at 10 RPM for 5 minutes. Label it as Mix C.
Add the pre-sifted magnesium stearate to the Mix C in the blender and
continue to blend at 10 RPM for 2 minutes.
Unload the final blend into a double LDPE polybag lined with a black
polybag on the outermost side. Displace the air inside each bag and tie
each one with a nylon tag. Keep 5 dessicant pillow pouches (each with
100 gm capacity) in the second outer bag before tying it up with a nylon
tie. Finally, put the double polybag with the Mix into a black polybag
and secure with a nylon tie. Label the final bag as “Lubricated Blend
ready for Capsule filling”. Use appropriate tooling and the Capsule
filling machine to fill the Lubricated Blend into capsules of appropriate
size such that the disintegration time (DT) is not more than 10 minutes.
Label them as Linished Capsules.
Fill the finished capsules into appropriate well-filled, opaque white/
coloured containers (of appropriate material) so that there is minimum
head space along with appropriate protectants against moisture and
oxygen.

Example 45 - Formulation No. 4: CHLOROQUINE PHOSPHATE TABLETS

| A | TABLET CORE | |
|---|---|---|
| 1 | Contains: | 50 or 100 or |
| | 50 mg chloroquine phosphate (equivalent to 30 mg base) or | 150 or 250 mg |
| | 100 mg chloroquine phosphate (equivalent to 60 mg base) or | |
| | 250 mg chloroquine phosphate (equivalent to 150 mg base) or | |
| | 500 mg chloroquine phosphate (equivalent to 300 mg base). | |
| 2 | Microcrystalline cellulose (Avicel PH 102)/ | 40% of the total core |
| | dibasic calcium phosphate (granular free flowing | weight |
| | grade) | |
| 3 | Corn starch | 10% of the total core |
| | | weight |
| 4 | Povidone (PVP K29/32) | 5% of the total core |
| | | weight |
| 5 | Sodium starch glycolate | 3% of the total core |
| | | weight |
| 6 | Colloidal silicon dioxide | 1% of the total core |
| | | weight |
| 7 | Magnesium stearate | 0.5% of the total tablet |
| | | core weight |
| B | FILM- COATING | |
| | Consists of Hypromellose, polyethylene glycol | |
| | 400, polyethylene glycol 3350, polysorbate 80, | |
| | talc, and titanium dioxide reconstituted to 10% | |
| | w/w dispersion in water-Iso-propyl alcohol blend | |
| | *or Iso-propyl alcohol* | |
| | * = Evaporates during tablet coating and is not | |
| | present substantially in the final product - The film | |
| | coated tablet. | |

Co-sift the Chloroquine phosphate, Microcrystalline cellulose/Dibasic
calcium phosphate, corn starch, Povidone and sodium starch glycolate
through ASTM # 40 mesh twice. Label it as Mix A.
Sift individually the colloidal silicon dioxide and the magnesium stearate
through ASTM # 40 and collect in separate polybags.
Transfer the Mix A to a V-blender of appropriate size allowing 60% of its
occupancy. Blend at 15 RPM for 10 minutes. Label it as Mix B.
Add the pre-sifted colloidal silicon dioxide the Mix B in the blender and
continue to blend at 10 RPM for 5 minutes. Label it as Mix C.
Add the pre-sifted magnesium stearate to the Mix C in the blender and
continue to blend at 10 RPM for 2 minutes.
Unload the final blend into a double LDPE polybag lined with a black
polybag on the outermost side. Displace the air inside each bag and tie each
one with a nylon tag. Keep 5 dessicant pillow pouches (each with 100 gm
capacity) in the second outer bag before tying it up with a nylon tie. Finally,
put the double polybag with the Mix into a black polybag and secure with a
nylon tie. Label the final bag as “Lubricated Blend ready for compression”.
Use appropriate compression tooling to compress the Lubricated Blend into
biconvex tablets of appropriate hardness so that the percent friability is less
than 0.5% w/w and the disintegration time (DT) is not more than 15
minutes.
Further coat the tablets in an appropriate tablet coater/coating machine
with the hydro-alcoholic or alcoholic tablet coating dispersion of
appropriate sprayable consistency to achieve a weight gain of 2.0-2.5% w/w
on the tablet core.
Fill the film-coated tablets into appropriate well-filled, opaque white/
coloured containers (of appropriate material) so that there is minimum head
space along with appropriate protectants against moisture and oxygen.

Example 46 Formulation No. 5: HYROXYCHLOROQUINE
SULPHATE TABLETS

| A | TABLET CORE | |
|---|---|---|
| 1 | Hydroxychloroquine sulphate | 50 mg to 500 mg |
| 2 | Lactose monohydrate (granular free | 40% of the total core weight |
| | flowing grade) | |
| 3 | Corn starch | 10% of the total core weight |
| 4 | Crospovidone | 5% of the total core weight |
| 5 | Hydroxypropyl methylcellulose | 3% of the total core weight |
| 6 | Magnesium stearate | 0.5% of the total tablet core |
| | | weight |
| B | FILM- COATING | |
| | Consists of polyethylene glycol, | |
| | polyvinyl alcohol, talc and titanium | |
| | dioxide reconstituted to 10% w/w | |
| | dispersion in water-Iso-propyl alcohol | |
| | blend *or Iso-propyl alcohol* | |
| | * = Evaporates during tablet coating and | |
| | is not present substantially in the final | |
| | product - The film coated tablet. | |

Co-sift the Hydroxychloroquine sulphate, Lactose monohydrate, corn starch,
Crospovidone and hydroxypropyl methylcellulose through ASTM # 40 mesh
twice. Label it as Mix A.
Sift individually the magnesium stearate through ASTM # 40 and collect in
separate polybag. Transfer the Mix A to a V-blender of appropriate size
allowing 60% of its occupancy. Blend at 15 RPM for 10 minutes. Label it as
Mix B.
Add the pre-sifted magnesium stearate to the Mix B in the blender and
continue to blend at 10 RPM for 2 minutes.
Unload the final blend into a double LDPE polybag lined with a black
polybag on the outermost side. Displace the air inside each bag and tie each
one with a nylon tag. Keep 5 dessicant pillow pouches (each with 100 gm
capacity) in the second outer bag before tying it up with a nylon tie. Finally,
put the double polybag with the Mix into a black polybag and secure with a
nylon tie. Label the final bag as “Lubricated Blend ready for compression”.
Use appropriate compression tooling to compress the Lubricated Blend into
biconvex tablets of appropriate hardness so that the percent friability is less
than 0.5% w/w and the disintegration time (DT) is not more than 15 minutes.
Further coat the tablets in an appropriate tablet coater/coating machine with
the hydro-alcoholic or alcoholic tablet coating dispersion of appropriate
sprayable consistency to achieve a weight gain of 2.0-2.5% w/w on the tablet
core.
Fill the film-coated tablets into appropriate well-filled, opaque white/
coloured containers (of appropriate material) so that there is minimum head
space along with appropriate protectants against moisture and oxygen.

Example 47 - Formulation No. 6: BILAYER TABLETS of
AZITHROMYCIN AND CANNABIDIOL
Using an appropriate tableting machine and compression tooling;
compress the Lubricated blends of formulation 1 and 2 into tablets of
suitable dose and size that are biconvex and having % Friability less than
0.5% and Disintegration time Not More Than 15 minutes.
Further coat the tablets in an appropriate tablet coater/coating machine
with the hydro-alcoholic or alcoholic tablet coating dispersion of
appropriate sprayable consistency to achieve a weight gain of 2.0-2.5%
w/w on the tablet core.
Fill the film-coated tablets into appropriate well-filled, opaque white/
coloured containers (of appropriate material) so that there is minimum
head space along with appropriate protectants against moisture and
oxygen.
Example 48 - Formulation No. 7: BILAYER TABLETS of
CANNABIDIOL AND CHLOROQUINE PHOSPHATE
Using an appropriate tableting machine and compression tooling; compress the
Lubricated blends of formulation 2 and 4 into tablets of suitable dose and size that
are biconvex and having % Friability less than 0.5% and Disintegration time Not
More Than 15 minutes.
Further coat the tablets in an appropriate tablet coater/coating machine with the
hydro-alcoholic or alcoholic tablet coating dispersion of appropriate sprayable
consistency to achieve a weight gain of 2.0-2.5% w/w on the tablet core.
Fill the film-coated tablets into appropriate well-filled, opaque white/coloured
containers (of appropriate material) so that there is minimum head space along with
appropriate protectants against moisture and oxygen

Example 49 - Formulation No. 8: TRILAYER TABLETS of AZITHROMYCIN,
CANNABIDIOL AND CHLOROQUINE PHOSPHATE.
Using an appropriate tableting machine and compression tooling; compress the
Lubricated blends of formulation 1, 2 and 4 into tablets of suitable dose and size
that are biconvex and having % Friability less than 0.5% and Disintegration time
Not More Than 15 minutes.
Further coat the tablets in an appropriate tablet coater/coating machine with the
hydro-alcoholic or alcoholic tablet coating dispersion of appropriate sprayable
consistency to achieve a weight gain of 2.0-2.5% w/w on the tablet core.
Fill the film-coated tablets into appropriate well-filled, opaque white/coloured
containers (of appropriate material) so that there is minimum head space along with
appropriate protectants against moisture and oxygen
Example 49 - Formulation No. 9: BILAYER TABLETS of CANNABIDIOL
AND HYDROXYCHLOROQUINE SULPHATE
Using an appropriate tableting machine and compression tooling; compress the
Lubricated blends of formulation 2 and 5 into tablets of suitable dose and size
that are biconvex and having % Friability less than 0.5% and Disintegration time
Not More Than 15 minutes.
Further coat the tablets in an appropriate tablet coater/coating machine with the
hydro-alcoholic or alcoholic tablet coating dispersion of appropriate sprayable
consistency to achieve a weight gain of 2.0-2.5% w/w on the tablet core.
Fill the film-coated tablets into appropriate well-filled, opaque white/coloured
containers (of appropriate material) so that there is minimum head space along
with appropriate protectants against moisture and oxygen

Example 50 - Formulation No. 10: TRILAYER TABLETS of AZITHROMYCIN,
CANNABIDIOL AND HYDROXYCHLOROQUINE SULPHATE

Using an appropriate tableting machine and compression tooling; compress the
Lubricated blends of formulation 1, 2 and 5 into tablets of suitable dose and size
that are biconvex and having % Friability less than 0.5% and Disintegration time
Not More Than 15 minutes.
Further coat the tablets in an appropriate tablet coater/coating machine with the
hydro-alcoholic or alcoholic tablet coating dispersion of appropriate sprayable
consistency to achieve a weight gain of 2.0-2.5% w/w on the tablet core.
Fill the film-coated tablets into appropriate well-filled, opaque white/coloured
containers (of appropriate material) so that there is minimum head space along
with appropriate protectants against moisture and oxygen

Example 51 - Formulation No. 11: AZITHROMYCIN SUSPENSION for oral use

| A | INGREDIENTS | |
|---|---|---|
| 1 | Azithromycin monohydrate equivalent to 100 mg or 200 mg of | |
| | Azithromycin/5 ml | |
| 2 | Sucrose | 40% w/v of 5 ml |
| 3 | Tribasic sodium phosphate | 0.5% w/v of 5 ml |
| | anhydrous | |
| 4 | Hydroxypropyl cellulose | 20% w/v of 5 ml |
| 5 | Xanthan gum | 10% w/v of 1 ml |
| 6 | Colloidal silicon dioxide | 30% w/v of 1 ml |
| 7 | Flavour - Orange or Vanilla | 0.2% w/v of 1 ml |
| | Purified water = Quantity sufficient | |
| | for reconstitution such that each 5 ml | |
| | of the suspension contains 100 mg or | |
| | 200 mg of Azithromycin. | |

| B | PROCESS |
|---|---|
| | Co-sift Azithromycin monohydrate, Sucrose, Tribasic sodium |
| | phosphate anhydrous, Hydroxypropyl cellulose, Xanthan gum, |
| | Colloidal silicon dioxide and Flavour through ASTM # 40 sieve twice. |
| | Label as Mix A. |
| | Load the Mix A to a V-blender of appropriate size allowing 60% of its |
| | occupancy. Blend at 15 RPM for 10 minutes. Label it as Mix B. |
| | Unload the final blend into a double LDPE polybag lined with a black |
| | polybag on the outermost side. Displace the air inside each bag and tie |
| | each one with a nylon tag. Keep 5 dessicant pillow pouches (each with |
| | 100 gm capacity) in the second outer bag before tying it up with a nylon |
| | tie. Finally, put the double polybag with the Mix into a black polybag |
| | and secure with a nylon tie. Label the final bag as “Lubricated Blend |
| | ready for bottle filling”. |
| | Fill appropriate quantity into opaque, HDPE bottles such that when |
| | reconstituted appropriately with freshly boiled and cooled water, it |
| | would result in a suspension of 100 mg or 200 mg/5ml. |
| | Note: All activity from dispensing of the Ingredients to the tablet |
| | compression and storage has to be carried out under strict control of |
| | ambient temperature and humidity conditions viz. Temperature of Not |
| | More Than (NMT) 18 degree centigrade and % Relative Humidity |
| | (% RH) of NMT 40%. |

Example 52 - Formulation No. 12: OSELTAMIVIR PHOSPHATE CAPSULES

| A | CORE INGREDIENTS | |
|---|---|---|
| 1 | Oseltamivir phosphate | 10 mg or 30 mg |
| 2 | Pregelatinized starch | 40% of the total capsule core weight |
| 3 | Croscarmellose sodium | 5% of the total capsule core weight |
| 4 | Povidone (PVP K29/32) | 2% of the total capsule core weight |
| 5 | Talc | 1% of the total capsule core weight |
| 6 | Sodium stearyl fumarate | 0.5% of the total capsule core weight |
| B | ENCAPSULATION | |
| | Consisting of opaque, coloured, | |
| | Hydroxy-propyl methyl | |
| | cellulose (HPMC) of | |
| | appropriate size viz. 00el to 5 to | |
| | encompass or encapsulate the | |
| | ingredients. | |

Co-sift Oseltamivir phosphate, Pregelatinized starch,
Croscarmellose sodium and Povidone (PVP K29/32) through ASTM
# 40 mesh twice. Label it as Mix A.
Sift individually the talc and the Sodium stearyl fumarate through
ASTM # 40 and collect in separate polybags.
Transfer the Mix A to a V-blender of appropriate size allowing 60%
of its occupancy. Blend at 15 RPM for 10 minutes. Label it as Mix
B.
Add the pre-sifted talc to the Mix B in the blender and continue to
blend at 10 RPM for 5 minutes. Label it as Mix C.
Add the pre-sifted sodium stearyl fumarate to the Mix C in the
blender and continue to blend at 10 RPM for 2 minutes.
Unload the final blend into a double LDPE polybag lined with a
black polybag on the outermost side. Displace the air inside each bag
and tie each one with a nylon tag. Keep 5 dessicant pillow pouches
(each with 100 gm capacity) in the second outer bag before tying it
up with a nylon tie. Finally, put the double polybag with the Mix into
a black polybag and secure with a nylon tie. Label the final bag as
“Lubricated Blend ready for Capsule filling”. Use appropriate
tooling and the Capsule filling machine to fill the Lubricated Blend
into capsules of appropriate size such that the disintegration time
(DT) is not more than 10 minutes. Label them as Finished Capsules.
Fill the finished capsules into appropriate well-filled, opaque white/
coloured containers (of appropriate material) so that there is
minimum head space along with appropriate protectants against
moisture and oxygen.

Example 53 - Formulation No. 13: OSELTAMIVIR
PHOSPHATE SUSPENSION for oral use.

| A | INGREDIENTS | |
|---|---|---|
| 1 | Oseltamivir phosphate | 60 mg |
| 2 | Sorbitol | 40% of total powder weight |
| 3 | Silicon dioxide | 40% of total powder weight |
| 4 | Sodium benzoate | 0.5% of total powder eight |
| 5 | Xanthan gum | 10% of total powder weight |
| 6 | Saccharin sodium | 0.5% of total powder weight |
| 7 | Flavour - Orange or Vanilla | 0.2% of total powder weight |
| B | PROCESS | |

Co-sift Oseltamivir phosphate, Sorbitol, Silicon dioxide, sodium benzoate,
xanthan gum, saccharin sodium and Flavour through ASTM # 40 sieve
twice. Label as Mix A. Load the Mix A to a V-blender of appropriate size
allowing 60% of its occupancy. Blend at 15 RPM for 10 minutes. Label it
as Mix B.
Unload the final Mix B into a double LDPE polybag lined with a black
polybag on the outermost side. Displace the air inside each bag and tie each
one with a nylon tag. Keep 5 dessicant pillow pouches (each with 100 gm
capacity) in the second outer bag before tying it up with a nylon tie. Finally,
put the double polybag with the Mix into a black polybag and secure with a
nylon tie. Label the final bag as “Lubricated Blend ready for bottle filling”.
Fill appropriate quantity into opaque, HDPE bottles such that when
reconstituted appropriately with freshly boiled and cooled water, it would
result in a suspension of 6 mg/ml of Oseltamivir base.
Note: All formulation activity from dispensing of the Ingredients to filling
and storage has to be carried out under strict control of ambient temperature
and humidity conditions viz. Temperature of Not More Than (NMT) 18
degree centigrade and % Relative Humidity (% RH) of NMT 40%.

Example 54 - Formulation No. 14: ATAZANAVIR SULPHATE CAPSULE

| A | CORE INGREDIENTS | |
|---|---|---|
| 1 | Atazanavir sulfate | 50 mg or 100 mg or 150 mg or 200 mg or 300 mg |
| 2 | Lactose monohydrate | 40% of the total capsule core weight |
| 3 | Crospovidone (15 MPA · S AT 5%) | 5% of the total capsule core weight |
| 4 | Magnesium stearate | 0.5% of the total capsule core weight |
| B | ENCAPSULATION | |
| | Consisting of opaque, coloured, | |
| | Hydroxy-propyl methyl cellulose | |
| | (HPMC) of appropriate size viz. | |
| | 00el to 5 to encompass or | |
| | encapsulate the ingredients. | |

Co-sift Atazanavir sulfate, Lactose monohydrate, Crospovidone through
ASTM # 40 mesh twice. Label it as Mix A.
Sift individually the magnesium stearate through ASTM # 40 and collect in
separate polybag.
Transfer the Mix A to a V-blender of appropriate size allowing 60% of its
occupancy. Blend at 15 RPM for 10 minutes. Label it as Mix B.
Add the pre-sifted magnesium stearate to the Mix B in the blender and
continue to blend at 10 RPM for 2 minutes. Label it as Mix C.
Unload the final Mix C into a double LDPE polybag lined with a black
polybag on the outermost side. Displace the air inside each bag and tie each
one with a nylon tag. Keep 5 dessicant pillow pouches (each with 100 gm
capacity) in the second outer bag before tying it up with a nylon tie. Finally,
put the double polybag with the Mix into a black polybag and secure with a
nylon tie. Label the final bag as “Lubricated Blend ready for Capsule filling”.
Use appropriate tooling and the Capsule filling machine to fill the Lubricated
Blend into capsules of appropriate size such that the disintegration time (DT)
is not more than 10 minutes. Label them as Finished Capsules.
Fill the finished capsules into appropriate well-filled, opaque white/coloured
containers (of appropriate material) so that there is minimum head space
along with appropriate protectants against moisture and oxygen.

Example 55 - Formulation No. 15: RIBAVIRIN CAPSULES

| A | CORE INGREDIENTS | |
|---|---|---|
| 1 | Ribavirin | 50 mg or 100 mg or 150 mg or 200 mg |
| 2 | Microcrystalline cellulose PH | 45% of the total capsule core weight |
| | 102 or PH 105 | |
| 3 | Lactose monohydrate | 45% of the total capsule core weight |
| 4 | Croscarmellose sodium | 9% of the total capsule core weight |
| 5 | Magnesium stearate | 0.5% of the total capsule core weight |
| 6 | ENCAPSULATION | |

Consisting of opaque, coloured, Hydroxy-propyl methyl cellulose
(HPMC) of appropriate size viz. 00el to 5 to encompass or encapsulate
the ingredients.
Co-sift Ribavirin, Microcrystalline cellulose, Lactose monohydrate,
Croscarmellose sodium through ASTM # 40 mesh twice. Label it as Mix
A. Sift individually the magnesium stearate through ASTM # 40 and
collect in separate polybag.
Transfer the Mix A to a V-blender of appropriate size allowing 60% of
its occupancy. Blend at 15 RPM for 10 minutes. Label it as Mix B.
Add the pre-sifted magnesium stearate to the Mix B in the blender and
continue to blend at 10 RPM for 2 minutes. Label it as Mix C.
Unload the final Mix C into a double LDPE polybag lined with a black
polybag on the outermost side. Displace the air inside each bag and tie
each one with a nylon tag. Keep 5 dessicant pillow pouches (each with
100 gm capacity) in the second outer bag before tying it up with a nylon
tie. Finally, put the double polybag with the Mix into a black polybag
and secure with a nylon tie. Label the final bag as “Lubricated Blend
ready for Capsule filling”. Use appropriate tooling and the Capsule filling
machine to fill the Lubricated Blend into capsules of appropriate size
such that the disintegration time (DT) is not more than 10 minutes. Label
them as Finished Capsules.
Fill the finished capsules into appropriate well-filled, opaque white/
coloured containers (of appropriate material) so that there is minimum
head space along with appropriate protectants against moisture and
oxygen.

Example 56 - Formulation No. 16: RIBAVIRIN ORAL SOLUTION

| A | INGREDIENTS | |
|---|---|---|
| 1 | Ribavirin | 10 mg or 20 mg or 30 mg or 40 mg |
| 2 | Sucrose | 20% w/v of the total quantity |
| 3 | Glycerine | 30% w/v of the total quantity |
| 4 | Sorbitol | 0.5% w/v of the total quantity |
| 5 | Propylene glycol | 30% w/v of the total quantity |
| 6 | sodium citrate | 0.5% w/v of the total quantity |
| 7 | Citric acid | 0.5% w/v of the total quantity |
| 8 | Sodium benzoate | 0.2% w/v of the total quantity |
| 9 | Flavour | 0.2% w/v of the total quantity |
| 10 | Water Qs to make (Total | 1 ml |
| | quantity) | |

Note: The afore-mentioned formula is a per millilitre formula
and needs appropriate scale up factoring for manufacturing.
Process:
Take water in a closed vessel and keep it under continuous stirring.
Add serially to it sucrose, glycerine, sorbitol, propylene glycol sodium
citrate, citric acid, sodium benzoate, flavour and the Ribavirin.
Continue to stir till a clear pale to light yellow solution is contained.
Label it as Ribavirin Solution.
Fill the solution into well filled containers of appropriate size.

Example 57 - Formulation No. 17:
CANNABIDIOL INJECTION or CANNABIDIOL nasal drops or CANNABIDIOL nasal
spray or CANNABIDIOL buccal drops or CANNABIDIOL buccal spray or
CANNABIDIOL sublingual drops or CANNABIDIOL sublingual spray

| | | |
|---|---|---|
| 1 | CANNABIDIOL | 0.5-100 mg/ml |
| 2 | Propylene glycol | 30% |
| 3 | Ethyl alcohol | 20% |
| 4 | Sodium benzoate/benzoic acid | 5% |
| 5 | Benzyl alcohol | 1.50% |
| 6 | Water for injection | ~43% |
| | It is a sterile, nonpyrogenic solution. The pH | |
| | range if reconstituted should be 5-9 preferably | |
| | 6.5-7.5 | |

Dissolve the CANNABIDIOL in ethanol under continuous stirring in a
closed vessel. Label it as Mix A.
Add the sodium benzoate/benzoic acid and benzyl alcohol to propylene
glycol under continuous stirring in a larger vessel. Slowly add water to it
under stirring. Label it as Mix B.
Add the Mix B to mix A under continuous stirring.
Continue stirring till a clear solution is formed. Filter the final clear
solution through a 0.2-micron filter to yield a sterile solution.
All activity is to be executed in a parenteral facility using aseptic process
only.
Using aseptic filling fill and seal the sterile solution into ampoules of 1 ml
capacity under nitrogen purging and under subdued light or under a sodium
vapour lamp.
The said formulation can be administered via the nasal route as nasal drops
or as nasal spray using appropriate medical device.
The said formulation can be administered via inhalation with or without
the aid of a medical device, metered or unmetered, and/or via nebulization.
The said formulation can be administered via the buccal route as buccal
drops or as buccal spray using appropriate medical device.
The said formulation can be administered via the sublingual route as
sublingual drops or as sublingual spray using appropriate medical device.

Example 58 - Formulation No. 18:
CANNABIDIOL INJECTION or CANNABIDIOL nasal drops or
CANNABIDIOL nasal spray or CANNABIDIOL buccal drops or
CANNABIDIOL buccal spray or CANNABIDIOL sublingual
drops or CANNABIDIOL sublingual spray

| | | |
|---|---|---|
| 1 | CANNABIDIOL | 0.5-100 mg/ml (active) |
| 2 | Ethyl alcohol | 20% of the active |
| 3 | Propylene glycol | 40% of the active |
| 4 | Water for injection | ~40% |

It is a sterile, nonpyrogenic solution with
pH range 4.0-7.0. The pH range if
reconstituted should be 5-9 preferably 6.5-
7.5
Dissolve the CANNABIDIOL in ethanol under continuous stirring in a small
vessel. Label it as Mix A.
Add the propylene glycol to mix A under continuous stirring in a larger
vessel. Slowly add water to it under stirring.
Continue stirring till a clear solution is formed. Filter the final clear solution
through a 0.2-micron filter to yield a sterile solution.
All activity is to be executed in a parenteral facility using aseptic process
only. Using aseptic filling fill and seal the sterile solution into ampoules of 1
ml capacity under nitrogen purging and under subdued light or under a
sodium vapour lamp.
The said formulation can be administered via the nasal route as nasal drops
or as nasal spray using appropriate medical device.
The said formulation can be administered via inhalation with or without the
aid of a medical device, metered or unmetered, and/or via nebulization.
The said formulation can be administered via the buccal route as buccal drops
or as buccal spray using appropriate medical device.
The said formulation can be administered via the sublingual route as
sublingual drops or as sublingual spray using appropriate medical device.

Example 59 - Formulation No. 19: AZITHROMYCIN INJECTION

| | | |
|---|---|---|
| 1 | Azithromycin dihydrate, USP equivalent to 100 mg | 100 mg |
| | azithromycin | |
| 2 | Edetate disodium | 5.4 mg |
| 3 | Polysorbate 80 | 75 mg |
| 4 | Lactose anhydrous | 375 mg |
| 5 | Sodium hydroxide and/or hydrochloric acid (for pH | qs |
| | adjustment). | |
| 6 | Water for injection | ~99% |
| | Supplied as white lyophilized cake or powder in a 10 mL | |
| | vial/ampoule | |
| | The same may be diluted in appropriate sterile saline, | |
| | dextrose or Ringer solution. The pH range if reconstituted | |
| | should be 5-9 preferably 6.5-7.5 | |

Dissolve the Edetate disodium, Polysorbate 80, lactose anhydrous and the
Azithromycin dihydrate in the water for injection under continuous stirring
in a vessel. Label it as Mix A. Dissolve the sodium hydroxide in a small
quantity of water. Adjust the pH of Mix A by adding the sodium hydroxide
solution or hydrochloric acid under continuous stirring till a pH of 6.5 to
7.5 is achieved.
Filter the final clear solution through a 0.2-micron filter to yield a sterile
solution.
All activity is to be executed in a parenteral facility using aseptic process
only.
Using aseptic filling fill and seal the sterile solution into ampoules of 1 ml
capacity under nitrogen purging and under subdued light or under a sodium
vapour lamp.

### Examples 60A-60I

### Pharmaceutical Compositions of CANNABIDIOL

### Processes of Preparation of Example 60A-60I

### Procedure(s)

### Sublingual Tablets (60A)

Co-sift and blend CANNABIDIOL, Lactose monohydrate and Mannitol through ASTM #40 mesh twice. Pre-label it as Mix A. Co-sift the starch and Polyvinyl pyrollidone each individually pre-sifted through ASTM #40 to the Mix A and blend in a V-cone blender at 20 RPM for 20 minutes. Label it as Mix B. Add the Sodium stearyl fumarate pre-sifted through ASTM #60 to the Mix B in the blender and continue to blend at 20 RPM for 2 minutes. Unload the final blend into a double LDPE polybag lined with a black polybag on the outermost side. Displace the air inside each bag and tie each one with a nylon tag. Keep 5 desiccant pillow pouches in the second outer bag before tying it up with a nylon tie. Finally, put the double polybag with the Mix into a black polybag and secure with a nylon tie. Label the final bag as “Lubricated Blend ready for compression”. Use a 9-10 mm Flat faced, oval shaped compression tooling to compress the final Lubricated blend into flat tablets of appropriate hardness and thickness preferably less than 2.0 mm and their disintegration time (DT) is not less than 3 minutes. Alternatively, the tablets may have 2 or more score lines to adjust the dosage in multiples of 5 mg/tablet segment.

### Fast Dissolving Tablets (60B)

Co-sift and blend CANNABIDIOL and Tween 20 (paste), Lactose monohydrate, sodium citrate and Mannitol through ASTM #40 mesh twice. Pre-label it as Mix A. Co-sift the Polyvinyl pyrollidone pre-sifted through ASTM #40 to the Mix A and blend in a V-cone blender at 20 RPM for 20 minutes. Label it as Mix B. Add the Carbowax pre-sifted through ASTM #60 to the Mix B in the blender and continue to blend at 20 RPM for 2 minutes. Unload the final blend into a double LDPE polybag lined with a black polybag on the outermost side. Displace the air inside each bag and tie each one with a nylon tag. Keep 5 desiccant pillow pouches in the second outer bag before tying it up with a nylon tie. Finally, put the double polybag with the Mix into a black polybag and secure with a nylon tie. Label the final bag as “Lubricated Blend ready for compression”. Use a 9-10 mm Flat faced, oval shaped compression tooling to compress the final Lubricated blend into flat tablets of appropriate hardness so that the percent friability is less than 0.5% w/w and the disintegration time (DT) is not more than 2 minutes. Alternatively, the tablets may have 2 or more score lines to adjust the dosage in multiples of 5 mg/tablet segment.

### Quick Dispersible Tablets (60C)

Co-sift and blend CANNABIDIOL, sodium citrate and Crospovidone Ultra through ASTM #40 mesh twice. Pre-label it as Mix A. Co-sift and add the starch and Polyvinyl pyrollidone each individually presifted through ASTM #40 to the Mix A and blend in a V cone blender at 20 RPM for 20 minutes. Label it as Mix B. Add the Sodium stearyl fumarate pre-sifted through ASTM #40 to the Mix B in the blender and continue to blend at 20 RPM for 2 minutes. Unload the final blend into a double LDPE polybag lined with a black polybag on the outermost side. Displace the air inside each bag and tie each one with a nylon tag. Keep 5 dessicant pillow pouches in the second outer bag before tying it up with a nylon tie. Finally, put the double polybag with the Mix into a black polybag. Label the final bag as Lubricated Blend ready for compression. Use a 9 mm Flat faced, oval shaped compression tooling to compress the final Lubricated blend into flat tablets of appropriate hardness so that their percent friability is less than 0.5% w/w and the disintegration time (DT) is not less than 3 minutes. Alternatively, the tablets may have 2 or more score lines to adjust the dosage in multiples of 5 mg/tablet segment.

### Mini Tablets (60D)

Co-sift and blend CANNABIDIOL, Sorbitan Monolaurate, Crospovidone Ultra and Sodium citrate through ASTM #40 mesh twice. Pre-label it as Mix A. Co-sift the starch and Polyvinyl pyrollidone each individually presifted through ASTM #40 to the Mix A and blend in a V cone blender at 20 RPM for 20 minutes. Label it as Mix B. Add the Sodium stearyl fumarate pre-sifted through ASTM #60 to the Mix B in the blender and continue to blend at 20 RPM for 2 minutes. Unload the final blend into a double LDPE polybag lined with a black polybag on the outermost side. Displace the air inside each bag and tie each one with a nylon tag. Keep 5 desiccant pillow pouches in the second outer bag before tying it up with a nylon tie. Finally, put the double polybag with the Mix into a black polybag. Label the final bag as Lubricated Blend ready for compression. Use a 1.5-2.0 mm flat-faced, round-shaped compression tooling to compress the final Lubricated blend into flat mini-tablets of appropriate hardness so that their percent friability is less than 0.5% w/w and the disintegration time (DT) is not less than 3 minutes.

### Orally Disintegrating Tables (60E)

Co-sift and blend CANNABIDIOL, Tween 20 and Crospovidone Ultra through ASTM #40 mesh twice. Pre-label it as Mix A. Co-sift the starch, sodium citrate and Polyvinyl pyrollidone each individually pre-sifted through ASTM #40 to the Mix A and blend in a V cone blender at 20 RPM for 20 minutes. Label it as Mix B. Add the Sodium stearyl fumarate pre-sifted through ASTM #60 to the Mix B in the blender and continue to blend at 20 RPM for 2 minutes. Unload the final blend into a double LDPE polybag lined with a black polybag on the outermost side. Displace the air inside each bag and tie each one with a nylon tag. Keep 5 desiccant pillow pouches in the second outer bag before tying it up with a nylon tie. Finally, put the double polybag with the Mix into a black polybag. Label the final bag as Lubricated Blend ready for compression. Use a 9 mm Flat faced beveled edge, oval shaped compression tooling to compress the final Lubricated blend into flat tablets of appropriate hardness so that their percent friability is less than 0.5% w/w and the disintegration time (DT) is less than 30 seconds as per the official Compendium—United States Pharmacopeia (USP).

### Immediate Release Tablets and Capsules (60F)

Co-sift and blend CANNABIDIOL, sorbitan monolaurate and Crospovidone Ultra through ASTM #40 mesh twice. Pre-label it as Mix A. Co-sift the starch, sodium citrate and Polyvinyl pyrollidone each individually presifted through ASTM #40 to the Mix A and blend in a V cone blender at 20 RPM for 20 minutes. Label it as Mix B. Add the Sodium stearyl fumarate pre-sifted through ASTM #60 to the Mix B in the blender and continue to blend at 20 RPM for 2 minutes. Unload the final blend into a double LDPE polybag lined with a black polybag on the outermost side. Displace the air inside each bag and tie each one with a nylon tag. Keep 5 desiccant pillow pouches in the second outer bag before tying it up with a nylon tie. Finally, put the double polybag with the Mix into a black polybag. Label the final bag as Lubricated Blend ready for compression. Use appropriate compression tooling to compress the final Lubricated blend into tablets of appropriate hardness so that their percent friability is less than 0.5% w/w and the disintegration time (DT) is not more than 10 minutes.

Alternatively, the Lubricated blend of appropriate quantity can be filled into hard gelatin capsules of appropriate size for oral administration.

### Immediate Release Pellets (60G)

Co-sift and blend CANNABIDIOL, Crospovidone Ultra, starch, PVP through ASTM #40 mesh twice. Pre-label it as Mix A. Load the Mix A into a rapid mixer granulator (RMG). Mix at 30 RPM for 15 min. Granulate it with Iso-propyl alcohol and extrude-spheronise it in an extruder-spheroniser fitted with the appropriate base plate and appropriate feed rate and speed to generate spheres of Minus ASTM #20 and Plus ASTM #40. Dry the spheres in a vacuum tray drier at an inner temperature of 45±2° C. and a vacuum of minus 40 mm of mercury pressure, for approximately 30 min or till an Loss on Drying reading of the crushed pellets of NMT 0.5% w/w is achieved. Their percent friability should be not more than 0.5% w/w and the disintegration time (DT) is not more than 5 minute. Pre-label it as Mix B. Load the Mix B into a V-cone blender. Add the Sodium stearyl fumarate and sodium citrate pre-sifted through ASTM #60 to the Mix B in the blender and continue to blend at 20 RPM for 2 minutes. Unload the final blend into a double LDPE polybag lined with a black polybag on the outermost side. Displace the air inside each bag and tie each one with a nylon tag. Keep 5 desiccant pillow pouches in the second outer bag before tying it up with a nylon tie. Finally, put the double polybag with the Mix into a black polybag. Label the final bag as Lubricated Pellets ready for processing. Use appropriate sachets or capsules to fill the pellets.

### SPRINKLE CAPSULE or SACHET for Immediate Release Pellets (60H)

Alternatively, the Immediate release pellets in a Capsule or a Sachet can be used as a SPRINKLE on soft food for ingestion via the oral route

### Self-Micro-Emulsifying Dispersible Tablets (60I)

Co-sift CANNABIDIOL and HP-Beta CD through ASTM #60 sieve. Add the co-sifted mixture to propylene glycol and sorbitan monolaurate under continues stirring till a mixture is effected. Label this as Mix A. Separately, co-mix PVP K29/32 and sodium citrate and add it to Mix A under stirring for 10 minutes. Label this as Mix B. Load this onto Crospovidone Ultra and MCC 102 mix loaded into a rapid mixer granulator (RMG). Granulate for 20 min at 30 rpm to get a mass of appropriate consistency. Unload and sift the granules through ASTM 40 mesh. Mill the retains using a multi-mill with ASTM #12 and pass the milled material through ASTM #40. Blend these final sized granules in a V—cone blender with Sodium stearyl fumarate pre-sifted through ASTM #60 sieve at 15 rpm for 2 minutes. This is the final “blend ready for compression” or consolidation into compressed tablets. Unload the final blend into a double LDPE polybag lined with a black polybag on the outermost side. Displace the air inside each bag and tie each one with a nylon tag. Keep 5 desiccant pillow pouches in the second outer bag before tying it up with a nylon tie. Finally, put the double polybag with the blend into a black polybag. Label the final bag as “Lubricated Blend ready for Compression”. Use appropriate compression tooling to achieve tablets of hardness such that their percent friability is less than 0.5% w/w and the disintegration time (DT) is less than 3 minutes. Alternatively, the tablets may have 2 or more score lines to adjust the dosage in multiples of 5 mg/tablet segment.

### Example 61

| | Buccal tablets | Buccal tablets |
|---|---|---|
| Ingredients | Percentage (w/w) | |
| CANNABIDIOL | 20 | 20 |
| Carbopol 934 | 20 | 20 |
| HydroxyPropylMethylCellulose | 45 | — |
| (HPMC) K4M | | |
| Mannitol | — | 45 |
| (Directly Compressible) | | |
| Magnesium Stearate | 13 | 13 |
| Talc | 1 | 1 |
| Talc | 1 | 1 |
| Total | 100 | 100 |

### Procedure

Co-sift and blend CANNABIDIOL, Carbopol 934 and HPMC K4M through ASTM #40 mesh twice. Pre-label it as Mix A. Co-sift the Mannitol presifted through ASTM #40 with the Mix A and blend in a V cone blender at 15 RPM for 20 minutes. Label it as Mix B. Add the Magnesium stearate pre-sifted through ASTM #60 to the Mix B in the blender and continue to blend at 15 RPM for 1.5 minutes. Further add the talc pre-sifted through ASTM #60 to the blend in the blender and continue to blend at 15 RPM for 1 minute. Unload the final blend into a double LDPE polybag lined with a black polybag on the outermost side. Displace the air inside each bag and tie each one with a nylon tag. Keep 5 desiccant pillow pouches in the second outer bag before tying it up with a nylon tie. Finally, put the double polybag with the Mix into a black polybag. Label the final bag as Lubricated Blend ready for compression. Use a 6×4 mm flat-faced, modified capsule-shaped compression tooling to compress the final Lubricated blend into flat mini-tablets of appropriate hardness so that their percent friability is less than 0.5% w/w and the disintegration time (DT) is not less than 5 minutes.

### Example 62

| Ingredients for Delayed release tablets | |
|---|---|
| and capsules | Percent (w/w) |
| CORE | |
| CANNABIDIOL | 20 |
| Mannitol | 20 |
| Microcrystalline cellulose (MCC | 17.5 |
| PH 102) | |
| Phosphate trisodium | 10 |
| Cellulose methylhydroxypropyl 15 cps | 10 |
| (HPMC 15 cps) | |
| Cellulose hydroxypropyl 5 cps (HPMC | 5 |
| 5 cps) | |
| Crospovidone ultra | 15 |
| Colloidal silicon dioxide | 2 |
| Magnesium stearate | 0.5 |
| Total | 100 |
| Seal-coating | Percent w/w to the core |
| Cellulose ethyl | 3 |
| Magnesium oxide | 1 |
| WATER OR Iso-propyl alcohol and | QS |
| Dichloromethane | |
| TOTAL | 4 |
| GASTRO-RESISTANT COATING | Percent w/w to the core |
| Eudragit 1100-55 | 15 |
| Triethyl citrate | 5 |
| Ferric oxide color | 1 |
| Talc | 4 |
| Titanium dioxide | 1 |
| WATER | QS |
| Total | 26 |
| Grand total | 130 |

### Procedure

Co-sift through ASTM #40 mesh twice and blend CANNABIDIOL, Mannitol, MCC PH 102, trisodium phosphate, HPMC 5 cps, HPMC 15 cps and Crospovidone in a V-cone blender at 20 RPM for 20 minutes. Label it as Mix A. Add the Colloidal silicon dioxide pre-sifted through ASTM #20 to delump the same to the Mix A in the blender and blend at 15 RPM for 3 minutes. Add the Magnesium stearate pre-sifted through ASTM #40 to it and blend further at 15 rpm for 2 minutes Unload the final blend into a double LDPE polybag lined with a black polybag on the outermost side. Displace the air inside each bag and tie each one with a nylon tag. Keep 5 desiccant pillow pouches in the second outer bag before tying it up with a nylon tie. Finally, put the double polybag with the Mix into a black polybag. Label the final bag as Lubricated Blend ready for compression. Use appropriate compression tooling preferably standard concave, to compress the final Lubricated blend into tablets of appropriate hardness so that their percent friability is less than 0.5% w/w and the disintegration time (DT) is more than 10 minutes. Coat the tablet cores with the Seal Coating pharmaceutical composition using an appropriate solvent system viz. aqueous, non-aqueous; preferably non-aqueous (Iso-propyl alcohol and Dichloromethane) to a 4-5% weight gain on the tablet cores. Label these as Seal Coated Tablets. Further coat the Seal Coated Tablets with the Gastro-resistant Coating Pharmaceutical composition to a total weight gain of 26-30% of the tablet cores. Label these as the Delayed Release Tablets.

### Example 63

| Ingredients | |
|---|---|
| Extended-release tablets and capsules | Percent (w/w) |
| CORE | |
| CANNABIDIOL | 20 |
| Microcrystalline cellulose | 20 |
| CELLULOSE METHYLHYDROXYPROPYL | 20 |
| K100M | |
| CELLULOSE METHYLHYDROXYPROPYL | 12.5 |
| K15M | |
| COLLOIDAL SILICON DIOXIDE | 2 |
| POLYVINYL PYROLLIDONE (PVP K29/32) | 2 |
| MAGNESIUM STEARATE | 0.5 |
| TOTAL | 75 |
| FILM-COATING | Percent w/w |
| Polyvinyl alcohol | 38-46 |
| polyethylene glycol (or glycerin) | 13-25 |
| talc | 09-20 |
| pigment/opacifier | 20-30 |
| lecithin | 0-4 |
| TOTAL | 100 |
| GRAND TOTAL | 175 |

### Procedure

Co-sift and blend CANNABIDIOL, Microcrystalline cellulose, CELLULOSE METHYLHYDROXYPROPYL K100M and CELLULOSEMETHYLHYDROXYPROPYL K15M through ASTM #40 mesh twice. Pre-label it as Mix A. Co-sift the POLYVINYL PYROLLIDONE (PVP K29/32) presifted through ASTM #40 to the Mix A and blend in a V cone blender at 20 RPM for 20 minutes. Label it as Mix B. Add the Sodium stearyl fumarate pre-sifted through ASTM #60 to the Mix B in the blender and continue to blend at 20 RPM for 2 minutes. Unload the final blend into a double LDPE polybag lined with a black polybag on the outermost side. Displace the air inside each bag and tie each one with a nylon tag. Keep 5 desiccant pillow pouches in the second outer bag before tying it up with a nylon tie. Finally, put the double polybag with the Mix into a black polybag. Label the final bag as Lubricated Blend ready for compression. Use appropriate compression tooling preferably standard concave, to compress the final Lubricated blend into tablets of appropriate hardness so that their percent friability is less than 0.5% w/w. Film coat the tablet cores to a weight gain of 2.5 to 3.0% w/w to the tablet core. The percent CANNABIDIOL release at initial 2 hrs is NMT 20%; at 4 hrs is 20-40%, at 8 hrs is 40-80% and at 12 hrs is not less than (NLT) 75%.

Alternatively, the Lubricated blend of appropriate quantity can be filled into hard gelatin capsules of appropriate size for oral administration.

### Example 64

| Ingredients for Effervescent tablets and | |
|---|---|
| effervescent pop sprinkle | Percent (w/w) |
| CANNABIDIOL | 20 |
| Potassium citrate | 27 |
| Citric acid | 8.5 |
| Sodium bicarbonate | 7.5 |
| Mannitol | 5 |
| Aspartame | 2 |
| Strawberry (flavour - encapsulated solid) | 5 |
| Sodium benzoate | 5 |
| PEG 6000 | 20 |
| TOTAL | 100 |

### Procedure

Co-sift the citric acid, sodium bicarbonate, potassium citrate and mannitol through ASTM #40 and mix at 20 RPM for about 15 minutes in a tumbling V-cone blender. Load the obtained mixture into a rapid mixer granulator (RMG) that is hot water jacketed. Circulate hot water at 65-70° C. through its jacket to obtain an inner temperature of 55±2° C. Granulate the powder mix within the RMG until the water of crystallization of citric acid is released and acts as a binder (approximately 30 minutes). Size the wet mass obtained through a multi-mill attached to the RMG and having ASTM sieve #20. Pass the entire mass of granular mass through sieve No. 20. Load this Mix into a V-Cone blender and mix at 20 RPM for 15 min. Label this mix as Mix A. Take about 10% w/w of Mix A and size it using a multi-mill fitted with an ASTM #40 mesh. Pass the fines obtained through an ASTM #60 mesh and collect separately. Co-sift the fines, CANNABIDIOL, Aspartame, Strawberry flavour, Sodium benzoate and PEG 6000 through ASTM #40 and add to Mix A. Blend it at 15 RPM for 10 min. Unload the final blend into a double LDPE polybag lined with a black polybag on the outermost side. Displace the air inside each bag and tie each one with a nylon tag. Keep 5 desiccant pillow pouches in the second outer bag before tying it up with a nylon tie. Finally, put the double polybag with the blend into a black polybag. Label the final bag as “Lubricated Blend ready for Compression”. Use appropriate compression tooling to achieve tablets of hardness such that their percent friability is less than 0.5% w/w and the disintegration time (DT) is less than 3 minutes. Alternatively, the tablets may have 2 or more score lines to adjust the dosage in multiples of 5 mg/tablet segment. Note: All activity to be carried out in an ambient of 25±2° C. and a % RH of NMT 40±5° C.

### Example 65

| Ingredients for OROS Tablets | |
|---|---|
| CORE | Percent (w/w) |
| CANNABIDIOL | 20 |
| Sorbitan Monolaurate | 10 |
| Sodium chloride | 20 |
| Microcrystalline cellulose | 15 |
| CELLULOSE | 20 |
| METHYLHYDROXYPROPYL K100M | |
| CELLULOSE | 12.5 |
| METHYLHYDROXYPROPYL K15M | |
| COLLOIDAL SILICON DIOXIDE | 2 |
| MAGNESIUM STEARATE | 0.5 |
| TOTAL | 100 |
| FILM-COATING | Percent w/w |
| Polyvinyl alcohol | 38-46 |
| polyethylene glycol (or glycerin) | 13-25 |
| talc | 09-20 |
| pigment/opacifier | 20-30 |
| lecithin | 0-4 |
| Iso-propyl alcohol | qs |
| TOTAL | 100 |
| FUNCTIONAL-COATING | Percent w/w |
| Cellulose Acetate | 20 |
| polyethylene glycol (or glycerin) | 5 |
| talc | 10 |
| pigment/opacifier | 2 |
| lecithin | 1 |
| Iso-propyl alcohol | qs |
| TOTAL | 100 |

### Procedure

Co-sift and blend CANNABIDIOL, Sorbitan Monolaurate and Sodium chloride through ASTM #40 mesh twice. Pre-label it as Mix A. Co-sift the MCC PH 102, CELLULOSE METHYLHYDROXYPROPYL K100M and CELLULOSE METHYLHYDROXYPROPYL K15M through ASTM #40 to the Mix A and blend in a V cone blender at 20 RPM for 20 minutes. Add the Colloidal silicon dioxide presifted through ASTM #20 and blend further at 10 RPM for 2 minutes. Label it as Mix B. Add the Magnesium stearate pre-sifted through ASTM #60 to the Mix B in the blender and continue to blend at 20 RPM for 2 minutes. Unload the final blend into a double LDPE polybag lined with a black polybag on the outermost side. Displace the air inside each bag and tie each one with a nylon tag. Keep 5 desiccant pillow pouches in the second outer bag before tying it up with a nylon tie. Finally, put the double polybag with the Mix into a black polybag. Label the final bag as Lubricated Blend ready for compression. Use appropriate compression tooling preferably standard concave, to compress the final Lubricated blend into tablets of appropriate hardness so that their percent friability is less than 0.5% w/w. Film coat the tablet cores to a weight gain of 2.5 to 3.0% w/w to the tablet core using a non-aqueous medium. Functional Coat the tablet with Cellulose acetate non-aqueous dispersion in Iso-propyl alcohol to a weight gain of 25-30% w/w of the tablet core. Laser drill the tablets with an orifice of 150-250 micron. Label these tablets as osmotic-controlled release oral delivery system (OROS) Tablets—OROS TABLETS.

The percent CANNABIDIOL release at initial 2 hrs is NMT 20%; at 4 hrs is 20-40%, at 8 hrs is 40-80% and at 12 hrs is not less than (NLT) 75%.

### Example 66

| Ingredients for Pastilles | Percent (w/w) |
|---|---|
| CANNABIDIOL | 20 |
| Gelatin | 50 |
| Glycerin | 17.8 |
| Simethicone (anti-foam) | 5 |
| Flavour | 5 |
| Sodium citrate | 2 |
| Colorant (water-soluble) | 0.2 |
| Water | QS |
| Ethanol | QS |
| Total | 100 |

### Procedure

Soak the Gelatin in about 90% w/w of its weight in water for about 30 minutes. Ensure that all of the dry gelatin granules are thoroughly wetted or soaked up and there are no dry lumps within the soak. Add the glycerin, simethicone, sodium citrate and the colorant to the soaked gelatin under stirring. Load the above mix into a steam kettle pre-heated to a temperature of 100±5° C. so that the temperature of the mix is between 90±5° C. Keep the mix under constant stirring for 30 minutes. Turn off the pre-heating. Continue stirring. Add the CANNABIDIOL dispersed in small amount of Ethanol under continuous stirring and into pastill moulds. Allow the mix to cool to room temperature at which they solidify into Pastills. Remove the pastilles from the respective moulds and store in air-tight, light-resistant containers till packaged in appropriate packing.

### Example 67

| Ingredients for Oral powder or Electuary | Percent (w/w) |
|---|---|
| CANNABIDIOL | 20 |
| Sorbitan monolaurate | 20 |
| Polyvinyl pyrrolidone K29/32 | 15 |
| Acesulfame potassium | 5 |
| Colloidal silicon dioxide | 10 |
| Mannitol | 28 |
| Sodium Stearyl Fumarate | 1 |
| Vanilla (powder flavour) | 1 |
| TOTAL | 100 |

### Procedure

Co-sift CANNABIDIOL, Sorbitan monolaurate, Polyvinyl pyrrolidone K29/32, Acesulfame potassium, Colloidal silicon dioxide and Mannitol through ASTM #40 mesh twice. Blend the mix in a V-cone blender at 15 RPM for 10 minutes. Pre-label it as Mix A. Sift the vanilla flavour through ASTM #40. Add to mix A and blend at 10 RPM for 5 minutes. Sift the Sodium stearyl fumarate through ASTM #40. Add to the mix in the blender and blend at 10 RPM for a further 2 minutes. Unload the final blend into a double LDPE polybag lined with a black polybag on the outermost side. Displace the air inside each bag and tie each one with a nylon tag. Keep 5 desiccant pillow pouches in the second outer bag before tying it up with a nylon tie. Finally, put the double polybag with the Mix into a black polybag. Label the final bag as Lubricated Blend ready for filling into sachets. This powder in a sachet can also be taken as an Electuary with Honey or a flavoured syrup base or in MILK POWDER as desired as per appropriate prescribed or needed dose.

### Example 68

| Ingredients for Oral Jelly | Percent (w/w) |
|---|---|
| CANNABIDIOL | 20 |
| Xanthan gum | 2.5 |
| Sodium citrate | 1 |
| Simethicone (anti-foam) | 4.8 |
| Flavour | 2 |
| Ethanol | 5 |
| Color (water-soluble) | 0.2 |
| Methyl paraben | 0.18 |
| Propyl paraben | 0.02 |
| Sucrose | 44.3 |
| Water | 20 |
| Total | 100 |

### Procedure

Soak the Xanthan gum in about half of its weight in water having simethicone and sodium citrate for about 60 minutes. Label as Mix A. Pre-heat the remaining water to near boiling and dissolve the parabens in it under continuous stirring. Stop the heating, add sucrose and continue stirring. Label as mix B. Mix A and B under stirring. Label it as Mix C. At 35-40° C., add the flavour, colour and CANNABIDIOL dissolved in the ethanol to Mix C and continue to stir. Add the remaining water and continue stirring till the mix attains room temperature.

Store in air-tight, light-resistant containers till packaged in appropriate packing.

### Example 69

| Ingredients for Compressed lozenges or Chews or | |
|---|---|
| Lollipop | Percent (w/w) |
| CANNABIDIOL | 20 |
| Polyoxyl 35 Castor Oil (Chremophore EL/Kolliphor EL) | 10 |
| Dextrate (Emdex) | 25.25 |
| Polyethylene glycol (PEG) 6000 | 10 |
| Microcrystalline cellulose MCC PH 102 | 24.5 |
| Polyvinyl pyrollidone (PVP K29/32) | 10 |
| Color - FD&C Yellow No. 6 | 0.25 |
| Magnesium stearate | |
| Total | 100 |

### Procedure

Co-sift the CANNABIDIOL with the Polyoxyl 35 Castor oil, Dextrate, PEG 6000 (pre-sifted through ASTM #40 sieve), MCC 102, PVP K29/32 and FD&C Yellow No. 6. Blend the co-sifted mix in a V-Cone blender at 20 RPM for 15 minutes.

Add the Magnesium stearate pre-sifted through ASTM #40 to the above mix and blend at 15 RPM for 3 minutes. Unload the final blend into a double LDPE polybag lined with a black polybag on the outermost side. Displace the air inside each bag and tie each one with a nylon tag. Keep 5 desiccant pillow pouches in the second outer bag before tying it up with a nylon tie. Finally, put the double polybag with the blend into a black polybag. Label the final bag as “Lubricated Blend ready for Compression”. Use appropriate compression toolings to achieve lozenges of hardness such that their percent friability is less than 0.5% w/w and the disintegration time (DT) is less than 15 minutes. Alternatively, the compressed lozenges may have 2 or more score lines to adjust the dosage in multiples of 5 mg/segment. Note: All activity to be carried out in an ambient of 25±2° C. and a % RH of NMT 40±5° C.

Alternatively, the segments can be used as CHEWS or as LOLLIPOP with the segment having a plastic radio-opaque holder inserted into each.

### Example 70

| Ingredients for Dragees | Percent (w/w) |
|---|---|
| CORE | |
| CANNABIDIOL | 20 |
| Sorbitan Monolaurate | 2 |
| Crospovidone Ultra | 5 |
| Lactose monohydrate (DC grade) | 20 |
| Microcrystalline cellulose MCC PH 102 | 15 |
| Glyceryl behenate | 38 |
| TOTAL | 100 |
| COATING | |
| Gum arabic | 2.5 |
| Sachharose | 5 |
| liquid glucose | 10 |
| sodium bicarbonate | 5 |
| sodium methyl para-hydroxybenzoate | 0.18 |
| sodium propyl para-hydroxybenzoate | 0.02 |
| Talc | 3 |
| Color | 1.8 |
| Water qs. To make 100. (Not present in final product) | 72.5 |
| TOTAL | 100 |

### Procedure

Co-sift CANNABIDIOL, Sorbitan Monolaurate and Crospovidone Ultra twice through ASTM #40 sieve. Label it as Mix A. separately, co-sift Lactose monohydrate (DC grade) and Microcrystalline cellulose MCC PH 102 through ASTM #20 sieve. Label it as Mix B. Sift Glyceryl behenate through ASTM #20 sieve twice, to delump any agglomerates. Label it as Ingredient C. Co-sift Mix A, Mix B and Ingredient C through ASTM #40 sieve. Label it as Mix D. Load and blend Mix D in a suitable V-cone blender at 15 RPM for 10 minutes. Unload the final blend into a double LDPE polybag lined with a black polybag on the outermost side. Displace the air inside each bag and tie each one with a nylon tag. Keep 5-6 desiccant pillow pouches in the second outer bag before tying it up with a nylon tie. Finally, put the double polybag with the blend into a black polybag. Label the final bag as “Lubricated Blend ready for Compression”. Use appropriate compression tooling to achieve bi-convex tablets of appropriate hardness such that their percent friability is less than 0.5% w/w. Using the Coating dispersion, coat the compressed tablets to a weight gain of 20% w/w on the tablet cores to yield the final product—DRAGEES.

### Example 71

| | |
|---|---|
| Oral solution or | Oral |
| sublingual drops | solution |

| | |
|---|---|
| Ingredients | Percent (w/w) |

| | | |
|---|---|---|
| CANNABIDIOL | 20 | 20 |
| Polyoxyl 35 Castor Oil | 10 | 10 |
| (Chremophore EL/Kolliphor EL) | | |
| Saccharin Sodium | 2 | 2 |
| Caramel | 1 | 1 |
| FD&C Yellow No. 6 | 0.05 | 0.05 |
| Peppermint oil | 0.05 | 0.05 |
| Alcohol - Ethanol | 10 | 10 |
| Sucrose | — | 20 |
| Water QS to make 100 | 56.9 | 36.9 |
| TOTAL | 100 | 100 |

### Procedure

### Oral Solution or Sublingual Drops

In a closed vessel, dissolve the CANNABIDIOL, Polyoxyl 35 Castor Oil and peppermint oil in Ethanol under continuous stirring. Label as Mix A. In the water taken in a separate closed tank (Double capacity than that used for Mix A), dissolve the saccahrin sodium, caramel and color. Label as Mix B. Blend the Mix A with Mix B in the latter closed tank under continuous stirring. This is the Oral solution of CANNABIDIOL to be stored in a well-filled, tightly closed container in dark or subdued light. This Solution concentrate can further be diluted with similar water and dispensed into dark amber colored glass bottles or appropriate 100% opaque containers viz. Opaque to light.

Alternatively, this solution can also be administered via the sublingual route as SUBLINGUAL DROPS in appropriate containers.

### Oral Syrup

In a closed vessel, dissolve the CANNABIDIOL, Polyoxyl 35 Castor Oil and peppermint oil in Ethanol under continuous stirring. Label it as Mix A. In the water taken in a separate closed tank (Double capacity than that used for Mix A), dissolve the sucrose, saccahrin sodium, caramel and color. Label it as Mix B. Blend the Mix A with Mix B under constant stirring in the latter closed tank. This is the Oral Syrup of CANNABIDIOL to be stored in a well-filled, tightly closed container in dark or subdued light. This Syrup can further be diluted with similar water and dispensed into dark amber colored glass bottles or appropriate 100% opaque containers viz. Opaque to light.

### Example 72

| Ingredients for chewing gum | mg/unit |
|---|---|
| CANNABIDIOL | 20 |
| Gum base | 1000 |
| Flavour - Cherry /Strawberry/Mint | 10 |
| Mono- Ammonium Glycerrhizinate (MAG) | 30 |
| Aspartame | 20 |
| Soyalecithin | 20 |
| Hydrogenated Castor Oil | 12 |
| Talc | 10 |
| Total | 1122 |

### Procedure

Co-sift the flavour and the CANNABIDIOL through ASTM #40 in a V-Cone blender for 30 min. Label it as Mix A. Co-sift the gum base, MAG, aspartame, soyalecithin and hydrogenated Castor oil through ASTM #40 sieve. Label it as Mix B. Add the Mix B to Mix A in the V-cone blender and blend at 15 RPM for 10 minutes.

Add the Hydrogenated Castor Oil pre-sifted through ASTM #40 to the above mix in the blender and blend at 15 RPM for 10 minutes. Label it as: Lubricated blend—ready for compression “The above blend can be consolidated into a chewing gum tablet using appropriate tooling's on a tablet compression machine. The chewing gum tablets obtained can be additionally be coated with a flavoured immediate release coating.

### Example 73

Soft gel capsules - immediate release

| Ingredient | mg/Capsule |
|---|---|
| CANNABIDIOL | 100 |
| Polyvinyl pyrollidone K29/32 | 50-150 |
| Ethanol + water (90:10) | 100 |
| Propylene glycol | 200-250 |
| Polyethylene glycol (PEG) 400 | 200-350 |
| Butylated Hydroxy Toluene (BHT) | 0.1 |
| TOTAL | 600 |

### Procedure

In a well closed container, mix and continuously stir the propylene glycol and PEG-400 to dissolve well. Add the Polyvinyl pyrrolidone K29/32 to the above mix under stirring to dissolve it. Add the CANNABIDIOL and the Ethanol-water to it under continuous stirring. Add the BHT to the mix and stir till a clear solution is obtained. Sonicate the mixture in the closed vessel to remove any air entrapments and store the mix in a well-filled, well-closed opaque container. Fill the said final mix into soft gelatin capsules. Use opaque and colored soft gelatin. Store these soft-gelatin capsules into dark amber colored glass or appropriate opaque containers.

### Example 74

Soft gel capsules - extended release

| Ingredient | mg/Capsule |
|---|---|
| CANNABIDIOL | 100 |
| Polyvinyl pyrollidone K29/32 | 50-150 |
| Ethanol + water (90:10) | 100 |
| Propylene glycol | 200-250 |
| Polyethylene glycol (PEG) 6000 | 200-350 |
| Butylated Hydroxy Toluene (BHT) | 0.1 |
| TOTAL | 600 |

### Procedure

In a well closed hot water circulation jacketed container, warmed to 70° C. mix and continuously stir the propylene glycol and PEG-6000 to dissolve well. Add the Polyvinyl pyrrolidone K29/32 to the above mix under stirring to dissolve it. Stop the warming and keep under continuous stirring till the mix attains room temperature. Label as Mix A. In another similar separate vessel add the CANNABIDIOL to the Ethanol-water. Continue stirring. Add the BHT to the mix and stir till a clear solution is obtained. Label as Mix B. Add Mix A to Mix B under stirring. Sonicate the mixture in the closed vessel to remove any air entrapments and store the mix in a well-filled, well-closed opaque container. Fill the said final mix into soft gelatin capsules. Use opaque and colored soft gelatin. Store these soft-gelatin capsules into dark amber colored glass or appropriate opaque containers. Label these capsules as Extended release Soft-gel Capsules.

The percent CANNABIDIOL release at initial 2 hrs. is NMT 40%; at 4 hrs. is 40-60%, at 8 hrs is not less than (NLT) 75%.

### Example 75

Quick dissolving film - Oral and or Sublingual

| Ingredient | Percent (w/w)/film strip |
|---|---|
| CANNABIDIOL | 20 |
| Polysorbate 80 | 0.2 |
| Pullulan | 8 |
| Sorbitol | 0.5 |
| Sucralose | 1 |
| Monoammonium glycerrhizinate | 0.1 |
| Peppermint powder | 0.8 |
| Ethanol:Water (80:20 v/v) qs to make 100 | qs |

### Procedure

In a well closed container, mix and continuously stir the Pullulan and sorbitol in the Ethanol:water to dissolve well. Add the CANNABIDIOL dispersed in the Polysorbate 80 to the above mix under stirring to dissolve it. Add the sucralose, Monoammonium glycyrrhizinate and peppermint powder mix and stir till a clear solution is obtained. Sonicate the mixture in the closed vessel to remove any air entrapments and store the mix in a well-filled, well-closed opaque container. Use the above solution on a film forming machine to lay out films that are dried at a temperature not exceeding 40° C. Cut the dried films into rectangular films of appropriate size to meet the dose required. Store these oral quick dissolving films into dark amber colored special food grade containers or in Alu-Alu pouches.

The film can be administered via the oral route on the tongue or sublingually.

### Example 76

Oro-Buccal muco-adhesive film

| Ingredient | mg/film strip |
|---|---|
| CANNABIDIOL | 20 |
| Sodium benzoate | 0.23 |
| Parahydroxybenzoate methyl | 0.24 |
| Parahydroxybenzoate propyl | 0.06 |
| Colorant - Ferric oxide red | 0.01 |
| Polyoxyl 35 Castor Oil (Chremophore EL/ | 10 |
| Kolliphor EL) | |
| Sodium citrate | 0.5 |
| Hydroxypropyl cellulose (HPC) | 5 |
| Hydroxyethyl cellulose (HEC) | 2 |
| Sodium carboxymethyl cellulose (Na CMC) | 2 |
| Sodium saccharine | 0.19 |
| Ethanol:Water = 80:10 v/v | |
| Total | 40.23 |

### Procedure

In a well closed container, mix and continuously stir the HPC, HEC and Na-CMC in the Ethanol:water to dissolve well. Add the CANNABIDIOL dissolved in the Polyoxyl 35 Castor Oil to the above mix under stirring to dissolve it. Add all the remaining ingredients and stir till a homogeneous solution is obtained. Sonicate the mixture in the closed vessel to remove any air entrapments and store the mix in a well-filled, well-closed opaque container. Use the above solution on a film forming machine to lay out films that are dried at a temperature not exceeding 40° C. Cut the dried films into rectangular films of appropriate size to meet the dose required.

Store these oral quick dissolving films into dark amber colored special food grade containers or in Alu-Alu pouches.

The film can be administered via the oral route as a buccal muco-adhesive film.

### Example 77

| Ingredients for oral emulsion | Percent (w/w) |
|---|---|
| CANNABIDIOL | 20 |
| Polyoxyl 35 Castor Oil (Chremophore EL/Kolliphor EL) | 10 |
| Saccharin Sodium | 2 |
| Caramel | 1 |
| FD&C Yellow No. 6 | 0.05 |
| Peppermint oil | 0.05 |
| Corn oil | 10 |
| Sucrose | 20 |
| Water* QS to make 100 | 36.9 |
| TOTAL | 100 |

*water = Freshly boiled and cooled in a closed container.

### Procedure

In a closed vessel, dissolve the CANNABIDIOL, Polyoxyl 35 Castor Oil and peppermint oil in Corn oil under continuous stirring. Label it as Mix A. In the water taken in a separate closed tank (Double capacity than that used for Mix A), dissolve the sucrose, saccahrin sodium, caramel and color. Label it as Mix B. Mix the Mix A with Mix B, both pre-heated to 70±2° C. for 5 misusing a high shear homogeniser in the latter closed tank. This is the Oral Emulsion of CANNABIDIOL to be stored in a well-filled, tightly closed container in dark or subdued light. This Syrup can further be diluted with similar water and dispensed into dark amber colored glass bottles or appropriate 100% opaque containers viz. Opaque to light.

### Example 78

| Ingredients for Inhalation spray or oral spray | mg per Millilitre |
|---|---|
| CANNABIDIOL | 20 |
| Polysorbate 20 | 20 |
| Anhydrous trisodium citrate | 2.5 |
| Sodium chloride | 18 |
| Water* QS to make 1 ml | |

*water = Freshly boiled and cooled in a closed container.

### Procedure

In a well-closed vessel, dissolve the CANNABIDIOL and polysorbate 20 under continuous stirring. Label it as solution A. Separately dissolve the anhydrous trisodium citrate and sodium chloride in the water under continuous stirring. Label it as solution B. Add solution B to solution A under continuous stirring. This is the Spray to be stored in a well-filled, tightly closed container. It can be administered as a Nasal Inhalation or an Oral Spray in appropriate containers suitable for administration.

### Example 79

INHALATION CAPSULES

| Ingredients | mg/Capsule |
|---|---|
| CANNABIDIOL | 0.2-2 mg |
| Magnesium stearate (MgSt) from Peter Greven | 88 mg |
| (Germany) [Inhalation 171 grade] | |

Hydroxypropyl methylcellulose (HPMC) capsules (QualiCaps, Spain) Size #3

### Procedure

Batches with a total amount of 40 g of powder (excipient and drug) are targeted. Both excipients individually with the CANNABIDIOL were subjected to:

(1) Blend under high stress viz. high shear blending.

Blend the CANNABIDIOL and Excipient viz. Lactose or Magnesium stearate, in a high shear blender—the Collette MicroGral 2 L (GEA Pharma Systems, Switzerland) for 10 minutes at 1200 rpm at a room temperature of 10-15±2° C., % Relative Humidity of ambient=NMT 20% and subdued light.

(2) Blend under low stress viz. low shear blending. Blend the CANNABIDIOL and Excipient viz. Lactose or Magnesium stearate, in a low shear blender—the Turbula® T2F mixer 2 L (Willy A. Bachofen AG, Switzerland) for 10 min at 25 rpm at a room temperature of 10-15±2° C., % Relative Humidity of ambient=NMT 20% and subdued light. The final powder in each case was filled manually into size #3 Hydroxypropyl methylcellulose (HPMC) capsules (QualiCaps, Spain) containing a strength of 200 mcg to 2 mg of CANNABIDIOL per capsule. The capsules selected were colored and opaque. Store the final filled capsules in a brown opaque HDPE container and with a desiccant pouch (1 gm) and fitted with a CRC cap. The same was stored at a temperature of NMT 25±2° C.

### Example 80

| Ingredients for vaginal gel | mg/2 gm |
|---|---|
| CANNABIDIOL | 20 |
| Polyoxyl 35 Castor Oil (Chremophore EL/Kolliphor EL) | 10 |
| Ascorbic acid | 5 |
| Glycerin or Propylene glycol | 10 |
| Hydroxypropyl Methylcellulose (HPMC E50) | 0.3 |
| Trisodium Citrate dihydrate | 0.0036 |
| Water QS to make 100 | 1965.7 |
| TOTAL | 2000 |

### Procedure

In a closed vessel, dissolve the CANNABIDIOL in Polyoxyl 35 Castor Oil under continuous stirring. Add glycerin or Propylene glycol and continue stirring. Label it as Mix A. In the water taken in a separate closed tank, dissolve the HPMC E50, Ascorbic acid and Trisodium Citrate dihydrate. Label it as Mix B. Add Mix B to Mix A for 15 min under continuous but gentle stirring to avoid any entrapment of air bubbles. This is the Vaginal Gel of CANNABIDIOL to be stored in a well-filled, tightly closed container. It can be filled into appropriate gel dispensing containers and or unitary sachets

### Example 81

| Ingredients for eye drops | mg per Milliliter |
|---|---|
| CANNABIDIOL | 20 |
| Polysorbate 20 | 20 |
| Benzalkonium chloride | 0.1 |
| disodium EDTATE | 18 |
| Sodium Carboxymethyl Cellulose (Na | 5 |
| CMC) | |
| Citric acid monohydrate | 0.5 |
| Sodium hydroxide* | QS |
| Hydrochloric acid* | QS |
| Water** QS to make 1 ml | |

*water = Freshly boiled and cooled in a closed container.

### Procedure

In a well-closed vessel, dissolve the CANNABIDIOL and polysorbate 20 under continuous stirring. Label it as solution A. Separately dissolve the Sodium Carboxymethyl Cellulose, Citric acid monohydrate, Disodium EDTATE and Benzalkonium chloride in the water under continuous stirring. Label it as solution B. Add solution B to solution A under continuous stirring. Adjust pH to 7-7.2 using sodium hydroxide or Hydrochloric acid. The above solution is to be stored in a well-filled, tightly closed container in dark or in dark amber colored glass containers away from light. It can be administered as eye drops in appropriate containers suitable for administration.

### Example 82

SUPPOSITORIES

| Ingredients | mg/suppository |
|---|---|
| CANNABIDIOL | 20 |
| Hard fat | 1920 |
| Ethanol* | 50 |
| TOTAL | 1940 |

*Evaporates on processing and is not present in the final product

### Procedure

In a closed vessel, disperse the CANNABIDIOL solubilised in Ethanol into the molten hard fat maintained at 80° C. under continues stirring for 30 min. Stop the heating and continue stirring. Pour the molten mass into suppository moulds of appropriate size and shape. The suppositories obtained are to be packed into white opaque blister packs.

All publications cited in this specification are incorporated as references.

### REFERENCES

1. Grisanti L A (2018). Diabetes and Arrhythmias: Pathophysiology, Mechanisms and Therapeutic Outcomes. Frontiers in physiology 9: 1669.
2. Napolitano C, Bloise R, & Priori S G (2006). Long QT syndrome and short QT syndrome: how to make correct diagnosis and what about eligibility for sports activity. Journal of cardiovascular medicine (Hagerstown, Md.) 7: 250-256.
3. Shimizu W, & Antzelevitch C (1999). Cellular basis for long QT, transmural dispersion of repolarization, and torsade de pointes in the long QT syndrome. Journal of electrocardiology 32 Suppl: 177-184.
4. Yu P, Hu L, Xie J, Chen S, Huang L, Xu Z, et al. (2018). O-GlcNAcylation of cardiac Nav1.5 contributes to the development of arrhythmias in diabetic hearts. International journal of cardiology 260: 74-81.
5. DeMarco K R and Clancy C E (2016). Cardiac Na Channels: Structure to Function. Current Topics in Membranes; 78: 287-311.
6. Yellen G (1998). The moving parts of voltage-gated ion channels. Quarterly Reviews of Biophysics. 31(3):239-295.
7. Kleber A G, Rudy Y (2004). Basic mechanisms of cardiac impulse propagation and associated arrhythmias. Physiological Reviews. 84(2):431-488.
8. Chen-Izu Y, et al (2015). Na+ channel function, regulation, structure, trafficking and sequestration. Journal of Physiology. 593(6):1347-1360.
9. Herren A W, Bers D M, Grandi E (2013). Post-translational modifications of the cardiac Na channel: contribution of CaMKII-dependent phosphorylation to acquired arrhythmias. American Journal of Physiology. Heart and Circulatory Physiology. 305(4):H431-H445.
10. Rook M B, et al (2012). Biology of cardiac sodium channel Nav1.5 expression. Cardiovascular Research. 93(1):12-23.
11. Balser J R (1999). Structure and function of the cardiac sodium channels. Cardiovascular research 42: 327-338.
12. Ruan Y, Liu N, & Priori S G (2009). Sodium channel mutations and arrhythmias. Nature reviews Cardiology 6: 337-348.
13. Thomas B F, Gilliam A F, Burch D F, Roche M J, & Seltzman H H (1998). Comparative receptor binding analyses of cannabinoid agonists and antagonists. The Journal of pharmacology and experimental therapeutics 285: 285-292.
14. Ghovanloo M R, Shuart N G, Mezeyova J, Dean R A, Ruben P C, & Goodchild S J (2018). Inhibitory effects of Cannabidiol on voltage-dependent sodium currents. The Journal of biological chemistry 293: 16546-16558.
15. Cunha J M, Carlini E A, Pereira A E, Ramos O L, Pimentel C, Gagliardi R, et al. (1980). Chronic administration of Cannabidiol to healthy volunteers and epileptic patients. Pharmacology 21: 175-185.
16. Izzo A A, Borrelli F, Capasso R, Di Marzo V, & Mechoulam R (2009). Non-psychotropic plant cannabinoids: new therapeutic opportunities from an ancient herb. Trends in pharmacological sciences 30: 515-527.
17. Rajesh M, Mukhopadhyay P, Batkai S, Patel V, Saito K, Matsumoto S, et al. (2010). Cannabidiol attenuates cardiac dysfunction, oxidative stress, fibrosis, and inflammatory and cell death signalling pathways in diabetic cardiomyopathy. Journal of the American College of Cardiology 56: 2115-2125.
18. Christopher A. Ahern (2013). What activates inactivation? Journal of General Physiology 142(2): 97-100.
19. Wang, Q., J. Shen, I. Splawski, D. Atkinson, Z. Li, J. L. Robinson, A. J. Moss, J. A. Towbin, and M. T. Keating (1995). SCNSA mutations associated with an inherited cardiac arrhythmia, long QT syndrome. Cell. 80:805-811.
20. Valdivia, C. R., W. W. Chu, J. Pu, J. D. Foell, R. A. Haworth, M. R. Wolff, T. J. Kamp, and J. C. Makielski (2005). Increased late sodium current in myocytes from a canine heart failure model and from failing human heart. Journal of Molecular and Cellular Cardiology 38:475-483.
21. Yang, Y., Y. Wang, S. Li, Z. Xu, H. Li, L. Ma, J. Fan, D. Bu, B. Liu, Z. Fan, et al. (2004). Mutations in SCN9A, encoding a sodium channel alpha subunit, in patients with primary erythermalgia. Journal of Medical Genetics. 41:171-174.
22. Fertleman, C. R., M. D. Baker, K. A. Parker, S. Moffatt, F. V. Elmslie, B. Abrahamsen, J. Ostman, N. Klugbauer, J. N. Wood, R. M. Gardiner, and M. Rees. (2006). SCN9A mutations in paroxysmal extreme pain disorder: allelic variants underlie distinct channel defects and phenotypes. Neuron. 52:767-774.
23. Ptacek, L. J., A. L. George Jr., R. C. Griggs, R. Tawil, R. G. Kallen, R. L. Barchi, M. Robertson, and M. F. Leppert. (1991). Identification of a mutation in the gene causing hyperkalemic periodic paralysis. Cell. 67:1021-1027.
24. Rojas, C. V., J. Z. Wang, L. S. Schwartz, E. P. Hoffman, B. R. Powell, and R. H. Brown Jr. (1991). A Met-to-Val mutation in the skeletal muscle Na+ channel alpha-subunit in hyperkalaemic periodic paralysis. Nature. 354:387-389.
25. McClatchey, A. I., P. Van den Bergh, M. A. Pericak-Vance, W. Raskind, C. Verellen, D. McKenna-Yasek, K. Rao, J. L. Haines, T. Bird, R. H. Brown Jr., et al. (1992). Temperature-sensitive mutations in the III-IV cytoplasmic loop region of the skeletal muscle sodium channel gene in paramyotonia congenita. Cell. 68:769-774.
26. Kahlig, K. M., T. H. Rhodes, M. Pusch, T. Freilinger, J. M. Pereira-Monteiro, M. D. Ferrari, A. M. van den Maagdenberg, M. Dichgans, and A. L. George Jr. (2008). Divergent sodium channel defects in familial hemiplegic migraine. Proceedings of the National Academy of Sciences of the United States of America. 105:9799-9804.
27. Weiss, L. A., A. Escayg, J. A. Kearney, M. Trudeau, B. T. MacDonald, M. Mori, J. Reichert, J. D. Buxbaum, and M. H. Meisler. (2003). Sodium channels SCN1A, SCN2A and SCN3A in familial autism. Molecular Psychiatry. 8:186-194.
28. Han, S., C. Tai, R. E. Westenbroek, F. H. Yu, C. S. Cheah, G. B. Potter, J. L. Rubenstein, T. Scheuer, H. O. de la Iglesia, and W. A. Catterall. (2012a). Autistic-like behaviour in Scn1a+/− mice and rescue by enhanced GABA-mediated neurotransmission. Nature. 489:385-390.
29. Han, S., F. H. Yu, M. D. Schwartz, J. D. Linton, M. M. Bosma, J. B. Hurley, W. A. Catterall, and H. O. de la Iglesia. (2012b). Na(V)1.1 channels are critical for intercellular communication in the suprachiasmatic nucleus and for normal circadian rhythms. Proceedings of the National Academy of Sciences of the United States of America. 109:E368-E377.
30. Craner, M. J., J. Newcombe, J. A. Black, C. Hartle, M. L. Cuzner, and S. G. Waxman. 2004. Molecular changes in neurons in multiple sclerosis: altered axonal expression of Nav1.2 and Nav1.6 sodium channels and Na+/Ca2+ exchanger. Proceedings of the National Academy of Sciences of the United States of America. 101:8168-8173.
Viskupicova J, Blaskovic D, Galiniak S, Soszynski M, Bartosz G, Horakova L, et al. (2015). Effect of high glucose concentrations on human erythrocytes in vitro. Redox biology 5: 381-387.
31. Liu Y, Sun L, Ma Y, Wei B, Gao M, & Shang L (2019). High glucose and bupivacaineinduced cytotoxicity is mediated by enhanced apoptosis and impaired autophagy via the PERKATF4CHOP and IRE1TRAF2 signaling pathways. Molecular medicine reports 20: 2832-2842.
32. Chen M, Zheng H, Wei T, Wang D, Xia H, Zhao L, et al. (2016). High Glucose-Induced PC12 Cell Death by Increasing Glutamate Production and Decreasing Methyl Group Metabolism. 2016: 4125731.
33. Fouda M A, & Abdel-Rahman A A (2017). Endothelin Confers Protection against High Glucose-Induced Neurotoxicity via Alleviation of Oxidative Stress. The Journal of pharmacology and experimental therapeutics 361: 130-139.
34. Fouda M A, El-Sayed S S, & Abdel-Rahman A A (2018). Restoration of Rostral Ventrolateral Medulla Cystathionine-gamma Lyase Activity Underlies Moxonidine-Evoked Neuroprotection and Sympathoinhibition in Diabetic Rats. The Journal of pharmacology and experimental therapeutics 364: 170-178.
35. Liu M, Gu L, Sulkin M S, Liu H, Jeong E M, Greener I, et al. (2013). Mitochondrial dysfunction causing cardiac sodium channel downregulation in cardiomyopathy. Journal of molecular and cellular cardiology 54: 25-34.
36. Nakajima T, Davies S S, Matafonova E, Potet F, Amarnath V, Tallman K A, et al. (2010). Selective gamma-ketoaldehyde scavengers protect Nav1.5 from oxidant-induced inactivation. Journal of molecular and cellular cardiology 48: 352-359.
37. Korystov Y N, Emel'yanov MO, Korystova A F, Levitman M K, & Shaposhnikova V V (2009). Determination of reactive oxygen and nitrogen species in rat aorta using the dichlorofluorescein assay. Free radical research 43: 149-155.
38. Nuss H B, Tomaselli G F, & Marban E (1995). Cardiac sodium channels (hH1) are intrinsically more sensitive to block by lidocaine than are skeletal muscle (mu 1) channels. The Journal of general physiology 106: 1193-1209.
39. Wang Y, Mi J, Lu K, Lu Y, & Wang K (2015). Comparison of Gating Properties and Use-Dependent Block of Nav1.5 and Nav1.7 Channels by Anti-Arrhythmics Mexiletine and Lidocaine. PloS one 10: e0128653.
40. Ghovanloo M R, Abdelsayed M, & Ruben P C (2016). Effects of Amiodarone and N-desethylamiodarone on Cardiac Voltage-Gated Sodium Channels. Frontiers in pharmacology 7: 39.
41. Wang Q, Shen J, Splawski I, Atkinson D, Li Z, Robinson J L, et al. (1995). SCNSA mutations associated with an inherited cardiac arrhythmia, long QT syndrome. Cell 80: 805-811.
42. El-Remessy A B, Abou-Mohamed G, Caldwell R W, & Caldwell R B (2003). High glucose-induced tyrosine nitration in endothelial cells: role of eNOS uncoupling and aldose reductase activation. Investigative ophthalmology & visual science 44: 3135-3143.
43. Sharifi A M, Eslami H, Larijani B, & Davoodi J (2009). Involvement of caspase-8, -9, and -3 in high glucose-induced apoptosis in PC12 cells. Neuroscience letters 459: 47-51.
44. O'Hara T, Virag L, Varro A, & Rudy Y (2011). Simulation of the undiseased human cardiac ventricular action potential: model formulation and experimental validation. PLoS computational biology 7: e1002061.
45. Curtis M J, Alexander S, Cirino G, Docherty J R, George C H, Giembycz M A, et al. (2018). Experimental design and analysis and their reporting II: updated and simplified guidance for authors and peer reviewers. British journal of pharmacology 175: 987-993.
46. Devinsky, O. et al. Trial of Cannabidiol for Drug-Resistant Seizures in the Dravet Syndrome. N. Engl. J. Med. 376, 2011-2020 (2017).
47. Ghovanloo, M.-R. et al. Inhibitory effects of Cannabidiol on voltage-dependent sodium currents. J. Biol. Chem. 293, 16546-16558 (2018).
48. De Petrocellis, L. et al. Effects of cannabinoids and cannabinoid-enriched Cannabis extracts on TRP channels and endocannabinoid metabolic enzymes. Br. J. Pharmacol. 163, 1479-1494 (2011).
49. Patel, R. R., Barbosa, C., Brustovetsky, T., Brustovetsky, N. & Cummins, T. R. Aberrant epilepsy-associated mutant Nav1.6 sodium channel activity can be targeted with Cannabidiol. Brain 139, 2164-2181 (2016).
50. Ghovanloo, M.-R., Aimar, K., Ghadiry-Tavi, R., Yu, A. & Ruben, P. C. Physiology and Pathophysiology of Sodium Channel Inactivation. Curr. Top. Membr. 78, 479-509 (2016).
51. Estacion, M., Gasser, A., Dib-Hajj, S. D. & Waxman, S. G. A sodium channel mutation linked to epilepsy increases ramp and persistent current of Nav1.3 and induces hyperexcitability in hippocampal neurons. Exp. Neurol. 224, 362-368
52. Cannon, S. C. PATHOMECHANISMS IN CHANNELOPATHIES OF SKELETAL MUSCLE AND BRAIN. Annu. Rev. Neurosci. 29, 387-415 (2006).
53. Lee, S., Goodchild, S. J. & Ahern, C. A. Local anesthetic inhibition of a bacterial sodium channel. J. Gen. Physiol. 139, 507-516 (2012).
54. Gamal El-Din, T. M., Lenaeus, M. J., Zheng, N. & Catterall, W. A. Fenestrations control resting-state block of a voltage-gated sodium channel. Proc. Natl. Acad. Sci. 115, 13111-13116 (2018).
55. Pan, X. et al. Structure of the human voltage-gated sodium channel Nav1.4 in complex with (31. Science (80-.). 362, eaau2486 (2018).
56. Lehmann-Horn, F., Jurkat-Rott, K. & Rude′, R. Diagnostics and therapy of muscle channelopathies—Guidelines of the Ulm Muscle Centre. Acta Myologica 27, 98-113 (2008).
57. Ghovanloo, M.-R., Abdelsayed, M., Peters, C. H. & Ruben, P. C. A Mixed Periodic Paralysis & Myotonia Mutant, P1158S, Imparts pH-Sensitivity in Skeletal Muscle Voltage-gated Sodium Channels. Sci. Rep. 8, 6304 (2018).
58. Lehmann-Horn, F. & Rudel, R. Hereditary nondystrophic myotonias and periodic paralyses. Curr. Opin. Neurol. 8, 402-410 (1995).
59. Tan, S. V. et al. Refined exercise testing can aid dna-based diagnosis in muscle channelopathies. Ann. Neurol. 69, 328-340 (2011).
60. Emery, A. E. H. Population frequencies of inherited neuromuscular diseases-A world survey. Neuromuscul. Disord. 1, 19-29 (1991).
61. Vicart, S., Sternberg, D., Fontaine, B. & Meola, G. Human skeletal muscle sodium channelopathies. Neurological Sciences 26, 194-202 (2005).
62. Jiang, D. et al. Structural basis for gating pore current in periodic paralysis. Nature 557, 590-594 (2018).
63. Wu, F. et al. A sodium channel knockin mutant (NaV1.4-R669H) mouse model of hypokalemic periodic paralysis. J. Clin. Invest. 121, 4082-4094 (2011).
64. Tombola, F., Pathak, M. M. & Isacoff, E. Y. Voltage-sensing arginines in a potassium channel permeate and occlude cation-selective pores. Neuron 45, 379-388 (2005).
65. Desaphy, J.-F. et al. Different flecainide sensitivity of hNav1.4 channels and myotonic mutants explained by state-dependent block. J. Physiol. 554, 321-34 (2004).
66. Lundbek, J. A. et al. Regulation of Sodium Channel Function by Bilayer Elasticity. J. Gen. Physiol. 123, 599-621 (2004).
67. Lafleur, M., Fine, B., Sternin, E., Cullis, P. R. & Bloom, M. Smoothed orientational order profile of lipid bilayers by 2H-nuclear magnetic resonance. Biophys. J. 56, 1037-1041 (1989).
68. Ingolfsson, H. I., Lea Sanford, R., Kapoor, R. & Andersen, 0. S. Gramicidin-based fluorescence assay; for determining small molecules potential for modifying lipid bilayer properties. J. Vis. Exp. (2010). doi:10.3791/2131
69. Kuo, C. C. & Bean, B. P. Slow binding of phenytoin to inactivated sodium channels in rat hippocampal neurons. Mol. Pharmacol. 46, 716-725 (1994).
70. Bean, B. P., Cohen, C. J. & Tsien, R. W. Lidocaine block of cardiac sodium channels. J. Gen. Physiol. 81, 613-642 (1983).
71. Nuss, H. B., Tomaselli, G. F. & Marban, E. Cardiac sodium channels (hH1) are intrinsically more sensitive to block by lidocaine than are skeletal muscle (μ1) channels. J Gen Physiol 106, 1193-209. (1995).
72. Cannon, S. C. Channelopathies of skeletal muscle excitability. Compr. Physiol. 5, 761-790 (2015).
73. Ghovanloo, M. R., Peters, C. H. & Ruben, P. C. Effects of acidosis on neuronal voltage-gated sodium channels: Nav1.1 and nav1.3. Channels 12, 367-377 (2018).
74. Cannon, S. C., Brown, R. H. & Corey, D. P. Theoretical reconstruction of myotonia and paralysis caused by incomplete inactivation of sodium channels. Biophys. J. 65, 270-88 (1993).
75. Khan, A. A., Shekh-Ahmad, T., Khalil, A., Walker, M. C. & Ali, A. B. Cannabidiol exerts antiepileptic effects by restoring hippocampal interneuron functions in a temporal lobe epilepsy model. Br. J. Pharmacol. 175, 2097-2115 (2018).
76. Alfonsi, E. et al. Efficacy of propafenone in paramyotonia congenita. Neurology 68, 1080-1081 (2007).
77. Trip, J. et al. In tandem analysis of CLCN1 and SCN4A greatly enhances mutation detection in families with non-dystrophic myotonia. Eur. J. Hum. Genet. 16, 921-929 (2008).
78. BULBRING, E. Observations on the isolated phrenic nerve diaphragm preparation of the rat. Br. J. Pharmacol. Chemother. 1, 38-61 (1946).
79. Jurkat-Rott, Karin & Holzherr, Boris & Fauler, Michael & Lehmann-Horn, Frank. (2010). Sodium channelopathies of skeletal muscle result from gain or loss of function. Pflügers Archiv: European journal of physiology. 460. 239-48. 10.1007/s00424-010-0814-4.
80. Nachimuthu S, Assar M D, Schussler J M. Drug-induced QT interval prolongation: mechanisms and clinical management. Ther Adv Drug Saf. 2012; 3(5):241-253. doi:10.1177/2042098612454283
81. Gauret et al (2020) Hydroxychloroquine and azithromycin as a treatment of COVID-19: results of an open-label non-randomized clinical trial. International Journal of Antimicrobial Agents. Available online 20 Mar. 2020, 105949
82. Retallack H, Di Lullo E, Arias C, Knopp K A, Laurie M T, Sandoval-Espinosa C, et al. Zika virus cell tropism in the developing human brain and inhibition by azithromycin. Proc Natl Acad Sci USA. 2016 Dec. 13; 113(50):14408-14413. Epub 2016 Nov. 29.
83. Madrid P B, Panchal R G, Warren T K, Shurtleff A C, Endsley A N, Green C E, Kolokoltsov A, et al. Evaluation of Ebola Virus Inhibitors for Drug Repurposing. ACS Infect Dis. 2015 Jul. 10; 1(7):317-26.
84. Bosseboeuf E, Aubry M, Nhan T, de Pina, JJ, Rolain J M, Raoult D, et al. Azithromycin inhibits the replication of Zika virus. J Antivirals Antiretrovirals. 2018 10(1):6-11. doi: 10.4172/1948-5964.1000173.
85. Chun-Yu Chen, M. D., Feng-Lin Wang, M. D., and Chih-Chuan Lin, M. D. Chronic Hydroxychloroquine Use Associated with QT Prolongation and Refractory Ventricular Arrhythmia. Clinical Toxicology, 44:173-175, 2006.
86. Assessment of Evidence for COVID-19-Related Treatments by American Society of Health-System Pharmacists, Inc. updated Mar. 21, 2020.
87. Francieli Vuolo; Fabricia Petronilho; Beatriz Sonai, Cristiane Ritter et al. Evaluation of Serum Cytokines Levels and the Role of Cannabidiol Treatment in Animal Model of Asthma. Mediators of Inflammation. Volume 2015, Article ID 538670, 5 pages.
88. Senthil Nachimuthu, Manish D. Assar and Jeffrey M. Schussler. Drug-induced QT interval prolongation:mechanisms and clinical management. Therapeutic Advances in Drug Safety. (2012) 3(5) 241-253.
89. MICHAEL J. A CKERMAN, M. D., P H.D. The Long QT Syndrome: Ion Channel Diseases of the Heart. Mayo CUn Proc 1998; 73:250-269.
90. R. G. Pertwee, “The diverse CB1 and CB2 receptor pharmacology of three plant cannabinoids: delta9-tetrahydrocannabinol, cannabidiol and delta9-tetrahydrocannabivarin,” British Journal of Pharmacology, vol. 153, no. 2, pp. 199-215, 2008.
91. Bacharier L B, Guilbert T W, Mauger D T, Boehmer S, Beigelman A, Fitzpatrick A M, et al. Early administration of azithromycin and prevention of severe lower respiratory tract illnesses in preschool children with a history of such illnesses: A randomized clinical trial. JAMA. 2015 Nov. 17; 314(19):2034-2044.
92. Jules C. Hancox, PhD. Azithromycin, cardiovascular risks, QTc interval prolongation, torsade de pointes, and regulatory issues: A narrative review based on the study of case reports. Therapeutic Advances in Infectious Disease. (2013) 1(5) 155-165.
93. Fouda M A, Ghovanloo M R, Ruben P C. Cannabidiol protects against high glucose-induced oxidative stress and cytotoxicity in cardiac voltage-gated sodium channels. Br J Pharmacol. 2020 Feb. 19.
94. Huang, C. et al. Clinical features of patients infected with 2019 novel coronavirus in Wuhan, China. Lancet 395, 497-506 (2020).
95. Abdelsayed M, Peters C H, & Ruben P C (2015). Differential thermosensitivity in mixed syndrome cardiac sodium channel mutants. The Journal of physiology 593: 4201-4223.
96. Abdelsayed M, Ruprai M, & Ruben P C (2018). The efficacy of Ranolazine on E1784K is altered by temperature and calcium. Scientific reports 8: 3643.
97. Adamo L, Rocha-Resende C, Prabhu S D, & Mann D L (2020). Reappraising the role of inflammation in heart failure. Nat Rev Cardiol 17: 269-285.
98. Akin E J, Higerd G P, Mis M A, Tanaka B S, Adi T, Liu S, et al. (2019). Building sensory axons: Delivery and distribution of Na(V)1.7 channels and effects of inflammatory mediators. Sci Adv 5: eaax4755-eaax4755.
99. Aromolaran A S, Chahine M, & Boutjdir M (2018). Regulation of Cardiac Voltage-Gated Sodium Channel by Kinases: Roles of Protein Kinases A and C. Handbook of experimental pharmacology 246: 161-184.
100. Astman N, Gutnick M J, & Fleidervish I A (1998). Activation of protein kinase C increases neuronal excitability by regulating persistent Na+ current in mouse neocortical slices. Journal of neurophysiology 80: 1547-1551.
101. Barnes M P (2006). Sativex: clinical efficacy and tolerability in the treatment of symptoms of multiple sclerosis and neuropathic pain. Expert opinion on pharmacotherapy 7: 607-615.
102. Bockus L B, & Humphries K M (2015). cAMP-dependent Protein Kinase (PKA) Signaling Is Impaired in the Diabetic Heart. 290: 29250-29258.
103. Climént-Palmer M, & Spiegelhalter D (2019). Hormone replacement therapy and the risk of breast cancer: How much should women worry about it? Post reproductive health 25: 175-178.
104. Cunha J M, Carlini E A, Pereira A E, Ramos O L, Pimentel C, Gagliardi R, et al. (1980). Chronic administration of cannabidiol to healthy volunteers and epileptic patients. Pharmacology 21: 175-185.
105. Devinsky O, Cross J H, Laux L, Marsh E, Miller I, Nabbout R, et al. (2017). Trial of Cannabidiol for Drug-Resistant Seizures in the Dravet Syndrome. The New England journal of medicine 376: 2011-2020.
106. Eisenhut M, & Wallace H (2011). Ion channels in inflammation. Pflugers Archiv: European journal of physiology 461: 401-421.
107. El-Lakany M A, Fouda M A, El-Gowelli H M, El-Gowilly S M, & El-Mas M M (2018). Gonadal hormone receptors underlie the resistance of female rats to inflammatory and cardiovascular complications of endotoxemia. European journal of pharmacology 823: 41-48.
108. El-Lakany M A, Fouda M A, El-Gowelli H M, & El-Mas M M (2020). Ovariectomy provokes inflammatory and cardiovascular effects of endotoxemia in rats: Dissimilar benefits of hormonal supplements. Toxicology and applied pharmacology 393: 114928.
109. Fouda M A, Leffler K E, & Abdel-Rahman A A (2020). Estrogen-dependent hypersensitivity to diabetes-evoked cardiac autonomic dysregulation: Role of hypothalamic neuroinflammation. Life Sci 250: 117598.
110. Franceschetti S, Taverna S, Sancini G, Panzica F, Lombardi R, & Avanzini G (2000). Protein kinase C-dependent modulation of Na+ currents increases the excitability of rat neocortical pyramidal neurones. The Journal of physiology 528 Pt 2: 291-304.
111. Ghovanloo M R, Abdelsayed M, & Ruben P C (2016). Effects of Amiodarone and N-desethylamiodarone on Cardiac Voltage-Gated Sodium Channels. Frontiers in pharmacology 7: 39.
112. Ghovanloo M R, Shuart N G, Mezeyova J, Dean R A, Ruben P C, & Goodchild S J (2018). Inhibitory effects of cannabidiol on voltage-dependent sodium currents. The Journal of biological chemistry 293: 16546-16558.
113. Goldin A L (2003). Mechanisms of sodium channel inactivation. Current opinion in neurobiology 13: 284-290.
114. Grisanti L A (2018). Diabetes and Arrhythmias: Pathophysiology, Mechanisms and Therapeutic Outcomes. Frontiers in physiology 9: 1669.
115. Gu Q, Kwong K, & Lee L Y (2003). Ca2+ transient evoked by chemical stimulation is enhanced by PGE2 in vagal sensory neurons: role of cAMP/PKA signaling pathway. Journal of neurophysiology 89: 1985-1993.
116. Hallaq H, Wang D W, Kunic J D, George A L, Jr., Wells K S, & Murray K T (2012). Activation of protein kinase C alters the intracellular distribution and mobility of cardiac Na+ channels. American journal of physiology Heart and circulatory physiology 302: H782-789.
117. Iorga A, Cunningham C M, Moazeni S, Ruffenach G, Umar S, & Eghbali M (2017). The protective role of estrogen and estrogen receptors in cardiovascular disease and the controversial use of estrogen therapy. Biology of sex differences 8: 33.
118. Iqbal S M, & Lemmens-Gruber R (2019). Phosphorylation of cardiac voltage-gated sodium channel: Potential players with multiple dimensions. Acta physiologica (Oxford, England) 225: e13210.
119. Izzo A A, Borrelli F, Capasso R, Di Marzo V, & Mechoulam R (2009). Non-psychotropic plant cannabinoids: new therapeutic opportunities from an ancient herb. Trends in pharmacological sciences 30: 515-527.
120. Karin M (2005). Inflammation-activated protein kinases as targets for drug development. Proceedings of the American Thoracic Society 2: 386-390; discussion 394-385.
121. Koya D, & King G L (1998). Protein kinase C activation and the development of diabetic complications. Diabetes 47: 859-866.
122. Lazzerini P E, Capecchi P L, & Laghi-Pasini F (2015). Long QT Syndrome: An Emerging Role for Inflammation and Immunity. Frontiers in cardiovascular medicine 2: 26.
123. Matheus A S, Tannus L R, Cobas R A, Palma C C, Negrato C A, & Gomes M B (2013). Impact of diabetes on cardiovascular disease: an update. International journal of hypertension 2013: 653789.
124. Mize A L, Shapiro R A, & Dorsa D M (2003). Estrogen receptor-mediated neuroprotection from oxidative stress requires activation of the mitogen-activated protein kinase pathway. Endocrinology 144: 306-312
125. Möller C, & Netzer R (2006). Effects of estradiol on cardiac ion channel currents. European journal of pharmacology 532: 44-49.
126. Moxley G, Stern A G, Carlson P, Estrada E, Han J, & Benson L L (2004). Premenopausal sexual dimorphism in lipopolysaccharide-stimulated production and secretion of tumor necrosis factor. The Journal of rheumatology 31: 686-694.
127. Murphy A J, Guyre P M, & Pioli P A (2010). Estradiol suppresses NF-kappa B activation through coordinated regulation of let-7a and miR-125b in primary human macrophages. Journal of immunology (Baltimore, Md.: 1950) 184: 5029-5037.
128. Nachimuthu S, Assar M D, & Schussler J M (2012). Drug-induced QT interval prolongation: mechanisms and clinical management. Therapeutic advances in drug safety 3: 241-253.
129. Nichols J M, & Kaplan B L F (2020). Immune Responses Regulated by Cannabidiol. Cannabis Cannabinoid Res 5: 12-31.
130. O'Hara T, Virag L, Varro A, & Rudy Y (2011). Simulation of the undiseased human cardiac ventricular action potential: model formulation and experimental validation. PLoS computational biology 7: e1002061.
131. Odening K E, & Koren G (2014). How do sex hormones modify arrhythmogenesis in long QT syndrome? Sex hormone effects on arrhythmogenic substrate and triggered activity. Heart rhythm 11: 2107-2115.
132. Ono K, Fozzard H A, & Hanck D A (1993). Mechanism of cAMP-dependent modulation of cardiac sodium channel current kinetics. Circulation research 72: 807-815.
133. Pistrosch F, Natali A, & Hanefeld M (2011). Is hyperglycemia a cardiovascular risk factor? Diabetes care 34 Suppl 2: S128-131.
134. Ruan Y, Liu N, & Priori S G (2009). Sodium channel mutations and arrhythmias. Nature reviews Cardiology 6: 337-348.
135. Seltzer E S, Watters A K, & MacKenzie D, Jr. (2020). Cannabidiol (CBD) as a Promising Anti-Cancer Drug. Cancers 12(11) E3203.
136. Shimizu W, & Antzelevitch C (1999). Cellular basis for long QT, transmural dispersion of repolarization, and torsade de pointes in the long QT syndrome. Journal of electrocardiology 32 Suppl: 177-184.
137. Shryock J C, Song Y, Rajamani S, Antzelevitch C, & Belardinelli L (2013). The arrhythmogenic consequences of increasing late INa in the cardiomyocyte. Cardiovascular research 99: 600-611.
138. Tateyama M, Rivolta I, Clancy C E, & Kass R S (2003). Modulation of cardiac sodium channel gating by protein kinase A can be altered by disease-linked mutation. The Journal of biological chemistry 278: 46718-46726.
139. Tsalamandris S, Antonopoulos A S, Oikonomou E, Papamikroulis G A, Vogiatzi G, Papaioannou S, et al. (2019). The Role of Inflammation in Diabetes: Current Concepts and Future Perspectives. European cardiology 14: 50-59.
140. Ukpabi O J, & Onwubere B J (2017). QTc prolongation in Black diabetic subjects with cardiac autonomic neuropathy. African health sciences 17: 1092-1100.
141. Viviani B, Corsini E, Binaglia M, Lucchi L, Galli C L, & Marinovich M (2002). The anti-inflammatory activity of estrogen in glial cells is regulated by the PKC-anchoring protein RACK-1. Journal of neurochemistry 83: 1180-1187.
142. Wang Q, Cao J, Hu F, Lu R, Wang J, Ding H, et al. (2013). Effects of estradiol on voltage-gated sodium channels in mouse dorsal root ganglion neurons. Brain research 1512: 1-8.
143. Wang Q, Shen J, Splawski I, Atkinson D, Li Z, Robinson J L, et al. (1995). SCNSA mutations associated with an inherited cardiac arrhythmia, long QT syndrome. Cell 80: 805-811.
145. Wang Y, Garro M, & Kuehl-Kovarik M C (2010). Estradiol attenuates multiple tetrodotoxin-sensitive sodium currents in isolated gonadotropin-releasing hormone neurons. Brain research 1345: 137-145.
146. Ward C A, Bazzazi H, Clark R B, Nygren A, & Giles W R (2006). Actions of emigrated neutrophils on Na(+) and K(+) currents in rat ventricular myocytes. Progress in biophysics and molecular biology 90: 249-269.
147. West J W, Patton D E, Scheuer T, Wang Y, Goldin A L, & Catterall W A (1992). A cluster of hydrophobic amino acid residues required for fast Na(+)-channel inactivation. Proceedings of the National Academy of Sciences of the United States of America 89: 10910-10914.
148. Whitsel E A, Boyko E J, Rautaharju P M, Raghunathan T E, Lin D, Pearce R M, et al. (2005). Electrocardiographic QT interval prolongation and risk of primary cardiac arrest in diabetic patients. Diabetes care 28: 2045-2047.
149. Xu Y, Lin J, Wang S, Xiong J, & Zhu Q (2014). Combined estrogen replacement therapy on metabolic control in postmenopausal women with diabetes mellitus. The Kaohsiung journal of medical sciences 30: 350-361.
150. Warner B., Hoffmann P. (2002). Investigation of the potential of clozapine to cause torsade de pointes. Adverse Drug React. Toxicol. Rev. 21, 189-203
151. Zequn Z, Yujia W, Dingding Q, Jiangfang L. Off-label use of chloroquine, hydroxychloroquine, azithromycin and lopinavir/ritonavir in COVID-19 risks prolonging the QT interval by targeting the hERG channel. Eur J Pharmacol. 2021; 893:173813. doi:10.1016/j.ejphar.2020.173813
152. Kuryshev Y A, Bruening-Wright A, Brown A M, Kirsch G E. Increased cardiac risk in concomitant methadone and diazepam treatment: pharmacodynamic interactions in cardiac ion channels. J Cardiovasc Pharmacol. 2010 October; 56(4):420-30.

We claim:

1. A pharmaceutical composition comprising therapeutically effective amount of cannabidiol for use in treatment of a cardiac disorder arising from a gating defect in sodium channel Nav1.5.

2. A pharmaceutical composition comprising therapeutically effective amount of cannabidiol for use in treatment of a cardiac disorders arising from a gating defect in sodium channel Nav1.5 wherein the gating defect includes at least one from i) less likely to activate; ii) inability to fast inactivate; iii) unstable fast inactivation; iv) late or persistent sodium current and v) prolongation of action potential.

3. A pharmaceutical composition comprising therapeutically effective amount of cannabidiol for use in treatment of a cardiac disorders arising from gating defects in sodium channel Nav 1.5 wherein the gating defect is selected from late or persistent sodium current and prolongation of action potential.

4. The pharmaceutical composition of the claim 1, 2 or 3 for use in treatment of a cardiac disorder selected from one or more of long QT syndrome, long QTc syndrome, long QRS syndrome, cardiomyopathy, heart failure, arrhythmia, ischemia, Hypertrophic cardiomyopathy and Hypoxia myocardial ischemia, myocardial infarction (MI), arrhythmias of ischemic and non-ischemic origin, inflammation, vascular dysfunction, cardiomyopathy, cardiac remodeling, maladaptation, anginas of different types, drug induced heart failure, iatrogenic heart and vascular diseases.

5. The pharmaceutical composition of the claim 1, 2 or 3 for use in treatment of a cardiac disorder selected from one or more of long QT syndrome, long QTc syndrome, long QRS syndrome, arrhythmia, ischemia, Heart Failure, Hypertrophic cardiomyopathy and Hypoxia.

6. A method of treating cardiac disorder in a patient suffering from such disorder wherein said method comprises administering a pharmaceutical composition comprising therapeutically effective amount of cannabidiol wherein the cardiac disorder arises from gating defects in sodium channel Nav1.5.

7. A method of treating cardiac disorder in a patient suffering from such disorder wherein said method comprises administering a pharmaceutical compositions comprising therapeutically effective amount of cannabidiol wherein the cardiac disorder arises from gating defects in sodium channel Nav1.5 wherein the gating defect includes at least one from i) less likely to activate; ii) inability to fast inactivate; iii) unstable fast inactivation; iv) late or persistent sodium current and v) prolongation of action potential.

8. A method of treating cardiac disorder in a patient suffering from such disorder wherein said method comprises administering a pharmaceutical compositions comprising therapeutically effective amount of cannabidiol wherein the cardiac disorder arises from gating defect wherein the gating defect is selected from late or persistent sodium current and prolongation of action potential.

9. The method of treating cardiac disorder according to the claim 6 wherein the cardiac disorder is selected from one or more of long QT syndrome, long QTc syndrome, long QRS syndrome, cardiomyopathy, heart failure, arrhythmia, ischemia, Heart Failure, Hypertrophic cardiomyopathy and Hypoxia myocardial ischemia, myocardial infarction (MI), arrhythmias of ischemic and non-ischemic origin, inflammation, vascular dysfunction, cardiomyopathy, cardiac remodeling, maladaptation, anginas of different types, drug induced heart failure, iatrogenic heart and vascular diseases.

10. The method of treating cardiac disorder according to the claim 6 wherein the cardiac disorder is selected from one or more of long QT syndrome, long QTc syndrome, long QRS syndrome, arrhythmia, ischemia, Heart Failure, Hypertrophic cardiomyopathy and Hypoxia.

11. A pharmaceutical compositions comprising therapeutically effective amount of cannabidiol for use in treatment of a cardiac disorder arising from gating defects in sodium channel Nav1.5 wherein the gating defect is induced by hyperglycemic or diabetic condition.

12. A pharmaceutical composition comprising a therapeutically effective amount of cannabidiol for use in avoiding or minimizing occurrence of a cardiac disorder arising from gating defects in sodium channel Nav1.5 wherein the gating defect is prone to be induced by hyperglycemic or diabetic condition.

13. The pharmaceutical composition of the claim 11 or 12 for use in treatment of a cardiac disorder selected from one or more of long QT syndrome, long QTc syndrome, long QRS syndrome, cardiomyopathy, heart failure, arrhythmia, ischemia, Heart Failure, Hypertrophic cardiomyopathy and Hypoxia myocardial ischemia, myocardial infarction (MI), arrhythmias of ischemic and non-ischemic origin, inflammation, vascular dysfunction, cardiomyopathy, cardiac remodeling, maladaptation, anginas of different types, drug induced heart failure, iatrogenic heart and vascular diseases.

14. The pharmaceutical composition of the claim 11 or 12 for use in treatment of a cardiac disorder selected from one or more of long QT syndrome, long QTc syndrome, long QRS syndrome, arrhythmia, ischemia, Heart Failure, Hypertrophic cardiomyopathy and Hypoxia.

15. A method of treating cardiac disorder in a patient suffering from such disorder wherein said method comprises administering a pharmaceutical composition comprising therapeutically effective amount of cannabidiol wherein the cardiac disorder arises from gating defects in sodium channel Nav1.5 and wherein the gating defect is induced by hyperglycemic or diabetic condition.

16. A method of avoiding or minimizing occurrence of a cardiac disorder wherein said method comprises administering a pharmaceutical composition comprising therapeutically effective amount of cannabidiol wherein the cardiac disorder arises from gating defects in sodium channel Nav1.5 wherein the gating defect is prone to be induced by hyperglycemic or diabetic condition.

17. The method of treating cardiac disorder according to the claim 15 or 16 wherein the cardiac disorder is selected from one or more of long QT syndrome, long QTc syndrome, long QRS syndrome, cardiomyopathy, heart failure, arrhythmia, ischemia, Heart Failure, Hypertrophic cardiomyopathy and Hypoxia myocardial ischemia, myocardial infarction (MI), arrhythmias of ischemic and non-ischemic origin, inflammation, vascular dysfunction, cardiomyopathy, cardiac remodeling, maladaptation, anginas of different types, drug induced heart failure, iatrogenic heart and vascular diseases.

18. The method of treating cardiac disorder according to the claim 15 or 16 wherein the cardiac disorder is selected from one or more of long QT syndrome, long QTc syndrome, long QRS syndrome, arrhythmia, ischemia, Heart Failure, Hypertrophic cardiomyopathy and Hypoxia.

19. A pharmaceutical composition comprising therapeutically effective amount of cannabidiol for use in treatment of a cardiac disorder arising from gating defect in sodium channel Nav1.5 wherein the gating defect includes at least one from i) less likely to activate; ii) inability to fast inactivate; iii) unstable fast inactivation; iv) late or persistent sodium current and v) prolongation of action potential; and wherein the gating defect arises due to treatment with another therapeutic agent.

20. The pharmaceutical composition of claim 19 wherein the gating defect is selected from late or persistent sodium current and prolongation of action potential.

21. The pharmaceutical composition of claim 19 wherein said cardiac disorder is selected from one or more of long QT syndrome, long QTc syndrome, long QRS syndrome.

22. The pharmaceutical composition of claim 21 wherein another therapeutic agent causing gating defect in sodium channel Nav1.5 is selected from opioid, azithromycin, chloroquine, hydroxychloroquine and an antiviral.

23. The pharmaceutical composition of claim 22 wherein another therapeutic agent causing gating defect in sodium channel Nav1.5 is azithromycin.

24. The pharmaceutical composition of claim 22 wherein another therapeutic agent causing gating defect in sodium channel Nav1.5 is selected from one or more of oseltamivir phosphate, atazanavir sulphate and ribavirin.

25. The pharmaceutical composition of claim 22 wherein another therapeutic agent causing gating defect in sodium channel Nav1.5 is selected from chloroquine and hydroxychloroquine.

26. The pharmaceutical composition of claim 22 wherein another therapeutic agent causing gating defect in sodium channel Nav1.5 is methadone.

27. The pharmaceutical composition of claims 22-26 wherein gating defect in sodium channel Nav1.5 is late or persistent sodium current.

28. A pharmaceutical composition comprising therapeutically effective amount of cannabidiol for use in avoiding or minimizing occurrence of a cardiac disorder arising from gating defects in sodium channel Nav1.5 wherein the gating defect includes at least one from i) less likely to activate; ii) inability to fast inactivate; iii) unstable fast inactivation; iv) late or persistent sodium current and v) prolongation of action potential; and wherein the gating defect is likely to be induced by administration of i) at least one another therapeutic agent or ii) Covid-19 vaccine.

29. The pharmaceutical composition of claim 28 wherein another therapeutic agent causing gating defect in sodium channel Nav1.5 is selected from an opioid, methadone, an antiviral, azithromycin, chloroquine, hydroxychloroquine, oseltamivir phosphate, atazanavir sulphate and ribavirin.

30. The pharmaceutical composition of claims 28 and 29 wherein the cardiac disorder is selected from one or more of long QT syndrome, long QTc syndrome, long QRS syndrome.

31. A method of treating cardiac disorder in a patient suffering from such disorder wherein said method comprises administering a pharmaceutical composition comprising therapeutically effective amount of cannabidiol wherein the cardiac disorder arises from gating defects in sodium channel Nav1.5 and wherein the gating defect is induced in such patient due to treatment with another therapeutic agent.

32. The method of treating cardiac disorder of claim 31 wherein the gating defect is selected from late or persistent sodium current and prolongation of action potential.

33. The method of treating cardiac disorder of claim 31 wherein said cardiac disorder is selected from one or more of long QT syndrome, long QTc syndrome, long QRS syndrome.

34. The method of treating cardiac disorder of claim 31 wherein another therapeutic agent causing gating defect in sodium channel Nav1.5 is selected from an opioid, methadone, an antiviral, azithromycin, chloroquine, hydroxychloroquine, oseltamivir phosphate, atazanavir sulphate and ribavirin.

35. The method of treating cardiac disorder of claim 34 wherein another therapeutic agent causing gating defect in sodium channel Nav1.5 is methadone;

36. The method of treating cardiac disorder of claim 34 wherein another therapeutic agent causing gating defect in sodium channel Nav1.5 is azithromycin.

37. The method of treating cardiac disorder of claim 34 wherein another therapeutic agent causing gating defect in sodium channel Nav1.5 is an antiviral.

38. The method of treating cardiac disorder of claim 34 wherein another therapeutic agent causing gating defect in sodium channel Nav1.5 is selected from chloroquine and hydroxychloroquine.

39. The method of treating cardiac disorder of claims 34-38 wherein gating defect in sodium channel Nav1.5 is late or persistent sodium current.

40. A method of avoiding or minimizing occurrence of a cardiac disorder wherein said method comprises administering a pharmaceutical composition comprising therapeutically effective amount of cannabidiol wherein the cardiac disorder arises from gating defects in sodium channel Nav1.5 and wherein the gating defect is likely to be induced by administration of i) at least one another therapeutic agent or ii) Covid-19 vaccine.

41. The method of avoiding or minimizing occurrence of a cardiac disorder of claim 40 wherein another therapeutic agent causing gating defect in sodium channel Nav1.5 is selected from an opioid, methadone, an antiviral, azithromycin, chloroquine, hydroxychloroquine, oseltamivir phosphate, atazanavir sulphate and ribavirin.

42. The method of avoiding or minimizing occurrence of a cardiac disorder of claims 40 and 41 wherein the cardiac disorder is selected from one or more of long QT syndrome, long QTc syndrome, long QRS syndrome.

43. A pharmaceutical composition comprising therapeutically effective amount of cannabidiol for use in avoiding or minimizing occurrence of a cardiac disorder arising from gating defects in sodium channel Nav1.5 wherein the gating defect is likely to be induced in Covid-19 epidemic or pandemic.

44. The pharmaceutical composition of claim 43 wherein the cardiac disorder is selected from one or more of long QT syndrome, long QTc syndrome, long QRS syndrome.

45. A method of avoiding or minimizing occurrence of a cardiac disorder wherein said method comprises administering a pharmaceutical composition comprising therapeutically effective amount of cannabidiol wherein the cardiac disorder arises from gating defects in sodium channel Nav1.5 wherein the gating defect is likely to be induced in Covid-19 epidemic or pandemic.

46. The method of avoiding or minimizing occurrence of a cardiac disorder of claim 45 wherein the cardiac disorder is selected from one or more of long QT syndrome, long QTc syndrome, long QRS syndrome.

47. A pharmaceutical composition comprising therapeutically effective amount of cannabidiol for use in prophylaxis or prophylactic treatment for avoiding or minimizing occurrence of a cardiac disorder arising from gating defects in sodium channel Nav1.5.

48. A method of prophylaxis or prophylactic treatment for avoiding or minimizing occurrence of a cardiac disorder wherein said method comprises administering a pharmaceutical composition comprising therapeutically effective amount of cannabidiol wherein the cardiac disorder arises from gating defects in sodium channel Nav1.5.

49. A pharmaceutical composition comprising therapeutically effective amount of cannabidiol for use in treatment of a cardiac disorder arising from adversely affected sodium channel Nav1.5 wherein the sodium channel Nav1.5 is adversely affected due to effects of formation of reactive oxygen species or due to oxidative stress/damage.

50. A method of treating cardiac disorder in a patient suffering from such disorder wherein said method comprises administering a pharmaceutical composition comprising therapeutically effective amount of cannabidiol wherein the cardiac disorder arises from adversely affected sodium channel Nav1.5 wherein the sodium channel Nav1.5 is adversely affected due to effects of formation of reactive oxygen species or due to oxidative stress/damage.

51. A pharmaceutical compositions comprising therapeutically effective amount of cannabidiol for use in treatment of a cardiac disorder arising from gating defect in sodium channel Nav1.5 induced or likely to be induced by inflammation.

52. The pharmaceutical composition of claim 51 wherein the gating defect in sodium channel Nav1.5 includes at least one from i) less likely to activate; ii) inability to fast inactivate; iii) unstable fast inactivation; iv) late or persistent sodium current and v) prolongation of action potential.

53. The pharmaceutical composition of the claim 51 for use in treatment of a cardiac disorder selected from one or more of long QT syndrome, long QTc syndrome, long QRS syndrome, cardiomyopathy, heart failure, arrhythmia, ischemia, Heart Failure, Hypertrophic cardiomyopathy and Hypoxia myocardial ischemia, myocardial infarction (MI), arrhythmias of ischemic and non-ischemic origin, inflammation, vascular dysfunction, cardiomyopathy, cardiac remodelling remodeling, maladaptation, anginas of different types, drug induced heart failure, iatrogenic heart and vascular diseases.

54. The pharmaceutical composition of the claim 51 for use in treatment of a cardiac disorder selected from one or more of long QT syndrome, long QTc syndrome, long QRS syndrome, arrhythmia, ischemia, Heart Failure, Hypertrophic cardiomyopathy and Hypoxia.

55. A method of treating cardiac disorder in a patient suffering from such disorder wherein said method comprises administering a pharmaceutical composition comprising therapeutically effective amount of cannabidiol wherein the cardiac disorder arises from gating defects in sodium channel Nav1.5 induced or likely to be induced by inflammation.

56. The method of treating cardiac disorder according to the claim 55 wherein the gating defect in sodium channel Nav1.5 includes at least one from i) inability to activateless likely to activate; ii) inability to fast inactivate; iii) unstable fast inactivation; iv) late or persistent sodium current and v) prolongation of action potential.

57. The method of treating cardiac disorder according to the claim 55 wherein the cardiac disorder is selected from one or more of long QT syndrome, long QTc syndrome, long QRS syndrome, cardiomyopathy, heart failure, arrhythmia, ischemia, Heart Failure, Hypertrophic cardiomyopathy and Hypoxia myocardial ischemia, myocardial infarction (MI), arrhythmias of ischemic and non-ischemic origin, inflammation, vascular dysfunction, cardiomyopathy, cardiac remodelling remodeling, maladaptation, anginas of different types, drug induced heart failure, iatrogenic heart and vascular diseases.

58. The method of treating cardiac disorder according to the claim 55 wherein the cardiac disorder is selected from one or more of long QT syndrome, long QTc syndrome, long QRS syndrome, arrhythmia, ischemia, Heart Failure, Hypertrophic cardiomyopathy and Hypoxia.

59. A pharmaceutical compositions comprising therapeutically effective amount of cannabidiol for use in treatment of a skeletal muscle disorder arising from adversely affected sodium channel Nav1.4.

60. The pharmaceutical composition of claim 55 wherein the skeletal muscle disorder is selected from one or more of muscle stiffness, pain, myotonia, gating-pore current in the VSD leading to periodic paralyses.

61. A method of treating skeletal muscle disorder in a patient suffering from such disorder wherein the said method comprises administering a pharmaceutical compositions comprising therapeutically effective amount of cannabidiol wherein the skeletal muscle disorder arises from adversely affected sodium channel Nav1.4.

62. The method of treating skeletal muscle disorder of claim 61 wherein the skeletal muscle disorder is selected from one or more of muscle stiffness, pain, myotonia, gating-pore current in the VSD leading to periodic paralyses.

63. A pharmaceutical composition comprising therapeutically effective amount of cannabidiol for use in treatment of a cardiac disorder or a skeletal muscle disorder arising from adversely affected sodium channel Nav1.5 or adversely affected sodium channel Nav1.4 wherein such composition comprises cannabidiol and at least one pharmaceutically acceptable carrier.

64. The pharmaceutical composition of claim 63 wherein at least one pharmaceutically acceptable carrier is selected from soluble excipient/diluent, solubilizer, stabilizer or bioavailability enhancer.

65. A pharmaceutical composition comprising therapeutically effective amount of cannabidiol for use in treatment of a cardiac disorder or a skeletal muscle disorder arising from adversely affected sodium channel Nav1.5 or adversely affected sodium channel Nav1.4 and an other therapeutic agent.

66. The pharmaceutical composition of claim 66 wherein the other therapeutic agent is one inducing or likely to induce Long QT or arrythmia.

67. The pharmaceutical composition of claim 66 wherein the other therapeutic agent is one or ore from an opioid, methadone, azithromycin, chloroquine, hydroxychloroquine, an antiviral, oseltamivir phosphate, atazanavir sulphate and ribavirin.

68. The pharmaceutical composition of claims 65-67 in the form of a bilayer or trilayer tablet; or in a form of a capsule having two types of pellets/beads/granules/slugs each having a different therapeutic agent; or a liquid having cannabidiol and the other therapeutic agent.

69. The pharmaceutical composition of claim 65 wherein the other therapeutic agent is one inducing or likely to induce inflammation.

70. The pharmaceutical composition of claim 69 in the form of a bilayer or a trilayer tablet; or in a form of a capsule having two types of pellets/beads/granules/slugs each having a different therapeutic agent; or a liquid having cannabidiol and the other therapeutic agent.

65. A kit comprising at least two pharmaceutical compositions wherein a first pharmaceutical composition comprises therapeutically effective amount of cannabidiol and the second composition comprises other therapeutic agent is one which induces or which is likely to induce long QT/arrythmia or inflammation.

66. A kit comprising at least two pharmaceutical compositions wherein a first pharmaceutical composition comprises therapeutically effective amount of cannabidiol and the second pharmaceutical composition is a Covid-19 vaccine.
